(19) United States
(12) Patent Application Publication
(10) Pub. No.: US 2022/0089636 A1
(43) Pub. Date: Mar. 24, 2022

(54) TETRAZOLONE SUBSTITUTED STEROIDS AND USE THEREOF

(71) Applicant: Beijing Xuanyi PharmaSciences Co., Ltd., Beijing (CN)

(72) Inventors: Yuntao SONG, Palo Alto, CA (US); Hui LI, Santa Clara, CA (US)

(21) Appl. No.: 17/422,635

(22) PCT Filed: Jan. 14, 2020
(86) PCT No.: PCT/US2020/013471

§ 371 Date: Jul. 13, 2021

Related U.S. Application Data
(60) Provisional application No. 62/792,243, filed on Jan. 14, 2019.

(51) Int. Cl. C07J 43/00; A61K 45/06

(52) U.S. Cl. CPC C07J 43/003; A61K 45/06

(57) ABSTRACT

The present disclosure relates to compounds of formula (AI), (I), (AII), and (II), or a pharmaceutically acceptable salt, solvate, stereoisomer, or tautomer thereof, a pharmaceutical composition comprising a compound of formula (AI), (I), (AII), and (II), and use thereof, wherein R2, R3, R4, R5, R6, R7, R10, R11a, R11b, R12, R16, R19a, R19b, and R20 are described herein. Such compounds are envisioned useful for the prevention and treatment of a variety of CNS-related conditions, for example, treatment of sleep disorders, mood disorders, movement disorders, convulsive disorders, schizophrenin spectrum disorders, disorders of memory and/or cognition, personality disorders, autism spectrum disorders, pain, traumatic brain injury, vascular diseases, substance abuse disorders and/or withdrawal syndromes, or tinnitus etc.

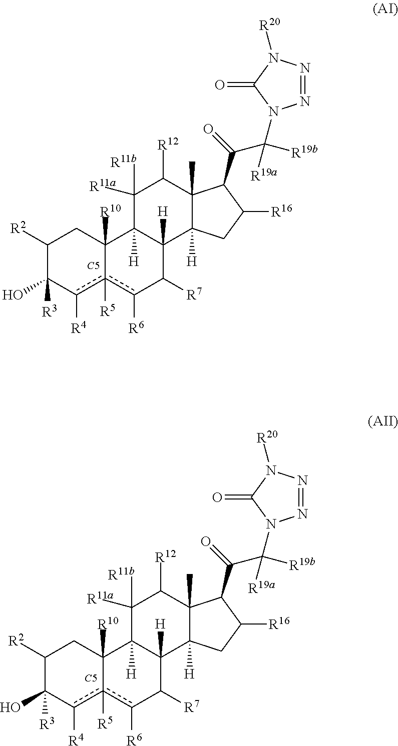

### CROSS-REFERENCE TO RELATED APPLICATIONS

This application claims the benefit of U.S. Provisional Application No. 62/792,243, filed Jan. 14, 2019, the disclosure of which is incorporated by reference herein in its entirety.

### BACKGROUND OF THE DISCLOSURE

The human brain contains about 100 billion neurons forming an intricate network of innumerable connections, which continuously adapt and rewire themselves following input from external and internal environments as well as the physiological synaptic, dendritic and axonal sculpture during brain maturation and throughout the life span. Brain excitability is defined as the level of arousal of an animal, a continuum that ranges from coma to convulsions, and is regulated by various neurotransmitters. In general, neurotransmitters are responsible for regulating the conductance of ions across neuronal membranes.

The GABA_{A} (γ-aminobutyric acid)_{A} receptors are the major inhibitory neurotransmitter receptors in mammalian brain. Each isoform consists of five homologous or identical subunits surrounding a central chloride ion-selective channel gated by GABA. The higher the chloride ion concentration in the neuron, the lower the brain excitability (the level of arousal). GABA has a profound influence on overall brain excitability because up to 40% of the neurons in the brain utilizing GABA as a neurotransmitter.

It is well-documented that GABA receptor complex influence a wide range of brain circuits that are central to a variety of behavioral states, such as anxiety levels, seizures, sleep, vigilance, and memory. Drugs that act like GABA, e.g. barbiturates and benzodiazepines, such as Valium, produce their therapeutically useful effects by interacting with specific regulatory sites on the GABA receptor complex. In addition to the benzodiazepine and barbiturate binding site, there is a distinct binding site on GABA receptor for neuroactive steroids (Lan, N. C. et al., Neurochem. Res. 16: 347-356 (1991)).

Neuroactive steroids (NASs) or neurosteroids are among the most potent and effective modulators of neuronal excitability. The term “neurosteroid” was first mentioned by Etienne Baulieu in 1980's and initially referred to endogenous steroids synthesized in the brain and central nervous system (CNS) from cholesterol. Neurosteroids have been shown to impact CNS function primarily through allosteric modulation of the GABA_{A} receptor (GABA_{A}R). The term neurosteroid has been expanded to include synthetic and naturally-derived analogs that have CNS actions similar to endogenous neurosteroids. Increasing evidence indicates that dysregulation of neurosteroid production plays a role in the pathophysiology of stress and stress-related psychiatric disorders, including mood and anxiety disorders.

Many NAS compounds have reached clinical development status since the 1970's. For example, alfaxalone is under human studies in a sulfobutyl ether-β-cyclodextrin formulation (“Phaxan”) as an intravenous anesthetic. Minaxolone was developed as a water-soluble anesthetic NAS. Marinus Pharmaceuticals has been developing ganaxolone, the 3βmethyl derivative of allopregnanolone for focalonset seizures in adults and in children with epilepsy. Sage Therapeutics is developing Brexanolone as a parenteral, continuous infusion formulation for treatment of postpartum disorder. Their orally available GABA_{A}R agonist SAGE-217 is under development for the treatment in mood disorders, movement disorders and Parkinson's disease. (Blanco, M. J. et al., Bioorg. Med. Chem. Lett. 28: 61-70 (2018)).

Many NAS therapeutic compounds suffer from toxicity, formulation issue, and not consistently effective in the treatment of the desired syndromes. Therefore, new and improved neuroactive steroids are needed that act as modulating agents for brain excitability, as well as for the prevention and treatment of CNS-related diseases. The compounds, compositions, and methods described herein are directed toward this end.

### SUMMARY OF THE DISCLOSURE

The present invention is based, in part, on the desire to provide novel compounds with good potency, pharmacokinetic (PK) properties, oral bioavailability, formulatability, stability, safety, clearance and/or metabolism. The improved overall PK parameters and reducing potential toxicities and side effects may allow, in certain embodiments, administration orally and/or chronically.

In one embodiment of the present disclosure, a pharmaceutical composition is provided comprising a compound of formula (AI) and (AII), and any subgenera thereof (e.g., Formula (I), (I-A)-(I-D), (I-A1)-(I-A8), (I-B1)-(I-B8), (I-C1)-(I-C7), (I-D1)-(I-D7), (I-E)-(I-G), (I-E1), (I-F1), (I-G1), (II), (II-A)-(II-D), (II-A1)-(II-A8), (II-B1)-(II-B8), (II-C1)-(II-C7), (II-D1)-(II-D7), etc), including compounds of Table A, or a pharmaceutically acceptable salt, solvate, ester, or prodrug thereof. In one embodiment, the compound of the present disclosure has the structure of formula (AI) or (AII):

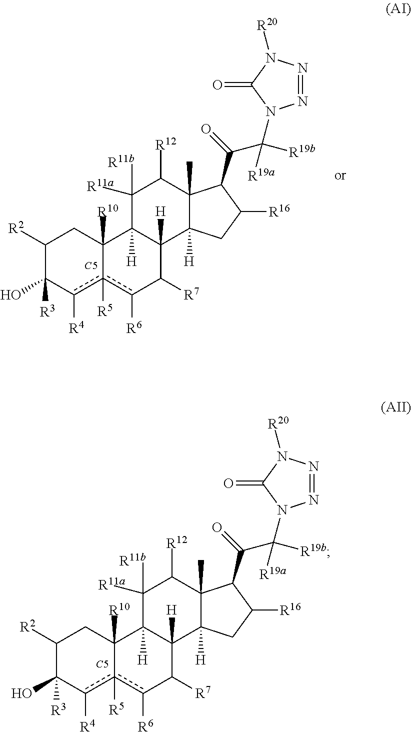

wherein,
[symbol] represents a single or double bond; and
when one of [symbol] is a double bond, the other [symbol] is a single bond and R⁵ is absent;
when both of [symbol] are single bonds, then R⁵ is hydrogen;
R², R⁴, R⁶, R⁷, R^{11a}, R^{11b}, R¹², and R¹⁶ are each independently hydrogen, halogen, cyano, nitro, substituted or unsubstituted alkyl, substituted or unsubstituted alkenyl, substituted or unsubstituted alkynyl, substituted or unsubstituted carbocylyl, substituted or unsubstituted heterocyclyl, substituted or unsubstituted aryl, substituted or unsubstituted heteroaryl, —OR^{A}, —C(═O)R^{A}, —C(═O)OR^{A}, —OC(═O)R^{A}, —OC(═O)OR^{A}, —C(═O)NR^{A}R^{A}, —NR^{A}R^{A}, —NR^{A}C(═O)R^{A}, —OC(═O)NR^{A}R^{A}, —NR^{A}C(═O)OR^{A}, —NR^{A}C(═O)NR^{A}R^{A}, —SR^{A}, —S(═O)R^{A}, —S(═O)₂R^{A}, —S(═O)₂OR^{A}, —OS(═O)₂R^{A}, —S(═O)₂NR^{A}R^{A}, or —NR^{A}S(═O)₂R^{A}, wherein R^{A} is independently hydrogen, substituted or unsubstituted alkyl, substituted or unsubstituted alkenyl, substituted or unsubstituted alkynyl, substituted or unsubstituted carbocylyl, substituted or unsubstituted heterocylyl, substituted or unsubstituted aryl, substituted or unsubstituted heteroaryl, haloalkyl, an oxygen protecting group when attached to oxygen, a sulfur protecting group when attached to sulfur, or a nitrogen protecting group when attached to nitrogen; or two R^{A} groups can be taken together with the atoms to which they are attached to, to form a substituted or unsubstituted heterocylyl or heteroaryl ring;
alternatively, R^{11a} and R^{11b}, taken together with the carbon atom to which they are both attached, form a 3-8 membered saturated, partially saturated, or unsaturated ring optionally containing one or more heteroatoms as a ring member selected from N, O, or S; or R^{11a} and R^{11b} are joined to form an oxo (═O) group;
R³ is hydrogen, substituted or unsubstituted alkyl, substituted or unsubstituted alkenyl, substituted or unsubstituted alkynyl, substituted or unsubstituted carbocylyl, substituted or unsubstituted heterocyclyl, substituted or unsubstituted aryl, substituted or unsubstituted heteroaryl;
R¹⁰ is hydrogen, halogen, cyano or substituted or unsubstituted alkyl;
R^{19a} is hydrogen, substituted or unsubstituted alkyl, or —OR^{A19}, wherein R^{A19} is hydrogen, substituted or unsubstituted alkyl, substituted or unsubstituted alkenyl, substituted or unsubstituted alkynyl, or substituted or unsubstituted carbocylyl;
R^{19b} is hydrogen or substituted or unsubstituted alkyl;
alternatively, R^{19a} and R^{19b} are joined to form an oxo (═O) group, or R^{19a} and R^{19b} together with the carbon atom to which they are both attached, form a 3-8 membered saturated, partially saturated, or unsaturated ring optionally containing one or more heteroatoms as a ring member selected from N, O, or S; and
R²⁰ is hydrogen, alkyl, haloalkyl, cycloalkyl, cycloalkylalkyl, heterocyclyl, aryl, C₆-C₁₂ aryl-C₁-C₆ alkyl-, heteroaryl, or heteroaryl-C₁-C₆ alkyl-, wherein alkyl, haloalkyl, cycloalkyl, cycloalkylalkyl, heterocyclyl, aryl, C₆-C₁₂ aryl-C₁-C₆ alkyl-, heteroaryl, and heteroaryl-C₁-C₆ alkyl- can be optionally substituted with substituents selected from substituted or unsubstituted alkyl, substituted or unsubstituted alkenyl, substituted or unsubstituted alkynyl, substituted or unsubstituted carbocylyl, substituted or unsubstituted heterocylyl, haloalkyl, substituted or unsubstituted —C₆-C₁₂ aryl, substituted or unsubstituted 5-12 membered heteroaryl, halogen, nitro, cyano, —OR^{A}, —C(═O)R^{A}, —C(═O)OR^{A}, —OC(═O)R^{A}, —OC(═O)OR^{A}, —C(═O)NR^{A}R^{A}, —NR^{A}R^{A}, —NR^{A}C(═O)R^{A}, — OC(═O)NR^{A}R^{A}, —NR^{A}C(═O)OR^{A}, —NR^{A}C(═O)NR^{A}R^{A}, —SR^{A}, —S(═O)R^{A}, —S(═O)₂R^{A}, —S(═O)₂OR^{A}, —OS(═O)₂R^{A}, —S(═O)₂NR^{A}R^{A}, or —NR^{A}S(═O)₂R^{A}, wherein R^{A} is independently hydrogen or substituted or unsubstituted alkyl, substituted or unsubstituted alkenyl, substituted or unsubstituted alkynyl, substituted or unsubstituted carbocylyl, substituted or unsubstituted heterocylyl, substituted or unsubstituted aryl, substituted or unsubstituted heteroaryl, haloalkyl, an oxygen protecting group when attached to oxygen, a sulfur protecting group when attached to sulfur, or a nitrogen protecting group when attached to nitrogen, or two R^{A} groups can be taken together with the atoms to which they are attached to, to form a substituted or unsubstituted heterocylyl or heteroaryl ring.

A compound of Formula (AI) or (AII), or any subgenera thereof (e.g., Formula (I), (I-A)-(I-D), (I-A1)-(I-A8), (I-B1)-(I-B8), (I-C1)-(I-C7), (I-D1)-(I-D7), (I-E)-(I-G), (I-E1), (I-F1), (I-G1), (II), (II-A)-(II-D), (II-A1)-(II-A8), (II-B1)-(II-B8), (II-C1)-(II-C7), (II-D1)-(II-D7), etc), including compounds of Table A, or a pharmaceutically acceptable salt, solvate, ester, or prodrug thereof are collectively referred to herein as “compounds of the present invention”.

In one embodiment of the compounds of the present invention, both of [symbol] are single bonds and R⁵ and R⁴ are in an alpha configuration. In one embodiment, both of [symbol] are single bonds and R⁵ and R⁴ are in a beta configuration. In one embodiment, both of [symbol] are single bonds and R⁵ and R⁶ are in an alpha configuration. In one embodiment, both of [symbol] are single bonds and R⁵ and R⁶ are in a beta configuration. In one embodiment, both [symbol] are single bonds and R⁴ and R⁶ are hydrogen. In one embodiment, one [symbol] is a single bond, and at least one of R⁴ or R⁶ is hydrogen, substituted or unsubstituted —C₁₋₆ alkyl, halogen, —CH₃, or —CF₃. In one embodiment, one [symbol] is a single bond, and at least one of R⁴ or R⁶ is F.

In one embodiment, the compound of the present invention is directed to compounds of Formula (I-A), Formula (I-B), Formula (II-A), or Formula (II-B), or a pharmaceutically acceptable salt, solvate, ester, or prodrug thereof.

In one embodiment of the compounds of the present invention, the substituents are selected from fluoro, chloro, bromo, cyano, nitro, hydroxy, methoxy, ethoxy, propoxy, isopropoxy, methyl, ethyl, propyl, isopropyl, —CH₂F, —CHF₂, —CF₃, -methylhydroxy, morpholine, pyrrolidine, piperidine, piperazine, phenyl, benzyl, pyridine, pyrimidine, cyclopropyl, cyclobutyl, cyclopentyl, cyclohexyl, —OCH₂F, —OCHF₂, —OCF₃, -ethylmethoxy, -methylcyclopropyl, —OR^{A}, —C(═O)R^{A}, —C(═O)OR^{A}, —OC(═O)R^{A}, —OC(═O)OR^{A}, —C(═O)NR^{A}R^{A}, —NR^{A}R^{A}, —NR^{A}C(═O)R^{A}, —OC(═O)NR^{A}R^{A}, —NR^{A}C(═O)OR^{A}, —NR^{A}C(═O)NR^{A}R^{A}, —SR^{A}, —S(═O)R^{A}, —S(═O)₂R^{A}, —S(═O)₂OR^{A}, —OS(═O)₂R^{A}, —S(═O)₂NR^{A}R^{A}, or —NR^{A}S(═O)₂R^{A}, wherein R^{A} is independently hydrogen, methyl, ethyl, propyl, isopropyl, cyclopropyl, cyclobutyl, cyclopentyl, cyclohexyl, —CH₂F, —CHF₂, —CF₃, phenyl, benzyl, pyridine, pyrimidine, -ethylmethoxy, -methylcyclopropyl, or two R^{A} groups can be taken together with the atoms to which they are attached to, to form a heterocylyl or heteroaryl ring.

In one embodiment of the compounds of the present invention, R² is hydrogen, —OH, —OCH₃, —OCH₂CH₃, —OCH₂CH₂CH₃, —CH₃, —CH₂CH₃, —CH₂CH₂CH₃, substituted or unsubstituted cyclopropyl, fluoro, or chloro.

In one embodiment of the compounds of the present invention, R⁴ and R⁶ are hydrogen.

In one embodiment of the compounds of the present invention, at least one of R⁴ or R⁶ is hydrogen, substituted or unsubstituted —C₁₋₆ alkyl, halogen, —CH₃, or —CF₃.

In one embodiment of the compounds of the present invention, R⁷ is hydrogen, —CH₃, —CH₂CH₃, —CH(CH₃)₂, —OH, —OCH₃, or —CH₂OCH₃.

In one embodiment of the compounds of the present invention, R^{11a} and Rill′ are both hydrogen. In one embodiment, R^{11a} and R^{11b} together form ═O (oxo). In one embodiment, R^{11a} is —OH, —OCH₃, —OCH₂CH₃ or —OCH₂CH₃CH₃ and R^{11b} is hydrogen.

In one embodiment of the compounds of the present invention, R¹² is hydrogen, —CH₃, —CH₂CH₃, —CH(CH₃)₂, —OH, —OCH₃, or —CH₂OCH₃.

In one embodiment of the compounds of the present invention, R¹⁶ is hydrogen, —CH₃, —CH₂CH₃, —CH(CH₃)₂, —OH, —OCH₃, or —CH₂OCH₃.

In one embodiment of the compounds of the present invention, R^{19a} and R^{19b} are both hydrogen; or R^{19a} is —C₁₋₆ alkyl and R^{19b} is hydrogen or —C₁₋₆ alkyl.

In one embodiment of the compounds of the present invention, R², R⁴, R⁵, R⁶, R⁷, R¹⁰, R^{11a}, R^{11b}, R¹², R¹⁶, R^{19a} and R^{19b} are all hydrogen.

In one embodiment of the compounds of the present invention, R³ is hydrogen, unsubstituted —C₁₋₆ alkyl, —C₃₋₁₂ cycloalkyl, or C₃₋₁₂ cycloalkyl-C₁₋₆ alkyl-.

In one embodiment of the compounds of the present invention, R²⁰ is hydrogen, substituted or unsubstituted —C₁₋₆ alkyl, —C₂₋₆ alkenyl, —C₂₋₆ alkynyl, —C₁₋₆ haloalkyl, —C₃₋₁₂ cycloalkyl, C₃₋₁₂ cycloalkyl-C₁-C₆ alkyl-, heterocyclyl, —C₆-C₁₂ aryl, C₆-C₁₂ aryl-C₁-C₆ alkyl-, 5-12 membered heteroaryl, or 5-12 membered heteroaryl-C₁-C₆ alkyl-.

In one embodiment of the compounds of the present invention, R¹⁰ is hydrogen, halogen, cyano, —CH₃, —CH₂F, —CHF₂, —CF₃, —CH₂CH₃, —CH₂OCH₃, or —CH₂SCH₃.

In one embodiment of the compounds of the present invention, the compound has the structure of Formula (I-E1) or (I-F1):

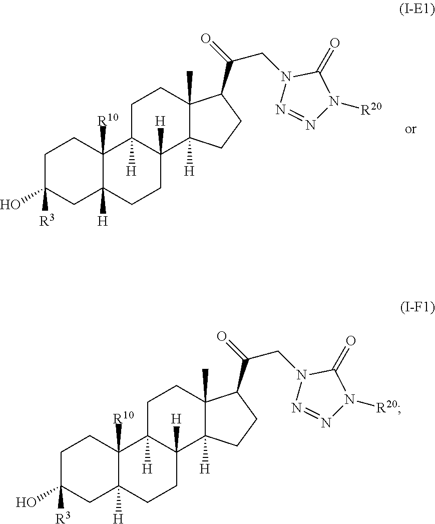

or a pharmaceutically acceptable salt, solvate, ester, or prodrug thereof, wherein:
R³ is hydrogen, —C₁₋₆ alkyl, —C₂₋₆ alkenyl, or —C₂₋₆ alkynyl, each optionally substituted with one to three groups selected from halogen or —C₁₋₆ alkoxy;
R¹⁰ is hydrogen, halogen, cyano or —C₁₋₆ alkyl wherein —C₁₋₆ alkyl is optionally substituted with halogen or —C₁₋₃ alkoxy; and
R²⁰ is hydrogen, —C₁₋₆ alkyl, —C₂₋₆ alkenyl, —C₂₋₆ alkynyl, —C₁₋₆ haloalkyl, —C₃₋₁₂ cycloalkyl, C₃₋₁₂ cycloalkyl-C₁-C₆ alkyl-, 3-12 membered heterocyclyl, —C₆-C₁₂ aryl, C₆-C₁₂ aryl-C₁-C₆ alkyl-, heteroaryl-C₁-C₆ alkyl-, or 5-12 membered heteroaryl, each of which can be optionally substituted with halogen, nitro, cyano, —C₁₋₆ alkyl, —C₂₋₆ alkenyl, —C₂₋₆ alkynyl, —C₃₋₁₂ cycloalkyl, 3-12 membered heterocylyl, —C₆-C₁₂ aryl, 5-12 membered heteroaryl, —OR^{A}, —C(═O)R^{A}, —C(═O)OR^{A}, —OC(═O)R^{A}, —OC(═O)OR^{A}, —C(═O)NR^{A}R^{A}, —NR^{A}R^{A}, —NR^{A}C(═O)R^{A}, —OC(═O)NR^{A}R^{A}, —NR^{A}C(═O)OR^{A}, —NR^{A}C(═O)NR^{A}R^{A}, —SR^{A}, —S(═O)R^{A}, —S(═O)₂R^{A}, —S(═O)₂OR^{A}, —OS(═O)₂R^{A}, —S(═O)₂NR^{A}R^{A}, or —NR^{A}S(═O)₂R^{A};
wherein R^{A} is independently hydrogen, alkyl, haloalkyl, alkenyl, alkynyl, cycloalkyl, cycloalkylalkyl, carbocylyl, heterocylyl, aryl, C₆-C₁₂ aryl-C₁-C₆ alkyl-, heteroaryl, heteroaryl-C₁-C₆ alkyl-, an oxygen protecting group when attached to oxygen, a sulfur protecting group when attached to sulfur, or a nitrogen protecting group when attached to nitrogen; or two R^{A} groups can be taken together with the atoms to which they are attached to, to form a substituted or unsubstituted heterocylyl or heteroaryl ring;
wherein the substituents are selected from fluoro, chloro, bromo, cyano, nitro, hydroxy, methoxy, ethoxy, propoxy, isopropoxy, methyl, ethyl, propyl, isopropyl, butyl, i-butyl, s-butyl, t-butyl, —CH₂CN, —CH₂CH₂CN, —CH₂F, —CHF₂, —CF₃, —CH₂OCH₃, —CH₂SCH₃, -methylhydroxy, morpholine, pyrrolidine, piperidine, piperazine, phenyl, benzyl, pyridine, pyrimidine, oxazole, pyrazole, cyclopropyl, cyclobutyl, cyclopentyl, cyclohexyl, —OCH₂F, —OCHF₂, —OCF₃, -ethylmethoxy, -methylcyclopropyl, —OR^{A}, —C(═O)R^{A}, —C(═O)OR^{A}, —OC(═O)R^{A}, —OC(═O)OR^{A}, —C(═O)NR^{A}R^{A}, —NR^{A}R^{A}, —NR^{A}C(═O)R^{A}, —OC(═O)NR^{A}R^{A}, —NR^{A}C(═O)OR^{A}, —NR^{A}C(═O)NR^{A}R^{A}, —SR^{A}, —S(═O)R^{A}, —S(═O)₂R^{A}, —S(═O)₂OR^{A}, —OS(═O)₂R^{A}, —S(═O)₂NR^{A}R^{A}, or —NR^{A}S(═O)₂R^{A}, wherein R^{A} is independently hydrogen, methyl, ethyl, propyl, isopropyl, cyclopropyl, cyclobutyl, cyclopentyl, cyclohexyl, —CH₂F, —CHF₂, —CF₃, phenyl, benzyl, pyridine, pyrimidine, -ethylmethoxy, or -methylcyclopropyl, or two R^{A} groups can be taken together with the atoms to which they are attached to, to form a heterocylyl or heteroaryl ring.

In one embodiment of the compounds of Formula (I-E1), (I-F1), (I-E) or (I-F), R³ is —C₁₋₆ alkyl optionally substituted with one to three groups selected from halogen or —C₁₋₆ alkoxy. In one embodiment, R³ is —C₁₋₆ alkyl. In one embodiment, R³ is —CH₃, —CH₂CH₃, —CH₂F, —CHF₂, —CF₃, —CH₂OCH₂CH₃, or —CH₂OCH₃. In one embodiment, R³ is —CH₃.

In one embodiment of the compounds of Formula (I-E1), (I-F1), (I-E) or (I-F), R¹⁰ is hydrogen, halogen, cyano, —CH₃, —CH₂CH₃, —CH₂F, —CHF₂, —CF₃, —CH₂OCH₃, or —CH₂SCH₃. In one embodiment of the compounds of Formula (I-E1), (I-F1), (I-E) or (I-F), R¹⁰ is hydrogen, —CH₃, —CH₂CH₃, or —CH₂OCH₃.

In one embodiment of the compounds of the present invention, the compound has the structure of Formula (I-G1):

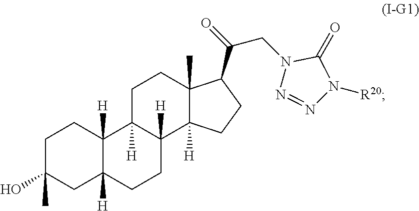

or a pharmaceutically acceptable salt, solvate, ester, or prodrug thereof, wherein:
R²⁰ is hydrogen, —C₁₋₆ alkyl, —C₃₋₁₂ cycloalkyl, C₃₋₁₂ cycloalkyl-C₁₋₆ alkyl-, —C₆-C₁₂ aryl, or 5-12 membered heteroaryl, each of which can be optionally substituted with halogen, nitro, cyano, —C₁₋₆ alkyl, —C₂₋₆ alkenyl, —C₂₋₆ alkynyl, —C₃₋₁₂ cycloalkyl, 3-12 membered heterocylyl, —C₆-C₁₂ aryl, 5-12 membered heteroaryl, —OR^{A}, —C(═O)R^{A}, —C(═O)OR^{A}, —OC(═O)R^{A}, —OC(═O)OR^{A}, —C(═O)NR^{A}R^{A}, —NR^{A}R^{A}, —NR^{A}C(═O)R^{A}, —OC(═O)NR^{A}R^{A}, —NR^{A}C(═O)OR^{A}, —NR^{A}C(═O)NR^{A}R^{A}, —SR^{A}, —S(═O)R^{A}, —S(═O)₂R^{A}, —S(═O)₂OR^{A}, —OS(═O)₂R^{A}, —S(═O)₂NR^{A}R^{A}, or —NR^{A}S(═O)₂R^{A};
wherein R^{A} is independently hydrogen, alkyl, haloalkyl, alkenyl, alkynyl, cycloalkyl, cycloalkylalkyl, carbocylyl, heterocylyl, aryl, C₆-C₁₂ aryl-C₁-C₆ alkyl-, heteroaryl, heteroaryl-C₁-C₆ alkyl-, an oxygen protecting group when attached to oxygen, a sulfur protecting group when attached to sulfur, or a nitrogen protecting group when attached to nitrogen; or two R^{A} groups can be taken together with the atoms to which they are attached to, to form a heterocylyl or heteroaryl ring;
wherein the substituents are selected from fluoro, chloro, bromo, cyano, nitro, hydroxy, methoxy, ethoxy, propoxy, isopropoxy, methyl, ethyl, propyl, isopropyl, butyl, i-butyl, s-butyl, t-butyl, —CH₂CN, —CH₂CH₂CN, —CH₂F, —CHF₂, —CF₃, —CH₂OCH₃, —CH₂SCH₃, -methylhydroxy, morpholine, pyrrolidine, piperidine, piperazine, phenyl, benzyl, pyridine, pyrimidine, oxazole, pyrazole, cyclopropyl, cyclobutyl, cyclopentyl, cyclohexyl, —OCH₂F, —OCHF₂, —OCF₃, -ethylmethoxy, -methylcyclopropyl, —OR^{A}, —C(═O)R^{A}, —C(═O)OR^{A}, —OC(═O)R^{A}, —OC(═O)OR^{A}, —C(═O)NR^{A}R^{A}, —NR^{A}R^{A}, —NR^{A}C(═O)R^{A}, —OC(═O)NR^{A}R^{A}, —NR^{A}C(═O)OR^{A}, —NR^{A}C(═O)NR^{A}R^{A}, —SR^{A}, —S(═O)R^{A}, —S(═O)₂R^{A}, —S(═O)₂OR^{A}, —OS(═O)₂R^{A}, —S(═O)₂NR^{A}R^{A}, or —NR^{A}S(═O)₂R^{A}, wherein R^{A} is independently hydrogen, methyl, ethyl, propyl, isopropyl, cyclopropyl, cyclobutyl, cyclopentyl, cyclohexyl, —CH₂F, —CHF₂, —CF₃, phenyl, benzyl, pyridine, pyrimidine, -ethylmethoxy, or -methylcyclopropyl, or two R^{A} groups can be taken together with the atoms to which they are attached to, to form a heterocylyl or heteroaryl ring.

In one embodiment of the compounds of Formula (I-E1), (I-F1), (I-G1), (I-E), (I-F), or (I-G), R²⁰ is hydrogen, —C₁₋₆ alkyl, —C₂₋₆ alkenyl, —C₂₋₆ alkynyl, —C₁₋₆ haloalkyl, —C₃₋₁₂ cycloalkyl, C₃₋₁₂ cycloalkyl-C₁-C₆ alkyl-, 3-12 membered heterocyclyl, —C₆-C₁₂ aryl, C₆-C₁₂ aryl-C₁-C₆ alkyl-, heteroaryl-C₁-C₆ alkyl-, or 5-12 membered heteroaryl, each of which can be optionally substituted with halogen, nitro, cyano, —C₁₋₆ alkyl, —C₂₋₆ alkenyl, —C₂₋₆ alkynyl, —C₃₋₁₂ cycloalkyl, 3-12 membered heterocylyl, —C₆-C₁₂ aryl, 5-12 membered heteroaryl, —OR^{A}, —C(═O)R^{A}, —C(═O)OR^{A}, —OC(═O)R^{A}, —OC(═O)OR^{A}, —C(═O)NR^{A}R^{A}, —NR^{A}R^{A}, —NR^{A}C(═O)R^{A}, —OC(═O)NR^{A}R^{A}, —NR^{A}C(═O)OR^{A}, —NR^{A}C(═O)NR^{A}R^{A}, —SR^{A}, —S(═O)R^{A}, —S(═O)₂R^{A}, —S(═O)₂OR^{A}, —OS(═O)₂R^{A}, —S(═O)₂NR^{A}R^{A}, or —NR^{A}S(═O)₂R^{A}. In one embodiment, R²⁰ is —C₁₋₆ alkyl, —C₃₋₁₂ cycloalkyl, C₃₋₁₂ cycloalkyl-C₁-C₆ alkyl-, —C₆-C₁₂ aryl, or 5-12 membered heteroaryl, each of which are optionally substituted. In one embodiment, R²⁰ is —CH₃, —CF₃, —CH₂CH₃, -i-Pr, -n-Pr, -i-Bu, -s-Bu, -t-Bu, —CH₂cyclopropyl, —CH₂CN, or —CH₂CH₂CN. In one embodiment, R²⁰ is —CH₃, —CH₂CH₃, -i-Pr, -n-Pr, -i-Bu, -s-Bu, -t-Bu, or —CH₂cyclopropyl. In one embodiment, R²⁰ is cyclopropyl, cyclobutyl, cyclopentyl, or cyclohexyl. In one embodiment, R²⁰ is phenyl. In one embodiment, R²⁰ is phenyl substituted with one or more halogen, cyano, or alkoxy. In one embodiment, R²⁰ is pyridine. In one embodiment, R²⁰ is pyridine substituted with one or more halogen, cyano, or alkoxy. In one embodiment, R²⁰ is oxazole, pyrazole, or N-methylpyrazole. In one embodiment, R²⁰ is hydrogen.

In one embodiment of the compounds of the present invention, the compound is selected from Table A or pharmaceutically acceptable salts, solvates, esters, or prodrugs thereof. In one embodiment of the compounds of the present invention, the compound is selected from the compounds prepared in any one of Examples 1-18, or pharmaceutically acceptable salts, solvates, esters, or prodrugs thereof. In one embodiment of the compounds of the present invention, the compound is selected from the compounds prepared in any one of Examples 19-29, or pharmaceutically acceptable salts, solvates, esters, or prodrugs thereof.

In another aspect, the present disclosure provides a pharmaceutical composition comprising a compound of the present invention and a pharmaceutically acceptable carrier or excipient. In certain embodiments, the compound of the present invention is provided in an effective amount in the pharmaceutical composition. In certain embodiments, the compound of the present invention is provided in a therapeutically effective amount. In certain embodiments, the compound of the present invention is provided in a prophylactically effective amount.

The compounds of the present invention as described herein, act, in certain embodiments, as GABA modulators, e.g., effecting the GABA_{A} receptor in either a positive or negative manner. As modulators of the excitability of the central nervous system (CNS), as mediated by their ability to modulate GABA_{A} receptor, such compounds are expected to have CNS-activity.

Thus, in another aspect, the present disclosure provides a method of treating a CNS-related disorder in a subject in need thereof, comprising administering to the subject an effective amount of the compound of the present invention. In certain embodiments, the CNS-related treatment is treatment for a sleep disorder, a mood disorder, a schizophrenia spectrum disorder, a convulsive disorder, a disorder of memory and/or cognition, a movement disorder, a personality disorder, autism spectrum disorder, pain, traumatic brain injury, a vascular disease, a substance abuse disorder and/or withdrawal syndrome, or tinnitus.

In one embodiment of the present disclosure, a method for inducing sedation and/or anesthesia in a subject in need thereof, is provided, comprising administering to the subject an effective amount of the compound of the present invention.

In certain embodiments, the compound of the present invention is administered orally, subcutaneously, intravenously, or intramuscularly. In certain embodiments, the compound is administered chronically.

Other objects and advantages will become apparent to those skilled in the art from a consideration of the ensuing Detailed Description, Examples, and Claims.

### DETAILED DESCRIPTION OF THE DISCLOSURE

All publications, patents and patent applications, including any drawings and appendices therein are incorporated by reference in their entirety for all purposes to the same extent as if each individual publication, patent or patent application, drawing, or appendix was specifically and individually indicated to be incorporated by reference in its entirety for all purposes.

While various embodiments of the present disclosure are described herein, it will be obvious to those skilled in the art that such embodiments are provided by way of example only. Numerous modifications and changes to, and variations and substitutions of, the embodiments described herein will be apparent to those skilled in the art without departing from the disclosure. It is understood that various alternatives to the embodiments described herein may be employed in practicing the disclosure. It is also understood that every embodiment of the disclosure may optionally be combined with any one or more of the other embodiments described herein which are consistent with that embodiment.

Where elements are presented in list format (e.g., in a Markush group), it is understood that each possible subgroup of the elements is also disclosed, and any one or more elements can be removed from the list or group.

It is also understood that, unless clearly indicated to the contrary, in any method described or claimed herein that includes more than one act, the order of the acts of the method is not necessarily limited to the order in which the acts of the method are recited, but the disclosure encompasses embodiments in which the order is so limited.

It is further understood that, in general, where an embodiment in the description or the claims is referred to as comprising one or more features, the disclosure also encompasses embodiments that consist of, or consist essentially of, such feature(s).

It is also understood that any embodiment of the disclosure, e.g., any embodiment found within the prior art, can be explicitly excluded from the claims, regardless of whether or not the specific exclusion is recited in the specification.

Headings are included herein for reference and to aid in locating certain sections. Headings are not intended to limit the scope of the embodiments and concepts described in the sections under those headings, and those embodiments and concepts may have applicability in other sections throughout the entire disclosure.

### I. Definitions

While the following terms are believed to be well understood by one of ordinary skill in the art, the following definitions are set forth to facilitate explanation of the presently disclosed subject matter.

Throughout the present specification, the terms “about” and/or “approximately” may be used in conjunction with numerical values and/or ranges. The term “about” is understood to mean those values near to a recited value. For example, “about 40 [units]” may mean within ±25% of 40 (e.g., from 30 to 50), within ±20%, ±15%, ±10%, ±9%, ±8%, ±7%, ±6%, ±5%, ±4%, ±3%, ±2%, ±1%, less than ±1%, or any other value or range of values therein or therebelow. Furthermore, the phrases “less than about [a value]” or “greater than about [a value]” should be understood in view of the definition of the term “about” provided herein. The terms “about” and “approximately” may be used interchangeably. Whenever the term “about” or “approximately” precedes the first numerical value in a series of two or more numerical values, the term “about” or “approximately” applies to all numerical values listed in the series. In certain embodiments, the term “about” or “approximately” means within one standard deviation.

Throughout the present specification, numerical ranges are provided for certain quantities. It is to be understood that these ranges comprise all subranges therein. Thus, the range “from 50 to 80” includes all possible ranges therein (e.g., 51-79, 52-78, 53-77, 54-76, 55-75, 60-70, etc.). Furthermore, all values within a given range may be an endpoint for the range encompassed thereby (e.g., the range 50-80 includes the ranges with endpoints such as 55-80, 50-75, etc.).

The term “a” or “an” or “the” refers to one or more of that entity. As such, the terms “a” (or “an”), “one or more” and “at least one” are used interchangeably herein. In addition, reference to “an agonist” by the indefinite article “a” or “an” does not exclude the possibility that more than one of the agonists is present, unless the context clearly requires that there is one and only one of the inhibitors.

When the terms “no more than” or “less than” precedes the first numerical value in a series of two or more numerical values, the term “no more than” or “less than” applies to each one of the numerical values in that series of numerical values.

As used herein, the verb “comprise” as is used in this description and in the claims and its conjugations are used in its non-limiting sense to mean that items following the word are included, but items not specifically mentioned are not excluded. The present invention may suitably “comprise”, “consist of”, or “consist essentially of”, the steps, elements, and/or reagents described in the claims.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

The term “exemplary” as used herein means “serving as an example, instance, or illustration”. Any embodiment characterized herein as “exemplary” is not necessarily to be construed as preferred or advantageous over other embodiments.

All weight percentages (i.e., “% by weight” and “wt. %” and w/w) referenced herein, unless otherwise indicated, are measured relative to the total weight of the pharmaceutical composition.

As used herein, “substantially” or “substantial” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking, the nearness of completion will be so as to have the same overall result as if absolute and total completion were obtained. The use of “substantially” is equally applicable when used in a negative connotation to refer to the complete or near complete lack of action, characteristic, property, state, structure, item, or result. For example, a composition that is “substantially free of” other active agents would either completely lack other active agents, or so nearly completely lack other active agents that the effect would be the same as if it completely lacked other active agents. In other words, a composition that is “substantially free of” an ingredient or element or another active agent may still contain such an item as long as there is no measurable effect thereof.

The terms below, as used herein, have the following meanings, unless indicated otherwise:

“Acyl” refers to —C(═O)-alkyl radical.

“Amino” refers to the —NH₂ radical.

“Cyano” refers to the —CN radical.

“Halo” “halide” or “halogen” refers to bromo, chloro, fluoro or iodo radical.

“Hydrogen” refers to H or D.

“Hydroxy” or “hydroxyl” refers to the —OH radical.

“Imino” refers to the ═NH substituent.

“Nitro” refers to the —NO₂ radical.

“Oxo” refers to the ═O substituent.

“Thioxo” refers to the ═S substituent.

“Sulfhydryl” and “mercapto” refers to —SH radical.

“Alkyl” or “alkyl group” refers to a fully saturated, straight (linear) or branched hydrocarbon chain radical having from one to twenty carbon atoms, and which is attached to the rest of the molecule by a single bond. Alkyls comprising any number of carbon atoms from 1 to 20 are included. An alkyl comprising up to 20 carbon atoms is a C₁-C₂₀ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted. The term “lower alkyl” refers to a C₁-C₆ alkyl, which can be linear or branched, for example including branched C₃-C₆ alkyl.

“Alkylene”, “-alkyl-” or “alkylene chain” refers to a fully saturated, straight or branched divalent hydrocarbon chain radical, and having from one to twenty carbon atoms. Non-limiting examples of C₁-C₂₀ alkylene include methylene, ethylene, propylene, n-butylene, ethenylene, propenylene, n-butenylene, propynylene, n-butynylene, and the like. The alkylene chain is attached to the rest of the molecule through a single bond and to the radical group through a single bond. The points of attachment of the alkylene chain to the rest of the molecule and to the radical group can be through one carbon or any two carbons within the chain. Unless stated otherwise specifically in the specification, an alkylene chain can be optionally substituted.

“Alkenyl” or “alkenyl group” refers to a straight or branched hydrocarbon chain radical having from two to twenty carbon atoms, and having one or more carbon-carbon double bonds. Each alkenyl group is attached to the rest of the molecule by a single bond. Alkenyl group comprising any number of carbon atoms from 2 to 20 are included. An alkenyl group comprising up to 20 carbon atoms is a C₂-C₂₀ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkyl group can be optionally substituted.

“Alkenylene” or “alkenylene chain” refers to a straight or branched divalent hydrocarbon chain radical, having from two to twenty carbon atoms, and having one or more carbon-carbon double bonds. Non-limiting examples of C₂-C₂₀ alkenylene include ethene, propene, butene, and the like. The alkenylene chain is attached to the rest of the molecule through a single bond and to the radical group through a single bond. The points of attachment of the alkenylene chain to the rest of the molecule and to the radical group can be through one carbon or any two carbons within the chain. Unless stated otherwise specifically in the specification, an alkenylene chain can be optionally substituted.

“Alkynyl” or “alkynyl group” refers to a straight or branched hydrocarbon chain radical having from two to twenty carbon atoms, and having one or more carbon-carbon triple bonds. Each alkynyl group is attached to the rest of the molecule by a single bond. Alkynyl group comprising any number of carbon atoms from 2 to 20 are included. An alkynyl group comprising up to 20 carbon atoms is a C₂-C₂₀ alkynyl, an alkynyl comprising up to 10 carbon atoms is a C₂-C₁₀ alkynyl, an alkynyl group comprising up to 6 carbon atoms is a C₂-C₆ alkynyl and an alkynyl comprising up to 5 carbon atoms is a C₂-C₅ alkynyl. A C₂-C₅ alkynyl includes C₅ alkynyls, C₄ alkynyls, C₃ alkynyls, and C₂ alkynyls. A C₂-C₆ alkynyl includes all moieties described above for C₂-C₅ alkynyls but also includes C₆ alkynyls. A C₂-C₁₀ alkynyl includes all moieties described above for C₂-C₅ alkynyls and C₂-C₆ alkynyls, but also includes C₇, C₈, C₉ and C₁₀ alkynyls. Similarly, a C₂-C₁₂ alkynyl includes all the foregoing moieties, but also includes C₁₁ and C₁₂ alkynyls. Non-limiting examples of C₂-C₁₂ alkenyl include ethynyl, propynyl, butynyl, pentynyl and the like. Unless stated otherwise specifically in the specification, an alkyl group can be optionally substituted.

“Alkynylene” or “alkynylene chain” refers to a straight or branched divalent hydrocarbon chain radical, having from two to twenty carbon atoms, and having one or more carbon-carbon triple bonds. Non-limiting examples of C₂-C₂₀ alkynylene include ethynylene, propargylene and the like. The alkynylene chain is attached to the rest of the molecule through a single bond and to the radical group through a single bond. The points of attachment of the alkynylene chain to the rest of the molecule and to the radical group can be through one carbon or any two carbons within the chain. Unless stated otherwise specifically in the specification, an alkynylene chain can be optionally substituted.

“Alkoxy” or “—O-alkyl” refers to a radical of the formula —ORₐ where Rₐ is an alkyl, alkenyl or alknyl radical as defined above containing one to twenty carbon atoms. Unless stated otherwise specifically in the specification, an alkoxy group can be optionally substituted.

“Alkylamino” refers to a radical of the formula —NHRₐ or —NRₐRₐ where each Rₐ is, independently, an alkyl, alkenyl or alkynyl radical as defined above containing one to twelve carbon atoms. Unless stated otherwise specifically in the specification, an alkylamino group can be optionally substituted.

“Alkylcarbonyl” refers to the —C(═O)Rₐ moiety, wherein Rₐ is an alkyl, alkenyl or alkynyl radical as defined above. A non-limiting example of an alkyl carbonyl is the methyl carbonyl (“acetal”) moiety. Alkylcarbonyl groups can also be referred to as “Cw-Cz acyl” where w and z depicts the range of the number of carbons in Rₐ, as defined above. For example, “C₁-C₁₀ acyl” refers to alkylcarbonyl group as defined above, where Rₐ is C₁-C₁₀ alkyl, C₁-C₁₀ alkenyl, or C₁-C₁₀ alkynyl radical as defined above. Unless stated otherwise specifically in the specification, an alkyl carbonyl group can be optionally substituted.

The term “aminoalkyl” refers to an alkyl group that is substituted with one or more —NH₂ groups. In certain embodiments, an aminoalkyl group is substituted with one, two, three, four, five or more —NH₂ groups. An aminoalkyl group may optionally be substituted with one or more additional substituents as described herein.

“Aryl” refers to a hydrocarbon ring system radical comprising hydrogen, 6 to 18 carbon atoms and at least one aromatic ring. For purposes of this invention, the aryl radical can be a monocyclic, bicyclic, tricyclic or tetracyclic ring system, which can include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” is meant to include aryl radicals that are optionally substituted.

“Aralkyl”, “arylalkyl” or “-alkylaryl” refers to a radical of the formula —R_{b}-R_{c} where R_{b} is an alkylene, alkenylene or alkynylene group as defined above and R_{c} is one or more aryl radicals as defined above, for example, benzyl, diphenylmethyl and the like. Unless stated otherwise specifically in the specification, an aralkyl group can be optionally substituted.

“Carbocyclyl,” “carbocyclic ring” or “carbocycle” refers to a rings structure, wherein the atoms which form the ring are each carbon. Carbocyclic rings can comprise from 3 to 20 carbon atoms in the ring. Carbocyclic rings include aryls and cycloalkyl. Cycloalkenyl and cycloalkynyl as defined herein. Unless stated otherwise specifically in the specification, a carbocyclyl group can be optionally substituted.

“Cycloalkyl” refers to a stable non-aromatic monocyclic or polycyclic fully saturated hydrocarbon radical consisting solely of carbon and hydrogen atoms, which can include fused or bridged ring systems, having from three to twenty carbon atoms, preferably having from three to ten carbon atoms, and which is attached to the rest of the molecule by a single bond. Monocyclic cycloalkyl radicals include, for example, cyclopropyl, cyclobutyl, cyclopentyl, cyclohexyl, cycloheptyl, and cyclooctyl. Polycyclic cycloalkyl radicals include, for example, adamantyl, norbornyl, decalinyl, 7,7-dimethyl-bicyclo[2.2.1]heptanyl, bicyclo[3.1.0]hexane, octahydropentalene, bicyclo[1.1.1]pentane, cubane, and the like. Unless otherwise stated specifically in the specification, a cycloalkyl group can be optionally substituted.

“Cycloalkenyl” refers to a stable non-aromatic monocyclic or polycyclic hydrocarbon radical consisting solely of carbon and hydrogen atoms, having one or more carbon-carbon double bonds, which can include fused or bridged ring systems, having from three to twenty carbon atoms, preferably having from three to ten carbon atoms, and which is attached to the rest of the molecule by a single bond. Monocyclic cycloalkenyl radicals include, for example, cyclopentenyl, cyclohexenyl, cycloheptenyl, cycloctenyl, and the like. Polycyclic cycloalkenyl radicals include, for example, bicyclo[2.2.1]hept-2-enyl and the like. Unless otherwise stated specifically in the specification, a cycloalkenyl group can be optionally substituted.

“Cycloalkynyl” refers to a stable non-aromatic monocyclic or polycyclic hydrocarbon radical consisting solely of carbon and hydrogen atoms, having one or more carbon-carbon triple bonds, which can include fused or bridged ring systems, having from three to twenty carbon atoms, preferably having from three to ten carbon atoms, and which is attached to the rest of the molecule by a single bond. Monocyclic cycloalkynyl radicals include, for example, cycloheptynyl, cyclooctynyl, and the like. Unless otherwise stated specifically in the specification, a cycloalkynyl group can be optionally substituted.

“Cycloalkylalkyl” or “-alkylcycloalkyl” refers to a radical of the formula —R_{b}-R_{d} where R_{b} is an alkylene, alkenylene, or alkynylene group as defined above and R_{d} is a cycloalkyl, cycloalkenyl, cycloalkynyl radical as defined above. Unless stated otherwise specifically in the specification, a cycloalkylalkyl group can be optionally substituted.

“Haloalkyl” refers to an alkyl radical, as defined above, that is substituted by one, two, three, four, five, six or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group can be optionally substituted.

“Haloalkenyl” refers to an alkenyl radical, as defined above, that is substituted by one, two, three, four, five, six or more halo radicals, as defined above, e.g., 1-fluoropropenyl, 1,1-difluorobutenyl, and the like. Unless stated otherwise specifically in the specification, a haloalkenyl group can be optionally substituted.

“Haloalkynyl” refers to an alkynyl radical, as defined above, that is substituted by one, two, three, four, five, six or more halo radicals, as defined above, e.g., 1-fluoropropynyl, 1-fluorobutynyl, and the like. Unless stated otherwise specifically in the specification, a haloalkenyl group can be optionally substituted.

“Heterocyclyl,” “heterocyclic ring” or “heterocycle” refers to a stable 3- to 20-membered non-aromatic ring radical which consists of two to twelve carbon atoms and from one to six heteroatoms selected from the group consisting of nitrogen, oxygen and sulfur. Heterocyclycl or heterocyclic rings include heteroaryls as defined below. Unless stated otherwise specifically in the specification, the heterocyclyl radical can be a monocyclic, bicyclic, tricyclic or tetracyclic ring system, which can include fused or bridged ring systems; and the nitrogen, carbon or sulfur atoms in the heterocyclyl radical can be optionally oxidized; the nitrogen atom can be optionally quaternized; and the heterocyclyl radical can be partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group can be optionally substituted.

The term “hydroxyalkyl” or “hydroxylalkyl” refers to an alkyl group that is substituted with one or more hydroxyl (—OH) groups. In certain embodiments, a hydroxyalkyl group is substituted with one, two, three, four, five or more —OH groups. A hydroxyalkyl group may optionally be substituted with one or more additional substituents as described herein.

The term “hydrocarbyl” refers to a monovalent hydrocarbon radical, whether aliphatic, partially or fully unsaturated, acyclic, cyclic or aromatic, or any combination of the preceding. In certain embodiments, a hydrocarbyl group has 1 to 40 or more, 1 to 30 or more, 1 to 20 or more, or 1 to 10 or more, carbon atoms. The term “hydrocarbylene” refers to a divalent hydrocarbyl group. A hydrocarbyl or hydrocarbylene group may optionally be substituted with one or more substituents as described herein.

The term “heterohydrocarbyl” refers to a hydrocarbyl group in which one or more of the carbon atoms are independently replaced by a heteroatom selected from oxygen, sulfur, nitrogen and phosphorus. In certain embodiments, a heterohydrocarbyl group has 1 to 40 or more, 1 to 30 or more, 1 to 20 or more, or 1 to 10 or more, carbon atoms, and 1 to 10 or more, or 1 to 5 or more, heteroatoms. The term “heterohydrocarbylene” refers to a divalent hydrocarbyl group. Examples of heterohydrocarbyl and heterohydrocarbylene groups include without limitation ethylene glycol and polyethylene glycol moieties, such as (—CH₂CH₂O—)ₙH (a monovalent heterohydrocarbyl group) and (—CH₂CH₂O—)ₙ (a divalent heterohydrocarbylene group) where n is an integer from 1 to 12 or more, and propylene glycol and polypropylene glycol moieties, such as (—CH₂CH₂CH₂O—)ₙH and (—CH₂CH(CH₃)O—)ₙH (monovalent heterohydrocarbyl groups) and (—CH₂CH₂CH₂O—)ₙ and (—CH₂CH(CH₃)O—)ₙ (divalent heterohydrocarbylene groups) where n is an integer from 1 to 12 or more. A heterohydrocarbyl or heterohydrocarbylene group may optionally be substituted with one or more substituents as described herein.

“N-heterocyclyl” refers to a heterocyclyl radical as defined above containing at least one nitrogen and where the point of attachment of the heterocyclyl radical to the rest of the molecule is through a nitrogen atom in the heterocyclyl radical. Unless stated otherwise specifically in the specification, a N-heterocyclyl group can be optionally substituted.

“Heterocyclylalkyl” or “-alkylheterocyclyl” refers to a radical of the formula —R_{b}-Rₑ where R_{b} is an alkylene, alkenylene, or alkynylene chain as defined above and Rₑ is a heterocyclyl radical as defined above, and if the heterocyclyl is a nitrogen-containing heterocyclyl, the heterocyclyl can be attached to the alkyl, alkenyl, alkynyl radical at the nitrogen atom. Unless stated otherwise specifically in the specification, a heterocyclylalkyl group can be optionally substituted.

“Heteroaryl” refers to a 5- to 20-membered ring system radical comprising hydrogen atoms, one to thirteen carbon atoms, one to six heteroatoms selected from the group consisting of nitrogen, oxygen and sulfur, and at least one aromatic ring. For purposes of this invention, the heteroaryl radical can be a monocyclic, bicyclic, tricyclic or tetracyclic ring system, which can include fused or bridged ring systems; and the nitrogen, carbon or sulfur atoms in the heteroaryl radical can be optionally oxidized; the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, tetrazolonyl, triazinyl, and thiophenyl (i.e. thienyl). Unless stated otherwise specifically in the specification, a heteroaryl group can be optionally substituted.

“N-heteroaryl” refers to a heteroaryl radical as defined above containing at least one nitrogen and where the point of attachment of the heteroaryl radical to the rest of the molecule is through a nitrogen atom in the heteroaryl radical. Unless stated otherwise specifically in the specification, an N-heteroaryl group can be optionally substituted.

“Heteroarylalkyl” or “-alkylheteroaryl” refers to a radical of the formula —R_{b}-R_{f} where R_{b} is an alkylene, alkenylene, or alkynylene chain as defined above and R_{f} is a heteroaryl radical as defined above. Unless stated otherwise specifically in the specification, a heteroarylalkyl group can be optionally substituted.

“Thioalkyl” refers to a radical of the formula —SRₐ where Rₐ is an alkyl, alkenyl, or alkynyl radical as defined above containing one to twelve carbon atoms. Unless stated otherwise specifically in the specification, a thioalkyl group can be optionally substituted.

The term “substituted” used herein means any of the above groups (i.e., alkyl, alkylene, alkenyl, alkenylene, alkynyl, alkynylene, alkoxy, alkylamino, alkylcarbonyl, thioalkyl, aryl, aralkyl, carbocyclyl, cycloalkyl, cycloalkenyl, cycloalkynyl, cycloalkylalkyl, haloalkyl, heterocyclyl, N-heterocyclyl, heterocyclylalkyl, heteroaryl, N-heteroaryl and/or heteroarylalkyl) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with halide, cyano, nitro, hydroxyl, sulfhydryl, amino, —OR_{g}, —SR_{g}, —NRₕRᵢ, alkyl, alkenyl, alkynyl, haloalkyl, hydroxyalkyl, aminoalkyl, -alkylcycloalkyl, -alkylheterocyclyl, -alkylaryl, -alkylheteroaryl, cycloalkyl, heterocyclyl, aryl, heteroaryl, —C(═O)R_{g}, —C(═NRⱼ)R_{g}, —S(═O)R_{g}, —S(═O)₂R_{g}, —S(═O)₂ORₖ, —C(═O)ORₖ, —OC(═O)R_{g}, —C(═O)NRₕRᵢ, —NR_{g}C(═O)R_{g}, —S(═O)₂NRₕRᵢ, —NR_{g}S(═O)₂R_{g}, —OC(═O)OR_{g}, —OC(═O)NRₕRᵢ, —NR_{g}C(═O)OR_{g}, —NR_{g}C(═O)NRₕRᵢ, —NR_{g}C(═NRⱼ)NRₕRᵢ, —P(═O)(R_{g})₂, —P(═O)(ORₖ)R_{g}, —P(═O)(ORₖ)₂, —OP(═O)(R_{g})₂, —OP(═O)(ORₖ)R_{g}, and —OP(═O)(ORₖ)₂, wherein: each occurrence of R_{g} is independently selected from hydrogen, alkyl, haloalkyl, hydroxyalkyl, aminoalkyl, -alkylcycloalkyl, -alkylheterocyclyl, -alkylaryl, -alkylheteroaryl, cycloalkyl, heterocyclyl, aryl or heteroaryl; each occurrence of Rₕ and Rᵢ is independently selected from hydrogen, alkyl, haloalkyl, hydroxyalkyl, aminoalkyl, -alkylcycloalkyl, -alkylheterocyclyl, -alkylaryl, -alkylheteroaryl, cycloalkyl, heterocyclyl, aryl or heteroaryl, or Rₕ and Rᵢ, together with the nitrogen atom to which they are attached, form a heterocyclic or heteroaryl ring; each occurrence of Rⱼ independently is hydrogen, —OR_{g}, alkyl, haloalkyl, hydroxyalkyl, aminoalkyl, -alkylcycloalkyl, -alkylheterocyclyl, -alkylaryl, -alkylheteroaryl, cycloalkyl, heterocyclyl, aryl or heteroaryl; and each occurrence of Rₖ independently is hydrogen, Z, alkyl, haloalkyl, hydroxyalkyl, aminoalkyl, -alkylcycloalkyl, -alkylheterocyclyl, -alkylaryl, -alkylheteroaryl, cycloalkyl, heterocyclyl, aryl or heteroaryl, wherein each occurrence of Z independently is H⁺, Li⁺, Na⁺, K⁺, Cs⁺, Mg⁺², Ca⁺², or —⁺N(R_{g})₂RₕRᵢ.

As used herein, the symbol “ [symbol] ” (hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example, “ [symbol] ” indicates that the chemical entity “XY” is bonded to another chemical entity via the point of attachment bond. Furthermore, the specific point of attachment to the non-depicted chemical entity can be specified by inference. For example, the compound CH₃—R³, wherein R³ is H or “XY [symbol] ” infers that when R³ is “XY”, the point of attachment bond is the same bond as the bond by which R³ is depicted as being bonded to CH₃.

“Fused” refers to any ring structure described herein which is fused to an existing ring structure in the compounds of the invention. When the fused ring is a heterocyclyl ring or a heteroaryl ring, any carbon atom on the existing ring structure which becomes part of the fused heterocyclyl ring or the fused heteroaryl ring can be replaced with a nitrogen atom.

“Oxygen Protecting Group” as used herein, refers to a group capable of protecting an oxygen atom of a free hydroxyl group which may be subsequently removed without disturbing the remainder of the molecule. In some embodiments, an oxygen protecting group can be selectively removed in good yield by readily available, preferably nontoxic reagents that do not react with the other functional groups present in a molecule and has a minimum of additional functionality to avoid further reactions at the protection site. Compatibility of the protecting groups will typically take into consideration the reaction conditions in subsequent steps. An oxygen protecting group can be alkoxycarbonyl, acyl, acetal, ether, ester, silyl ether, alkylsulfonyl, or arylsulfonyl. Non-limiting examples of an oxygen protecting groups include allyl, triphenylmethyl (trityl or Tr), benzyl, methanesulfonyl, p-toluenesulfonyl, p-methoxybenzyl (PMB), p-methoxyphenyl (PMP), methoxymethyl (MOM), β-methoxyethoxymethyl (MEM), tetrahydropyranyl (THP), ethoxy ethyl (EE), methylthiomethyl (MTM)₁ 2-methoxy-2-propyl (MOP), 2-trimethylsilylethoxymethyl (SEM), benzoate (Bz)₁ allyl carbonate, 2,2,2-trichloroethyl carbonate (Troc), 2-trimethylsilylethyl carbonate, trimethylsilyl (TMS), triethylsilyl (TES), triisopropylsilyl (TIPS), triphenylsilyl (TPS), t-butyldimethylsilyl (TBDMS)₁ and t-butyldiphenylsilyl (TBDPS). A variety of protecting groups for the oxygen and the synthesis thereof may be found in “Protective Groups in Organic Synthesis” by T. W. Greene and P. G. M. Wuts, John Wiley & Sons, 1999.

“Nitrogen Protecting Group” as used herein, refers to a group capable of protecting a nitrogen atom of a free amino or NH group which may be subsequently removed without disturbing the remainder of the molecule. In some embodiments, a nitrogen protecting group can be selectively removed in good yield by readily available, preferably nontoxic reagents that do not react with the other functional groups present in a molecule and has a minimum of additional functionality to avoid further reactions at the protection site. Compatibility of the protecting groups will typically take into consideration the reaction conditions in subsequent steps. A nitrogen protecting group can be silyl, substituted silyl, alkyl ether, substituted alkyl ether, cycloalkyl ether, substituted cycloalkyl ether, alkyl, substituted alkyl, carbamate, urea, amide, imide, enamine, sulfenyl, sulfonyl, nitro, nitroso, oxide, phosphinyl, phosphoryl, silyl, organometallic, borinic acid and boronic acid groups. Non-limiting examples of a nitrogen protecting group includes silyl protecting groups (e.g., SEM: trimethylsilylethoxymethyl, TBDMS: tert-butyldimethylsilyl); alkyl ether protecting groups such as cycloalkyl ethers (e.g., THP: tetrahydropyran); carbamate protecting groups such as alkyloxycarbonyl (e.g., Boc: t-butyloxycarbonyl), aryloxycarbonyl (e.g., Cbz: benzyloxycarbonyl, and FMOC: fluorene-9-methyloxy carbonyl), alkyloxycarbonyl (e.g., methyloxycarbonyl), alkylcarbonyl or arylcarbonyl, substituted alkyl, especially arylalkyl (e.g., trityl (triphenylmethyl), benzyl and substituted benzyl), acetyl, pivaloyl, and the like. A variety of protecting groups for the nitrogen and the synthesis thereof may be found in “Protective Groups in Organic Synthesis” by T. W. Greene and P. G. M. Wuts, John Wiley & Sons, 1999.

“Sulfur Protecting Group” as used herein, refers to a group capable of protecting a sulfur atom of a free thiol group which may be subsequently removed without disturbing the remainder of the molecule. In some embodiments, a sulfur protecting group can be selectively removed in good yield by readily available, preferably nontoxic reagents that do not react with the other functional groups present in a molecule and has a minimum of additional functionality to avoid further reactions at the protection site. Compatibility of the protecting groups will typically take into consideration the reaction conditions in subsequent steps. Non-limiting examples of a sulfur protecting group includes ACM (acetamidomethyl) and the like, picolyl, trityl and the like, dimethylphenyl and the like, xanthyl, phenacyl and the like, benzyl, fluorenylmethyl (FM) and the like, 2-pyranyl and the like, or lower alkyl carbonyl, and disulfide moieties.

The invention disclosed herein is also meant to encompass the in vivo metabolic products of the disclosed compounds. Such products can result from, for example, the oxidation, reduction, hydrolysis, amidation, esterification, and the like of the administered compound, primarily due to enzymatic processes. Accordingly, the invention includes compounds produced by a process comprising administering a compound of this invention to a mammal for a period of time sufficient to yield a metabolic product thereof. Such products are typically identified by administering a radiolabelled compound of the invention in a detectable dose to an animal, such as rat, mouse, guinea pig, monkey, or to human, allowing sufficient time for metabolism to occur, and isolating its conversion products from the urine, blood or other biological samples.

“Stable compound” and “stable structure” are meant to indicate a compound that is sufficiently robust to survive isolation to a useful degree of purity from a reaction mixture, and formulation into an efficacious therapeutic agent.

As used herein, a “subject” can be a human (i.e., a male or female of any age group, e.g., a pediatric subject (e.g., infant, child, adolescent) or adult subject (e.g., young adult, middle-aged adult or senior adult)), non-human primate (e.g., cynomolgus monkeys, rhesus monkeys), mammal, rat, mouse, cow, horse, pig, sheep, goat, dog, cat and the like. The terms “human”, “subject” and “patient” are used interchangeably herein in reference, e.g., to a mammalian subject, such as a human subject.

“Mammal” includes humans and both domestic animals such as laboratory animals and household pets (e.g., cats, dogs, swine, cattle, sheep, goats, horses, rabbits), and non-domestic animals such as wildlife and the like.

“Optional” or “optionally” means that the subsequently described event of circumstances can or cannot occur, and that the description includes instances where said event or circumstance occurs and instances in which it does not. For example, “optionally substituted aryl” means that the aryl radical can or cannot be substituted and that the description includes both substituted aryl radicals and aryl radicals having no substitution.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, l-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

Often crystallizations produce a solvate of the compound of the invention. As used herein, the term “solvate” refers to an aggregate that comprises one or more molecules of a compound of the invention with one or more molecules of solvent. The solvent can be water, in which case the solvate can be a hydrate. Alternatively, the solvent can be an organic solvent. Thus, the compounds of the present invention can exist as a hydrate, including a monohydrate, dihydrate, hemihydrate, sesquihydrate, trihydrate, tetrahydrate and the like, as well as the corresponding solvated forms. The compound of the invention can be true solvates, while in other cases, the compound of the invention can merely retain adventitious water or be a mixture of water plus some adventitious solvent.

A “pharmaceutical composition” refers to a formulation of a compound of the invention and a medium generally accepted in the art for the delivery of the biologically active compound to mammals, e.g., humans. Such a medium includes all pharmaceutically acceptable carriers, diluents or excipients therefor.

The term “pharmaceutically acceptable” refers to a substance (e.g., an active ingredient or an excipient) that is suitable for use in contact with the tissues and organs of a subject without excessive irritation, allergic response, immunogenicity and toxicity, is commensurate with a reasonable benefit/risk ratio, and is effective for its intended use. A “pharmaceutically acceptable” carrier or excipient of a pharmaceutical composition is also compatible with the other ingredients of the composition.

An “effective amount” refers to a therapeutically effective amount or a prophylactically effective amount. A “therapeutically effective amount” refers to an amount effective, at dosages and for periods of time necessary, to achieve the desired therapeutic result, such as reduced tumor size, increased life span, increased life expectancy, or sufficient to prevent development of, or to alleviate to some extent, or to abrogate, the disease or disorder being treated. The term “therapeutically effective amount” also refers to an amount of a compound that is sufficient to elicit a biological or medical response of a cell, tissue, organ, system, animal or human which is sought by a researcher, veterinarian, medical doctor or clinician. A therapeutically effective amount of a compound can vary according to factors such as the disease state, age, sex, and weight of the subject, and the ability of the compound to elicit a desired response in the subject. Dosage regimens can be adjusted to provide the optimum therapeutic response. A therapeutically effective amount is also one in which any toxic or detrimental effects of the compound are outweighed by the therapeutically beneficial effects. A “prophylactically effective amount” refers to an amount effective, at dosages and for periods of time necessary, to achieve the desired prophylactic result, such as smaller tumors, increased life span, increased life expectancy or prevention of the progression of prostate cancer to a castration-resistant form. Typically, a prophylactic dose is used in subjects prior to or at an earlier stage of disease, so that a prophylactically effective amount can be less than a therapeutically effective amount.

“Treating” or “treatment” as used herein covers the treatment of the disease or condition of interest in a mammal, preferably a human, having the disease or condition of interest, and includes:

1. preventing the disease or condition from occurring in a mammal, in particular, when such mammal is predisposed to the condition but has not yet been diagnosed as having it;
2. inhibiting the disease or condition, i.e., arresting its development;
3. relieving the disease or condition, i.e., causing regression of the disease or condition; or
4. relieving the symptoms resulting from the disease or condition, i.e., relieving pain without addressing the underlying disease or condition. As used herein, the terms “disease”, “disorder”, and “condition” can be used interchangeably or can be different in that the particular malady or condition cannot have a known causative agent (so that etiology has not yet been worked out) and it is therefore not yet recognized as a disease but only as an undesirable condition or syndrome, wherein a more or less specific set of symptoms have been identified by clinicians.

“Therapeutic treatment” contemplates an action that occurs while a subject is suffering from the specific disease, disorder or condition, which reduces the severity of the disease, disorder or condition, or retards or slows the progression of the disease, disorder or condition. “Prophylactic treatment” contemplates an action that occurs before a subject begins to suffer from the specified disease, disorder or condition.

The terms “prevent”, “preventing”, and “prevention” include delaying or precluding the onset of a disease or disorder, precluding a subject from acquiring a disease or disorder, and reducing a subject's risk of acquiring a disease or disorder.

The compounds of the invention, or their pharmaceutically acceptable salts can contain one or more asymmetric centers and can thus give rise to enantiomers, diastereomers, and other stereoisomeric forms that can be defined, in terms of absolute stereochemistry, as (R)- or (S)- or, as (D)- or (L)- for amino acids. The present invention is meant to include all such possible isomers, as well as their racemic and optically pure forms whether or not they are specifically depicted herein. Optically active (+) and (−), (R)- and (S)-, or (D)- and (L)-isomers can be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present invention contemplates various stereoisomers and mixtures thereof and includes “enantiomers”, which refers to two stereoisomers whose molecules are nonsuperimposable mirror images of one another.

A “tautomer” refers to a proton shift from one atom of a molecule to another atom of the same molecule. The present invention includes tautomers of any said compounds.

A “counterion” or “anionic counterion” is a negatively charged group associated with a cationic quaternary amino group in order to maintain electronic neutrality. Exemplary counterions include halide ions (e.g., F⁻, Cl⁻, Br⁻, I⁻), NO₃⁻, ClO₄⁻, OH⁻, H₂PO₄⁻, HSO₄⁻, SO₄²⁻ sulfonate ions (e.g. methanesulfonate, trifluoromethanesulfonate, p-toluenesulfonate, benzenesulfonate, 10-camphorsulfonate, naphthalene-2-sulfonate, naphthalene-1-sulfonic acid-5-sulfonate, ethan-1-sulfonic acid-2-sulfonate, and the like), and carboxylate ions (e.g., acetate, ethanoate, propanoate, benzoate, glycerate, lactate, tartrate, glycolate, and the like).

The terms “pharmaceutical combination,” “therapeutic combination” or “combination” as used herein, refers to a single dosage form comprising at least two therapeutically active agents, or separate dosage forms comprising at least two therapeutically active agents together or separately for use in combination therapy. For example, one therapeutically active agent may be formulated into one dosage form and the other therapeutically active agent may be formulated into a single or different dosage forms. For example, one therapeutically active agent may be formulated into a solid oral dosage form whereas the second therapeutically active agent may be formulated into a solution dosage form for parenteral administration.

The chemical naming protocol and structure diagrams used herein are a modified form of the I.U.P.A.C. nomenclature system, using the ACD/Name Version 9.07 software program, ChemDraw Ultra Version 11.0.1 and/or ChemDraw Ultra Version 14.0 software naming program (CambridgeSoft). For complex chemical names employed herein, a substituent group is named before the group to which it attaches. For example, cyclopropylmethyl comprises a methyl backbone with cyclopropyl substituent. Except as described below, all bonds are identified in the chemical structure diagrams herein, except for some carbon atoms, which are assumed to be bonded to sufficient hydrogen atoms to complete the valency.

### II. Compounds of the Present Disclosure

One embodiment of the present disclosure relates to novel tetrazolone substituted neuroactive steroid compounds of Formula (AI) or (AII):

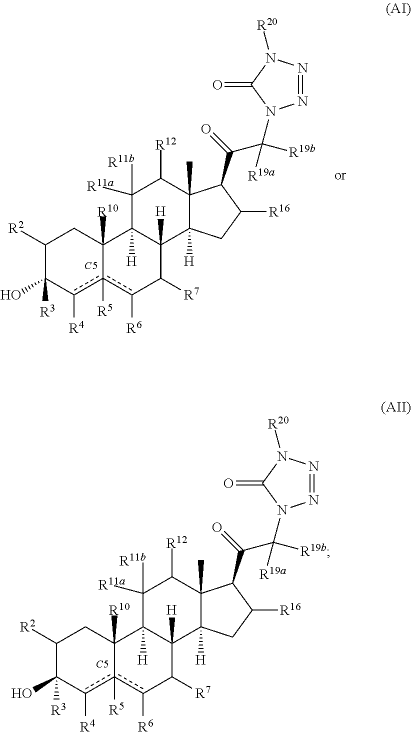

or a pharmaceutically acceptable salt, solvate, ester, or prodrug thereof; wherein:
[symbol] represents a single or double bond;
when one of [symbol] is a double bond, the other [symbol] is a single bond and R⁵ is absent;
when both of [symbol] are single bonds, then R⁵ is hydrogen;
R², R⁴, R⁶, R⁷, R^{11a}, R^{11b}, R¹², and R¹⁶ are each independently hydrogen, halogen, cyano, nitro, substituted or unsubstituted alkyl, substituted or unsubstituted alkenyl, substituted or unsubstituted alkynyl, substituted or unsubstituted carbocylyl, substituted or unsubstituted heterocyclyl, substituted or unsubstituted aryl, substituted or unsubstituted heteroaryl, —OR^{A}, —C(═O)R^{A}, —C(═O)OR^{A}, —OC(═O)R^{A}, —OC(═O)OR^{A}, —C(═O)NR^{A}R^{A}, —NR^{A}R^{A}, —NR^{A}C(═O)R^{A}, —OC(═O)NR^{A}R^{A}, —NR^{A}C(═O)OR^{A}, —NR^{A}C(═O)NR^{A}R^{A}, —SR^{A}, —S(═O)R^{A}, —S(═O)₂R^{A}, —S(═O)₂OR^{A}, —OS(═O)₂R^{A}, —S(═O)₂NR^{A}R^{A}, or —NR^{A}S(═O)₂R^{A}, wherein R^{A} is independently hydrogen, substituted or unsubstituted alkyl, substituted or unsubstituted alkenyl, substituted or unsubstituted alkynyl, substituted or unsubstituted carbocylyl, substituted or unsubstituted heterocylyl, substituted or unsubstituted aryl, substituted or unsubstituted heteroaryl, haloalkyl, an oxygen protecting group when attached to oxygen, a sulfur protecting group when attached to sulfur, or a nitrogen protecting group when attached to nitrogen, or two R^{A} groups can be taken together with the atoms to which they are attached to, to form a substituted or unsubstituted heterocylyl or heteroaryl ring;
alternatively, R^{11a} and R^{11b}, taken together with the carbon atom to which they are both attached, form a 3-8 membered saturated, partially saturated, or unsaturated ring optionally containing one or more heteroatoms as a ring member selected from N, O, or S (e.g., carbocycyl or heterocyclyl ring); or R^{11a} and R^{11b} are joined to form an oxo (═O) group;
R³ is hydrogen, substituted or unsubstituted alkyl, substituted or unsubstituted alkenyl, substituted or unsubstituted alkynyl, substituted or unsubstituted carbocylyl, substituted or unsubstituted heterocyclyl, substituted or unsubstituted aryl, substituted or unsubstituted heteroaryl;
R¹⁰ is hydrogen, halogen, cyano, or substituted or unsubstituted alkyl;
R^{19a} is hydrogen, substituted or unsubstituted alkyl, or —OR^{A19}, wherein R^{A19} is hydrogen, substituted or unsubstituted alkyl, substituted or unsubstituted alkenyl, substituted or unsubstituted alkynyl, or substituted or unsubstituted carbocylyl;
R^{19b} is hydrogen or substituted or unsubstituted alkyl;

alternatively, R^{19a} and R^{19b} are joined to form an oxo (═O) group, or R^{19a} and R^{19b} together with the carbon atom to which they are both attached, form a 3-8 membered saturated, partially saturated, or unsaturated ring optionally containing one or more heteroatoms as a ring member selected from N, O, or S (e.g., carbocycyl or heterocyclyl ring); and
R²⁰ is hydrogen, alkyl, haloalkyl, cycloalkyl, cycloalkylalkyl, heterocyclyl, aryl, C₆-C₁₂ aryl-C₁-C₆ alkyl-, heteroaryl, or heteroaryl-C₁-C₆ alkyl-, wherein alkyl, haloalkyl, cycloalkyl, cycloalkylalkyl, heterocyclyl, aryl, C₆-C₁₂ aryl-C₁-C₆ alkyl-, heteroaryl, and heteroaryl-C₁-C₆ alkyl- can be optionally substituted with substituents selected from substituted or unsubstituted alkyl, substituted or unsubstituted alkenyl, substituted or unsubstituted alkynyl, substituted or unsubstituted carbocylyl, substituted or unsubstituted heterocylyl, haloalkyl, substituted or unsubstituted —C₆-C₁₂ aryl, substituted or unsubstituted 5-12 membered heteroaryl, halogen, nitro, cyano, —OR^{A}, —C(═O)R^{A}, —C(═O)OR^{A}, —OC(═O)R^{A}, —OC(═O)OR^{A}, —C(═O)NR^{A}R^{A}, —NR^{A}R^{A}, —NR^{A}C(═O)R^{A}, —OC(═O)NR^{A}R^{A}, —NR^{A}C(═O)OR^{A}, —NR^{A}C(═O)NR^{A}R^{A}, —SR^{A}, —S(═O)R^{A}, —S(═O)₂R^{A}, —S(═O)₂OR^{A}, —OS(═O)₂R^{A}, —S(═O)₂NR^{A}R^{A}, or —NR^{A}S(═O)₂R^{A}, wherein R^{A} is independently hydrogen or substituted or unsubstituted alkyl, substituted or unsubstituted alkenyl, substituted or unsubstituted alkynyl, substituted or unsubstituted carbocylyl, substituted or unsubstituted heterocylyl, substituted or unsubstituted aryl, substituted or unsubstituted heteroaryl, haloalkyl, an oxygen protecting group when attached to oxygen, a sulfur protecting group when attached to sulfur, or a nitrogen protecting group when attached to nitrogen, or two R^{A} groups can be taken together with the atoms to which they are attached to, to form a substituted or unsubstituted heterocylyl or heteroaryl ring.

One embodiment of the present disclosure relates to novel tetrazolone substituted neuroactive steroid compounds of Formula (I) or (II):

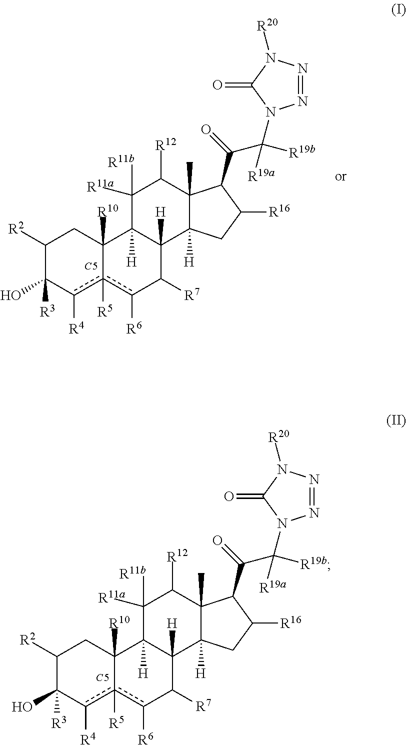

or a pharmaceutically acceptable salt, solvate, ester, or prodrug thereof; wherein:
[symbol] represents a single or double bond;
when one of [symbol] is a double bond, the other [symbol] is a single bond and R⁵ is absent;
when both of [symbol] are single bonds, then R⁵ is hydrogen;
R², R⁴, R⁶, R⁷, R^{11a}, R^{11b}, R¹², and R¹⁶ are each independently hydrogen, halogen, cyano, nitro, substituted or unsubstituted alkyl, substituted or unsubstituted alkenyl, substituted or unsubstituted alkynyl, substituted or unsubstituted carbocylyl, substituted or unsubstituted heterocyclyl, substituted or unsubstituted aryl, substituted or unsubstituted heteroaryl, —OR^{A}, —C(═O)R^{A}, —C(═O)OR^{A}, —OC(═O)R^{A}, —OC(═O)OR^{A}, —C(═O)NR^{A}R^{A}, —NR^{A}R^{A}, —NR^{A}C(═O)R^{A}, —OC(═O)NR^{A}R^{A}, —NR^{A}C(═O)OR^{A}, —NR^{A}C(═O)NR^{A}R^{A}, —SR^{A}, —S(═O)R^{A}, —S(═O)₂R^{A}, —S(═O)₂OR^{A}, —OS(═O)₂R^{A}, —S(═O)₂NR^{A}R^{A}, or —NR^{A}S(═O)₂R^{A}, wherein R^{A} is independently hydrogen, substituted or unsubstituted alkyl, substituted or unsubstituted alkenyl, substituted or unsubstituted alkynyl, substituted or unsubstituted carbocylyl, substituted or unsubstituted heterocylyl, substituted or unsubstituted aryl, substituted or unsubstituted heteroaryl, haloalkyl, an oxygen protecting group when attached to oxygen, a sulfur protecting group when attached to sulfur, or a nitrogen protecting group when attached to nitrogen, or two R^{A} groups can be taken together with the atoms to which they are attached to, to form a substituted or unsubstituted heterocylyl or heteroaryl ring;
alternatively, R^{11a} and R^{11b}, taken together with the carbon atom to which they are both attached, form a 3-8 membered saturated, partially saturated, or unsaturated ring optionally containing one or more heteroatoms as a ring member selected from N, O, or S (e.g., carbocycyl or heterocyclyl ring); or R^{11a} and R^{11b} are joined to form an oxo (═O) group;
R³ is hydrogen, substituted or unsubstituted alkyl, substituted or unsubstituted alkenyl, substituted or unsubstituted alkynyl, substituted or unsubstituted carbocylyl, substituted or unsubstituted heterocyclyl, substituted or unsubstituted aryl, substituted or unsubstituted heteroaryl;
R¹⁰ is hydrogen or substituted or unsubstituted alkyl;
R^{19a} is hydrogen, substituted or unsubstituted alkyl, or —OR^{A19}, wherein R^{A19} is hydrogen, substituted or unsubstituted alkyl, substituted or unsubstituted alkenyl, substituted or unsubstituted alkynyl, or substituted or unsubstituted carbocylyl;
R^{19b} is hydrogen or substituted or unsubstituted alkyl;
alternatively, R^{19a} and R^{19b} are joined to form an oxo (═O) group, or R^{19a} and R^{19b} together with the carbon atom to which they are both attached, form a 3-8 membered saturated, partially saturated, or unsaturated ring optionally containing one or more heteroatoms as a ring member selected from N, O, or S (e.g., carbocycyl or heterocyclyl ring); and
R²⁰ is hydrogen, alkyl, haloalkyl, cycloalkyl, cycloalkylalkyl, heterocyclyl, aryl, C₆-C₁₂ aryl-C₁-C₆ alkyl-, heteroaryl, or heteroaryl-C₁-C₆ alkyl-, wherein alkyl, haloalkyl, cycloalkyl, cycloalkylalkyl, heterocyclyl, aryl, C₆-C₁₂ aryl-C₁-C₆ alkyl-, heteroaryl, and heteroaryl-C₁-C₆ alkyl- can be optionally substituted with substituents selected from substituted or unsubstituted alkyl, substituted or unsubstituted alkenyl, substituted or unsubstituted alkynyl, substituted or unsubstituted carbocylyl, substituted or unsubstituted heterocylyl, haloalkyl, substituted or unsubstituted —C₆-C₁₂ aryl, substituted or unsubstituted 5-12 membered heteroaryl, halogen, nitro, cyano, —OR^{A}, —C(═O)R^{A}, —C(═O)OR^{A}, —OC(═O)R^{A}, —OC(═O)OR^{A}, —C(═O)NR^{A}R^{A}, —NR^{A}R^{A}, —NR^{A}C(═O)R^{A}, —OC(═O)NR^{A}R^{A}, —NR^{A}C(═O)OR^{A}, —NR^{A}C(═O)NR^{A}R^{A}, —SR^{A}, —S(═O)R^{A}, —S(═O)₂R^{A}, —S(═O)₂OR^{A}, —OS(═O)₂R^{A}, —S(═O)₂NR^{A}R^{A}, or —NR^{A}S(═O)₂R^{A}, wherein R^{A} is independently hydrogen or substituted or unsubstituted alkyl, substituted or unsubstituted alkenyl, substituted or unsubstituted alkynyl, substituted or unsubstituted carbocylyl, substituted or unsubstituted heterocylyl, substituted or unsubstituted aryl, substituted or unsubstituted heteroaryl, haloalkyl, an oxygen protecting group when attached to oxygen, a sulfur protecting group when attached to sulfur, or a nitrogen protecting group when attached to nitrogen, or two R^{A} groups can be taken together with the atoms to which they are attached to, to form a substituted or unsubstituted heterocylyl or heteroaryl ring.

In one embodiment, R³ is optionally substituted —C₁₋₆ alkyl. In another embodiment, R³ is —C₁₋₆ alkyl optionally substituted with one or more of alkoxy or halogen. In certain embodiments, R³ is C₁₋₆ alkyl optionally substituted with alkoxy or one or two halo groups (e.g., fluoro), and R²⁰ is hydrogen, substituted or unsubstituted —C₁₋₆ alkyl, substituted or unsubstituted —C₃₋₁₂ cycloalkyl, substituted or unsubstituted C₃₋₁₂ cycloalkyl(C₁₋₁₆ alkyl)-, or substituted or unsubstituted aryl or heteroaryl. In certain embodiments, R³ is methyl, R²⁰ is hydrogen, substituted or unsubstituted —C₁₋₆ alkyl, substituted or unsubstituted —C₃₋₁₂ cycloalkyl, substituted or unsubstituted C₃₋₁₂ cycloalkyl(C₁₋₁₆ alkyl)-, or substituted or unsubstituted aryl or heteroaryl.

In one embodiment, R²⁰ is hydrogen, substituted or unsubstituted —C₁₋₆ alkyl, substituted or unsubstituted —C₃₋₁₂ cycloalkyl, substituted or unsubstituted C₃₋₁₂ cycloalkyl(C₁₋₁₆ alkyl)-, or substituted or unsubstituted aryl or heteroaryl.

In one embodiment, the substituents are selected from halogen, cyano, nitro, hydroxy, alkyl, haloalkyl, alkenyl, alkynyl, cycloalkyl, cycloalkylalkyl, —(C₁-C₆ alkyl)-OH, carbocylyl, heterocyclyl, aryl, C₆-C₁₂ aryl-(C₁-C₆ alkyl)-, heteroaryl, heteroaryl-(C₁-C₆ alkyl)-, —OR^{A}, —(C₁-C₆ alkyl)-OR^{A}, —C(═O)R^{A}, —C(═O)OR^{A}, —OC(═O)R^{A}, —OC(═O)OR^{A}, —C(═O)NR^{A}R^{A}, —NR^{A}R^{A}, —NR^{A}C(═O)R^{A}, —OC(═O)NR^{A}R^{A}, —NR^{A}C(═O)OR^{A}, —NR^{A}C(═O)NR^{A}R^{A}, —SR^{A}, —S(═O)R^{A}, —S(═O)₂R^{A}, —S(═O)₂OR^{A}, —OS(═O)₂R^{A}, —S(═O)₂NR^{A}R^{A}, or —NR^{A}S(═O)₂R^{A}, wherein R^{A} is independently hydrogen, alkyl, haloalkyl, alkenyl, alkynyl, cycloalkyl, cycloalkylalkyl, carbocylyl, heterocylyl, aryl, C₃₋₁₂ aryl-(C₁₋₁₆ alkyl)-, heteroaryl, heteroaryl-(C₁-C₆ alkyl)-, an oxygen protecting group when attached to an oxygen, a sulfur protecting group when attached to a sulfur, or a nitrogen protecting group when attached to a nitrogen; or two R^{A} groups can be taken together with the atoms to which they are attached to, to form a substituted or unsubstituted heterocylyl or heteroaryl ring.

In one embodiment, the substituents are selected from halogen, cyano, nitro, hydroxy, alkyl, haloalkyl, alkenyl, alkynyl, cycloalkyl, cycloalkylalkyl, —(C₁-C₆ alkyl)-OH, carbocylyl, heterocyclyl, aryl, C₆-C₁₂ aryl-(C₁-C₆ alkyl)-, heteroaryl, heteroaryl-(C₁-C₆ alkyl)-, —OR^{A}, —(C₁-C₆ alkyl)-OR^{A}, —C(═O)R^{A}, —C(═O)OR^{A}, —OC(═O)R^{A}, —OC(═O)OR^{A}, —C(═O)NR^{A}R^{A}, —NR^{A}R^{A}, —NR^{A}C(═O)R^{A}, —OC(═O)NR^{A}R^{A}, —NR^{A}C(═O)OR^{A}, —NR^{A}C(═O)NR^{A}R^{A}, —SR^{A}, —S(═O)R^{A}, —S(═O)₂R^{A}, —S(═O)₂OR^{A}, —OS(═O)₂R^{A}, —S(═O)₂NR^{A}R^{A}, or —NR^{A}S(═O)₂R^{A}, wherein R^{A} is independently hydrogen, alkyl, haloalkyl, alkenyl, alkynyl, cycloalkyl, cycloalkylalkyl, carbocylyl, heterocylyl, aryl, C₆-C₁₂ aryl-(C₁-C₆ alkyl)-, heteroaryl, heteroaryl-(C₁-C₆ alkyl)-, an oxygen protecting group when attached to an oxygen, a sulfur protecting group when attached to a sulfur, or a nitrogen protecting group when attached to a nitrogen; or two R^{A} groups can be taken together with the atoms to which they are attached to, to form a heterocylyl or heteroaryl ring.

In one embodiment, the substituents are selected from fluoro, chloro, bromo, cyano, nitro, hydroxy, methoxy, ethoxy, propoxy, isopropoxy, methyl, ethyl, propyl, isopropyl, butyl, i-butyl, s-butyl, t-butyl, —CH₂CN, —CH₂CH₂CN, —CH₂F, —CHF₂, —CF₃, —CH₂OCH₃, —CH₂SCH₃, -methylhydroxy, morpholine, pyrrolidine, piperidine, piperazine, phenyl, benzyl, pyridine, pyrimidine, oxazole, pyrazole, cyclopropyl, cyclobutyl, cyclopentyl, cyclohexyl, —OCH₂F, —OCHF₂, —OCF₃, -ethylmethoxy, -methylcyclopropyl, —OR^{A}, —C(═O)R^{A}, —C(═O)OR^{A}, —OC(═O)R^{A}, —OC(═O)OR^{A}, —C(═O)NR^{A}R^{A}, —NR^{A}R^{A}, —NR^{A}C(═O)R^{A}, —OC(═O)NR^{A}R^{A}, —NR^{A}C(═O)OR^{A}, —NR^{A}C(═O)NR^{A}R^{A}, —SR^{A}, —S(═O)R^{A}, —S(═O)₂R^{A}, —S(═O)₂OR^{A}, —OS(═O)₂R^{A}, —S(═O)₂NR^{A}R^{A}, or —NR^{A}S(═O)₂R^{A}, wherein R^{A} is independently hydrogen, methyl, ethyl, propyl, isopropyl, cyclopropyl, cyclobutyl, cyclopentyl, cyclohexyl, —CH₂F, —CHF₂, —CF₃, phenyl, benzyl, pyridine, pyrimidine, -ethylmethoxy, or -methylcyclopropyl, or two R^{A} groups can be taken together with the atoms to which they are attached to, to form a heterocylyl or heteroaryl ring.

In one embodiment, the substituents are selected from fluoro, chloro, bromo, cyano, nitro, hydroxy, methoxy, ethoxy, propoxy, isopropoxy, methyl, ethyl, propyl, isopropyl, —CH₂F, —CHF₂, —CF₃, -methylhydroxy, morpholine, pyrrolidine, piperidine, piperazine, phenyl, benzyl, pyridine, pyrimidine, cyclopropyl, cyclobutyl, cyclopentyl, cyclohexyl, —OCH₂F, —OCHF₂, —OCF₃, -ethylmethoxy, -methylcyclopropyl, —OR^{A}, —C(═O)R^{A}, —C(═O)OR^{A}, —OC(═O)R^{A}, —OC(═O)OR^{A}, —C(═O)NR^{A}R^{A}, —NR^{A}R^{A}, —NR^{A}C(═O)R^{A}, —OC(═O)NR^{A}R^{A}, —NR^{A}C(═O)OR^{A}, —NR^{A}C(═O)NR^{A}R^{A}, —SR^{A}, —S(═O)R^{A}, —S(═O)₂R^{A}, —S(═O)₂OR^{A}, —OS(═O)₂R^{A}, —S(═O)₂NR^{A}R^{A}, or —NR^{A}S(═O)₂R^{A}, wherein R^{A} is independently hydrogen, methyl, ethyl, propyl, isopropyl, cyclopropyl, cyclobutyl, cyclopentyl, cyclohexyl, —CH₂F, —CHF₂, —CF₃, phenyl, benzyl, pyridine, pyrimidine, -ethylmethoxy, or -methylcyclopropyl, or two R^{A} groups can be taken together with the atoms to which they are attached to, to form a heterocylyl or heteroaryl ring.

In one embodiment of the compounds of the present invention, both of [symbol] are single bonds and R⁵ and R⁴ are in an alpha configuration. In one embodiment, both of [symbol] are single bonds and R⁵ and R⁴ are in a beta configuration. In one embodiment, both of [symbol] are single bonds and R⁵ and R⁶ are in an alpha configuration. In one embodiment, both of [symbol] are single bonds and R⁵ and R⁶ are in a beta configuration. In one embodiment, both [symbol] are single bonds and R⁴ and R⁶ are hydrogen. In one embodiment, on [symbol] is a single bond, and at least one of R⁴ or R⁶ is hydrogen, substituted or unsubstituted —C₁₋₆ alkyl, halogen, —CH₃, or —CF₃. In one embodiment, one [symbol] is a single bond, and at least one of R⁴ or R⁶ is F.

It is understood, based on the aforementioned description, that the compounds (e.g., steroids) of Formula (AI), (I), (AII), or (II) encompass tetrazolone substituted neuroactive steroids wherein the A/B ring system of the compound is cis (as provided in Formula (I-A) and Formula (II-A)), wherein the A/B ring system of the compound is trans (as provided in Formula (I-B) and Formula (II-B)), and the A or B ring of the compound comprises a double bond (as provided in Formula (I-C), (I-D) and Formula (II-C), (II-D)):

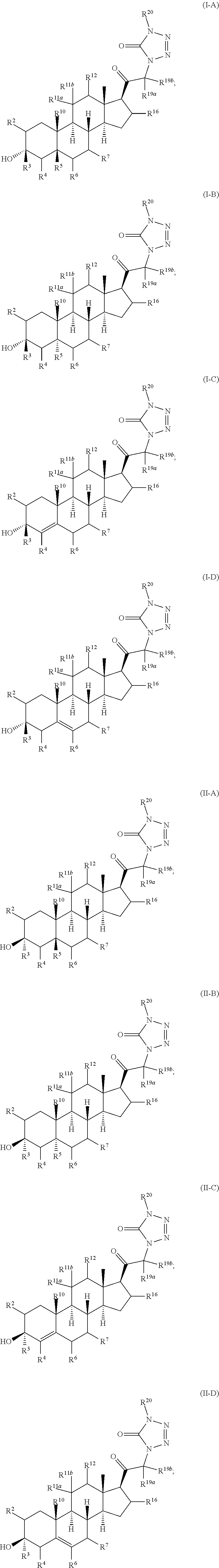

or a pharmaceutically acceptable salt, solvate, ester, or prodrug thereof.

In one embodiment, the present disclosure relates to novel tetrazolone substituted neuroactive steroid compounds of Formula (I-E1) or (I-F1):

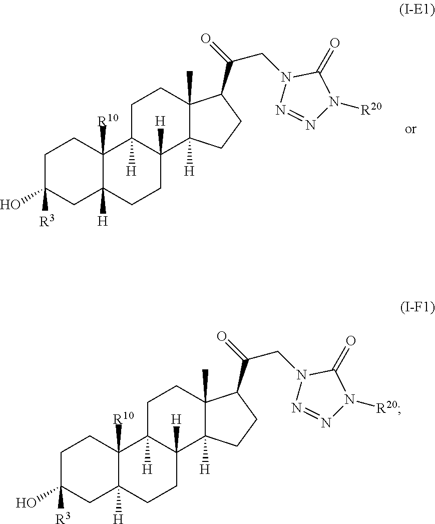

or a pharmaceutically acceptable salt, solvate, ester, or prodrug thereof, wherein:
R³ is hydrogen, —C₁₋₆ alkyl, —C₂₋₆ alkenyl, or —C₂₋₆ alkynyl, each optionally substituted with one to three groups selected from halogen or —C₁₋₆ alkoxy;
R¹⁰ is hydrogen, halogen, cyano, or —C₁₋₆ alkyl wherein —C₁₋₆ alkyl is optionally substituted with halogen or —C₁₋₃ alkoxy; and
R²⁰ is hydrogen, —C₁₋₆ alkyl, —C₂₋₆ alkenyl, —C₂₋₆ alkynyl, —C₁₋₆ haloalkyl, —C₃₋₁₂ cycloalkyl, C₃₋₁₂ cycloalkyl-C₁-C₆ alkyl-, 3-12 membered heterocyclyl, —C₆-C₁₂ aryl, C₆-C₁₂ aryl-C₁-C₆ alkyl-, heteroaryl-C₁-C₆ alkyl-, or 5-12 membered heteroaryl, each of which can be optionally substituted with halogen, nitro, cyano, —C₁₋₆ alkyl, —C₂₋₆ alkenyl, —C₂₋₆ alkynyl, —C₃₋₁₂ cycloalkyl, 3-12 membered heterocylyl, —C₆-C₁₂ aryl, 5-12 membered heteroaryl, —OR^{A}, —C(═O)R^{A}, —C(═O)OR^{A}, —OC(═O)R^{A}, —OC(═O)OR^{A}, —C(═O)NR^{A}R^{A}, —NR^{A}R^{A}, —NR^{A}C(═O)R^{A}, —OC(═O)NR^{A}R^{A}, —NR^{A}C(═O)OR^{A}, —NR^{A}C(═O)NR^{A}R^{A}, —SR^{A}, —S(═O)R^{A}, —S(═O)₂R^{A}, —S(═O)₂OR^{A}, —OS(═O)₂R^{A}, —S(═O)₂NR^{A}R^{A}, or —NR^{A}S(═O)₂R^{A};
wherein R^{A} is independently hydrogen, alkyl, haloalkyl, alkenyl, alkynyl, cycloalkyl, cycloalkylalkyl, carbocylyl, heterocylyl, aryl, C₆-C₁₂ aryl-C₁-C₆ alkyl-, heteroaryl, heteroaryl-C₁-C₆ alkyl-, an oxygen protecting group when attached to oxygen, a sulfur protecting group when attached to sulfur, or a nitrogen protecting group when attached to nitrogen; or two R^{A} groups can be taken together with the atoms to which they are attached to, to form a substituted or unsubstituted heterocylyl or heteroaryl ring;
wherein the substituents are selected from fluoro, chloro, bromo, cyano, nitro, hydroxy, methoxy, ethoxy, propoxy, isopropoxy, methyl, ethyl, propyl, isopropyl, butyl, i-butyl, s-butyl, t-butyl, —CH₂CN, —CH₂CH₂CN, —CH₂F, —CHF₂, —CF₃, —CH₂OCH₃, —CH₂SCH₃, -methylhydroxy, morpholine, pyrrolidine, piperidine, piperazine, phenyl, benzyl, pyridine, pyrimidine, oxazole, pyrazole, cyclopropyl, cyclobutyl, cyclopentyl, cyclohexyl, —OCH₂F, —OCHF₂, —OCF₃, -ethylmethoxy, -methylcyclopropyl, —OR^{A}, —C(═O)R^{A}, —C(═O)OR^{A}, —OC(═O)R^{A}, —OC(═O)OR^{A}, —C(═O)NR^{A}R^{A}, —NR^{A}R^{A}, —NR^{A}C(═O)R^{A}, —OC(═O)NR^{A}R^{A}, —NR^{A}C(═O)OR^{A}, —NR^{A}C(═O)NR^{A}R^{A}, —SR^{A}, —S(═O)R^{A}, —S(═O)₂R^{A}, —S(═O)₂OR^{A}, —OS(═O)₂R^{A}, —S(═O)₂NR^{A}R^{A}, or —NR^{A}S(═O)₂R^{A}, wherein R^{A} is independently hydrogen, methyl, ethyl, propyl, isopropyl, cyclopropyl, cyclobutyl, cyclopentyl, cyclohexyl, —CH₂F, —CHF₂, —CF₃, phenyl, benzyl, pyridine, pyrimidine, -ethylmethoxy, or -methylcyclopropyl, or two R^{A} groups can be taken together with the atoms to which they are attached to, to form a heterocylyl or heteroaryl ring.

In one embodiment, the present disclosure relates to novel tetrazolone substituted neuroactive steroid compounds of Formula (I-E) or (I-F):

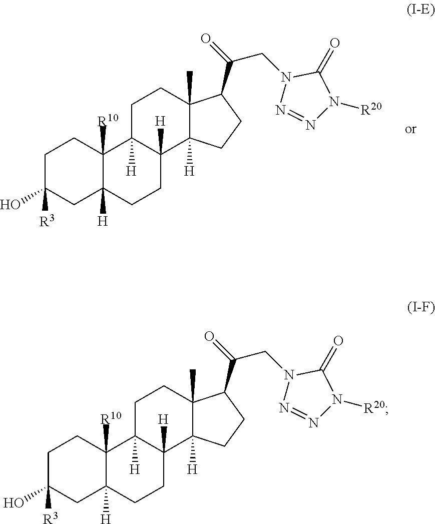

or a pharmaceutically acceptable salt, solvate, ester, or prodrug thereof, wherein:
R³ is hydrogen, —C₁₋₆ alkyl, —C₂₋₆ alkenyl, or —C₂₋₆ alkynyl, each optionally substituted with one to three groups selected from halogen or —C₁₋₆ alkoxy;
R¹⁰ is hydrogen or —C₁₋₆ alkyl wherein —C₁₋₆ alkyl is optionally substituted with halogen or —C₁₋₃ alkoxy; and
R²⁰ is hydrogen, —C₁₋₆ alkyl, —C₂₋₆ alkenyl, —C₂₋₆ alkynyl, —C₁₋₆ haloalkyl, —C₃₋₁₂ cycloalkyl, C₃₋₁₂ cycloalkyl-C₁-C₆ alkyl-, 3-12 membered heterocyclyl, —C₆-C₁₂ aryl, C₆-C₁₂ aryl-C₁-C₆ alkyl-, heteroaryl-C₁-C₆ alkyl-, or 5-12 membered heteroaryl, each of which can be optionally substituted with halogen, nitro, cyano, —C₁₋₆ alkyl, —C₂₋₆ alkenyl, —C₂₋₆ alkynyl, —C₃₋₁₂ cycloalkyl, 3-12 membered heterocylyl, —C₆-C₁₂ aryl, 5-12 membered heteroaryl, —OR^{A}, —C(═O)R^{A}, —C(═O)OR^{A}, —OC(═O)R^{A}, —OC(═O)OR^{A}, —C(═O)NR^{A}R^{A}, —NR^{A}R^{A}, —NR^{A}C(═O)R^{A}, —OC(═O)NR^{A}R^{A}, —NR^{A}C(═O)OR^{A}, —NR^{A}C(═O)NR^{A}R^{A}, —SR^{A}, —S(═O)R^{A}, —S(═O)₂R^{A}, —S(═O)₂OR^{A}, —OS(═O)₂R^{A}, —S(═O)₂NR^{A}R^{A}, or —NR^{A}S(═O)₂R^{A};

wherein R^{A} is independently hydrogen, alkyl, haloalkyl, alkenyl, alkynyl, cycloalkyl, cycloalkylalkyl, carbocylyl, heterocylyl, aryl, C₆-C₁₂ aryl-C₁-C₆ alkyl-, heteroaryl, heteroaryl-C₁-C₆ alkyl-, an oxygen protecting group when attached to oxygen, a sulfur protecting group when attached to sulfur, or a nitrogen protecting group when attached to nitrogen; or two R^{A} groups can be taken together with the atoms to which they are attached to, to form a substituted or unsubstituted heterocylyl or heteroaryl ring;
wherein the substituents are selected from fluoro, chloro, bromo, cyano, nitro, hydroxy, methoxy, ethoxy, propoxy, isopropoxy, methyl, ethyl, propyl, isopropyl, —CH₂F, —CHF₂, —CF₃, -methylhydroxy, morpholine, pyrrolidine, piperidine, piperazine, phenyl, benzyl, pyridine, pyrimidine, cyclopropyl, cyclobutyl, cyclopentyl, cyclohexyl, —OCH₂F, —OCHF₂, —OCF₃, -ethylmethoxy, -methylcyclopropyl, —OR^{A}, —C(═O)R^{A}, —C(═O)OR^{A}, —OC(═O)R^{A}, —OC(═O)OR^{A}, —C(═O)NR^{A}R^{A}, —NR^{A}R^{A}, —NR^{A}C(═O)R^{A}, —OC(═O)NR^{A}R^{A}, —NR^{A}C(═O)OR^{A}, —NR^{A}C(═O)NR^{A}R^{A}, —SR^{A}, —S(═O)R^{A}, —S(═O)₂R^{A}, —S(═O)₂OR^{A}, —OS(═O)₂R^{A}, —S(═O)₂NR^{A}R^{A}, or —NR^{A}S(═O)₂R^{A}, wherein R^{A} is independently hydrogen, methyl, ethyl, propyl, isopropyl, cyclopropyl, cyclobutyl, cyclopentyl, cyclohexyl, —CH₂F, —CHF₂, —CF₃, phenyl, benzyl, pyridine, pyrimidine, -ethylmethoxy, or -methylcyclopropyl, or two R^{A} groups can be taken together with the atoms to which they are attached to, to form a heterocylyl or heteroaryl ring.

In one embodiment, the present disclosure relates to novel tetrazolone substituted neuroactive steroid compounds of Formula (I-G1):

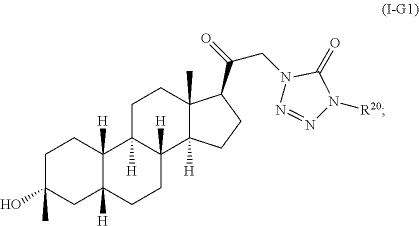

or a pharmaceutically acceptable salt, solvate, ester, or prodrug thereof, wherein:
R²⁰ is hydrogen, —C₁₋₆ alkyl, —C₃₋₁₂ cycloalkyl, C₃₋₁₂ cycloalkyl-C₁₋₆ alkyl-, —C₆-C₁₂ aryl, or 5-12 membered heteroaryl, each of which can be optionally substituted with halogen, nitro, cyano, —C₁₋₆ alkyl, —C₂₋₆ alkenyl, —C₂₋₆ alkynyl, —C₃₋₁₂ cycloalkyl, 3-12 membered heterocylyl, —C₆-C₁₂ aryl, 5-12 membered heteroaryl, —OR^{A}, —C(═O)R^{A}, —C(═O)OR^{A}, —OC(═O)R^{A}, —OC(═O)OR^{A}, —C(═O)NR^{A}R^{A}, —NR^{A}R^{A}, —NR^{A}C(═O)R^{A}, —OC(═O)NR^{A}R^{A}, —NR^{A}C(═O)OR^{A}, —NR^{A}C(═O)NR^{A}R^{A}, —SR^{A}, —S(═O)R^{A}, —S(═O)₂R^{A}, —S(═O)₂OR^{A}, —OS(═O)₂R^{A}, —S(═O)₂NR^{A}R^{A}, or —NR^{A}S(═O)₂R^{A};
wherein R^{A} is independently hydrogen, alkyl, haloalkyl, alkenyl, alkynyl, cycloalkyl, cycloalkylalkyl, carbocylyl, heterocylyl, aryl, C₆-C₁₂ aryl-C₁-C₆ alkyl-, heteroaryl, heteroaryl-C₁-C₆ alkyl-, an oxygen protecting group when attached to oxygen, a sulfur protecting group when attached to sulfur, or a nitrogen protecting group when attached to nitrogen; or two R^{A} groups can be taken together with the atoms to which they are attached to, to form a heterocylyl or heteroaryl ring;
wherein the substituents are selected from fluoro, chloro, bromo, cyano, nitro, hydroxy, methoxy, ethoxy, propoxy, isopropoxy, methyl, ethyl, propyl, isopropyl, butyl, i-butyl, s-butyl, t-butyl, —CH₂CN, —CH₂CH₂CN, —CH₂F, —CHF₂, —CF₃, —CH₂OCH₃, —CH₂SCH₃, -methylhydroxy, morpholine, pyrrolidine, piperidine, piperazine, phenyl, benzyl, pyridine, pyrimidine, oxazole, pyrazole, cyclopropyl, cyclobutyl, cyclopentyl, cyclohexyl, —OCH₂F, —OCHF₂, —OCF₃, -ethylmethoxy, -methylcyclopropyl, —OR^{A}, —C(═O)R^{A}, —C(═O)OR^{A}, —OC(═O)R^{A}, —OC(═O)OR^{A}, —C(═O)NR^{A}R^{A}, —NR^{A}R^{A}, —NR^{A}C(═O)R^{A}, —OC(═O)NR^{A}R^{A}, —NR^{A}C(═O)OR^{A}, —NR^{A}C(═O)NR^{A}R^{A}, —SR^{A}, —S(═O)R^{A}, —S(═O)₂R^{A}, —S(═O)₂OR^{A}, —OS(═O)₂R^{A}, —S(═O)₂NR^{A}R^{A}, or —NR^{A}S(═O)₂R^{A}, wherein R^{A} is independently hydrogen, methyl, ethyl, propyl, isopropyl, cyclopropyl, cyclobutyl, cyclopentyl, cyclohexyl, —CH₂F, —CHF₂, —CF₃, phenyl, benzyl, pyridine, pyrimidine, -ethylmethoxy, or -methylcyclopropyl, or two R^{A} groups can be taken together with the atoms to which they are attached to, to form a heterocylyl or heteroaryl ring.

In one embodiment, the present disclosure relates to novel tetrazolone substituted neuroactive steroid compounds of Formula (I-G):

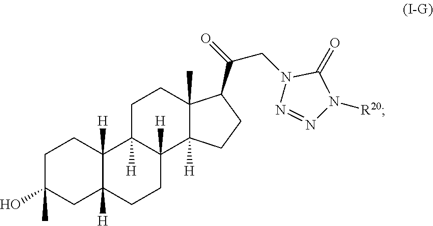

or a pharmaceutically acceptable salt, solvate, ester, or prodrug thereof, wherein:
R²⁰ is hydrogen, —C₁₋₆ alkyl, —C₃₋₁₂ cycloalkyl, C₃₋₁₂ cycloalkyl-C₁₋₆ alkyl-, —C₆-C₁₂ aryl, or 5-12 membered heteroaryl, each of which can be optionally substituted with halogen, nitro, cyano, —C₁₋₆ alkyl, —C₃₋₁₂ cycloalkyl, 3-12 membered heterocylyl, —C₆-C₁₂ aryl, 5-12 membered heteroaryl, —OR^{A}, —C(═O)R^{A}, —C(═O)OR^{A}, —OC(═O)R^{A}, —OC(═O)OR^{A}, —C(═O)NR^{A}R^{A}, —NR^{A}R^{A}, —NR^{A}C(═O)R^{A}, —OC(═O)NR^{A}R^{A}, —NR^{A}C(═O)OR^{A}, —NR^{A}C(═O)NR^{A}R^{A}, —SR^{A}, —S(═O)R^{A}, —S(═O)₂R^{A}, —S(═O)₂OR^{A}, —OS(═O)₂R^{A}, —S(═O)₂NR^{A}R^{A}, or —NR^{A}S(═O)₂R^{A};
wherein R^{A} is independently hydrogen, alkyl, haloalkyl, alkenyl, alkynyl, cycloalkyl, cycloalkylalkyl, carbocylyl, heterocylyl, aryl, C₆-C₁₂ aryl-C₁-C₆ alkyl-, heteroaryl, heteroaryl-C₁-C₆ alkyl-, an oxygen protecting group when attached to oxygen, a sulfur protecting group when attached to sulfur, or a nitrogen protecting group when attached to nitrogen; or two R^{A} groups can be taken together with the atoms to which they are attached to, to form a heterocylyl or heteroaryl ring;
wherein the substituents are selected from fluoro, chloro, bromo, cyano, nitro, hydroxy, methoxy, ethoxy, propoxy, isopropoxy, methyl, ethyl, propyl, isopropyl, —CH₂F, —CHF₂, —CF₃, -methylhydroxy, morpholine, pyrrolidine, piperidine, piperazine, phenyl, benzyl, pyridine, pyrimidine, cyclopropyl, cyclobutyl, cyclopentyl, cyclohexyl, —OCH₂F, —OCHF₂, —OCF₃, -ethylmethoxy, -methylcyclopropyl, —OR^{A}, —C(═O)R^{A}, —C(═O)OR^{A}, —OC(═O)R^{A}, —OC(═O)OR^{A}, —C(═O)NR^{A}R^{A}, —NR^{A}R^{A}, —NR^{A}C(═O)R^{A}, —OC(═O)NR^{A}R^{A}, —NR^{A}C(═O)OR^{A}, —NR^{A}C(═O)NR^{A}R^{A}, —SR^{A}, —S(═O)R^{A}, —S(═O)₂R^{A}, —S(═O)₂OR^{A}, —OS(═O)₂R^{A}, —S(═O)₂NR^{A}R^{A}, or —NR^{A}S(═O)₂R^{A}, wherein R^{A} is independently hydrogen, methyl, ethyl, propyl, isopropyl, cyclopropyl, cyclobutyl, cyclopentyl, cyclohexyl, —CH₂F, —CHF₂, —CF₃, phenyl, benzyl, pyridine, pyrimidine, -ethylmethoxy, or -methylcyclopropyl, or two R^{A} groups can be taken together with the atoms to which they are attached to, to form a heterocylyl or heteroaryl ring.

In one embodiment of the compounds of Formula (AI), (I), (AII), or (II), or any subgenera thereof, two R^{A} groups on the same nitrogen atom are taken together to form a substituted or unsubstituted heterocylyl or heteroaryl ring. In one embodiment, two R^{A} groups on the same nitrogen atom are taken together to form a heterocylyl or heteroaryl ring.

In one embodiment of the compounds of Formula (AI), (I), (AII), or (II), or any subgenera thereof, the nitrogen protecting group, the sulfur protecting group, or the oxygen protecting group is benzyl.

In the following sections, various embodiments are separately discussed, which can be applied to any of the structures disclosed herein, including Formula (AI) or (AII), or any subgenera thereof (e.g., Formula (I), (I-A)-(I-D), (I-A1)-(I-A8), (I-B1)-(I-B8), (I-C1)-(I-C7), (I-D1)-(I-D7), (I-E)-(I-G), (I-E1), (I-F1), (I-G1), (II), (II-A)-(II-D), (II-A1)-(II-A8), (II-B1)-(II-B8), (II-C1)-(II-C7), (II-D1)-(II-D7), etc), including compounds in Table A.

Group R³

As generally defined herein, R³ is hydrogen, substituted or unsubstituted alkyl, substituted or unsubstituted alkenyl, substituted or unsubstituted alkynyl, substituted or unsubstituted carbocylyl, substituted or unsubstituted heterocyclyl, substituted or unsubstituted aryl, substituted or unsubstituted heteroaryl.

In certain embodiments, R³ is substituted or unsubstituted —C₁₋₆ alkyl, e.g, substituted or unsubstituted —C₁₋₂ alkyl, substituted or unsubstituted —C₂₋₃ alkyl, substituted or unsubstituted —C₃₋₄ alkyl, substituted or unsubstituted —C₄₋₅ alkyl, substituted or unsubstituted —C₅₋₆ alkyl. Exemplary —C₁₋₆ alkyl groups for R³ include, but are not limited to, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), n-hexyl (C₆), C₁₋₆ alkyl substituted with 1, 2, 3, 4, 5, 6, 7, 8, 9, 10, or more fluoro groups (e.g., —CF₃, —CH₂F, —CHF₂, difluoroethyl, and 2,2,2-trifluoro-1,1-dimethyl-ethyl), C₁₋₆ alkyl substituted with 1, 2, 3, 4, 5, 6, 7, 8, 9, 10, or more chloro groups (e.g., —CH₂Cl, —CHCl₂), and C₁₋₆ alkyl substituted with alkoxy groups (e.g., —CH₂OCH₃ and —CH₂OCH₂CH₃). In certain embodiments, R³ is substituted —C₁₋₆ alkyl, e.g., R³ is haloalkyl, alkoxy alkyl, or aminoalkyl. In certain embodiments, R³ is Me, Et, n-Pr, n-Bu, i-Bu, fluoromethyl, chloromethyl, difluoromethyl, trifluoromethyl, trifluoroethyl, difluoroethyl, 2,2,2-trifluoro-1,1-dimethyl-ethyl, methoxymethyl, methoxyethyl, or ethoxymethyl. In one embodiment, R³ is —CH₃, —CH₂CH₃, —CH₂F, —CHF₂, —CF₃, —CH₂OCH₂CH₃, or —CH₂OCH₃.

In certain embodiments, R³ is unsubstituted —C₁₋₃ alkyl, e.g., R³ is —CH₃, —CH₂CH₃, or —CH₂CH₂CH₃. In one embodiment, R³ is —CH₃.

In one embodiment, R³ is —C₁₋₆ alkyl optionally substituted with one to three groups selected from halogen or —C₁₋₆ alkoxy.

In certain embodiments, R³ is —C₁₋₆ alkyl substituted with one or more fluorine atoms, e.g., R³ is —CH₂F, —CHF₂, or —CF₃. In certain embodiments, R³ is C₁₋₆ alkyl substituted with one or two fluorine atoms, e.g., R³ is —CH₂F, —CHF₂.

In certain embodiments, R³ is —C₁₋₆ alkyl substituted with one or more —OR^{A3} groups, where R^{A3} is hydrogen or substituted or unsubstituted alkyl. In certain embodiments, R³ is —CH₂OR^{A3}, e.g., wherein R^{A3} is hydrogen, —CH₃, —CH₂CH₃, or —CH₂CH₂CH₃, e.g., to provide a group R³ of formula —CH₂OH, —CH₂OCH₃, —CH₂OCH₂CH₃ or —CH₂OCH₂CH₃CH₃.

In certain embodiments, R³ is substituted or unsubstituted —C₂₋₆ alkenyl, e.g, substituted or unsubstituted —C₂₋₃ alkenyl, substituted or unsubstituted —C₃₋₄ alkenyl, substituted or unsubstituted —C₄₋₅ alkenyl, substituted or unsubstituted —C₅₋₆ alkenyl. In certain embodiments, R³ is ethenyl (C₂), propenyl (C₃), or butenyl (C₄), substituted or unsubstituted with one or more substituents selected from the group consisting of alkyl, halo, haloalkyl, alkoxyalkyl, or hydroxyl. In certain embodiments, R³ is ethenyl, propenyl, or butenyl, substituted or unsubstituted with alkyl, halo, haloalkyl, alkoxyalkyl, or hydroxyl. In certain embodiments, R³ is ethenyl.

In certain embodiments, R³ is substituted or unsubstituted —C₂₋₆ alkynyl, e.g, substituted or unsubstituted —C₂₋₃ alkynyl, substituted or unsubstituted —C₃₋₄ alkynyl, substituted or unsubstituted —C₄₋₅ alkynyl, substituted or unsubstituted —C₅₋₆ alkynyl. In certain embodiments, R³ is ethynyl, propynyl, or butynyl, substituted or unsubstituted with alkyl, halo, haloalkyl (e.g., CF₃), alkoxyalkyl, cycloalkyl (e.g., cyclopropyl or cyclobutyl), or hydroxyl. In certain embodiments, R³ is selected from the group consisting of trifluoroethynyl, cyclopropylethynyl, cyclobutylethynyl, and propynyl, fluoropropynyl, and chloropropynyl. In certain embodiments, R³ is ethynyl (C₂), propynyl (C₃), or butynyl (C₄), substituted or unsubstituted with one or more substituents selected from the group consisting of substituted or unsubstituted aryl, substituted or unsubstituted heteroaryl, substituted or unsubstituted carbocyclyl, and substituted or unsubstituted heterocyclyl. In certain embodiments, R³ is ethynyl (C₂), propynyl (C₃), or butynyl (C₄), substituted with substituted phenyl. In certain embodiments, the phenyl substituent is further substituted with one or more substituents selected from the group consisting of alkyl, halo, trifluoroalkyl, alkoxy, acyl, amino, or amido. In certain embodiments, R³ is ethynyl (C₂), propynyl (C₃), or butynyl (C₄), substituted with substituted or unsubstituted pyrrolyl, imidazolyl, pyrazolyl, oxazoyl, thiazolyl, isoxazoyl, 1,2,3-triazolyl, 1,2,4-triazolyl, oxadiazolyl, thiadiazolyl, tetrazolyl, or tetrazolonyl.

In certain embodiments, R³ is ethynyl, propynyl, or butynyl, substituted or unsubstituted with alkyl, halo, haloalkyl, alkoxyalkyl, or hydroxyl. In certain embodiments, R³ is ethynyl or propynyl, substituted with substituted or unsubstituted aryl. In certain embodiments, R³ is ethynyl or propynyl, substituted with phenyl substituted or unsubstituted with alkyl, halo, haloalkyl, alkoxy, trihaloalkyl, or acyl. In certain embodiments, R³ is ethynyl or propynyl, substituted with substituted or unsubstituted carbocyclyl. In certain embodiments, R³ is ethynyl or propynyl, substituted with substituted or unsubstituted cyclopropyl, cyclobutyl, cyclopentyl, or cyclohexyl. In certain embodiments, R³ is ethynyl or propynyl, substituted with substituted or unsubstituted heteroaryl. In certain embodiments, R³ is ethynyl or propynyl, substituted with substituted or unsubstituted pyridinyl, or pyrimidinyl. In certain embodiments, R³ is ethynyl or propynyl, substituted with substituted or unsubstituted pyrrolyl, imidazolyl, pyrazolyl, oxazoyl, thiazolyl, isoxazoyl, 1,2,3-triazolyl, 1,2,4-triazolyl, oxadiazolyl, thiadiazolyl, tetrazolyl, or tetrazolonyl. In certain embodiments, R³ is ethynyl or propynyl, substituted with substituted or unsubstituted heterocyclyl. In certain embodiments, R³ is ethynyl or propynyl, substituted with substituted or unsubstituted pyrrolidinyl, piperidinyl, piperazinyl, or morpholinyl. In certain embodiments, R³ is propynyl or butynyl, substituted with hydroxyl or alkoxy. In certain embodiments, R³ is propynyl or butynyl, substituted with methoxy or ethoxy. In certain embodiments, R³ is ethynyl or propynyl, substituted with chloro. In certain embodiments, R³ is ethynyl or propynyl, substituted with trifluoromethyl.

In certain embodiments, R³ is substituted or unsubstituted C₃₋₆ carbocyclyl, e.g. substituted or unsubstituted C₃₋₄ carbocyclyl, substituted or unsubstituted C₄₋₅ carbocyclyl, or substituted or unsubstituted C₅₋₆ carbocyclyl. In certain embodiments, R³ is substituted or unsubstituted cyclopropyl or substituted or unsubstituted cyclobutyl.

Groups [symbol] , R², R⁴, R⁶, R⁷, R^{11a}, R^{11b}, R¹², and R¹⁶

As generally defined herein, R², R⁴, R⁶, R⁷, R^{11a}, R^{11b}, R¹², and R¹⁶ are each independently hydrogen, halogen, cyano, nitro, substituted or unsubstituted alkyl, substituted or unsubstituted alkenyl, substituted or unsubstituted alkynyl, substituted or unsubstituted carbocylyl, substituted or unsubstituted heterocyclyl, substituted or unsubstituted aryl, substituted or unsubstituted heteroaryl, —OR^{A}, —C(═O)R^{A}, —C(═O)OR^{A}, —OC(═O)R^{A}, —OC(═O)OR^{A}, —C(═O)NR^{A}R^{A}, —NR^{A}R^{A}, —NR^{A}C(═O)R^{A}, —OC(═O)NR^{A}R^{A}, —NR^{A}C(═O)OR^{A}, —NR^{A}C(═O)NR^{A}R^{A}, —SR^{A}, —S(═O)R^{A}, —S(═O)₂R^{A}, —S(═O)₂OR^{A}, —OS(═O)₂R^{A}, —S(═O)₂NR^{A}R^{A}, or —NR^{A}S(═O)₂R^{A}, wherein R^{A} is independently hydrogen, substituted or unsubstituted alkyl, substituted or unsubstituted alkenyl, substituted or unsubstituted alkynyl, substituted or unsubstituted carbocylyl, substituted or unsubstituted heterocylyl, substituted or unsubstituted aryl, substituted or unsubstituted heteroaryl, haloalkyl, an oxygen protecting group when attached to oxygen, a sulfur protecting group when attached to sulfur, or a nitrogen protecting group when attached to nitrogen, or two R^{A} groups can be taken together with the atoms to which they are attached to, to form a substituted or unsubstituted heterocylyl or heteroaryl ring. In one embodiment, the protecting group is a benzyl group.

In certain embodiments, R² is hydrogen. In certain embodiments, R² is halogen, e.g., fluoro, chloro, bromo, or iodo. In certain embodiments, R² is fluoro or chloro. In certain embodiments, R² is substituted or unsubstituted —C₁₋₆ alkyl, e.g., substituted or unsubstituted —C₁₋₂ alkyl, substituted or unsubstituted —C₂₋₃ alkyl, substituted or unsubstituted —C₃₋₄ alkyl, substituted or unsubstituted —C₄₋₅ alkyl, or substituted or unsubstituted —C₅₋₆ alkyl. For example, in some embodiments, R² is —C₁₋₆ alkyl optionally substituted with halo (e.g., fluoro, chloro, bromo (i.e., to provide a group R² of formula —CH₂F, —CHF₂, —CF₃)) or —OR^{A2}. In certain embodiments, R^{A2} is hydrogen, —CH₃, —CH₂CH₃, or —CH₂CH₂CH₃, i.e., to provide a group R² of formula —OH, —OCH₃, —OCH₂CH₃ or —OCH₂CH₃CH₃. In certain embodiments, R² is substituted or unsubstituted —C₂₋₆ alkenyl, e.g, substituted or unsubstituted —C₂₋₃ alkenyl, substituted or unsubstituted —C₃₋₄ alkenyl, substituted or unsubstituted —C₄₋₅ alkenyl, or substituted or unsubstituted —C₅₋₆ alkenyl. In certain embodiments, R² is substituted or unsubstituted —C₂₋₆ alkynyl, e.g, substituted or unsubstituted —C₂₋₃ alkynyl, substituted or unsubstituted —C₃₋₄ alkynyl, substituted or unsubstituted —C₄₋₅ alkynyl, or substituted or unsubstituted —C₅₋₆ alkynyl. In certain embodiments, R² is substituted or unsubstituted —C₃₋₆ carbocyclyl, e.g, substituted or unsubstituted —C₃₋₄ carbocyclyl, substituted or unsubstituted —C₄₋₅ carbocyclyl, or substituted or unsubstituted —C₅₋₆ carbocyclyl. In certain embodiments, R² is substituted or unsubstituted cyclopropyl or substituted or unsubstituted cyclobutyl. In certain embodiments, R² is hydrogen, —CH₃, —CH₂CH₃, or —CH₂CH₂CH₃, or substituted or unsubstituted cyclopropyl. In certain embodiments, R² is —OR^{A2}. In certain embodiments, R^{A2} is hydrogen. In certain embodiments, R^{A2} is substituted or unsubstituted alkyl, e.g., substituted or unsubstituted —C₁₋₆ alkyl, substituted or unsubstituted —C₁₋₂ alkyl, substituted or unsubstituted —C₂₋₃ alkyl, substituted or unsubstituted —C₃₋₄ alkyl, substituted or unsubstituted —C₄₋₅ alkyl, or substituted or unsubstituted —C₅₋₆ alkyl. In certain embodiments, R^{A2} is hydrogen, —CH₃, —CH₂CH₃, or —CH₂CH₂CH₃, i.e., to provide a group R² of formula —OH, —OCH₃, —OCH₂CH₃ or —OCH₂CH₃CH₃. In certain embodiments, R² is a non-hydrogen substituent in the alpha configuration. In certain embodiments, R² is a non-hydrogen substituent in the beta configuration.

In certain embodiments, R^{11a} and R^{11b} are each independently hydrogen, or —OR^{A11}, wherein R^{A11} is hydrogen or substituted or unsubstituted —C₁₋₆ alkyl, substituted or unsubstituted —C₂₋₆ alkenyl, substituted or unsubstituted —C₂₋₆ alkynyl, or substituted or unsubstituted —C₃₋₆ carbocylyl; or R^{11a} and R^{11b} are joined to form an oxo (═O) group.

In certain embodiments, both R^{11a} and R^{11b} are hydrogen.

In certain embodiments, R^{11a} and R^{11b} are joined to form an oxo (═O) group.

In one embodiment, R^{11a} is —OH, —OCH₃, —OCH₂CH₃ or —OCH₂CH₃CH₃ and R^{11b} is hydrogen.

In certain embodiments, R^{11a} is —OR^{A11} and R^{11b} is hydrogen. In certain embodiments, wherein R^{11a} is —OR^{A11} and R^{11a} is in the alpha or beta configuration. In certain embodiments, wherein R^{11a} is —OR^{A11} and R^{11a} is in the alpha configuration. In certain embodiments, wherein R^{11a} is —OR^{A11} and R^{11a} is in the beta configuration. In certain embodiments, R^{A11} is hydrogen. In certain embodiments, R^{A11} is substituted or unsubstituted —C₁₋₆ alkyl, e.g., substituted or unsubstituted —C₁₋₂ alkyl, substituted or unsubstituted —C₂₋₃ alkyl, substituted or unsubstituted —C₃₋₄ alkyl, substituted or unsubstituted —C₄₋₅ alkyl, or substituted or unsubstituted —C₅₋₆ alkyl. In certain embodiments, R^{A11} is hydrogen, —CH₃, —CH₂CH₃, or —CH₂CH₂CH₃, i.e., to provide a group R^{11a} of formula —OH, —OCH₃, —OCH₂CH₃ or —OCH₂CH₃CH₃.

As generally defined herein, [symbol] represents a single or double bond; wherein when one of [symbol] is a double bond, the other [symbol] is a single bond; and R⁵ is absent; or when both of [symbol] are single bonds, then R⁵ is hydrogen.

In one embodiment of the compound of Formula (AI), (I), (AII), or (II) or any subgenera thereof, both of [symbol] are single bonds and R⁵ and R⁴ are in an alpha configuration. In other embodiments, both of [symbol] are single bonds and R⁵ and R⁴ are in a beta configuration. In some embodiments, both of [symbol] are single bonds and R⁵ and R⁶ are in an alpha configuration. In other embodiments, both of [symbol] are single bonds and R⁵ and R⁶ are in a beta configuration.

In certain embodiments, R⁴ and R⁶ is independently hydrogen. In certain embodiments, each of R⁴ and R⁶ is independently substituted or unsubstituted —C₁₋₆ alkyl, e.g., substituted or unsubstituted —C₁₋₂ alkyl, substituted or unsubstituted —C₂₋₃ alkyl, substituted or unsubstituted —C₃₋₄ alkyl, substituted or unsubstituted —C₄₋₅ alkyl, or substituted or unsubstituted —C₅₋₆ alkyl. In certain embodiments, each of R⁴ and R⁶ is independently —C₁ alkyl, e.g., —CH₃, or —CF₃. In certain embodiments, each of R⁴ and R⁶ is independently halogen, e.g., fluoro.

In one embodiment, at least one of R⁴ or R⁶ is hydrogen, substituted or unsubstituted —C₁₋₆ alkyl, halogen, —CH₃, or —CF₃.

In certain embodiments, both of R⁴ and R⁶ are hydrogen. In certain embodiments, both of R⁴ and R⁶ are independently substituted or unsubstituted —C₁₋₆ alkyl, e.g., substituted or unsubstituted —C₁₋₂ alkyl, substituted or unsubstituted —C₂₋₃ alkyl, substituted or unsubstituted —C₃₋₄ alkyl, substituted or unsubstituted —C₄₋₅ alkyl, or substituted or unsubstituted —C₅₋₆ alkyl. In certain embodiments, both of R⁴ and R⁶ are independently —C₁ alkyl, e.g., —CH₃, or —CF₃. In certain embodiments, both of R⁴ and R⁶ are halogen, e.g., fluoro.

In certain embodiments, wherein [symbol] represents a single bond, R⁴ or R⁶ is a non-hydrogen substituent in the alpha configuration. In certain embodiments, wherein [symbol] represents a single bond, R⁴ or R⁶ is a non-hydrogen substituent in the beta configuration.

In certain embodiments, R⁷ is hydrogen. In certain embodiments, R⁷ is alkyl (e.g., unsubstituted alkyl or —CH₂OR^{A7}) or —OR^{A7}, wherein R^{A7} is independently hydrogen, substituted or unsubstituted alkyl, substituted or unsubstituted alkenyl, substituted or unsubstituted alkynyl, substituted or unsubstituted carbocylyl, substituted or unsubstituted heterocylyl, substituted or unsubstituted aryl, substituted or unsubstituted heteroaryl, haloalkyl, an oxygen protecting group. In certain embodiments, R⁷ is —CH₃, —CH₂CH₃, —CH(CH₃)₂, —OH, —OCH₃, or —CH₂OCH₃.

In certain embodiments, R¹² is hydrogen. In certain embodiments, R¹² is alkyl (e.g., unsubstituted alkyl or —CH₂OR^{A12}) or —OR^{A12}, wherein R^{A12} is independently hydrogen, substituted or unsubstituted alkyl, substituted or unsubstituted alkenyl, substituted or unsubstituted alkynyl, substituted or unsubstituted carbocylyl, substituted or unsubstituted heterocylyl, substituted or unsubstituted aryl, substituted or unsubstituted heteroaryl, haloalkyl, an oxygen protecting group. In certain embodiments, R¹² is —CH₃, —CH₂CH₃, —CH(CH₃)₂, —OH, —OCH₃, or —CH₂OCH₃.

In certain embodiments, R¹⁶ is hydrogen. In certain embodiments, R¹⁶ is alkyl (e.g., unsubstituted alkyl or —CH₂OR^{A16}) or —OR^{A16}, wherein R^{A16} is independently hydrogen, substituted or unsubstituted alkyl, substituted or unsubstituted alkenyl, substituted or unsubstituted alkynyl, substituted or unsubstituted carbocylyl, substituted or unsubstituted heterocylyl, substituted or unsubstituted aryl, substituted or unsubstituted heteroaryl, haloalkyl, an oxygen protecting group. In certain embodiments, R¹⁶ is —CH₃, —CH₂CH₃, —CH(CH₃)₂, —OH, —OCH₃, or —CH₂OCH₃.

In certain embodiments, at least one of R², R⁴, R⁶, R⁷, R^{11a}, R^{11b}, R¹², and R¹⁶ is hydrogen. In certain embodiments, at least two of R², R⁴, R⁶, R⁷, R^{11a}, R^{11b}, R¹², and R¹⁶ are hydrogen. In certain embodiments, at least three of R², R⁴, R⁶, R⁷, R^{11a}, R^{11b}, R¹², and R¹⁶ are hydrogen. In certain embodiments, at least four of R², R⁴, R⁶, R⁷, R^{11a}, R^{11b}, R¹², and R¹⁶ are hydrogen. In certain embodiments, at least five of R², R⁴, R⁶, R⁷, R^{11a}, R^{11b}, R¹², and R¹⁶ are hydrogen. In certain embodiments, at least six of R², R⁴, R⁶, R⁷, R^{11a}, R^{11b}, R¹², and R¹⁶ are hydrogen. In certain embodiments, at least seven of R², R⁴, R⁶, R⁷, R^{11a}, R^{11b}, R¹², and R¹⁶ are hydrogen.

In certain embodiments, R², R⁴, R⁶, R⁷, R^{11a}, R^{11b}, R¹², and R¹⁶ are all hydrogen.

Group R¹⁰

As generally defined herein, R¹⁰ is hydrogen, halogen, cyano, or substituted or unsubstituted alkyl. In some embodiments, R¹⁰ is hydrogen or substituted or unsubstituted alkyl. In certain embodiments, R¹⁰ is hydrogen. In certain embodiments, R¹⁰ is substituted or unsubstituted —C₁₋₆ alkyl (e.g., unsubstituted alkyl or —CH₂OR^{A10}), wherein R^{A10} is hydrogen, substituted or unsubstituted —C₁₋₆ alkyl, or substituted or unsubstituted —C₁₋₆ alkoxy. In one embodiment, R¹⁰ is methyl, substituted with one or more fluorines. In one embodiment, R¹⁰ is halogen, cyano, —CH₃, —CH₂CH₃, —CH₂F, —CHF₂, —CF₃, —CH₂OCH₃ or —CH₂SCH₃. In one embodiment, R¹⁰ is hydrogen, —CH₃, —CH₂CH₃, or —CH₂OCH₃. In one embodiment of the compounds of the present invention, R¹⁰ is hydrogen, halogen, cyano, —CH₃, —CH₂F, —CHF₂, —CF₃, —CH₂CH₃, —CH₂OCH₃, or —CH₂SCH₃. In one embodiment, R¹⁰ is hydrogen, fluoro, cyano, —CH₃, —CH₂F, —CHF₂, —CF₃, —CH₂CH₃, —CH₂OCH₃ or —CH₂SCH₃. In one embodiment, R¹⁰ is —CH₂OCH₃ or —CH₂SCH₃. In certain embodiments, R¹⁰ is —CH₃, —CH₂CH₃, or —CH₂OCH₃. In some embodiments, R¹⁰ is halogen. In certain embodiments, R¹⁰ is fluoro. In some embodiments, R¹⁰ is —CH₃ or fluoro.

Group R¹⁹

As generally defined herein, R^{19a} is hydrogen, substituted or unsubstituted alkyl, or —OR^{A19}, wherein R^{A19} is hydrogen, substituted or unsubstituted alkyl, substituted or unsubstituted alkenyl, substituted or unsubstituted alkynyl, or substituted or unsubstituted carbocylyl; and R^{19b} is hydrogen or substituted or unsubstituted alkyl; R^{19a} and R^{19b} are joined to form an oxo (═O) group; or R^{19a} and R^{19b} together with the carbon atom to which they are both attached, form a 3-8 membered saturated, partially saturated, or unsaturated ring optionally containing one or more heteroatoms as a ring member selected from N, O, or S (e.g., carbocycyl or heterocyclyl ring).

In certain embodiments, R^{19a} and R^{19b} are both hydrogen. In certain embodiments, one of R^{19a} and R^{19b} is not hydrogen. In certain embodiments, both of R^{19a} and R^{19b} are not hydrogen. In certain embodiments, R^{19a} is C₁₋₆ alkyl and R^{19b} is hydrogen or C₁₋₆ alkyl. In certain embodiments, R^{19a} is methyl and R^{19b} is hydrogen.

Group R²⁰

As generally defined herein, R²⁰ is hydrogen, alkyl, haloalkyl, cycloalkyl, cycloalkylalkyl, heterocyclyl, aryl, C₆-C₁₂ aryl-C₁-C₆ alkyl-, heteroaryl, or heteroaryl-C₁-C₆ alkyl-, wherein alkyl, haloalkyl, cycloalkyl, cycloalkylalkyl, heterocyclyl, aryl, C₆-C₁₂ aryl-C₁-C₆ alkyl-, heteroaryl, and heteroaryl-C₁-C₆ alkyl- can be optionally further substituted with substituents selected from substituted or unsubstituted alkyl, substituted or unsubstituted alkenyl, substituted or unsubstituted alkynyl, substituted or unsubstituted carbocylyl, substituted or unsubstituted heterocylyl, haloalkyl, substituted or unsubstituted —C₆-C₁₂ aryl, substituted or unsubstituted 5-12 membered heteroaryl, halogen, nitro, cyano, —OR^{A}, —C(═O)R^{A}, —C(═O)OR^{A}, —OC(═O)R^{A}, —OC(═O)OR^{A}, —C(═O)NR^{A}R^{A}, —NR^{A}R^{A}, —NR^{A}C(═O)R^{A}, —OC(═O)NR^{A}R^{A}, —NR^{A}C(═O)OR^{A}, —NR^{A}C(═O)NR^{A}R^{A}, —SR^{A}, —S(═O)R^{A}, —S(═O)₂R^{A}, —S(═O)₂OR^{A}, —OS(═O)₂R^{A}, —S(═O)₂NR^{A}R^{A}, or —NR^{A}S(═O)₂R^{A}, wherein R^{A} is independently hydrogen or substituted or unsubstituted alkyl, substituted or unsubstituted alkenyl, substituted or unsubstituted alkynyl, substituted or unsubstituted carbocylyl, substituted or unsubstituted heterocylyl, substituted or unsubstituted aryl, substituted or unsubstituted heteroaryl, haloalkyl, an oxygen protecting group when attached to oxygen, a sulfur protecting group when attached to sulfur, or a nitrogen protecting group when attached to nitrogen, or two R^{A} groups can be taken together with the atoms to which they are attached to, to form a substituted or unsubstituted heterocylyl or heteroaryl ring.

In one embodiment, R²⁰ is hydrogen, —C₁₋₆ alkyl, —C₃₋₁₂ cycloalkyl, C₃₋₁₂ cycloalkyl-C₁₋₆ alkyl-, —C₆-C₁₂ aryl, or 5-12 membered heteroaryl, each of which can be optionally substituted with halogen, nitro, cyano, —C₁₋₆ alkyl, —C₃₋₁₂ cycloalkyl, 3-12 membered heterocylyl, —C₆-C₁₂ aryl, 5-12 membered heteroaryl, —OR^{A}, —C(═O)R^{A}, —C(═O)OR^{A}, —OC(═O)R^{A}, —OC(═O)OR^{A}, —C(═O)NR^{A}R^{A}, —NR^{A}R^{A}, —NR^{A}C(═O)R^{A}, —OC(═O)NR^{A}R^{A}, —NR^{A}C(═O)OR^{A}, —NR^{A}C(═O)NR^{A}R^{A}, —SR^{A}, —S(═O)R^{A}, —S(═O)₂R^{A}, —S(═O)₂OR^{A}, —OS(═O)₂R^{A}, —S(═O)₂NR^{A}R^{A}, or —NR^{A}S(═O)₂R^{A}; wherein R^{A} is independently hydrogen, alkyl, haloalkyl, alkenyl, alkynyl, cycloalkyl, cycloalkylalkyl, carbocylyl, heterocylyl, aryl, C₆-C₁₂ aryl-C₁-C₆ alkyl-, heteroaryl, heteroaryl-C₁-C₆ alkyl-, an oxygen protecting group when attached to oxygen, a sulfur protecting group when attached to sulfur, or a nitrogen protecting group when attached to nitrogen; or two R^{A} groups can be taken together with the atoms to which they are attached to, to form a substituted or unsubstituted heterocylyl or heteroaryl ring.

In one embodiment of the compounds of the present invention, R²⁰ is hydrogen, substituted or unsubstituted —C₁₋₆ alkyl, —C₂₋₆ alkenyl, —C₂₋₆ alkynyl, —C₁₋₆ haloalkyl, —C₃₋₁₂ cycloalkyl, C₃₋₁₂ cycloalkyl-C₁-C₆ alkyl-, heterocyclyl, —C₆-C₁₂ aryl, C₆-C₁₂ aryl-C₁-C₆ alkyl-, 5-12 membered heteroaryl, or 5-12 membered heteroaryl-C₁-C₆ alkyl-. In one embodiment, R²⁰ is —C₁₋₆ alkyl, —C₁₋₆ haloalkyl, —C₃₋₁₂ cycloalkyl, C₃₋₁₂ cycloalkyl-C₁-C₆ alkyl-, —C₆-C₁₂ aryl, or 5-12 membered heteroaryl, which can be optionally substituted with halogen, cyano, nitro, substituted or unsubstituted alkyl, alkenyl, alkynyl, carbocylyl, 3-12 membered heterocylyl, —C₆-C₁₂ aryl, 5-12 membered heteroaryl, —OR^{A}, —C(═O)R^{A}, —C(═O)OR^{A}, —OC(═O)R^{A}, —OC(═O)OR^{A}, —C(═O)NR^{A}R^{A}, —NR^{A}R^{A}, —NR^{A}C(═O)R^{A}, —OC(═O)NR^{A}R^{A}, —NR^{A}C(═O)OR^{A}, —NR^{A}C(═O)NR^{A}R^{A}, —SR^{A}, —S(═O)R^{A}, —S(═O)₂R^{A}, —S(═O)₂OR^{A}, —OS(═O)₂R^{A}, —S(═O)₂NR^{A}R^{A}, or —NR^{A}S(═O)₂R^{A}, wherein R^{A} is independently hydrogen or substituted or unsubstituted alkyl, alkenyl, alkynyl, substituted or unsubstituted carbocylyl, substituted or unsubstituted heterocylyl, substituted or unsubstituted aryl, substituted or unsubstituted heteroaryl, or two R^{A} groups can be taken together with the atoms to which they are attached to, to form a substituted or unsubstituted heterocylyl or heteroaryl ring. In one embodiment, R²⁰ is —C₁₋₆ alkyl, —C₃₋₁₂ cycloalkyl, C₃₋₁₂ cycloalkyl-C₁-C₆ alkyl-, —C₆-C₁₂ aryl, or 5-12 membered heteroaryl, which can be optionally substituted with halogen, cyano, substituted or unsubstituted alkyl, substituted or unsubstituted carbocylyl, or alkoxy.

In certain embodiments, each instance of R^{A} is independently hydrogen, substituted or unsubstituted —C₁₋₆ alkyl (e.g., substituted or unsubstituted —C₁₋₂ alkyl, substituted or unsubstituted —C₂₋₃ alkyl, substituted or unsubstituted —C₃₋₄ alkyl, substituted or unsubstituted —C₄₋₅ alkyl, or substituted or unsubstituted —C₅₋₆ alkyl), substituted or unsubstituted cycloalkylalkyl, substituted or unsubstituted aryl, or substituted or unsubstituted heteroaryl. In certain embodiments, each instance of R^{A} is hydrogen, —CH₃, —CH₂CH₃, or substituted or unsubstituted phenyl.

In certain embodiments, R²⁰ is hydrogen. In certain embodiments, R²⁰ is a non-hydrogen substituent. As used herein, a R²⁰ “non-hydrogen substituent” means that R²⁰ is not hydrogen, but are any one of —C₁₋₆ alkyl, —C₂₋₆ alkenyl, —C₂₋₆ alkynyl, —C₁₋₆ haloalkyl, —C₃₋₁₂ cycloalkyl, C₃₋₁₂ cycloalkyl-C₁-C₆ alkyl-, heterocyclyl, —C₆-C₁₂ aryl, —C₆₋₁₂ aryl-C₁-C₆ alkyl-, 5-12 membered heteroaryl, or 5-12 membered heteroaryl-C₁-C₆ alkyl-.

In one embodiment, R²⁰ is —C₁₋₆ alkyl, —C₃₋₁₂ cycloalkyl, C₃₋₁₂ cycloalkyl-C₁-C₆ alkyl-, —C₆-C₁₂ aryl, or 5-12 membered heteroaryl, each of which are optionally substituted. In certain embodiments, R²⁰ is any one of —C₁₋₆ alkyl, —C₂₋₆ alkenyl, —C₂₋₆ alkynyl, —C₁₋₆ haloalkyl, —C₃₋₁₂ cycloalkyl, C₃₋₁₂ cycloalkyl-C₁-C₆ alkyl-, heterocyclyl, —C₆-C₁₂ aryl, C₆-C₁₂ aryl-C₁-C₆ alkyl-, 5-12 membered heteroaryl, or 5-12 membered heteroaryl-C₁-C₆ alkyl- that are optionally substituted with halogens (e.g., fluoro, bromo, iodo, or chloro), nitro, cyano.

In certain embodiments, R²⁰ is any one of —C₁₋₆ alkyl, —C₂₋₆ alkenyl, —C₂₋₆ alkynyl, —C₁₋₆ haloalkyl, —C₃₋₁₂ cycloalkyl, C₃₋₁₂ cycloalkyl-C₁-C₆ alkyl-, heterocyclyl, —C₆-C₁₂ aryl, C₆-C₁₂ aryl-C₁-C₆ alkyl-, 5-12 membered heteroaryl, or 5-12 membered heteroaryl-C₁-C₆ alkyl- that are optionally substituted with —OR^{A}, wherein R^{A} is hydrogen or substituted or unsubstituted —C₁₋₆ alkyl (e.g., —CH₃ or —CF₃). In certain embodiments, —OR^{A} is —OH, —OCH₃, —OCF₃.

In certain embodiments, R²⁰ is any one of —C₁₋₆ alkyl, —C₂₋₆ alkenyl, —C₂₋₆ alkynyl, —C₁₋₆ haloalkyl, —C₃₋₁₂ cycloalkyl, C₃₋₁₂ cycloalkyl-C₁-C₆ alkyl-, heterocyclyl, —C₆-C₁₂ aryl, C₆-C₁₂ aryl-C₁-C₆ alkyl-, 5-12 membered heteroaryl, or 5-12 membered heteroaryl-C₁-C₆ alkyl- that are optionally substituted with —N(R^{A})₂, wherein R^{A} is hydrogen or substituted or unsubstituted —C₁₋₆ alkyl (e.g., —CH₃ or —CF₃). In certain embodiments, —N(R^{A})₂ is —NH₂, —NHCH₃, —N(CH₃)₂.

In certain embodiments, R²⁰ is any one of —C₁₋₆ alkyl, —C₂₋₆ alkenyl, —C₂₋₆ alkynyl, —C₁₋₆ haloalkyl, —C₃₋₁₂ cycloalkyl, C₃₋₁₂ cycloalkyl-C₁-C₆ alkyl-, heterocyclyl, —C₆-C₁₂ aryl, C₆-C₁₂ aryl-C₁-C₆ alkyl-, 5-12 membered heteroaryl, or 5-12 membered heteroaryl-C₁-C₆ alkyl- that are optionally substituted with —C(═O)R^{A}, —C(═O)OR^{A} or —C(═O)N(R^{A})₂, e.g., wherein R^{A} is hydrogen or substituted or unsubstituted —C₁₋₆ alkyl (e.g., —CH₃ or —CF₃). In certain embodiments, —C(═O)R^{A} is —CHO, —C(═O)CH₃, or —C(═O)CH₂CH₃. In certain embodiments, —C(═O)OR^{A} is —C(═O)OH, —C(═O)OCH₃, or —C(═O)OCH₂CH₃. In certain embodiments, —C(═O)N(R^{A})₂ is —C(═O)NH₂, —C(═O)NHCH₃, or —C(═O)N(CH₃)₂.

In certain embodiments, R²⁰ is any one of —C₁₋₆ alkyl, —C₂₋₆ alkenyl, —C₂₋₆ alkynyl, —C₁₋₆ haloalkyl, —C₃₋₁₂ cycloalkyl, C₃₋₁₂ cycloalkyl-C₁-C₆ alkyl-, heterocyclyl, —C₆-C₁₂ aryl, C₆-C₁₂ aryl-C₁-C₆ alkyl-, 5-12 membered heteroaryl, or 5-12 membered heteroaryl-C₁-C₆ alkyl- that are optionally substituted with —OC(═O)R^{A}, —OC(═O)OR^{A} or —OC(═O)N(R^{A})₂, e.g., wherein R^{A} is hydrogen or substituted or unsubstituted —C₁₋₆ alkyl (e.g., —CH₃ or —CF₃). In certain embodiments, —OC(═O)R^{A} is —OC(═O)CH₃. In certain embodiments, —OC(═O)OR^{A} is —OC(═O)OCH₃. In certain embodiments, —OC(═O)N(R^{A})₂ is —OC(═O)NHCH₃, or —OC(═O)N(CH₃)₂.

In certain embodiments, R²⁰ is any one of —C₁₋₆ alkyl, —C₂₋₆ alkenyl, —C₂₋₆ alkynyl, —C₁₋₆ haloalkyl, —C₃₋₁₂ cycloalkyl, C₃₋₁₂ cycloalkyl-C₁-C₆ alkyl-, heterocyclyl, —C₆-C₁₂ aryl, C₆-C₁₂ aryl-C₁-C₆ alkyl-, 5-12 membered heteroaryl, or 5-12 membered heteroaryl-C₁-C₆ alkyl- that are optionally substituted with —N(R^{A})C(═O)R^{A}, —N(R^{A})C(═O)OR^{A} or —N(R^{A})C(═O)N(R^{A})₂, e.g., wherein R^{A} is hydrogen or substituted or unsubstituted —C₁₋₆ alkyl (e.g., —CH₃ or —CF₃). In certain embodiments, —N(R^{A})C(═O)R^{A} is —NHC(═O)CH₃. In certain embodiments, —N(R^{A})C(═O)OR^{A} is —NHC(═O)OCH₃. In certain embodiments, —N(R^{A})C(═O)N(R^{A})₂ is —NHC(═O)NH₂, or —NHC(═O)N(CH₃)₂.

In certain embodiments, R²⁰ is any one of —C₁₋₆ alkyl, —C₂₋₆ alkenyl, —C₂₋₆ alkynyl, —C₁₋₆ haloalkyl, —C₃₋₁₂ cycloalkyl, C₃₋₁₂ cycloalkyl-C₁-C₆ alkyl-, heterocyclyl, —C₆-C₁₂ aryl, C₆-C₁₂ aryl-C₁-C₆ alkyl-, 5-12 membered heteroaryl, or 5-12 membered heteroaryl-C₁-C₆ alkyl- that are optionally substituted with —SR^{A}, —S(═O)R^{A}, —S(═O)₂R^{A}, —S(═O)₂OR^{A}, —OS(═O)₂R^{A}, —S(═O)₂NR^{A}R^{A}, or —NR^{A}S(═O)₂R^{A}, e.g., wherein R^{A} is hydrogen, substituted or unsubstituted —C₁₋₆ alkyl (e.g., —CH₃ or —CF₃), substituted or unsubstituted aryl, or substituted or unsubstituted heteroaryl. In certain embodiments, —SR^{A} is —SCH₃ or —S-Aryl, wherein Aryl is substituted or unsubstituted aryl or heteroaryl. In certain embodiments, —S(═O)R^{A} is —S(═O)CH₃, —S(═O)CF₃, or —S(═O)-Aryl, wherein Aryl is substituted or unsubstituted aryl or heteroaryl. In certain embodiments, —S(═O)₂R^{A} is —S(═O)₂CH₃, —S(═O)₂CF₃, or —S(═O)₂-Aryl, wherein Aryl is substituted or unsubstituted aryl or heteroaryl. In certain embodiments, —S(═O)₂OR^{A} is —S(═O)₂OCH₃, —S(═O)₂OCF₃, or —S(═O)₂O-Aryl, wherein Aryl is substituted or unsubstituted aryl or heteroaryl. In certain embodiments, —OS(═O)₂R^{A} is —OS(═O)₂CH₃, —OS(═O)₂CF₃, or —OS(═O)₂-Aryl, wherein Aryl is substituted or unsubstituted aryl or heteroaryl. In certain embodiments, —S(═O)₂N(R^{A})₂ is —S(═O)₂NHCH₃, —S(═O)₂NHCF₃, or —S(═O)₂NH-Aryl, wherein Aryl is substituted or unsubstituted aryl or heteroaryl. In certain embodiments, —N(R^{A})S(═O)₂R^{A} is —NHS(═O)₂CH₃, —NHS(═O)₂CF₃, or —NHS(═O)₂-Aryl, wherein Aryl is substituted or unsubstituted aryl or heteroaryl.

In certain embodiments, R²⁰ or R^{A} is substituted or unsubstituted —C₁₋₆ alkyl, e.g., substituted or unsubstituted —C₁₋₂ alkyl, substituted or unsubstituted —C₂₋₃ alkyl, substituted or unsubstituted —C₃₋₄ alkyl, substituted or unsubstituted —C₄₋₅ alkyl, or substituted or unsubstituted —C₅₋₆ alkyl. Exemplary —C₁₋₆ alkyl groups include, but are not limited to, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), n-hexyl (C₆), C₁₋₆ alkyl substituted with 1, 2, 3, 4, 5, 6, 7, 8, 9, 10, or more fluoro groups (e.g., —CF₃, —CH₂F, —CHF₂, difluoroethyl, and 2,2,2-trifluoro-1,1-dimethyl-ethyl), C₁₋₆ alkyl substituted with 1, 2, 3, 4, 5, 6, 7, 8, 9, 10, or more chloro groups (e.g., —CH₂Cl, —CHCl₂), and C₁₋₆ alkyl substituted with alkoxy groups (e.g., —CH₂OCH₃ and —CH₂OCH₂CH₃). In certain embodiments, R²⁰ or R^{A} is substituted —C₁₋₆ alkyl, e.g., haloalkyl, alkoxyalkyl, or aminoalkyl. In certain embodiments, R²⁰ or R^{A} is Me, Et, n-Pr, i-Pr, n-Bu, i-Bu, t-Bu, fluoromethyl, chloromethyl, difluoromethyl, trifluoromethyl, trifluoroethyl, difluoroethyl, 2,2,2-trifluoro-1,1-dimethyl-ethyl, methoxymethyl, methoxyethyl, or ethoxymethyl.

In one embodiment, R²⁰ is —CH₃, —CF₃, —CH₂CH₃, -i-Pr, -n-Pr, -i-Bu, -s-Bu, -t-Bu, —CH₂cyclopropyl, —CH₂CN, or —CH₂CH₂CN. In one embodiment, R²⁰ is —CH₃, —CH₂CH₃, -i-Pr, -n-Pr, -i-Bu, -s-Bu, -t-Bu, or —CH₂cyclopropyl. In one embodiment, R²⁰ is —CH₃, —CH₂CH₃, -i-Pr, -n-Pr, -i-Bu, -s-Bu, or -t-Bu.

In one embodiment, R²⁰ is substituted or unsubstituted C₃₋₁₂ cycloalkyl-C₁-C₆ alkyl-. In one embodiment, R²⁰ is substituted or unsubstituted cyclopropylmethyl-. In one embodiment, R²⁰ is —CH₂cyclopropyl.

In certain embodiments, R²⁰ or R^{A} is substituted or unsubstituted —C₂₋₆ alkenyl, e.g, substituted or unsubstituted —C₂₋₃ alkenyl, substituted or unsubstituted —C₃₋₄ alkenyl, substituted or unsubstituted —C₄₋₅ alkenyl, substituted or unsubstituted —C₅₋₆ alkenyl. In certain embodiments, R²⁰ or R^{A} is ethenyl (C₂), propenyl (C₃), or butenyl (C₄), substituted or unsubstituted with one or more substituents selected from the group consisting of alkyl, halo, haloalkyl, alkoxy, alkoxyalkyl, or hydroxyl. In certain embodiments, R²⁰ or R^{A} is ethenyl, propenyl, or butenyl, substituted or unsubstituted with alkyl, halo, haloalkyl, alkoxy, alkoxyalkyl, or hydroxyl.

In certain embodiments, R²⁰ or R^{A} is substituted or unsubstituted —C₂₋₆ alkynyl, e.g, substituted or unsubstituted —C₂₋₃ alkynyl, substituted or unsubstituted —C₃₋₄ alkynyl, substituted or unsubstituted —C₄₋₅ alkynyl, substituted or unsubstituted —C₅₋₆ alkynyl. In certain embodiments, R²⁰ or R^{A} is ethynyl, propynyl, or butynyl, substituted or unsubstituted with alkyl, halo, haloalkyl (e.g., CF₃), alkoxyalkyl, cycloalkyl (e.g., cyclopropyl or cyclobutyl), or hydroxyl.

In certain embodiments, R²⁰ or R^{A} is substituted or unsubstituted C₃₋₆ carbocyclyl, e.g. substituted or unsubstituted C₃₋₄ carbocyclyl, substituted or unsubstituted C₄₋₅ carbocyclyl, or substituted or unsubstituted C₅₋₆ carbocyclyl. In certain embodiments, R²⁰ or R^{A} is substituted or unsubstituted cyclopropyl, substituted or unsubstituted cyclopropylmethyl-, substituted or unsubstituted cyclobutyl, substituted or unsubstituted cyclopentyl, substituted or unsubstituted cyclohexyl.

In one embodiment, R²⁰ is cyclopropyl, cyclobutyl, cyclopentyl, or cyclohexyl.

In certain embodiments, R²⁰ or R^{A} is substituted or unsubstituted 3- to 6-membered heterocyclyl, e.g. substituted or unsubstituted 3-4 membered heterocyclyl, substituted or unsubstituted 4-5 membered heterocyclyl, or substituted or unsubstituted 5-6 membered heterocyclyl.

In one embodiment, R²⁰ or R^{A} is substituted or unsubstituted —C₆-C₁₂ aryl. In one embodiment, R²⁰ is phenyl. In certain embodiments, R²⁰ is phenyl substituted or unsubstituted with cyano, alkyl, halo, haloalkyl, alkoxy, alkoxyalkyl, or hydroxyl.

In one embodiment, R²⁰ or R^{A} is substituted or unsubstituted 5-12 membered heteroaryl. In certain embodiments, R²⁰ is substituted or unsubstituted pyridyl. In certain embodiments, R²⁰ is substituted or unsubstituted oxazole. In certain embodiments, R²⁰ is substituted or unsubstituted pyrazole.

In one embodiment, R²⁰ is pyrazole substituted with alkyl. In some embodiments, R²⁰ is pyrazole substituted with methyl. In some embodiments, R²⁰ is N-methylpyrazole.

In one embodiment of the compounds of the present invention, R², R⁴, R⁵, R⁶, R⁷, R¹⁰, R^{11a}, R^{11b}, R¹², R¹⁶, R^{19a} and R^{19b} are all hydrogen, R³ is methyl, and R²⁰ is hydrogen, substituted or unsubstituted —C₁₋₆ alkyl, —C₂₋₆ alkenyl, —C₂₋₆ alkynyl, —C₁₋₆ haloalkyl, —C₃₋₁₂ cycloalkyl, C₃₋₁₂ cycloalkyl-C₁-C₆ alkyl-, heterocyclyl, —C₆-C₁₂ aryl, C₆-C₁₂ aryl-C₁-C₆ alkyl-, 5-12 membered heteroaryl, or 5-12 membered heteroaryl-C₁-C₆ alkyl-. In one embodiment, R²⁰ is —C₁₋₆ alkyl, —C₁₋₆ haloalkyl, —C₃₋₁₂ cycloalkyl, C₃₋₁₂ cycloalkyl-C₁-C₆ alkyl-, —C₆-C₁₂ aryl, or 5-12 membered heteroaryl, which can be optionally substituted with halogen, cyano, nitro, substituted or unsubstituted alkyl, alkenyl, alkynyl, carbocylyl, 3-12 membered heterocylyl, —C₆-C₁₂ aryl, 5-12 membered heteroaryl, —OR^{A}, —C(═O)R^{A}, —C(═O)OR^{A}, —OC(═O)R^{A}, —OC(═O)OR^{A}, —C(═O)NR^{A}R^{A}, —NR^{A}R^{A}, —NR^{A}C(═O)R^{A}, —OC(═O)NR^{A}R^{A}, —NR^{A}C(═O)OR^{A}, —NR^{A}C(═O)NR^{A}R^{A}, —SR^{A}, —S(═O)R^{A}, —S(═O)₂R^{A}, —S(═O)₂OR^{A}, —OS(═O)₂R^{A}, —S(═O)₂NR^{A}R^{A}, or —NR^{A}S(═O)₂R^{A}, wherein R^{A} is independently hydrogen or substituted or unsubstituted alkyl, alkenyl, or alkynyl, substituted or unsubstituted carbocylyl, substituted or unsubstituted heterocylyl, substituted or unsubstituted aryl, substituted or unsubstituted heteroaryl, or two R^{A} groups can be taken together with the atoms to which they are attached to, to form a substituted or unsubstituted heterocylyl or heteroaryl ring. In one embodiment, R²⁰ is —C₁₋₆ alkyl, —C₃₋₁₂ cycloalkyl, C₃₋₁₂ cycloalkyl-C₁-C₆ alkyl-, —C₆-C₁₂ aryl, or 5-12 membered heteroaryl, which can be optionally substituted with halogen, cyano, substituted or unsubstituted alkyl, substituted or unsubstituted carbocylyl, or alkoxy. In one embodiment, R²⁰ is substituted or unsubstituted —C₁₋₆ alkyl. In one embodiment, R²⁰ is —CH₃, —CF₃, —CH₂CH₃, -i-Pr, -n-Pr, -i-Bu, -s-Bu, -t-Bu, —CH₂cyclopropyl, —CH₂CN, or —CH₂CH₂CN. In one embodiment, R²⁰ is —CH₃, —CH₂CH₃, -i-Pr, -n-Pr, -i-Bu, -s-Bu, -t-Bu, —CH₂cycpropyl. In one embodiment, R²⁰ is substituted or unsubstituted —C₃₋₁₂ cycloalkyl. In one embodiment, R²⁰ is cyclopropyl, cyclobutyl, cyclopentyl, or cyclohexyl. In one embodiment, R²⁰ is substituted or unsubstituted —C₆-C₁₂ aryl. In one embodiment, R²⁰ is phenyl. In one embodiment, R²⁰ is phenyl substituted with one or more halogen, cyano, or alkoxy. In one embodiment, R²⁰ is substituted or unsubstituted 5-12 membered heteroaryl. In one embodiment, R²⁰ is pyridine. In one embodiment, R²⁰ is pyridine substituted with one or more halogen, cyano, alkoxy. In one embodiment, R²⁰ is substituted or unsubstituted oxazole or pyrazole. In one embodiment, R²⁰ is oxazole, pyrazole, or N-methylpyrazole.

In one embodiment, R²⁰ is R²⁰ is hydrogen, —CH₃, —CF₃, —CH₂CH₃, -i-Pr, -t-Bu, cyclopropyl, —CH₂cyclopropyl, cyclopentyl, cyclohexyl, —CH₂CN, —CH₂CH₂CN, phenyl, pyridyl, oxazole, or pyrazole, wherein phenyl, pyridyl, oxazole, and pyrazole is each optionally substituted. In one embodiment, R²⁰ is R²⁰ is hydrogen, —CH₃, —CF₃, —CH₂CH₃, -i-Pr, -t-Bu, cyclopropyl, —CH₂cyclopropyl, cyclopentyl, cyclohexyl, —CH₂CN, —CH₂CH₂CN, phenyl, pyridyl, oxazole, or pyrazole, wherein phenyl, pyridyl, oxazole, and pyrazole is each optionally substituted with halogen, cyano, —O(C₁-C₃ alkyl), or —C₁-C₃ alkyl.

In one embodiment, R²⁰ is R²⁰ is hydrogen, —CH₃, —CF₃, —CH₂CH₃, -i-Pr, -t-Bu, cyclopropyl, —CH₂cyclopropyl, cyclopentyl, cyclohexyl, —CH₂CN, —CH₂CH₂CN, phenyl,

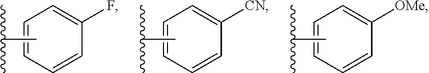

pyridyl, oxazole, pyrazole, or N-methylpyrazole. yl, —CH₂cyclopropyl, cyclopentyl, cyclohexyl, —CH₂CN, —CH₂CH₂CN, phenyl,

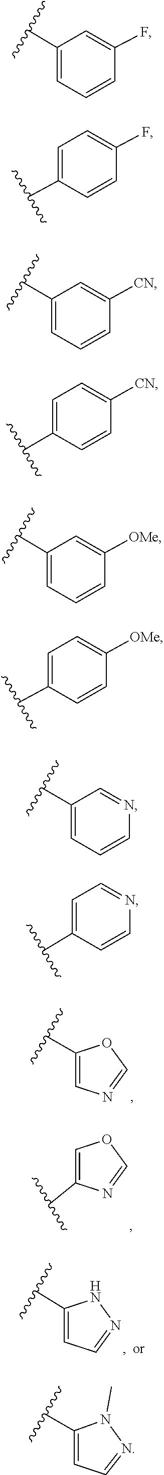

In one embodiment, the nitrogen protecting group, the sulfur protecting group, or the oxygen protecting group is benzyl.

### Various Combinations of Certain Embodiments

Various combinations of certain embodiments are further contemplated herein.

For example, in certain embodiments, wherein R² is hydrogen or a non-hydrogen alpha substituent, provided is a steroid of Formula (I-A1), (I-B1), (I-C1), (I-D1), (II-A1), (II-B1), (II-C1), or (II-D1):

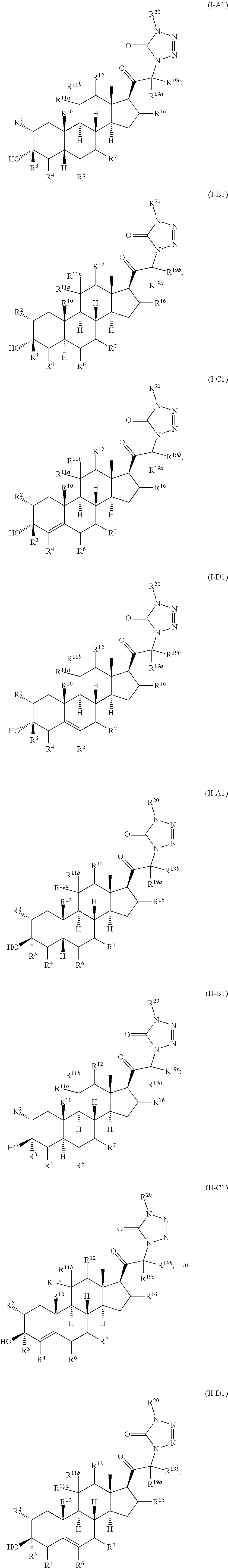

or a pharmaceutically acceptable salt, solvate, ester, or prodrug thereof.

In certain embodiments, R³ is hydrogen, —CH₃, —CH₂CH₃, —CH₂F, —CHF₂, —CF₃, —CH₂OCH₂CH₃, —CH₂OCH₃, or substituted or unsubstituted cyclopropyl. In certain embodiments, R² is —OH, —OCH₃, —OCH₂CH₃, —OCH₂CH₂CH₃, —CH₃, —CH₂CH₃, —CH₂CH₂CH₃, substituted or unsubstituted cyclopropyl, fluoro, or chloro. In certain embodiments, R^{11a} and R^{11b} are both hydrogen. In certain embodiments, R^{11a} and R^{11b} are joined to form ═O (oxo). In certain embodiments, R^{11a} is —OH, —OCH₃, —OCH₂CH₃ or —OCH₂CH₃CH₃, and R^{11b} is hydrogen. In certain embodiments, R⁴ and R⁶ are independently hydrogen, fluoro, —CH₃, or —CF₃. In one embodiment of the compounds of the present invention, at least one of R⁴ or R⁶ is hydrogen, substituted or unsubstituted —C₁₋₆ alkyl, halogen, —CH₃, or —CF₃. In certain embodiments, both R⁴ and R⁶ are hydrogen. In certain embodiments, R⁷, R¹² and R¹⁶ are independently hydrogen, —CH₃, —CH₂CH₃, —CH(CH₃)₂, —OH, —OCH₃, or —CH₂OCH₃. In certain embodiments, R⁴, R⁶, R⁷, R¹² and R¹⁶ are independently a non-hydrogen substituent in the alpha configuration. In certain embodiments, R⁴, R⁶, R⁷, R¹² and R¹⁶ are independently a non-hydrogen substituent in the beta configuration. In certain embodiments, R¹⁰ is hydrogen, halogen, cyano, —CH₃, —CH₂CH₃, —CH₂F, —CHF₂, —CF₃, —CH₂OCH₃, or —CH₂SCH₃. In some embodiments, R¹⁰ is hydrogen, —CH₃, —CH₂CH₃, or —CH₂OCH₃. In certain embodiments, R^{19a} and R^{19b} are both hydrogen. In certain embodiments, one of R^{19a} and R^{19b} is not hydrogen. In certain embodiments, both of R^{19a} and R^{19b} are not hydrogen. In certain embodiments, R^{19a} is C₁₋₆ alkyl. In certain embodiments, R^{19b} is hydrogen or C₁₋₆ alkyl. In certain embodiments, R²⁰ is hydrogen, substituted or unsubstituted —C₁₋₆ alkyl (e.g. —CH₃, —CF₃, —CH₂CH₃, -i-Pr, -n-Pr, -i-Bu, -s-Bu, -t-Bu, —CH₂cyclopropyl, —CH₂CN, or —CH₂CH₂CN), —C₂₋₆ alkenyl, —C₂₋₆ alkynyl, —C₁₋₆ haloalkyl, —C₃₋₁₂ cycloalkyl (e.g. cyclopropyl, cyclobutyl, cyclopentyl, cyclohexyl), C₃₋₁₂ cycloalkyl-C₁-C₆ alkyl-, heterocyclyl, —C₆-C₁₂ aryl (e.g. phenyl), C₆-C₁₂ aryl-C₁-C₆ alkyl-, 5-12 membered heteroaryl (e.g. pyridine, oxazole, or pyrazole), or 5-12 membered heteroaryl-C₁-C₆ alkyl-.

In certain embodiments, wherein R² is hydrogen or a non-hydrogen beta substituent, provided is a steroid of Formula (I-A2), (I-B2), (I-C2), (I-D2), (II-A2), (II-B2), (II-C2) or (II-D2):

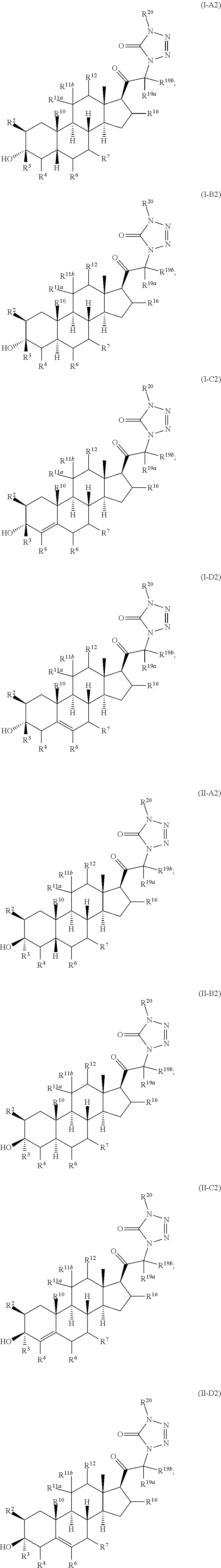

or a pharmaceutically acceptable salt, solvate, ester, or prodrug thereof.

In certain embodiments, R³ is hydrogen, —CH₃, —CH₂CH₃, —CH₂F, —CHF₂, —CF₃, —CH₂OCH₂CH₃, —CH₂OCH₃, or substituted or unsubstituted cyclopropyl. In certain embodiments, R² is —OH, —OCH₃, —OCH₂CH₃, —OCH₂CH₂CH₃, —CH₃, —CH₂CH₃, —CH₂CH₂CH₃, substituted or unsubstituted cyclopropyl, fluoro, or chloro. In certain embodiments, R^{11a} and R^{11b} are both hydrogen. In certain embodiments, R^{11a} and R^{11b} are joined to form ═O (oxo). In certain embodiments, R^{11a} is —OH, —OCH₃, —OCH₂CH₃ or —OCH₂CH₃CH₃, and R^{11b} is hydrogen. In certain embodiments, R⁴ and R⁶ are independently hydrogen, fluoro, —CH₃, or —CF₃. In one embodiment of the compounds of the present invention, at least one of R⁴ or R⁶ is hydrogen, substituted or unsubstituted —C₁₋₆ alkyl, halogen, —CH₃, or —CF₃. In certain embodiments, both R⁴ and R⁶ are hydrogen. In certain embodiments, R⁷, R¹² and R¹⁶ are independently hydrogen, —CH₃, —CH₂CH₃, —CH(CH₃)₂, —OH, —OCH₃, or —CH₂OCH₃. In certain embodiments, R⁴, R⁶, R⁷, R¹² and R¹⁶ are independently a non-hydrogen substituent in the alpha configuration. In certain embodiments, R⁴, R⁶, R⁷, R¹² and R¹⁶ are independently a non-hydrogen substituent in the beta configuration. In certain embodiments, R¹⁰ is hydrogen, halogen, cyano, —CH₃, —CH₂CH₃, —CH₂F, —CHF₂, —CF₃, —CH₂OCH₃, or —CH₂SCH₃. In some embodiments, R¹⁰ is hydrogen, —CH₃, —CH₂CH₃, or —CH₂OCH₃. In certain embodiments, R^{19a} and R^{19b} are both hydrogen. In certain embodiments, one of R^{19a} and R^{19b} is not hydrogen. In certain embodiments, both of R^{19a} and R^{19b} are not hydrogen. In certain embodiments, R^{19a} is C₁₋₆ alkyl. In certain embodiments, R^{19b} is hydrogen or C₁₋₆ alkyl. In certain embodiments, R²⁰ is hydrogen, substituted or unsubstituted —C₁₋₆ alkyl (e.g. —CH₃, —CF₃, —CH₂CH₃, -i-Pr, -n-Pr, -i-Bu, -s-Bu, -t-Bu, —CH₂cyclopropyl, —CH₂CN, or —CH₂CH₂CN), —C₂₋₆ alkenyl, —C₂₋₆ alkynyl, —C₁₋₆ haloalkyl, —C₃₋₁₂ cycloalkyl (e.g. cyclopropyl, cyclobutyl, cyclopentyl, cyclohexyl), heterocyclyl, —C₆-C₁₂ aryl (e.g. phenyl), C₆₋C₁₂ aryl-C₁-C₆ alkyl-, 5-12 membered heteroaryl (e.g. pyridine, oxazole, or pyrazole), or 5-12 membered heteroaryl-C₁-C₆ alkyl-.

In certain embodiments, wherein R^{11a} is hydrogen or a non-hydrogen alpha substituent, and R^{11b} is hydrogen, provided is a steroid of Formula (I-A3), (I-B3), (I-C3), (I-D3), (II-A3), (II-B3), (II-C3) or (II-D3):

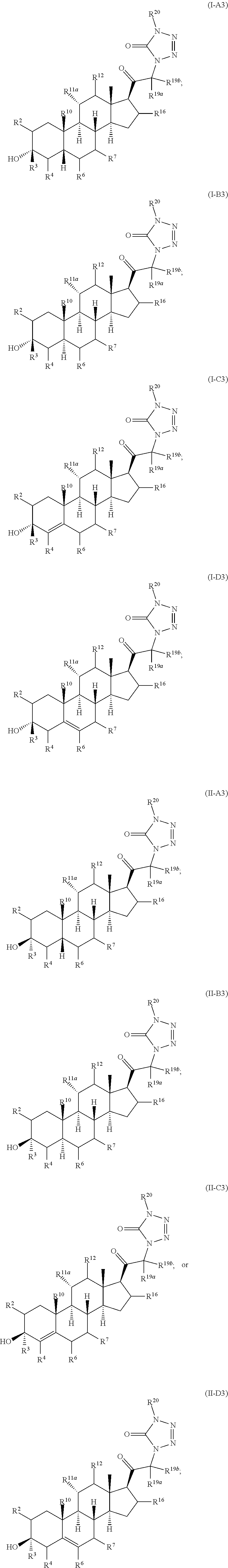

or a pharmaceutically acceptable salt, solvate, ester, or prodrug thereof.

In certain embodiments, R³ is hydrogen, —CH₃, —CH₂CH₃, —CH₂F, —CHF₂, —CF₃, —CH₂OCH₂CH₃, —CH₂OCH₃, or substituted or unsubstituted cyclopropyl. In certain embodiments, R² is hydrogen, —OH, —OCH₃, —OCH₂CH₃, —OCH₂CH₂CH₃, —CH₃, —CH₂CH₃, —CH₂CH₂CH₃, substituted or unsubstituted cyclopropyl, fluoro, or chloro. In certain embodiments, R^{11a} is hydrogen, —OH, —OCH₃, —OCH₂CH₃ or —OCH₂CH₃CH₃. In certain embodiments, R⁴ and R⁶ are independently hydrogen, fluoro, —CH₃, or —CF₃. In one embodiment of the compounds of the present invention, at least one of R⁴ or R⁶ is hydrogen, substituted or unsubstituted —C₁₋₆ alkyl, halogen, —CH₃, or —CF₃. In certain embodiments, both R⁴ and R⁶ are hydrogen. In certain embodiments, R⁷, R¹² and R¹⁶ are independently hydrogen, —CH₃, —CH₂CH₃, —CH(CH₃)₂, —OH, —OCH₃, or —CH₂OCH₃. In certain embodiments, R², R⁴, R⁶, R⁷, R¹² and R¹⁶ are independently a non-hydrogen substituent in the alpha configuration. In certain embodiments, R², R⁴, R⁶, R⁷, R¹² and R¹⁶ are independently a non-hydrogen substituent in the beta configuration. In certain embodiments, R¹⁰ is hydrogen, halogen, cyano, —CH₃, —CH₂CH₃, —CH₂F, —CHF₂, —CF₃, —CH₂OCH₃, or —CH₂SCH₃. In some embodiments, R¹⁰ is hydrogen, —CH₃, —CH₂CH₃, or —CH₂OCH₃. In certain embodiments, R^{19a} and R^{19b} are both hydrogen. In certain embodiments, one of R^{19a} and R^{19b} is not hydrogen. In certain embodiments, both of R^{19a} and R^{19b} are not hydrogen. In certain embodiments, R^{19a} is C₁₋₆ alkyl. In certain embodiments, R^{19b} is hydrogen or C₁₋₆ alkyl. In certain embodiments, R²⁰ is hydrogen, substituted or unsubstituted —C₁₋₆ alkyl (e.g. —CH₃, —CF₃, —CH₂CH₃, -i-Pr, -n-Pr, -i-Bu, -s-Bu, -t-Bu, —CH₂cyclopropyl, —CH₂CN, or —CH₂CH₂CN), —C₂₋₆ alkenyl, —C₂₋₆ alkynyl, —C₁₋₆ haloalkyl, —C₃₋₁₂ cycloalkyl (e.g. cyclopropyl, cyclobutyl, cyclopentyl, cyclohexyl), heterocyclyl, —C₆-C₁₂ aryl (e.g. phenyl), C₆-C₁₂ aryl-C₁-C₆ alkyl-, 5-12 membered heteroaryl (e.g. pyridine, oxazole, or pyrazole), or 5-12 membered heteroaryl-C₁-C₆ alkyl-.

In certain embodiments, wherein R^{11a} is hydrogen or a non-hydrogen beta substituent, and R^{11b} is hydrogen, provided is a steroid of Formula (I-A4), (I-B4), (I-C4), (I-D4), (II-A4), (II-B4), (II-C4) or (II-D4):

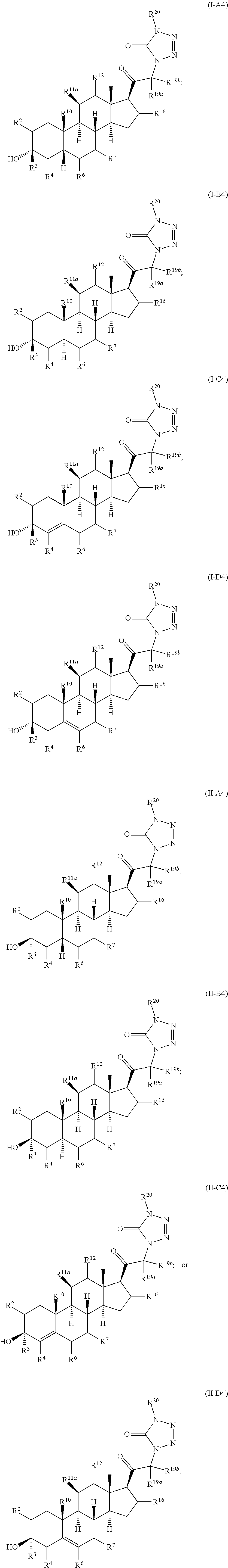

or a pharmaceutically acceptable salt, solvate, ester, or prodrug thereof.

In certain embodiments, R³ is hydrogen, —CH₃, —CH₂CH₃, —CH₂F, —CHF₂, —CF₃, —CH₂OCH₂CH₃, —CH₂OCH₃, or substituted or unsubstituted cyclopropyl. In certain embodiments, R² is hydrogen, —OH, —OCH₃, —OCH₂CH₃, —OCH₂CH₂CH₃, —CH₃, —CH₂CH₃, —CH₂CH₂CH₃, substituted or unsubstituted cyclopropyl, fluoro, or chloro. In certain embodiments, R^{11a} is hydrogen, —OH, —OCH₃, —OCH₂CH₃ or —OCH₂CH₃CH₃. In certain embodiments, R⁴ and R⁶ are independently hydrogen, fluoro, —CH₃, or —CF₃. In one embodiment of the compounds of the present invention, at least one of R⁴ or R⁶ is hydrogen, substituted or unsubstituted —C₁₋₆ alkyl, halogen, —CH₃, or —CF₃. In certain embodiments, both R⁴ and R⁶ are hydrogen. In certain embodiments, R⁷, R¹² and R¹⁶ are independently hydrogen, —CH₃, —CH₂CH₃, —CH(CH₃)₂, —OH, —OCH₃, or —CH₂OCH₃. In certain embodiments, R², R⁴, R⁶, R⁷, R¹² and R¹⁶ are independently a non-hydrogen substituent in the alpha configuration. In certain embodiments, R², R⁴, R⁶, R⁷, R¹² and R¹⁶ are independently a non-hydrogen substituent in the beta configuration. In certain embodiments, R¹⁰ is hydrogen, halogen, cyano, —CH₃, —CH₂CH₃, —CH₂F, —CHF₂, —CF₃, —CH₂OCH₃, or —CH₂SCH₃. In some embodiments, R¹⁰ is hydrogen, —CH₃, —CH₂CH₃, or —CH₂OCH₃. In certain embodiments, R^{19a} and R^{19b} are both hydrogen. In certain embodiments, one of R^{19a} and R^{19b} is not hydrogen. In certain embodiments, both of R^{19a} and R^{19b} are not hydrogen. In certain embodiments, R^{19a} is C₁₋₆ alkyl. In certain embodiments, R^{19b} is hydrogen or C₁₋₆ alkyl. In certain embodiments, R²⁰ is hydrogen, substituted or unsubstituted —C₁₋₆ alkyl (e.g. —CH₃, —CF₃, —CH₂CH₃, -i-Pr, -t-Bu, —CH₂cyclopropyl, —CH₂CN, or —CH₂CH₂CN), —C₂₋₆ alkenyl, —C₂₋₆ alkynyl, —C₁₋₆ haloalkyl, —C₃₋₁₂ cycloalkyl (e.g. cyclopropyl, cyclobutyl, cyclopentyl, cyclohexyl), heterocyclyl, —C₆-C₁₂ aryl (e.g. phenyl), C₆-C₁₂ aryl-C₁-C₆ alkyl-, 5-12 membered heteroaryl (e.g. pyridine, oxazole, or pyrazole), or 5-12 membered heteroaryl-C₁-C₆ alkyl-.

In certain embodiments, wherein R^{11a} and R^{11b} are joined to form an oxo group, provided is a steroid of Formula (I-A5), (I-B5), (I-C5), (I-D5), (II-A5), (II-B5), (II-C5), (II-D5):

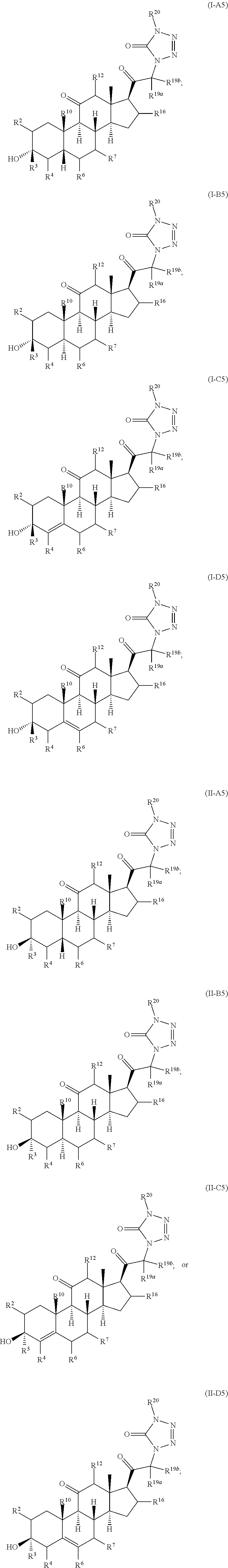

or a pharmaceutically acceptable salt, solvate, ester, or prodrug thereof.

In certain embodiments, R³ is hydrogen, —CH₃, —CH₂CH₃, —CH₂F, —CHF₂, —CF₃, —CH₂OCH₂CH₃, —CH₂OCH₃, or substituted or unsubstituted cyclopropyl. In certain embodiments, R² is hydrogen, —OH, —OCH₃, —OCH₂CH₃, —OCH₂CH₂CH₃, —CH₃, —CH₂CH₃, —CH₂CH₂CH₃, substituted or unsubstituted cyclopropyl, fluoro, or chloro. In certain embodiments, R⁴ and R⁶ are independently hydrogen, fluoro, —CH₃, or —CF₃. In one embodiment of the compounds of the present invention, at least one of R⁴ or R⁶ is hydrogen, substituted or unsubstituted —C₁₋₆ alkyl, halogen, —CH₃, or —CF₃. In certain embodiments, both R⁴ and R⁶ are hydrogen. In certain embodiments, R⁷, R¹² and R¹⁶ are independently hydrogen, —CH₃, —CH₂CH₃, —CH(CH₃)₂, —OH, —OCH₃, or —CH₂OCH₃. In certain embodiments, R², R⁴, R⁶, R⁷, R¹² and R¹⁶ are independently a non-hydrogen substituent in the alpha configuration. In certain embodiments, R², R⁴, R⁶, R⁷, R¹² and R¹⁶ are independently a non-hydrogen substituent in the beta configuration. In certain embodiments, R¹⁰ is hydrogen, halogen, cyano, —CH₃, —CH₂CH₃, —CH₂F, —CHF₂, —CF₃, —CH₂OCH₃, or —CH₂SCH₃. In certain embodiments, R¹⁰ is hydrogen, —CH₃, —CH₂CH₃ or —CH₂OCH₃. In certain embodiments, R^{19a} and R^{19b} are both hydrogen. In certain embodiments, one of R^{19a} and R^{19b} is not hydrogen. In certain embodiments, both of R^{19a} and R^{19b} are not hydrogen. In certain embodiments, R^{19a} is C₁₋₆ alkyl. In certain embodiments, R^{19b} is hydrogen or C₁₋₆ alkyl. In certain embodiments, R²⁰ is hydrogen, substituted or unsubstituted —C₁₋₆ alkyl (e.g. —CH₃, —CF₃, —CH₂CH₃, -i-Pr, -t-Bu, —CH₂cyclopropyl, —CH₂CN, or —CH₂CH₂CN), —C₂₋₆ alkenyl, —C₂₋₆ alkynyl, —C₁₋₆ haloalkyl, —C₃₋₁₂ cycloalkyl (e.g. cyclopropyl, cyclobutyl, cyclopentyl, cyclohexyl), heterocyclyl, —C₆-C₁₂ aryl (e.g. phenyl), C₆-C₁₂ aryl-C₁-C₆ alkyl-, 5-12 membered heteroaryl (e.g. pyridine, oxazole, or pyrazole), or 5-12 membered heteroaryl-C₁-C₆ alkyl-.

In certain embodiments, wherein R^{19a} is a non-hydrogen substitute, and R^{19b} is hydrogen, provided is a steroid of Formula (I-A6), (I-B6), (I-C6), (I-D6), (II-A6), (II-B6), (II-C6), (II-D6):

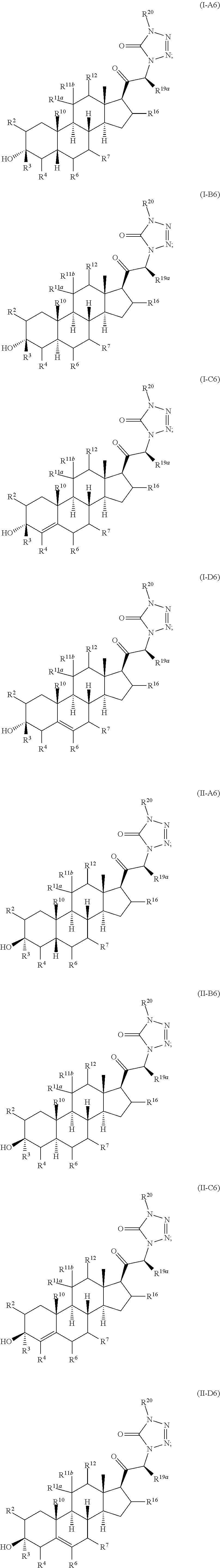

or a pharmaceutically acceptable salt, solvate, ester, or prodrug thereof.

In certain embodiments, R³ is hydrogen, —CH₃, —CH₂CH₃, —CH₂F, —CHF₂, —CF₃, —CH₂OCH₂CH₃, —CH₂OCH₃, or substituted or unsubstituted cyclopropyl. In certain embodiments, R² is hydrogen, —OH, —OCH₃, —OCH₂CH₃, —OCH₂CH₂CH₃, —CH₃, —CH₂CH₃, —CH₂CH₂CH₃, substituted or unsubstituted cyclopropyl, fluoro, or chloro. In certain embodiments, R^{11a} and R^{11b} are both hydrogen. In certain embodiments, R^{11a} and R^{11b} are joined to form ═O (oxo). In certain embodiments, R^{11a} is —OH, —OCH₃, —OCH₂CH₃ or —OCH₂CH₃CH₃, and R^{11b} is hydrogen. In certain embodiments, R⁴ and R⁶ are independently hydrogen, fluoro, —CH₃, or —CF₃. In one embodiment of the compounds of the present invention, at least one of R⁴ or R⁶ is hydrogen, substituted or unsubstituted —C₁₋₆ alkyl, halogen, —CH₃, or —CF₃. In certain embodiments, both R⁴ and R⁶ are hydrogen. In certain embodiments, R⁷, R¹² and R¹⁶ are independently hydrogen, —CH₃, —CH₂CH₃, —CH(CH₃)₂, —OH, —OCH₃, or —CH₂OCH₃. In certain embodiments, R⁴, R⁶, R⁷, R¹² and R¹⁶ are independently a non-hydrogen substituent in the alpha configuration. In certain embodiments, R⁴, R⁶, R⁷, R¹² and R¹⁶ are independently anon-hydrogen substituent in the beta configuration. In certain embodiments, R¹⁰ is hydrogen, halogen, cyano, —CH₃, —CH₂CH₃, —CH₂F, —CHF₂, —CF₃, —CH₂OCH₃, or —CH₂SCH₃. In some embodiments, R¹⁰ is hydrogen, —CH₃, —CH₂CH₃, or —CH₂OCH₃. In certain embodiments, R^{19a} is C₁₋₆ alkyl. In certain embodiments, R^{19a} is methyl. In certain embodiments, R²⁰ is hydrogen, substituted or unsubstituted —C₁₋₆ alkyl (e.g. —CH₃, —CF₃, —CH₂CH₃, -i-Pr, -t-Bu, —CH₂cyclopropyl, —CH₂CN, or —CH₂CH₂CN), —C₂₋₆ alkenyl, —C₂₋₆ alkynyl, —C₁₋₆ haloalkyl, —C₃₋₁₂ cycloalkyl (e.g. cyclopropyl, cyclobutyl, cyclopentyl, cyclohexyl), heterocyclyl, —C₆-C₁₂ aryl (e.g. phenyl), C₆-C₁₂ aryl-C₁-C₆ alkyl-, 5-12 membered heteroaryl (e.g. pyridine, oxazole, or pyrazole), or 5-12 membered heteroaryl-C₁-C₆ alkyl-.

In certain embodiments, wherein R^{19a} is a non-hydrogen substitute, and R^{19b} is hydrogen, provided is a steroid of Formula (I-A7), (I-B7), (I-C7), (I-D7), (II-A7), (II-B7), (II-C7), (II-D7):

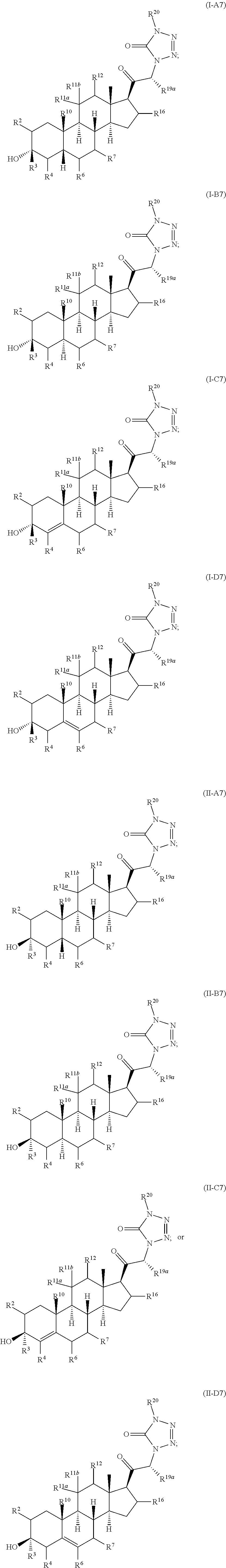

or a pharmaceutically acceptable salt, solvate, ester, or prodrug thereof.

In certain embodiments, R³ is hydrogen, —CH₃, —CH₂CH₃, —CH₂F, —CHF₂, —CF₃, —CH₂OCH₂CH₃, —CH₂OCH₃, or substituted or unsubstituted cyclopropyl. In certain embodiments, R² is hydrogen, —OH, —OCH₃, —OCH₂CH₃, —OCH₂CH₂CH₃, —CH₃, —CH₂CH₃, —CH₂CH₂CH₃, substituted or unsubstituted cyclopropyl, fluoro, or chloro. In certain embodiments, R^{11a} and R^{11b} are both hydrogen. In certain embodiments, R^{11a} and R^{11b} are joined to form ═O (oxo). In certain embodiments, R^{11a} is —OH, —OCH₃, —OCH₂CH₃ or —OCH₂CH₃CH₃, and R^{11b} is hydrogen. In certain embodiments, R⁴ and R⁶ are independently hydrogen, fluoro, —CH₃, or —CF₃. In one embodiment of the compounds of the present invention, at least one of R⁴ or R⁶ is hydrogen, substituted or unsubstituted —C₁₋₆ alkyl, halogen, —CH₃, or —CF₃. In certain embodiments, both R⁴ and R⁶ are hydrogen. In certain embodiments, R⁷, R¹² and R¹⁶ are independently hydrogen, —CH₃, —CH₂CH₃, —CH(CH₃)₂, —OH, —OCH₃, or —CH₂OCH₃. In certain embodiments, R⁴, R⁶, R⁷, R¹² and R¹⁶ are independently a non-hydrogen substituent in the alpha configuration. In certain embodiments, R⁴, R⁶, R⁷, R¹² and R¹⁶ are independently anon-hydrogen substituent in the beta configuration. In certain embodiments, R¹⁰ is hydrogen, halogen, cyano, —CH₃, —CH₂CH₃, —CH₂F, —CHF₂, —CF₃, —CH₂OCH₃, or —CH₂SCH₃. In some embodiments, R¹⁰ is hydrogen, —CH₃, —CH₂CH₃, or —CH₂OCH₃. In certain embodiments, R^{19a} is C₁₋₆ alkyl. In certain embodiments, R^{19a} is methyl. In certain embodiments, R²⁰ is hydrogen, substituted or unsubstituted —C₁₋₆ alkyl (e.g. —CH₃, —CF₃, —CH₂CH₃, -i-Pr, -t-Bu, —CH₂cyclopropyl, —CH₂CN, or —CH₂CH₂CN), —C₂₋₆ alkenyl, —C₂₋₆ alkynyl, —C₁₋₆ haloalkyl, —C₃₋₁₂ cycloalkyl (e.g. cyclopropyl, cyclobutyl, cyclopentyl, cyclohexyl), heterocyclyl, —C₆-C₁₂ aryl (e.g. phenyl), C₆-C₁₂ aryl-C₁-C₆ alkyl-, 5-12 membered heteroaryl (e.g. pyridine, oxazole, or pyrazole), or 5-12 membered heteroaryl-C₁-C₆ alkyl-.

In certain embodiments, wherein R², R⁴, R⁵, R⁶, R⁷, R¹⁰, R^{11a}, R^{11b}, R¹², R¹⁶, R^{19a} and R^{19b} are hydrogen, provided is a steroid of Formula (I-A8), (I-B8), (II-A8), or (II-B8):

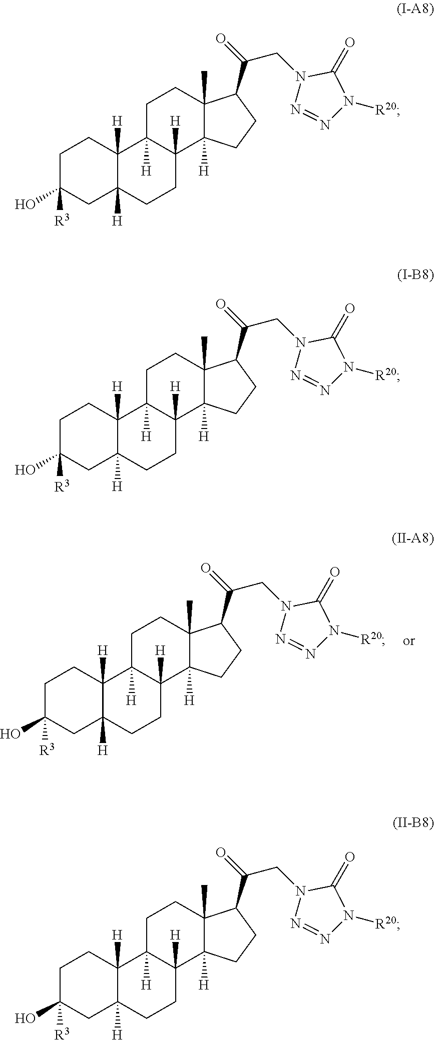

or a pharmaceutically acceptable salt, solvate, ester, or prodrug thereof.

In certain embodiments, R³ is hydrogen, —CH₃, —CH₂CH₃, —CH₂F, —CHF₂, —CF₃, —CH₂OCH₂CH₃, —CH₂OCH₃, or substituted or unsubstituted cyclopropyl. In certain embodiments, R²⁰ is hydrogen, substituted or unsubstituted —C₁₋₆ alkyl (e.g. —CH₃, —CF₃, —CH₂CH₃, -i-Pr, -t-Bu, —CH₂cyclopropyl, —CH₂CN, or —CH₂CH₂CN), —C₂₋₆ alkenyl, —C₂₋₆ alkynyl, —C₁₋₆ haloalkyl, —C₃₋₁₂ cycloalkyl (e.g. cyclopropyl, cyclobutyl, cyclopentyl, cyclohexyl), heterocyclyl, —C₆-C₁₂ aryl (e.g. phenyl), C₆-C₁₂ aryl-C₁-C₆ alkyl-, 5-12 membered heteroaryl (e.g. pyridine, oxazole, or pyrazole), or 5-12 membered heteroaryl-C₁-C₆ alkyl-.

In certain embodiments, R³ is methyl.

In one embodiment of the compounds of Formula (I-A8), (I-B8), (II-A8), or (II-B8), or or a pharmaceutically acceptable salt, solvate, ester, or prodrug thereof:
R³ is methyl; and
R²⁰ is hydrogen, —CH₃, —CF₃, —CH₂CH₃, -i-Pr, -t-Bu, cyclopropyl, —CH₂cyclopropyl, cyclopentyl, cyclohexyl, —CH₂CN, —CH₂CH₂CN, phenyl, pyridyl, oxazole, or pyrazole;
wherein phenyl, pyridyl, oxazol, and pyrazole are optionally substituted with halogen, cyano, —O(C₁-C₃ alkyl), or —C₁-C₃ alkyl.

In certain embodiments, a steroid of Formula (AI), (I), (AII), and (II) is selected from Table A or a pharmaceutically acceptable salt, solvate, ester, or prodrug thereof. In one embodiment of the compounds of the present invention, the compound is selected from the compounds prepared in any one of Examples 1-18, or pharmaceutically acceptable salts, solvates, esters, or prodrugs thereof. In one embodiment of the compounds of the present invention, the compound is selected from the compounds prepared in any one of Examples 19-29, or pharmaceutically acceptable salts, solvates, esters, or prodrugs thereof.

TABLE A

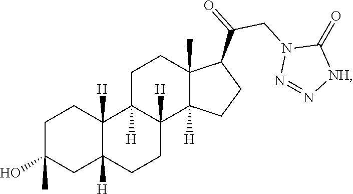

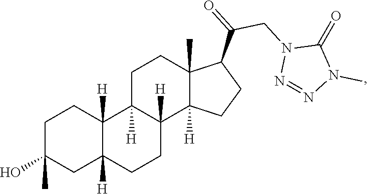

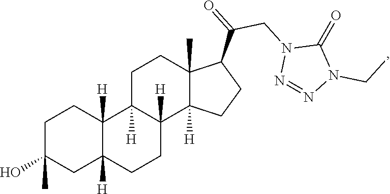

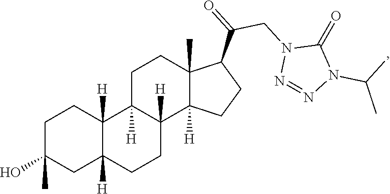

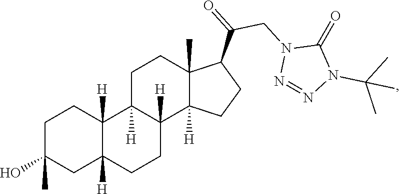

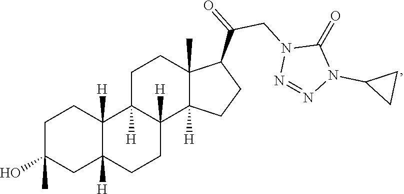

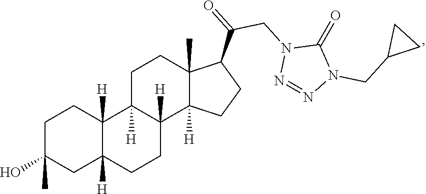

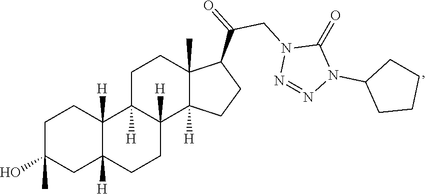

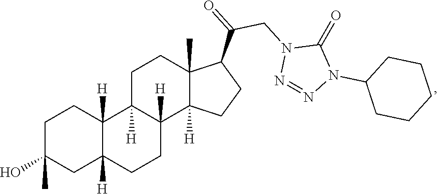

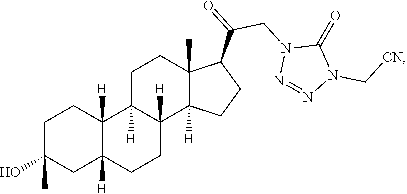

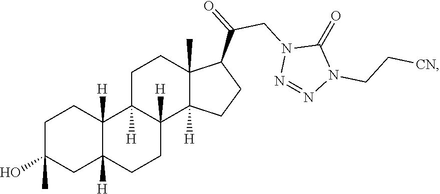

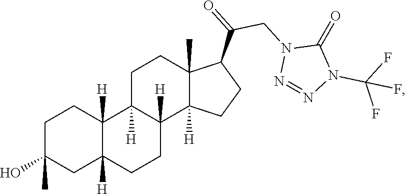

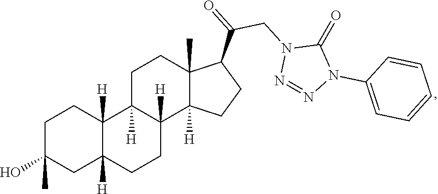

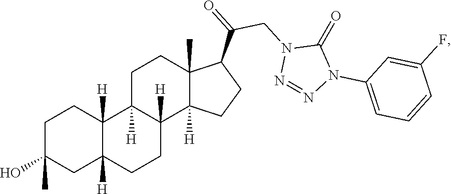

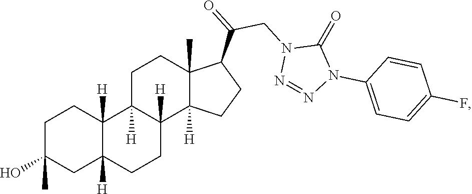

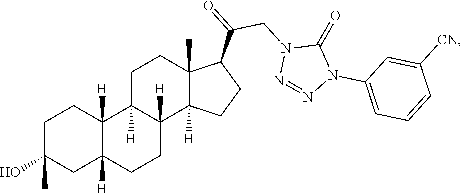

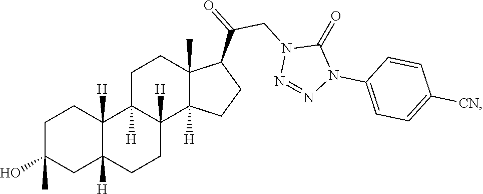

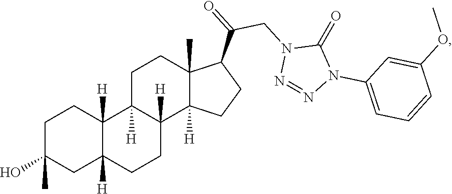

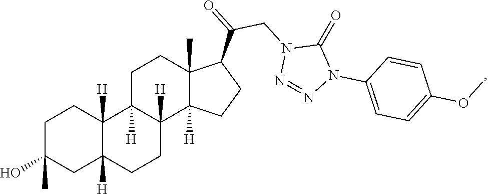

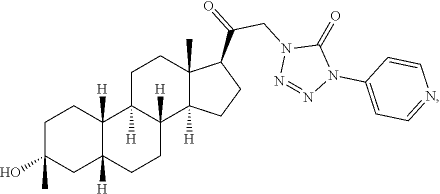

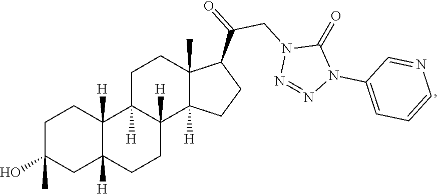

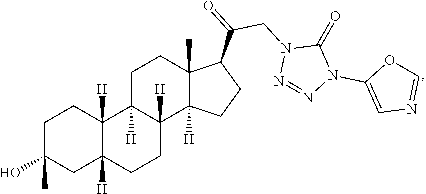

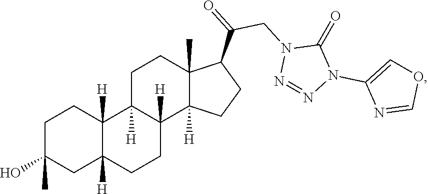

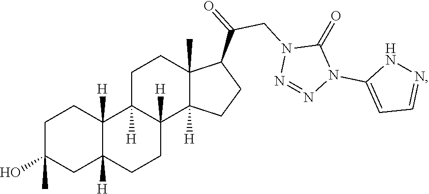

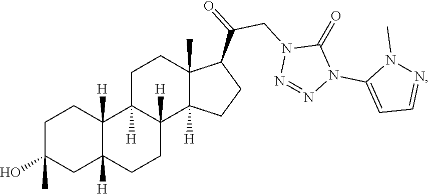

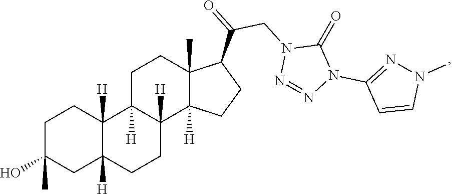

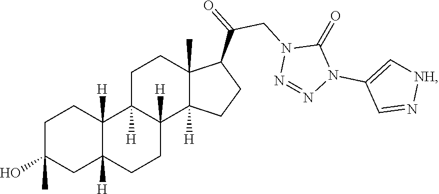

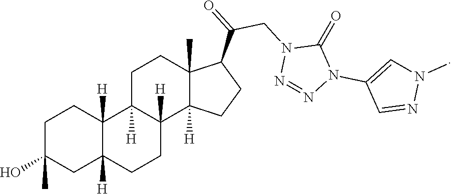

In one embodiment, the compound of the present disclosure provides a pharmaceutical composition comprising a pharmaceutically acceptable excipient or carrier and any compound of formula (AI), (I), (AII), or (II), or any subgenera thereof or a pharmaceutically acceptable salt, a solvate, a stereoisomer, or tautomer thereof. In some embodiments, the pharmaceutical composition comprises at least one additional therapeutically active agent. In some embodiments, the pharmaceutical composition comprises at least one additional therapeutically active agent.

### III. Methods of Use and Treatment

The present disclosure also includes method of using a compound of formula (AI), (I), (AII), and (II), and any subgenera thereof, or a pharmaceutically acceptable salt, solvate, ester, or prodrug thereof, and/or a pharmaceutical composition comprising one or more compounds of formula (AI), (I), (AII), and (II), and any subgenera thereof, or a pharmaceutically acceptable salt, solvate, ester, or prodrug thereof, as described herein. In some embodiments, the present disclosure provides method of modulating GABA_{A} receptors by contacting one or more compounds of formula (AI), (I), (AII), and (II), and any subgenera thereof, or a pharmaceutically acceptable salt, solvate, ester, or prodrug thereof, with a cell, an organ, or a subject, in need thereof. In one embodiment, a method of modulating GABA_{A} receptors comprises administering to a subject, a therapeutically effective amount of one or more compounds of formula (AI), (I), (AII), and (II), and any subgenera thereof, or a pharmaceutically acceptable salt, solvate, ester, or prodrug thereof. In one embodiment, the subject is a mammal including human.

In some embodiments, the present disclosure provides a method of treating, ameliorating, or preventing a condition which responds to modulation of GABA_{A} receptors, wherein one or more compounds of formula (AI), (I), (AII), and (II), and any subgenera thereof, or a pharmaceutically acceptable salt, solvate, ester, or prodrug thereof, is contacted with a cell, an organ, or a subject, in need thereof. In one embodiment, a method of treating, ameliorating, or preventing a condition which responds to modulation of GABA_{A} receptors comprises administering to a subject, a therapeutically effective amount of one or more compounds of formula (AI), (I), (AII), and (II), and any subgenera thereof, or a pharmaceutically acceptable salt, solvate, ester, or prodrug thereof.

In some embodiments, the present invention provides a method of inducing sedation and/or anesthesia in a subject, comprising administering to the subject an effective amount of a compound of the present invention or a composition thereof. In some embodiments, the present invention provides a method of use a compound of the present invention or a composition thereof as therapeutic agents for treating a CNS-related disorder (e.g., sleep disorder, a mood disorder, a schizophrenia spectrum disorder, a convulsive disorder, a disorder of memory and/or cognition, a movement disorder, a personality disorder, autism spectrum disorder, pain, traumatic brain injury, a vascular disease, a substance abuse disorder and/or withdrawal syndrome, or tinnitus) in a subject in need (e.g., a subject with Rett syndrome, Fragile X syndrome, or Angelman syndrome). In certain embodiments, the compound is administered by intravenous administration. In certain embodiments, the compound is administered by oral administration.

As generally described herein, the present invention is directed to tetrazolone substituted neuroactive steroids designed, for example, to act as GABA modulators. Previously, synthetic neuroactive steroids have been prepared, for example, U.S. Pat. No. 5,232,917, which discloses neuroactive steroid compounds useful in treating stress, anxiety, insomnia, seizure disorders, and mood disorders, such as depression, in a therapeutically beneficial manner.

Compounds of the present invention, as described herein, are generally designed to modulate GABA function, and therefore to act as neuroactive steroids for the treatment and prevention of CNS-related conditions in a subject. Modulation, as used herein, refers to the inhibition or potentiation of GABA receptor function. Accordingly, the compounds and pharmaceutical compositions provided herein find use as therapeutics for preventing and/or treating CNS conditions in mammals including humans and non-human mammals. Thus, the present invention includes within its scope, and extends to, the recited methods of treatment, as well as to the compounds for such methods, and to the use of such compounds for the preparation of medicaments useful for such methods.

Exemplary CNS conditions related to GABA-modulation include, but are not limited to, sleep disorders [e.g., insomnia], mood disorders [e.g., depression, dysthymic disorder (e.g., mild depression), bipolar disorder (e.g., I and/or II), anxiety disorders (e.g., generalized anxiety disorder (GAD), social anxiety disorder), stress, post-traumatic stress disorder (PTSD), compulsive disorders (e.g., obsessive compulsive disorder (OCD))], schizophrenia spectrum disorders [e.g., schizophrenia, schizoaffective disorder], convulsive disorders [e.g., epilepsy (e.g., status epilepticus (SE)), seizures], disorders of memory and/or cognition [e.g., attention disorders (e.g., attention deficit hyperactivity disorder (ADHD)), dementia (e.g., Alzheimer's type dementia, Lewis body type dementia, vascular type dementia)], movement disorders [e.g., Huntington's disease, Parkinson's disease], personality disorders [e.g., anti-social personality disorder, obsessive compulsive personality disorder], autism spectrum disorders (ASD) [e.g., autism, monogenetic causes of autism such as synaptophathy's, e.g., Rett syndrome, Fragile X syndrome, Angelman syndrome], pain [e.g., neuropathic pain, injury related pain syndromes, acute pain, chronic pain], traumatic brain injury (TBI), vascular diseases [e.g., stroke, ischemia, vascular malformations], substance abuse disorders and/or withdrawal syndromes [e.g., addiction to opiates, cocaine, and/or alcohol], and tinnitus.

In yet another aspect, provided is a combination of a compound of the present invention and another pharmacologically active agent. The compounds provided herein can be administered as the sole active agent or they can be administered in combination with other agents. Administration in combination can proceed by any technique apparent to those of skill in the art including, for example, separate, sequential, concurrent and alternating administration.

In another aspect, provided is a method of treating or preventing brain excitability in a subject susceptible to or afflicted with a condition associated with brain excitability, comprising administering to the subject an effective amount of a compound of the present invention to the subject.

In yet another aspect, provided is a method of treating or preventing stress or anxiety in a subject, comprising administering to the subject in need of such treatment an effective amount of a compound of the present invention, or a composition thereof.

In yet another aspect, provided is a method of alleviating or preventing seizure activity in a subject, comprising administering to the subject in need of such treatment an effective amount of a compound of the present invention.

In yet another aspect, provided is a method of alleviating or preventing insomnia in a subject, comprising administering to the subject in need of such treatment an effective amount of a compound of the present invention, or a composition thereof.

In yet another aspect, provided is a method of inducing sleep and maintaining substantially the level of REM sleep that is found in normal sleep, wherein substantial rebound insomnia is not induced, comprising administering an effective amount of a compound of the present invention.

In yet another aspect, provided is a method of alleviating or preventing PMS or PND in a subject, comprising administering to the subject in need of such treatment an effective amount of a compound of the present invention.

In yet another aspect, provided is a method of treating or preventing mood disorders in a subject, comprising administering to the subject in need of such treatment an effective amount of a compound of the present invention. In certain embodiments the mood disorder is depression.

In yet another aspect, provided is a method of inducing anesthesia in a subject, comprising administering to the subject an effective amount of the compound of the present invention.

In yet another aspect, provided is a method of cognition enhancement or treating memory disorder by administering to the subject a therapeutically effective amount of a compound of the present invention. In certain embodiments, the disorder is Alzheimer's disease. In certain embodiments, the disorder is Rett syndrome.

In yet another aspect, provided is a method of treating attention disorders by administering to the subject a therapeutically effective amount of a compound of the present invention. In certain embodiments, the attention disorder is ADHD.

In certain embodiments, the compound is administered to the subject chronically. In certain embodiments, the compound is administered to the subject orally, subcutaneously, intramuscularly, or intravenously.

Anesthesia/Sedation

Anesthesia is a pharmacologically induced and reversible state of amnesia, analgesia, loss of responsiveness, loss of skeletal muscle reflexes, decreased stress response, or all of these simultaneously. These effects can be obtained from a single drug which alone provides the correct combination of effects, or occasionally with a combination of drugs (e.g., hypnotics, sedatives, paralytics, analgesics) to achieve very specific combinations of results. Anesthesia allows patients to undergo surgery and other procedures without the distress and pain they would otherwise experience.

Sedation is the reduction of irritability or agitation by administration of a pharmacological agent, generally to facilitate a medical procedure or diagnostic procedure.

Sedation and analgesia include a continuum of states of consciousness ranging from minimal sedation (anxiolysis) to general anesthesia.

Minimal sedation is also known as anxiolysis. Minimal sedation is a drug-induced state during which the patient responds normally to verbal commands. Cognitive function and coordination may be impaired. Ventilatory and cardiovascular functions are typically unaffected.

Moderate sedation/analgesia (conscious sedation) is a drug-induced depression of consciousness during which the patient responds purposefully to verbal command, either alone or accompanied by light tactile stimulation. No interventions are usually necessary to maintain a patent airway. Spontaneous ventilation is typically adequate. Cardiovascular function is usually maintained.

Deep sedation/analgesia is a drug-induced depression of consciousness during which the patient cannot be easily aroused, but responds purposefully (not a reflex withdrawal from a painful stimulus) following repeated or painful stimulation. Independent ventilatory function may be impaired and the patient may require assistance to maintain a patent airway. Spontaneous ventilation may be inadequate. Cardiovascular function is usually maintained.

General anesthesia is a drug-induced loss of consciousness during which the patient is not arousable, even to painful stimuli. The ability to maintain independent ventilatory function is often impaired and assistance is often required to maintain a patent airway. Positive pressure ventilation may be required due to depressed spontaneous ventilation or drug-induced depression of neuromuscular function. Cardiovascular function may be impaired.

Sedation in the intensive care unit (ICU) allows the depression of patients' awareness of the environment and reduction of their response to external stimulation. It can play a role in the care of the critically ill patient, and encompasses a wide spectrum of symptom control that will vary between patients, and among individuals throughout the course of their illnesses. Heavy sedation in critical care has been used to facilitate endotracheal tube tolerance and ventilator synchronization, often with neuromuscular blocking agents.

In some embodiments, sedation (e.g., long-term sedation, continuous sedation) is induced and maintained in the ICU for a prolonged period of time (e.g., 1 day, 2 days, 3 days, 5 days, 1 week, 2 weeks, 3 weeks, 1 month, 2 months). Long-term sedation agents may have long duration of action. Sedation agents in the ICU may have short elimination half-life.

Procedural sedation and analgesia, also referred to as conscious sedation, is a technique of administering sedatives or dissociative agents with or without analgesics to induce a state that allows a subject to tolerate unpleasant procedures while maintaining cardiorespiratory function.

Anxiety Disorders

Anxiety disorder is a blanket term covering several different forms of abnormal and pathological fear and anxiety. Current psychiatric diagnostic criteria recognize a wide variety of anxiety disorders.

Generalized anxiety disorder is a common chronic disorder characterized by long-lasting anxiety that is not focused on any one object or situation. Those suffering from generalized anxiety experience non-specific persistent fear and worry and become overly concerned with everyday matters. Generalized anxiety disorder is the most common anxiety disorder to affect older adults.

In panic disorder, a person suffers from brief attacks of intense terror and apprehension, often marked by trembling, shaking, confusion, dizziness, nausea, difficulty breathing. These panic attacks, defined by the APA as fear or discomfort that abruptly arises and peaks in less than ten minutes, can last for several hours and can be triggered by stress, fear, or even exercise; although the specific cause is not always apparent. In addition to recurrent unexpected panic attacks, a diagnosis of panic disorder also requires that said attacks have chronic consequences: either worry over the attacks' potential implications, persistent fear of future attacks, or significant changes in behavior related to the attacks. Accordingly, those suffering from panic disorder experience symptoms even outside of specific panic episodes. Often, normal changes in heartbeat are noticed by a panic sufferer, leading them to think something is wrong with their heart or they are about to have another panic attack. In some cases, a heightened awareness (hypervigilance) of body functioning occurs during panic attacks, wherein any perceived physiological change is interpreted as a possible life threatening illness (i.e. extreme hypochondriasis).

Obsessive compulsive disorder is a type of anxiety disorder primarily characterized by repetitive obsessions (distressing, persistent, and intrusive thoughts or images) and compulsions (urges to perform specific acts or rituals). The OCD thought pattern may be likened to superstitions insofar as it involves a belief in a causative relationship where, in reality, one does not exist. Often the process is entirely illogical; for example, the compulsion of walking in a certain pattern may be employed to alleviate the obsession of impending harm. And in many cases, the compulsion is entirely inexplicable, simply an urge to complete a ritual triggered by nervousness. In a minority of cases, sufferers of OCD may only experience obsessions, with no overt compulsions; a much smaller number of sufferers experience only compulsions.

The single largest category of anxiety disorders is that of Phobia, which includes all cases in which fear and anxiety is triggered by a specific stimulus or situation. Sufferers typically anticipate terrifying consequences from encountering the object of their fear, which can be anything from an animal to a location to a bodily fluid.

Post-traumatic stress disorder or PTSD is an anxiety disorder which results from a traumatic experience. Post-traumatic stress can result from an extreme situation, such as combat, rape, hostage situations, or even serious accident. It can also result from long term (chronic) exposure to a severe stressor, for example soldiers who endure individual battles but cannot cope with continuous combat. Common symptoms include flashbacks, avoidant behaviors, and depression.

Neurodegenerative Diseases and Disorders

The term “neurodegenerative disease” includes diseases and disorders that are associated with the progressive loss of structure or function of neurons, or death of neurons. Neurodegenerative diseases and disorders include, but are not limited to, Alzheimer's disease (including the associated symptoms of mild, moderate, or severe cognitive impairment); amyotrophic lateral sclerosis (ALS); anoxic and ischemic injuries; ataxia and convulsion (including for the treatment and prevention of seizures that are caused by schizoaffective disorder or by drugs used to treat schizophrenia); benign forgetfulness; brain edema; cerebellar ataxia including McLeod neuroacanthocytosis syndrome (MLS); closed head injury; coma; contusive injuries (e.g., spinal cord injury and head injury); dementias including multi-infarct dementia and senile dementia; disturbances of consciousness; Down syndrome; drug-induced or medication-induced Parkinsonism (such as neuroleptic-induced acute akathisia, acute dystonia, Parkinsonism, or tardive dyskinesia, neuroleptic malignant syndrome, or medication-induced postural tremor); epilepsy; fragile X syndrome; Gilles de la Tourette's syndrome; head trauma; hearing impairment and loss; Huntington's disease; Lennox syndrome; levodopa-induced dyskinesia; mental retardation; movement disorders including akinesias and akinetic (rigid) syndromes (including basal ganglia calcification, corticobasal degeneration, multiple system atrophy, Parkinsonism-ALS dementia complex, Parkinson's disease, postencephalitic parkinsonism, and progressively supranuclear palsy); muscular spasms and disorders associated with muscular spasticity or weakness including chorea (such as benign hereditary chorea, drug-induced chorea, hemiballism, Huntington's disease, neuroacanthocytosis, Sydenham's chorea, and symptomatic chorea), dyskinesia (including tics such as complex tics, simple tics, and symptomatic tics), myoclonus (including generalized myoclonus and focal cyloclonus), tremor (such as rest tremor, postural tremor, and intention tremor) and dystonia (including axial dystonia, dystonic writer's cramp, hemiplegic dystonia, paroxysmal dystonia, and focal dystonia such as blepharospasm, oromandibular dystonia, and spasmodic dysphonia and torticollis); neuronal damage including ocular damage, retinopathy or macular degeneration of the eye; neurotoxic injury which follows cerebral stroke, thromboembolic stroke, hemorrhagic stroke, cerebral ischemia, cerebral vasospasm, hypoglycemia, amnesia, hypoxia, anoxia, perinatal asphyxia and cardiac arrest; Parkinson's disease; seizure; status epilecticus; stroke; tinnitus; tubular sclerosis, and viral infection induced neurodegeneration (e.g., caused by acquired immunodeficiency syndrome (AIDS) and encephalopathies). Neurodegenerative diseases also include, but are not limited to, neurotoxic injury which follows cerebral stroke, thromboembolic stroke, hemorrhagic stroke, cerebral ischemia, cerebral vasospasm, hypoglycemia, amnesia, hypoxia, anoxia, perinatal asphyxia and cardiac arrest. Methods of treating or preventing a neurodegenerative disease also include treating or preventing loss of neuronal function characteristic of neurodegenerative disorder.

Epilepsy

Epilepsy is a brain disorder characterized by repeated seizures over time. Types of epilepsy can include, but are not limited to generalized epilepsy, e.g., childhood absence epilepsy, juvenile nyoclonic epilepsy, epilepsy with grand-mal seizures on awakening, West syndrome, Lennox-Gastaut syndrome, partial epilepsy, e.g., temporal lobe epilepsy, frontal lobe epilepsy, benign focal epilepsy of childhood.

Status Epilepticus (SE)

Status epilepticus (SE) can include, e.g., convulsive status epilepticus, e.g., early status epilepticus, established status epilepticus, refractory status epilepticus, super-refractory status epilepticus; non-convulsive status epilepticus, e.g., generalized status epilepticus, complex partial status epilepticus; generalized periodic epileptiform discharges; and periodic lateralized epileptiform discharges. Convulsive status epilepticus is characterized by the presence of convulsive status epileptic seizures, and can include early status epilepticus, established status epilepticus, refractory status epilepticus, super-refractory status epilepticus. Early status epilepticus is treated with a first line therapy. Established status epilepticus is characterized by status epileptic seizures which persist despite treatment with a first line therapy, and a second line therapy is administered. Refractory status epilepticus is characterized by status epileptic seizures which persist despite treatment with a first line and a second line therapy, and a general anesthetic is generally administered. Super refractory status epilepticus is characterized by status epileptic seizures which persist despite treatment with a first line therapy, a second line therapy, and a general anesthetic for 24 hours or more.

Non-convulsive status epilepticus can include, e.g., focal non-convulsive status epilepticus, e.g., complex partial non-convulsive status epilepticus, simple partial non-convulsive status epilepticus, subtle non-convulsive status epilepticus; generalized non-convulsive status epilepticus, e.g., late onset absence non-convulsive status epilepticus, atypical absence non-convulsive status epilepticus, or typical absence non-convulsive status epilepticus.

Compositions described herein can also be administered as a prophylactic to a subject having a CNS disorder e.g., atraumatic brain injury, status epilepticus, e.g., convulsive status epilepticus, e.g., early status epilepticus, established status epilepticus, refractory status epilepticus, superrefractory status epilepticus; non-convulsive status epilepticus, e.g., generalized status epilepticus, complex partial status epilepticus; generalized periodic epileptiform discharges; and periodic lateralized epileptiform discharges; prior to the onset of a seizure.

Seizure

A seizure is the physical findings or changes in behavior that occur after an episode of abnormal electrical activity in the brain. The term “seizure” is often used interchangeably with “convulsion”. Convulsions are when a person's body shakes rapidly and uncontrollably. During convulsions, the person's muscles contract and relax repeatedly.

Based on the type of behavior and brain activity, seizures are divided into two broad categories: generalized and partial (also called local or focal). Classifying the type of seizure helps doctors diagnose whether or not a patient has epilepsy.

Generalized seizures are produced by electrical impulses from throughout the entire brain, whereas partial seizures are produced (at least initially) by electrical impulses in a relatively small part of the brain. The part of the brain generating the seizures is sometimes called the focus.

There are six types of generalized seizures. The most common and dramatic, and therefore the most well known, is the generalized convulsion, also called the grand-mal seizure. In this type of seizure, the patient loses consciousness and usually collapses. The loss of consciousness is followed by generalized body stiffening (called the “tonic” phase of the seizure) for 30 to 60 seconds, then by violent jerking (the “clonic” phase) for 30 to 60 seconds, after which the patient goes into a deep sleep (the “postictal” or after-seizure phase). During grand-mal seizures, injuries and accidents may occur, such as tongue biting and urinary incontinence.

Absence seizures cause a short loss of consciousness (just a few seconds) with few or no symptoms. The patient, most often a child, typically interrupts an activity and stares blankly. These seizures begin and end abruptly and may occur several times a day. Patients are usually not aware that they are having a seizure, except that they may be aware of “losing time.”

Myoclonic seizures consist of sporadic jerks, usually on both sides of the body. Patients sometimes describe the jerks as brief electrical shocks. When violent, these seizures may result in dropping or involuntarily throwing objects.

Clonic seizures are repetitive, rhythmic jerks that involve both sides of the body at the same time.

Tonic seizures are characterized by stiffening of the muscles.

Atonic seizures consist of a sudden and general loss of muscle tone, particularly in the arms and legs, which often results in a fall.

Seizures described herein can include epileptic seizures; acute repetitive seizures; cluster seizures; continuous seizures; unremitting seizures; prolonged seizures; recurrent seizures; status epilepticus seizures, e.g., refractory convulsive status epilepticus, non-convulsive status epilepticus seizures; refractory seizures; myoclonic seizures; tonic seizures; tonic-clonic seizures; simple partial seizures; complex partial seizures; secondarily generalized seizures; atypical absence seizures; absence seizures; atonic seizures; benign Rolandic seizures; febrile seizures; emotional seizures; focal seizures; gelastic seizures; generalized onset seizures; infantile spasms; Jacksonian seizures; massive bilateral myoclonus seizures; multifocal seizures; neonatal onset seizures; nocturnal seizures; occipital lobe seizures; post traumatic seizures; subtle seizures; Sylvan seizures; visual reflex seizures; or withdrawal seizures.

### IV. Pharmaceutical Compositions and Formulations

The present disclosure also includes pharmaceutical compositions comprising one or more compounds of formula (AI), (I), (AII), and (II), and any subgenera thereof, or a pharmaceutically acceptable salt, solvate, ester, or prodrug thereof. In some embodiments, one or more compounds of formula (AI), (I), (AII), and (II), and any subgenera thereof, or a pharmaceutically acceptable salt, solvate, ester, or prodrug thereof, in a pharmaceutical composition as described herein modulates GABA_{A} receptors. In other embodiments, one or more compounds of formula (AI), (I), (AII), and (II), and any subgenera thereof, or a pharmaceutically acceptable salt, solvate, ester, or prodrug thereof, in a pharmaceutical composition as described herein can be useful in a method to treat, ameliorate or prevent a condition, which responds to GABA_{A} receptors modulation or in a method of treating CNS-related disorders.

In one aspect, the invention provides a pharmaceutical composition comprising a compound of the present invention (also referred to as the “active ingredient”) and a pharmaceutically acceptable excipient. In certain embodiments, the pharmaceutical composition comprises an effective amount of the active ingredients. In certain embodiments, the pharmaceutical composition comprises a therapeutically effective amount of the active ingredients. In certain embodiments, the pharmaceutical composition comprises a prophylactically effective amount of the active ingredients.

Appropriate pharmaceutical compositions of the present disclosure can be determined according to any clinically-acceptable route of administration of the composition to the subject. The manner in which the composition is administered is dependent, in part, upon the cause and/or location. One skilled in the art will recognize the advantages of certain routes of administration. The method includes administering an effective amount of the agent or compound (or composition comprising the agent or compound) to achieve a desired biological response, e.g., an amount effective to alleviate, ameliorate, or prevent, in whole or in part, a symptom of a condition to be treated, e.g., CNS disorders. In various aspects, the route of administration is systemic, e.g., oral or by injection. The agents or compounds, or pharmaceutically acceptable salts or derivatives thereof, are administered by a variety of means including, but not limited to, oral, nasal, transdermal, intradermal, pulmonary, inhalational, buccal, sublingual, intraperitoneal, subcutaneous, intramuscular, intravenous, rectal, intrapleural, intrathecal, intraportal, and parenteral administration in formulations containing pharmaceutically acceptable carriers, adjuvants and vehicles. The term parenteral as used here includes subcutaneous (SC), intravenous (IV), intramuscular (IM), and intraarterial injections with a variety of infusion techniques. Intraarterial and intravenous injection as used herein includes administration through catheters. Alternatively or in addition, the route of administration is local, e.g., topical. In some embodiments, the compound is administered orally.

Generally, the compounds provided herein are administered in an effective amount. The amount of the compound actually administered will typically be determined by a physician, in the light of the relevant circumstances, including the condition to be treated, the chosen route of administration, the actual compound administered, the age, weight, and response of the individual patient, the severity of the patient's symptoms, and the like.

When used to prevent the onset of a CNS-disorder, the compounds provided herein will be administered to a subject at risk for developing the condition, typically on the advice and under the supervision of a physician, at the dosage levels described above. Subjects at risk for developing a particular condition generally include those that have a family history of the condition, or those who have been identified by genetic testing or screening to be particularly susceptible to developing the condition.

The pharmaceutical compositions provided herein can also be administered chronically (“chronic administration”). Chronic administration refers to administration of a compound or pharmaceutical composition thereof over an extended period of time, e.g., for example, over 3 months, 6 months, 1 year, 2 years, 3 years, 5 years, etc, or may be continued indefinitely, for example, for the rest of the subject's life. In certain embodiments, the chronic administration is intended to provide a constant level of the compound in the blood, e.g., within the therapeutic window over the extended period of time.

The pharmaceutical compositions of the present invention may be further delivered using a variety of dosing methods. For example, in certain embodiments, the pharmaceutical composition may be given as a bolus, e.g., in order to raise the concentration of the compound in the blood to an effective level. The placement of the bolus dose depends on the systemic levels of the active ingredient desired throughout the body, e.g., an intramuscular or subcutaneous bolus dose allows a slow release of the active ingredients, while a bolus delivered directly to the veins (e.g. through an IV drip) allows a much faster delivery. In other embodiments, the pharmaceutical composition may be administered as a continuous infusion, e.g., by IV drip, to provide maintenance of a steady-state concentration of the active ingredient in the subject's body. In other embodiments, the pharmaceutical composition may be administered as first as a bolus dose, followed by continuous infusion.

The compounds disclosed herein can be formulated in accordance with the routine procedures adapted for desired administration route. Accordingly, the compounds disclosed herein can take such forms as suspensions, solutions or emulsions in oily or aqueous vehicles, and can contain formulatory agents such as suspending, stabilizing and/or dispersing agents. The compounds disclosed herein can also be formulated as a preparation for implantation or injection. Thus, for example, the compounds can be formulated with suitable polymeric or hydrophobic materials (e.g., as an emulsion in an acceptable oil) or ion exchange resins, or as sparingly soluble derivatives (e.g., as a sparingly soluble salt). Alternatively, the active ingredient can be in powder form for constitution with a suitable vehicle, e.g., sterile pyrogen-free water, before use. Suitable formulations for each of these methods of administration can be found, for example, in Remington: The Science and Practice of Pharmacy, A. Gennaro, ed., 20th edition, Lippincott, Williams & Wilkins, Philadelphia, Pa., the entire disclosure of which is incorporated by reference herein for all purposes.

In certain embodiments, a pharmaceutical composition of the present disclosure is prepared for oral administration. In certain of such embodiments, a pharmaceutical composition is formulated by combining one or more agents and pharmaceutically acceptable carriers. Certain of such carriers enable pharmaceutical compositions to be formulated as tablets, pills, dragees, capsules, liquids, gels, syrups, slurries, suspensions and the like, for oral ingestion by a subject. Suitable excipients include, but are not limited to, fillers, such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). In certain embodiments, such a mixture is optionally grounded and auxiliaries are optionally added. In certain embodiments, pharmaceutical compositions are formed to obtain tablets or dragee cores. In certain embodiments, disintegrating agents (e.g., cross-linked polyvinyl pyrrolidone, agar, or alginic acid or a salt thereof, such as sodium alginate) are added.

In certain embodiments, dragee cores are provided with coatings. In certain such embodiments, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to tablets or dragee coatings.

In certain embodiments, pharmaceutical compositions for oral administration are push-fit capsules made of gelatin. Certain of such push-fit capsules comprise one or more pharmaceutical agents of the present invention in admixture with one or more filler such as lactose, binders such as starches, and/or lubricants such as talc or magnesium stearate and, optionally, stabilizers. In certain embodiments, pharmaceutical compositions for oral administration are soft, sealed capsules made of gelatin and a plasticizer, such as glycerol or sorbitol. In certain soft capsules, one or more pharmaceutical agents of the present invention are to be dissolved or suspended in suitable liquids, such as fatty oils, liquid paraffin, or liquid polyethylene glycols. In addition, stabilizers may be added.

The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the compound is usually a minor component (from about 0.1 to about 50% by weight or preferably from about 1 to about 40% by weight) with the remainder being various vehicles or excipients and processing aids helpful for forming the desired dosing form.

With oral dosing, one to five and especially two to four and typically three oral doses per day are representative regimens. Using these dosing patterns, each dose provides from about 0.01 to about 20 mg/kg of the compound provided herein, with preferred doses each providing from about 0.1 to about 10 mg/kg, and especially about 1 to about 5 mg/kg.

Transdermal doses are generally selected to provide similar or lower blood levels than are achieved using injection doses, generally in an amount ranging from about 0.01 to about 20% by weight, preferably from about 0.1 to about 20% by weight, preferably from about 0.1 to about 10% by weight, and more preferably from about 0.5 to about 15% by weight.

In other embodiments, the compounds of the present disclosure are administered by the intravenous route. In further embodiments, the parenteral administration may be provided in a bolus or by infusion.

Injection dose levels range from about 0.1 mg/kg/hour to at least 10 mg/kg/hour, all for from about 1 to about 120 hours and especially 24 to 96 hours. A preloading bolus of from about 0.1 mg/kg to about 10 mg/kg or more may also be administered to achieve adequate steady state levels. The maximum total dose is not expected to exceed about 2 g/day for a 40 to 80 kg human patient.

In certain embodiments, a pharmaceutical composition of the present disclosure is prepared using known techniques, including, but not limited to mixing, dissolving, granulating, dragee-making, levigating, emulsifying, encapsulating, entrapping or tableting processes.

In one embodiment, the present disclosure provides a pharmaceutical composition comprising a compound of formula (AI), (I), (AII), and (II), and any subgenera thereof, or a pharmaceutically acceptable salt, solvate, ester, or prodrug thereof, as disclosed herein, combined with a pharmaceutically acceptable carrier. In one embodiment, suitable pharmaceutically acceptable carriers include, but are not limited to, inert solid fillers or diluents and sterile aqueous or organic solutions. Pharmaceutically acceptable carriers are well known to those skilled in the art and include, but are not limited to, from about 0.01 to about 0.1 M and preferably 0.05M phosphate buffer or 0.8% saline. Such pharmaceutically acceptable carriers can be aqueous or non-aqueous solutions, suspensions and emulsions. Examples of non-aqueous solvents suitable for use in the present application include, but are not limited to, propylene glycol, polyethylene glycol, vegetable oils such as olive oil, and injectable organic esters such as ethyl oleate.

Aqueous carriers suitable for use in the present application include, but are not limited to, water, ethanol, alcoholic/aqueous solutions, glycerol, emulsions or suspensions, including saline and buffered media. Oral carriers can be elixirs, syrups, capsules, tablets and the like.

Liquid carriers suitable for use in the present application can be used in preparing solutions, suspensions, emulsions, syrups, elixirs and pressurized compounds. The active ingredient can be dissolved or suspended in a pharmaceutically acceptable liquid carrier such as water, an organic solvent, a mixture of both or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators.

Liquid carriers suitable for use in the present application include, but are not limited to, water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the carrier can also include an oily ester such as ethyl oleate and isopropyl myristate. Sterile liquid carriers are useful in sterile liquid form comprising compounds for parenteral administration. The liquid carrier for pressurized compounds disclosed herein can be halogenated hydrocarbon or other pharmaceutically acceptable propellent.

Solid carriers suitable for use in the present application include, but are not limited to, inert substances such as lactose, starch, glucose, methyl-cellulose, magnesium stearate, dicalcium phosphate, mannitol and the like. A solid carrier can further include one or more substances acting as flavoring agents, lubricants, solubilizers, suspending agents, fillers, glidants, compression aids, binders or tablet-disintegrating agents; it can also be an encapsulating material. In powders, the carrier can be a finely divided solid which is in admixture with the finely divided active compound. In tablets, the active compound is mixed with a carrier having the necessary compression properties in suitable proportions and compacted in the shape and size desired. The powders and tablets preferably contain up to 99% of the active compound. Suitable solid carriers include, for example, calcium phosphate, magnesium stearate, talc, sugars, lactose, dextrin, starch, gelatin, cellulose, polyvinylpyrrolidine, low melting waxes and ion exchange resins. A tablet may be made by compression or molding, optionally with one or more accessory ingredients. Compressed tablets may be prepared by compressing in a suitable machine the active ingredient in a free-flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein using, for example, hydroxypropyl methylcellulose in varying proportions to provide the desired release profile. Tablets may optionally be provided with an enteric coating, to provide release in parts of the gut other than the stomach.

Parenteral carriers suitable for use in the present application include, but are not limited to, sodium chloride solution, Ringer's dextrose, dextrose and sodium chloride, lactated Ringer's and fixed oils. Intravenous carriers include fluid and nutrient replenishers, electrolyte replenishers such as those based on Ringer's dextrose and the like. Preservatives and other additives can also be present, such as, for example, antimicrobials, antioxidants, chelating agents, inert gases and the like.

Carriers suitable for use in the present application can be mixed as needed with disintegrants, diluents, granulating agents, lubricants, binders and the like using conventional techniques known in the art. The carriers can also be sterilized using methods that do not deleteriously react with the compounds, as is generally known in the art.

Diluents may be added to the formulations of the present invention. Diluents increase the bulk of a solid pharmaceutical composition and/or combination and may make a pharmaceutical dosage form containing the composition and/or combination easier for the patient and care giver to handle. Diluents for solid compositions and/or combinations include, for example, microcrystalline cellulose (e.g., AVICEL), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., EUDRAGIT(r)), potassium chloride, powdered cellulose, sodium chloride, sorbitol, and talc.

Additional embodiments relate to the pharmaceutical formulations wherein the formulation is selected from the group consisting of a solid, powder, liquid and a gel. In certain embodiments, a pharmaceutical composition of the present invention is a solid (e.g., a powder, tablet, a capsule, granulates, and/or aggregates). In certain of such embodiments, a solid pharmaceutical composition comprising one or more ingredients known in the art, including, but not limited to, starches, sugars, diluents, granulating agents, lubricants, binders, and disintegrating agents.

Solid pharmaceutical compositions that are compacted into a dosage form, such as a tablet, may include excipients whose functions include helping to bind the active ingredient and other excipients together after compression. Binders for solid pharmaceutical compositions and/or combinations include acacia, alginic acid, carbomer (e.g., carbopol), carboxymethylcellulose sodium, dextrin, ethyl cellulose, gelatin, guar gum, gum tragacanth, hydrogenated vegetable oil, hydroxyethyl cellulose, hydroxypropyl cellulose (e.g., KLUCEL), hydroxypropyl methyl cellulose (e.g., METHOCEL), liquid glucose, magnesium aluminum silicate, maltodextrin, methylcellulose, polymethacrylates, povidone (e.g., KOLLIDON, PLASDONE), pregelatinized starch, sodium alginate, and starch. An excipient may include such as starch or lactose.

The dissolution rate of a compacted solid pharmaceutical composition in the patient's stomach may be increased by the addition of a disintegrant to the composition and/or combination. Disintegrants include alginic acid, carboxymethylcellulose calcium, carboxymethylcellulose sodium (e.g., AC-DI-SOL and PRIMELLOSE), colloidal silicon dioxide, croscarmellose sodium, crospovidone (e.g., KOLLIDON and POLYPLASDONE), guar gum, magnesium aluminum silicate, methyl cellulose, microcrystalline cellulose, polacrilin potassium, powdered cellulose, pregelatinized starch, sodium alginate, sodium starch glycolate (e.g., EXPLOTAB), potato starch, corn starch, starch, and Primogel.

Glidants can be added to improve the flowability of a non-compacted solid composition and/or combination and to improve the accuracy of dosing. Excipients that may function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition and/or combination to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that may be included in the composition and/or combination of the present invention include maltol, vanillin, ethyl vanillin, menthol, citric acid, fumaric acid, ethyl maltol, peppermint, methyl salicylate, orange flavoring and tartaric acid. A sweetening agent may include such as sucrose or saccharin.

Solid and liquid compositions may also be dyed using any pharmaceutically acceptable colorant to improve their appearance and/or facilitate patient identification of the product and unit dosage level.

In certain embodiments, a pharmaceutical composition of the present invention is a liquid (e.g., a suspension, elixir and/or solution). In certain of such embodiments, a liquid pharmaceutical composition is prepared using ingredients known in the art, including, but not limited to, water, buffer, glycols, oils, alcohols, suspending and dispending agents, flavoring agents, preservatives, and coloring agents.

Liquid pharmaceutical compositions can be prepared using compounds of formula (AI), (I), (AII), and (II), and any subgenera thereof, or a pharmaceutically acceptable salt, solvate, ester, or prodrug thereof, and any other solid excipients where the components are dissolved or suspended in a liquid carrier such as water, buffer, vegetable oil, alcohol, polyethylene glycol, propylene glycol, or glycerin.

For example, formulations for parenteral administration can contain as common excipients sterile water or saline, polyalkylene glycols such as polyethylene glycol, oils of vegetable origin, hydrogenated naphthalenes and the like. In particular, biocompatible, biodegradable lactide polymer, lactide/glycolide copolymer, or polyoxyethylene-polyoxypropylene copolymers can be useful excipients to control the release of active compounds. Other potentially useful parenteral delivery systems include ethylene-vinyl acetate copolymer particles, osmotic pumps, implantable infusion systems, and liposomes. Formulations for inhalation administration contain as excipients, for example, lactose, or can be aqueous solutions containing, for example, polyoxyethylene-9-auryl ether, glycocholate and deoxycholate, or oily solutions for administration in the form of nasal drops, or as a gel to be applied intranasally. Formulations for parenteral administration can also include glycocholate for buccal administration, methoxysalicylate for rectal administration, or citric acid for vaginal administration.

Liquid pharmaceutical compositions can contain emulsifying agents to disperse uniformly throughout the composition and/or combination an active ingredient or other excipient that is not soluble in the liquid carrier. Emulsifying agents that may be useful in liquid compositions and/or combinations of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions can also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, and xanthan gum.

Sweetening agents such as aspartame, lactose, sorbitol, saccharin, sodium saccharin, sucrose, aspartame, fructose, mannitol, and invert sugar may be added to improve the taste.

Preservatives and chelating agents such as alcohol, sodium benzoate, butylated hydroxyl toluene, butylated hydroxyanisole, and ethylenediamine tetraacetic acid may be added at levels safe for ingestion to improve storage stability.

A liquid composition can also contain a buffer such as guconic acid, lactic acid, citric acid or acetic acid, sodium guconate, sodium lactate, sodium citrate, or sodium acetate. Selection of excipients and the amounts used may be readily determined by the formulation scientist based upon experience and consideration of standard procedures and reference works in the field.

In one embodiment, a pharmaceutical composition is prepared for administration by injection (e.g., intravenous, subcutaneous, intramuscular, etc.). In certain of such embodiments, a pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes. Aqueous injection suspensions may contain substances that increase the viscosity of the suspension, such as sodium carboxymethyl cellulose, sorbitol, or dextran. Optionally, such suspensions may also contain suitable stabilizers or agents that increase the solubility of the pharmaceutical agents to allow for the preparation of highly concentrated solutions.

The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, sterile saline, phosphate-buffered saline, Ringer's solution and isotonic sodium chloride solution. In addition, sterile fixed oils may conventionally be employed as a solvent or suspending medium. For this purpose, any bland fixed oil may be employed including synthetic mono- or diglycerides. In addition, fatty acids such as oleic acid may likewise be used in the preparation of injectables. Formulations for intravenous administration can comprise solutions in sterile isotonic aqueous buffer. Where necessary, the formulations can also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachet indicating the quantity of active agent. Where the compound is to be administered by infusion, it can be dispensed in a formulation with an infusion bottle containing sterile pharmaceutical grade water, saline or dextrose/water. Where the compound is administered by injection, an ampule of sterile water for injection or saline can be provided so that the ingredients can be mixed prior to administration.

Suitable formulations further include aqueous and non-aqueous sterile injection solutions that can contain antioxidants, buffers, bacteriostats, bactericidal antibiotics and solutes that render the formulation isotonic with the bodily fluids of the intended recipient; and aqueous and non-aqueous sterile suspensions, which can include suspending agents and thickening agents.

In certain embodiments, a transdermal composition of the present invention is formulated as a topical ointment or cream containing the active ingredient(s). When formulated as an ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration of stability of the active ingredients or formulation. All such known transdermal formulations and ingredients are included within the scope provided herein.

The compounds provided herein can also be administered by a transdermal device. Accordingly, transdermal administration can be accomplished using a patch either of the reservoir or porous membrane type, or of a solid matrix variety.

In certain embodiments, a pharmaceutical composition of the present invention is formulated as a depot preparation. Certain such depot preparations are typically longer acting than non-depot preparations. In certain embodiments, such preparations are administered by implantation (for example subcutaneously or intramuscularly) or by intramuscular injection. In certain embodiments, depot preparations are prepared using suitable polymeric or hydrophobic materials (for example an emulsion in an acceptable oil) or ion exchange resins, or as sparingly soluble derivatives, for example, as a sparingly soluble salt.

In certain embodiments, a pharmaceutical composition of the present invention comprises a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, a pharmaceutical composition of the present invention comprises a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80 and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, a pharmaceutical composition of the present invention comprises a sustained-release system. A non-limiting example of such a sustained-release system is a semi-permeable matrix of solid hydrophobic polymers. In certain embodiments, sustained-release systems may, depending on their chemical nature, release pharmaceutical agents over a period of hours, days, weeks or months.

In certain embodiments, pharmaceutical compositions are prepared for buccal administration. Certain of such pharmaceutical compositions are tablets or lozenges formulated in conventional manner.

In certain embodiments, a pharmaceutical composition is prepared for transmucosal administration. In certain of such embodiments, penetrants appropriate to the barrier to be permeated are used in the formulation. Such penetrants are generally known in the art.

In certain embodiments, a pharmaceutical composition is prepared for administration by inhalation. Certain of such pharmaceutical compositions for inhalation are prepared in the form of an aerosol spray in a pressurized pack or a nebulizer. Certain of such pharmaceutical compositions comprise a propellant, e.g., dichlorodifluoromethane, trichlorofluoromethane, dichlorotetrafluoroethane, carbon dioxide or other suitable gas. In certain embodiments using a pressurized aerosol, the dosage unit may be determined with a valve that delivers a metered amount. In certain embodiments, capsules and cartridges for use in an inhaler or insufflator may be formulated. Certain of such formulations comprise a powder mixture of a pharmaceutical agent of the invention and a suitable powder base such as lactose or starch.

In certain embodiments, a pharmaceutical composition is prepared for rectal administration, such as a suppository or retention enema. Certain of such pharmaceutical compositions comprise known ingredients, such as cocoa butter and/or other glycerides.

In certain embodiments, a pharmaceutical composition is prepared for topical administration. Certain of such pharmaceutical compositions comprise bland moisturizing bases, such as ointments or creams. Exemplary suitable ointment bases include, but are not limited to, petrolatum, petrolatum plus volatile silicones, and lanolin and water in oil emulsions. Exemplary suitable cream bases include, but are not limited to, cold cream and hydrophilic ointment.

In certain embodiments, one or more compounds of formula (AI), (I), (AII), and (II) and any subgenera thereof, or a pharmaceutically acceptable salt, solvate, ester, or prodrug thereof are formulated as a prodrug. In certain embodiments, upon in vivo administration, a prodrug is chemically converted to the biologically, pharmaceutically or therapeutically more active form. In certain embodiments, prodrugs are useful because they are easier to administer than the corresponding active form. For example, in certain instances, a prodrug may be more bioavailable (e.g., through oral administration) than is the corresponding active form. In certain instances, a prodrug may have improved solubility compared to the corresponding active form. In certain embodiments, prodrugs are less water soluble than the corresponding active form. In certain instances, such prodrugs possess superior transmittal across cell membranes, where water solubility is detrimental to mobility. In certain embodiments, a prodrug is an ester. In certain such embodiments, the ester is metabolically hydrolyzed to carboxylic acid or equivalent upon administration. In certain instances, the carboxylic acid or acid equivalent containing compound is the corresponding active form. In certain embodiments, a prodrug comprises a short peptide (polyaminoacid) bound to an acid or acid equivalent group. In certain of such embodiments, the peptide is cleaved upon administration to form the corresponding active form.

In certain embodiments, a prodrug is produced by modifying a pharmaceutically active compound such that the active compound will be regenerated upon in vivo administration. The prodrug can be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound (see, e.g., Nogrady (1985) Medicinal Chemistry A Biochemical Approach, Oxford University Press, New York, pages 388-392).

In certain embodiments, the therapeutically effective amount is sufficient to prevent, alleviate or ameliorate symptoms of a disease. Determination of a therapeutically effective amount is well within the capability of those skilled in the art.

In various aspects, the amount of the compounds of formula (AI), (I), (AII), and (II), and any subgenera thereof, or a pharmaceutically acceptable salt, solvate, ester, or prodrug thereof, can be administered at about 0.001 mg/kg to about 100 mg/kg body weight (e.g., about 0.01 mg/kg to about 10 mg/kg or about 0.1 mg/kg to about 5 mg/kg).

The concentration of a disclosed compound in a pharmaceutically acceptable mixture will vary depending on several factors, including the dosage of the compound to be administered, the pharmacokinetic characteristics of the compound(s) employed, and the route of administration. The agent may be administered in a single dose or in repeat doses. The dosage regimen utilizing the compounds of the present invention is selected in accordance with a variety of factors including type, species, age, weight, sex and medical condition of the patient; the severity of the condition to be treated; the route of administration; the renal and hepatic function of the patient; and the particular compound or salt thereof employed. Treatments may be administered daily or more frequently depending upon a number of factors, including the overall health of a patient, and the formulation and route of administration of the selected compound(s). An ordinarily skilled physician or veterinarian can readily determine and prescribe the effective amount of the drug required to prevent, counter or arrest the progress of the condition.

The compounds or pharmaceutical compositions of the present disclosure may be manufactured and/or administered in single or multiple unit dose forms, for example as tablets, capsules, powders, solutions, suspensions, emulsions, granules, or suppositories. In such form, the pharmaceutical composition can be sub-divided in unit dose(s) containing appropriate quantities of the compound. The unit dosage forms can be packaged compositions, for example, packeted powders, vials, ampoules, prefilled syringes or sachets containing liquids. Alternatively, the unit dosage form can be a capsule or tablet itself, or it can be the appropriate number of any such compositions in package form, such unit dosage form can contain from about 1 mg/kg of compound to about 500 mg/kg of compound, and can be given in a single dose or in two or more doses. Such doses can be administered in any manner useful in directing the compound(s) to the recipient's bloodstream, including orally, via implants, parenterally (including intravenous, intraperitoneal and subcutaneous injections), rectally, vaginally, trans-dermally, or any other methods as disclosed herein.

The present invention also relates to the pharmaceutically acceptable formulations of a compound of the present invention. In one embodiment, the formulation comprises water. In another embodiment, the formulation comprises a cyclodextrin derivative. The most common cyclodextrins are α-, β- and γ-cyclodextrins consisting of 6, 7 and 8 α-1,4-linked glucose units, respectively, optionally comprising one or more substituents on the linked sugar moieties, which include, but are not limited to, methylated, hydroxyalkylated, acylated, and sulfoalkylether substitution. In certain embodiments, the cyclodextrin is a sulfoalkyl ether β-cyclodextrin, e.g., for example, sulfobutyl ether β-cyclodextrin, also known as Captisol®. See, e.g., U.S. Pat. No. 5,376,645. In certain embodiments, the formulation comprises hexapropyl-β-cyclodextrin (e.g., 10-50% in water).

The present invention also relates to the pharmaceutically acceptable acid addition salt of a compound of the present invention. The acid which may be used to prepare the pharmaceutically acceptable salt is that which forms a non-toxic acid addition salt, i.e., a salt containing pharmacologically acceptable anions such as the hydrochloride, hydroiodide, hydrobromide, nitrate, sulfate, bisulfate, phosphate, acetate, lactate, citrate, tartrate, succinate, maleate, fumarate, benzoate, para-toluenesulfonate, and the like.

The compounds or pharmaceutical compositions of the present disclosure may be co-administered with one or more therapeutically active agent. The term “co-administration” or “coadministration” refers to administration of (a) compound of formula (AI), (I), (AII), and (II), and any subgenera thereof, or a pharmaceutically acceptable salt, solvate, ester, or prodrug thereof, and (b) at least one additional therapeutically active agent, together in a coordinated fashion. For example, the co-administration can be simultaneous administration, sequential administration, overlapping administration, interval administration, continuous administration, or a combination thereof. In one embodiment, the compound of the present disclosure and at least one additional therapeutically active agent are formulated into a single dosage form. In another embodiment, the compound of the present disclosure and at least one additional therapeutically active agent are provided in a separate dosage form.

In one embodiment, the co-administration is carried out for one or more treatment cycles. By “treatment cycle”, it is meant a pre-determined period of time for co-administering the compound of the present disclosure (e.g., a compound of formula (AI), (I), (AII), and (II), and any subgenera thereof, or a pharmaceutically acceptable salt, solvate, ester, or prodrug thereof) and at least one therapeutically active agent. Typically, the patient is examined at the end of each treatment cycle to evaluate the effect of the present combination therapy.

Depending on the patient's condition and the intended therapeutic effect, the dosing frequency for each of the compound of the present disclosure (e.g., a compound of formula (AI), (I), (AII), and (II), and any subgenera thereof, or a pharmaceutically acceptable salt, solvate, ester, or prodrug thereof) and at least one therapeutically active agent may vary from once per day to six times per day. That is, the dosing frequency may be once per day, twice per day, three times per day, four times per day, five times per day, or six times per day. In some embodiments, dosing frequency may be one to six times per week or one to four times per month. In one embodiment, dosing frequency may be once a week, once every two weeks, once every three weeks, once every four weeks, or once a month.

There may be one or more void days in a treatment cycle. By “void day”, it is meant a day when neither the compound of the present disclosure (e.g., a compound of formula (AI), (I), (AII), and (II), and any subgenera thereof, or a pharmaceutically acceptable salt, solvate, ester, or prodrug thereof) or at least one therapeutically active agent is administered. In other words, none of the compound of the present disclosure and at least one therapeutically active agent is administered on a void day. Any treatment cycle must have at least one non-void day. By “non-void day”, it is meant a day when at least one of the compounds of the present disclosure and at least one therapeutically active agent is administered.

By “simultaneous administration”, it is meant that the compound of the present disclosure e.g., a compound of formula (AI), (I), (AII), and (II), and any subgenera thereof, or a pharmaceutically acceptable salt, solvate, ester, or prodrug thereof) and at least one therapeutically active agent are administered on the same day. For the simultaneous administration, the compound of the present disclosure and at least one therapeutically active agent can be administered at the same time or one at a time. The administration of the compound of the present disclosure and at least one therapeutically active agent occurs within 24 hours or less.

In one embodiment of the simultaneous administration, the compound of the present disclosure (e.g., a compound of formula (AI), (I), (AII), and (II), and any subgenera thereof, or a pharmaceutically acceptable salt, solvate, ester, or prodrug thereof), is administered from 1 to 4 times per day, 1 to 4 times per week, once every two weeks, once every three weeks, once every four weeks or 1 to 4 times per month; and the at least one additional therapeutically active agent is administered 1 to 4 times per day, 1 to 4 times per week, once every two weeks, once every three weeks, once every four weeks or 1 to 4 times per month. In another embodiment of the simultaneous administration, the compound of the present disclosure, is administered once a week, once every two weeks, once every three weeks, once every four weeks, or once a month; and the at least one additional therapeutically active agent is administered 1 to 4 times per day, 1 to 4 times per week, once every two weeks, once every three weeks, once every four weeks or 1 to 4 times per month.

By “sequential administration”, it is meant that during a period of two or more days of continuous co-administration without any void day, only one of the compound of the present disclosure (e.g., a compound of formula (AI), (I), (AII), and (II), and any subgenera thereof, or a pharmaceutically acceptable salt, solvate, ester, or prodrug thereof) and at least one therapeutically active agent is administered on any given day.

In one embodiment of the sequential administration, the compound of the present disclosure (e.g., a compound of formula (AI), (I), (AII), and (II), and any subgenera thereof, or a pharmaceutically acceptable salt, solvate, ester, or prodrug thereof), is administered from 1 to 4 times per day, 1 to 4 times per week, once every two weeks, once every three weeks, once every four weeks or 1 to 4 times per month; and at least one additional therapeutically active agent is administered 1 to 4 times per day, 1 to 4 times per week, once every two weeks, once every three weeks, once every four weeks or 1 to 4 times per month. In another embodiment of the sequential administration, the compound of the present disclosure, is administered from once a week, once every two weeks, once every three weeks, once every four weeks, or once a month; and at least one additional therapeutically active agent is administered 1 to 4 times per day, 1 to 4 times per week, once every two weeks, once every three weeks, once every four weeks or 1 to 4 times per month.

By “overlapping administration”, it is meant that during a period of two or more days of continuous co-administration without any void day, there is at least one day of simultaneous administration and at least one day when only one of the compound of the present disclosure (e.g., a compound of formula (AI), (I), (AII), and (II), and any subgenera thereof, or a pharmaceutically acceptable salt, solvate, ester, or prodrug thereof) and at least one therapeutically active agent is administered.

By “interval administration”, it is meant a period of co-administration with at least one void day. By “continuous administration”, it is meant a period of co-administration without any void day. The continuous administration may be simultaneous, sequential, or overlapping, as described above.

In the present method, the co-administration comprises oral administration, parenteral administration, or a combination thereof. Examples of the parenteral administration include, but are not limited to intravenous (IV) administration, intraarterial administration, intramuscular administration, subcutaneous administration, intraosseous administration, intrathecal administration, or a combination thereof. The compound of the present disclosure (e.g., a compound of formula (AI), (I), (AII), and (II), and any subgenera thereof, or a pharmaceutically acceptable salt, solvate, ester, or prodrug thereof) and at least one therapeutically active agent can be independently administered orally or parenterally. In one embodiment, the compound of the present disclosure and at least one therapeutically active agent is administered parenterally. The parenteral administration may be conducted via injection or infusion.

When administered for the treatment of a particular disease state or disorder, such as CNS disorders, it is understood that an effective dosage can be depending upon the particular compound utilized, the mode of administration, and severity of the condition being treated, as well as the various physical factors related to the individual being treated. In therapeutic applications, a compound of the present teachings can be provided to a patient already suffering from a disease in an amount sufficient to cure or at least partially ameliorate the symptoms of the disease and its complications. The dosage to be used in the treatment of a specific individual typically must be subjectively determined by the attending physician. The variables involved include the specific condition and its state as well as the size, age and response pattern of the patient.

Having now generally described the invention, the same will be more readily understood through reference to the following examples, which are provided by way of illustration and are not intended to be limiting of the present invention.

### EXAMPLES

In order that the invention described herein may be more fully understood, the following examples are set forth. The synthetic and biological examples described in this application are offered to illustrate the compounds, pharmaceutical compositions and methods provided herein and are not to be construed in any way as limiting their scope.

Materials and Methods

The compounds provided herein may be prepared from known or commercially available starting materials and reagents by one skilled in the art of organic synthesis. The compounds provided herein can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in T. W. Greene and P. G. M. Wuts, Protecting Groups in Organic Synthesis, Second Edition, Wiley, New York, 1991, and references cited therein.

The compounds provided herein may be isolated and purified by known standard procedures. Such procedures include (but are not limited to) recrystallization, column chromatography, HPLC, or supercritical fluid chromatography (SFC).

Synthetic Procedures

The following schemes provide exemplary synthetic routes for preparing compounds of the present disclosure. These general schemes together with the specific Examples below provide guidance for the synthesis.

Compounds of formula (AI), (I), (AII), and (II) may be prepared according to the methods outlined in Schemes 1-4.

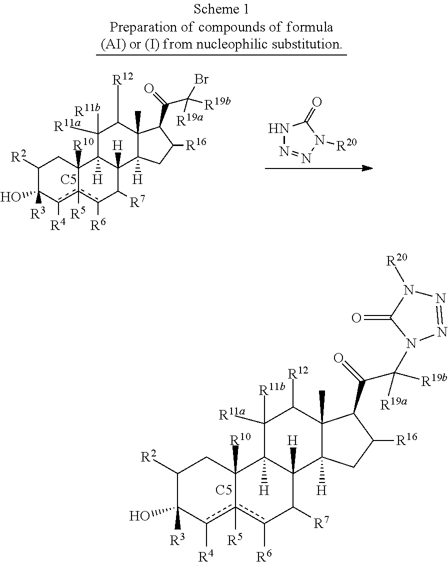

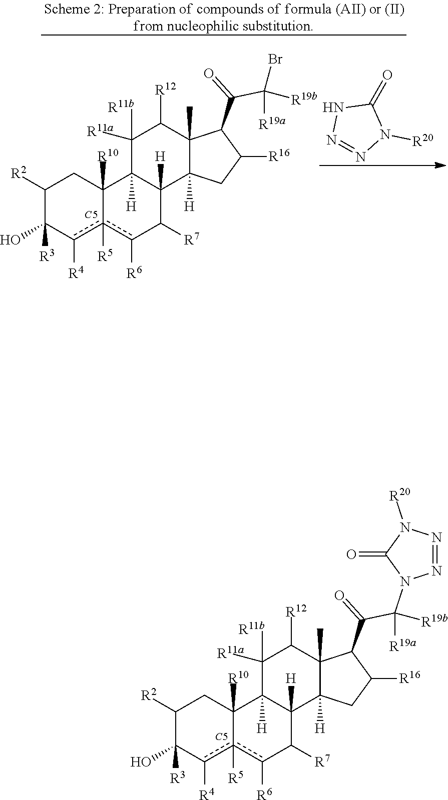

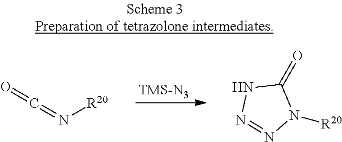

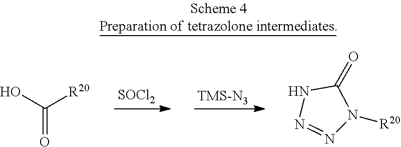

### Example 1: Synthesis of 2-bromo-1-((3R,5R,8R,9R,10S,13S,14S,17S)-3-hydroxy-3,13-dimethylhexadecahydro-1H-cyclopenta[a]phenanthren-17-yl)ethan-1-one (Compound 7)

Compound 7 was synthesized according to published procedures in Patent PCT/CN2014/075594 and J. Med. Chem. 2017, 60, 7810-7819.

### Step 1: Synthesis of (5R,8R,9R,10S,13S,14S)-13-methyltetradecahydro-3H-cyclopenta[a]phenanthrene-3,17(2H)-dione (Compound 2)

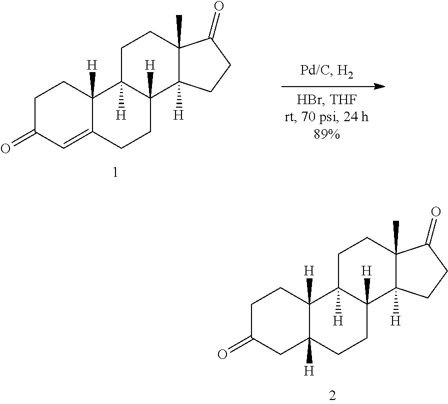

A mixture of compound 1 (50.0 g, 184.56 mmol, 1.0 eq), palladium black (2.5 g, 5% w/w) in tetrahydrofuran (300 mL) and concentrated hydrobromic acid (1 mL) was hydrogenated with 70 psi hydrogen. The mixture was stirring at room temperature for 24 h. The progress of the reaction mixture was monitored by TLC. After completion of the reaction, the mixture was filtered through a pad of celite and sintered funnel and the filtrate was concentrated under reduced pressure. The residue was recrystallized from acetone to afford compound 2 (45.0 g, 89%). TLC: hexane/ethyl acetate (3:1); Rf: (Compound 1)=0.4; Rf: (Compound 2)=0.3.

### Step 2: Synthesis of (3R,5R,8R,9R,10S,13S,14S)-3-hydroxy-3,13-dimethylhexadecahydro-17H-cyclopenta[a]phenanthren-17-one (Compound 3)

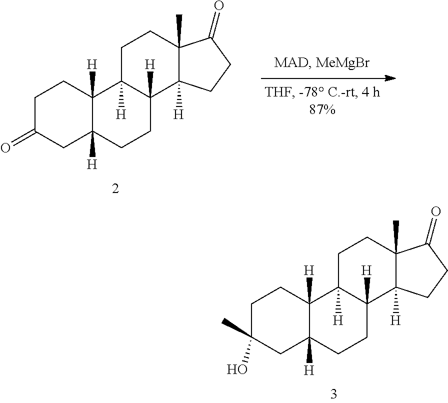

To a solution of methyl aluminum bis(2, 6-di-tert-butyl-4-methylphenoxide (MAD) (0.192 mol, 3.5 eq, freshly prepared) in THF, was added dropwise a solution of compound 2 (25.0 g, 54.74 mmol, 1.0 eq) in anhydrous toluene (150 mL) at −78° C. under nitrogen atmosphere. The reaction mixture was stirred at −78° C. for 1 h. Then methyl magnesium bromide (3 M in diethyl ether, 63.9 mL, 0.192 mol, 3.5 eq) was slowly added dropwise to the above mixture at −78° C. under nitrogen atmosphere. Then the reaction mixture was stirred for 3 h at −78° C. The progress of the reaction mixture was monitored by TLC. After completion of the reaction, saturated aqueous ammonium chloride solution was slowly added dropwise to the above mixture at −78° C. Then the mixture was filtered and the filter cake was washed with ethyl acetate. The organic layer was washed with water and brine, dried over anhydrous sodium sulfate, filtered and concentrated under reduced pressure, purified by silica gel chromatography (5-25% ethyl acetate in petroleum ether) to afford compound 3 (23.2 g 87%). TLC: hexane/ethyl acetate (5:1); Rf: (Compound 2)=0.5; Rf: (Compound 3)=0.2;

### Step 3: Synthesis of (3R,5R,8R,9R,10S,13S,14S)-17-ethylidene-3,13-dimethylhexadecahydro-1H-cyclopenta[a]phenanthren-3-ol (Compound 4)

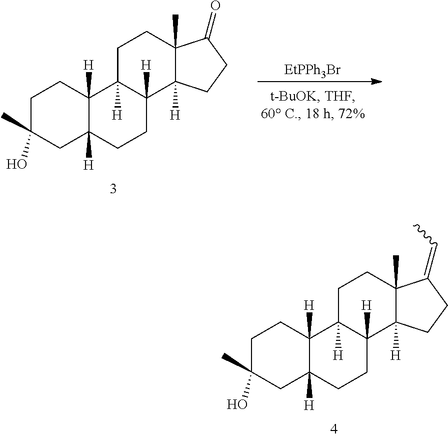

To a solution of ethyltriphenylphosphonium bromide (296.6 g, 0.799 mol, 10.0 eq) in tetrahydrofuran (800 mL), was added potassium tert-butoxide (1 M in THF, 79.9 mL, 0.799 mol, 10.0 eq) at 0° C. After the addition was completed, the reaction mixture was stirred for 1 h at 60° C., then a solution of compound 3 (23.2 g, 79.94 mmol, 1.0 eq) in tetrahydrofuran (232 mL) was added dropwise at 60° C. The reaction mixture was stirred at 60° C. for 18 h. The progress of the reaction mixture was monitored by TLC. After completion of the reaction, the reaction mixture was cooled to room temperature, quenched with saturated aqueous ammonium chloride solution and extracted with ethyl acetate (3×800 mL). The combined organic layers were washed with brine, dried and concentrated under reduced pressure. The residue was purified by silica gel chromatography (2-10% ethyl acetate in petroleum ether) to afford compound 4 (17.6 g, 72%). TLC: hexane/ethyl acetate (6:1); Rf: (Compound 3)=1; Rf: (Compound 4)=0.5.

### Step 4: Synthesis of (3R,5R,8R,9R,10S,13S,14S,17S)-17-(l-hydroxyethyl)-3,13-dimethylhexadecahydro-1H-cyclopenta[a]phenanthren-3-ol (Compound 5)

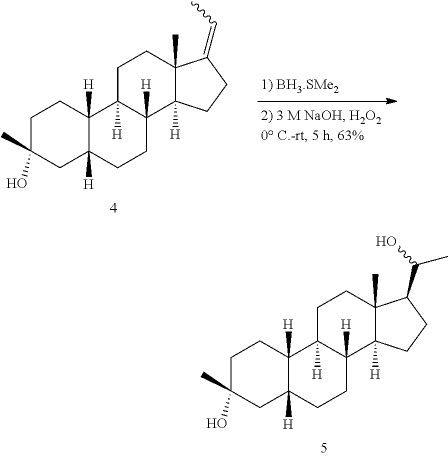

To a solution of compound 4 (17.6 g, 58.28 mmol, 1.0 eq) in anhydrous tetrahydrofuran (200 mL), was added dropwise a solution of borane-methyl sulfide complex (2 M in tetrahydrofuran, 437.1 mL, 0.874 mol, 15.0 eq) under ice-bath. After the addition was completed, the reaction mixture was stirred for 3 h at room temperature (14-20° C.). The progress of the reaction mixture was monitored by TLC. After completion of the reaction, the mixture was cooled to 0° C. and 3.0 M aqueous sodium hydroxide solution (291.4 mL, 0.874 mol, 15.0 eq) followed by 30% aqueous hydrogen peroxide (30%, 138.9 mL) was added. The mixture was stirred for 2 h at room temperature (14-20° C.), and then filtered, extracted with ethyl acetate (3×500 mL). The combined organic layers were washed with saturated aqueous sodium thiosulfate, brine, dried over sodium sulfate and concentrated in vacuum to give crude compound 5 (11.8 g, 63%) which was used in the next step without further purification. TLC: hexane/ethyl acetate (1:1); Rf: (Compound 4)=1; Rf: (Compound 5)=0.7.

### Step 5: Synthesis of 1-((3R,5R,8R,9R,10S,13S,14S,17S)-3-hydroxy-3,13-dimethylhexadecahydro-1H-cyclopenta[a]phenanthren-17-yl)ethan-1-one (Compound 6)

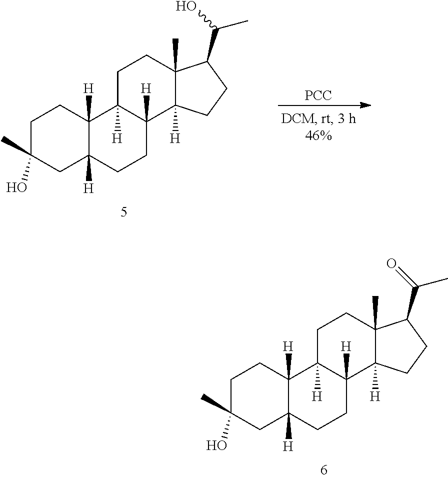

To a solution of compound 5 (11.8 g, 36.88 mmol, 1.0 eq) in dichloromethane (100 mL) at 0° C. was added pyridinium chlorochromate (15.9 g, 73.75 mmol, 2.0 eq) in portions. Then the reaction mixture was stirred at room temperature (16-22° C.) for 3 h. The progress of the reaction mixture was monitored by TLC. After completion of the reaction, the reaction mixture was filtered and washed with dichloromethane. The organic phase was washed with saturated aqueous sodium thiosulfate, brine, dried over sodium sulfate and concentrated in vacuum. The residue was purified by silica gel chromatography (0-11% ethyl acetate in petroleum ether) to afford compound 6 (5.45 g, 46%). TLC: hexane/ethyl acetate (15:1); Rf: (Compound 5)=0.7; Rf: (Compound 6)=0.5.

### Step 6: Synthesis of 2-bromo-1-((3R,5R,8R,9R,10S,13S,14S,17S)-3-hydroxy-3,13-dimethylhexadecahydro-1H-cyclopenta[a]phenanthren-17-yl)ethan-1-one (Compound 7)

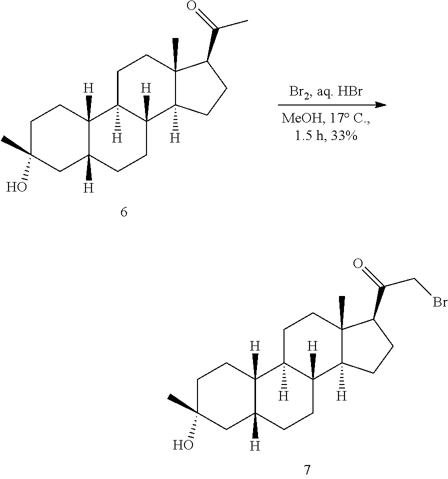

To a solution of compound 6 (3.45 g, 10.85 mmol, 1.0 eq) and aq. hydrogen bromide (2 drops, 48% in water) in methanol (30 mL) was added bromine (1.9 g, 11.93 mmol, 1.1 eq). Then the reaction mixture was stirred at 17° C. for 1.5 h. The progress of the reaction mixture was monitored by TLC. After completion of the reaction, the reaction mixture was quenched with saturated aqueous sodium bicarbonate at 0° C. and extracted with ethyl acetate. The organic phase was washed with saturated aqueous sodium thiosulfate, brine, dried over sodium sulfate and concentrated in vacuum. The residue was purified by silica gel chromatography (5-15% ethyl acetate in petroleum ether) to afford compound 7 (1.42 g, 33%). TLC: hexane/ethyl acetate (3:1); Rf: (Compound 6)=0.2; Rf: (Compound 7)=0.4.

### Example 2: Synthesis of 1-(2-((3R,5R,8R,9R,10S,13S,14S,17S)-3-hydroxy-3,13-dimethylhexadecahydro-1H-cyclopenta[a]phenanthren-17-yl)-2-oxoethyl)-4-methyl-1,4-dihydro-5H-tetrazol-5-one (Compound 8)

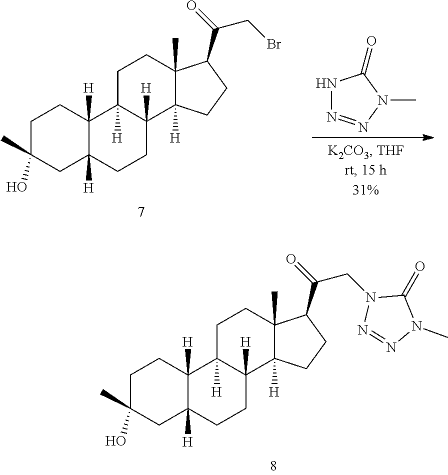

To a suspension of potassium carbonate (42 mg, 0.303 mmol, 0.8 eq) in tetrahydrofuran (5 mL) was added 1-methyl-1,4-dihydro-5H-tetrazol-5-one (114 mg, 1.14 mmol, 3.0 eq) and compound 7 (150 mg, 0.379 mmol, 1.0 eq). The mixture was stirred at room temperature for 15 h. The progress of the reaction mixture was monitored by TLC (petroleum ether/ethyl acetate=3:1). After completion of the reaction, the mixture was poured into 10 mL water and extracted with ethyl acetate (2×20 mL). The combined organic layers were washed with brine (10 mL), dried over anhydrous sodium sulfate, filtered and concentrated under reduced pressure. The residual mixture was purified by prep-HPLC to afford compound 8 (50 mg, 31%) as a white solid. TLC: PE/EA=3/1, 254 nm; Rf (Compound 7)=0.4; Rf (Compound 8)=0.2; LC-MS: 417 (M+1)⁺; ¹H NMR (400 MHz, CDCl₃) δ 4.76-4.62 (m, 2H), 3.62 (s, 3H), 2.58 (d, J=9.1 Hz, 1H), 2.19 (d, J=9.1 Hz, 1H), 2.06 (t, J=12.9 Hz, 1H), 1.89-1.68 (m, 4H), 1.63 (s, 1H), 1.55-1.35 (m, 8H), 1.28 (d, J=19.6 Hz, 6H), 1.08 (s, 3H), 0.67 (s, 3H).

### Example 3: Synthesis of 1-ethyl-4-(2-((3R,5R,8R,9R,10S,13S,14S,17S)-3-hydroxy-3,13-dimethylhexadecahydro-1H-cyclopenta[a]phenanthren-17-yl)-2-oxoethyl)-1,4-dihydro-5H-tetrazol-5-one (Compound 11)

### Step 1: Synthesis of 1-ethyl-1,4-dihydro-5H-tetrazol-5-one (Compound 10)

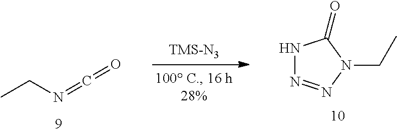

A stirred mixture of isocyanatoethane (1.0 g, 14.08 mmol, 1.0 eq) and azidotrimethylsilane (3.5 ml, 26.76 mmol, 1.9 eq) was heated to 100° C. and stirred for 16 h. The progress of the reaction mixture was monitored by TLC. After completion of the reaction, the mixture was cooled to room temperature and concentrated. The residue was diluted with ethyl acetate (10 mL) and extracted with saturated aqueous sodium bicarbonate (3×10 mL). The combined aqueous layers were added 6 M hydrochloric acid to adjust pH<3 with efficient stirring and extracted with ethyl acetate (3×20 mL). The combined organic layers were dried over anhydrous sodium sulfate and concentrated under reduced pressure to afford compound 10 (460 mg, 28%) as a white solid. TLC: dichloromethane/methanol (10:1); Rf: (Compound 10)=0.5.

### Step 2: Synthesis of 1-ethyl-4-(2-((3R,5R,8R,9R,10S,13S,14S,17S)-3-hydroxy-3,13-dimethylhexadecahydro-1H-cyclopenta[a]phenanthren-17-yl)-2-oxoethyl)-1,4-dihydro-5H-tetrazol-5-one (Compound 11)

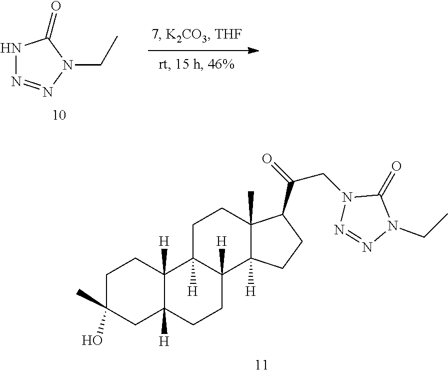

To a suspension of potassium carbonate (697 mg, 5.05 mmol, 10 eq) in tetrahydrofuran (10 mL), was added compound 10 (173 mg, 1.52 mmol, 3.0 eq) and compound 7 (200 mg, 0.505 mmol, 1.0 eq). The mixture was stirred at room temperature for 15 h, monitored by TLC. After completion, the mixture was poured into water (10 mL) and extracted with ethyl acetate (2×20 mL). The combined organic layers were washed with brine (10 mL), dried over anhydrous sodium sulfate, filtered and concentrated under reduced pressure. The residual mixture was purified by prep-HPLC. The desired product 11 was obtained as a white solid, 100 mg, in 46% yield. TLC: PE/EA=3/1, 254 nm; Rf (Compound 7)=0.4; Rf (Compound 11)=0.2; LC-MS: 413.25 [M−18]⁻; ¹H NMR (400 MHz, CDCl₃): δ 4.69 (d, J=2.9 Hz, 2H), 4.04-3.95 (m, 2H), 2.58 (d, J=8.9 Hz, 1H), 2.24-2.14 (m, 1H), 2.11-2.04 (m, 1H), 1.76 (dt, J=13.8, 12.9 Hz, 4H), 1.60 (s, 3H), 1.44 (dt, J=14.4, 6.3 Hz, 11H), 1.25 (s, 5H), 1.11 (dd, J=16.7, 7.9 Hz, 4H), 0.68 (d, J=5.1 Hz, 3H).

### Example 4: Synthesis of 1-(2-((3R,5R,8R,9R,10S,13S,14S,17S)-3-hydroxy-3,13-dimethylhexadecahydro-1H-cyclopenta[a]phenanthren-17-yl)-2-oxoethyl)-4-isopropyl-1,4-dihydro-5H-tetrazol-5-one (Compound 14)

### Step 1: Synthesis of 1-isopropyl-1,4-dihydro-5H-tetrazol-5-one (Compound 13)

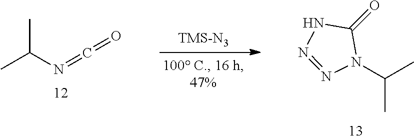

A stirred mixture of 2-isocyanatopropane (700 mg, 8.24 mmol, 1.0 eq) and azidotrimethylsilane (2.0 ml, 15.65 mmol, 1.9 eq) was heated to 100° C. and stirred for 16 h. The progress of the reaction mixture was monitored by TLC. After completion of the reaction, the mixture was cooled to room temperature and concentrated. The residue was diluted with ethyl acetate (10 mL) and extracted with saturated aqueous sodium bicarbonate (3×10 mL). The combined aqueous layers were added 6 M hydrochloric acid to adjust pH<3 with efficient stirring and extracted with ethyl acetate (3×20 mL). The combined organic layers were dried over anhydrous sodium sulfate and concentrated under reduced pressure to afford compound 13 (500 mg, 47%) as a white solid. TLC: dichloromethane/methanol (10:1); Rf: (Compound 13)=0.5; Compound 12: ¹H NMR (400 MHz, CDCl₃) δ 3.76-3.62 (m, 1H), 1.27 (s, 3H), 1.25 (s, 3H); Compound 13: ¹H NMR (400 MHz, CDCl₃) δ 4.50 (dt, J=13.5, 6.7 Hz, 1H), 1.51 (s, 3H), 1.49 (s, 3H).

### Step 2: Synthesis of 1-(2-((3R,5R,8R,9R,10S,13S,14S,17S)-3-hydroxy-3,13-dimethylhexadecahydro-1H-cyclopenta[a]phenanthren-17-yl)-2-oxoethyl)-4-isopropyl-1,4-dihydro-5H-tetrazol-5-one (Compound 14)

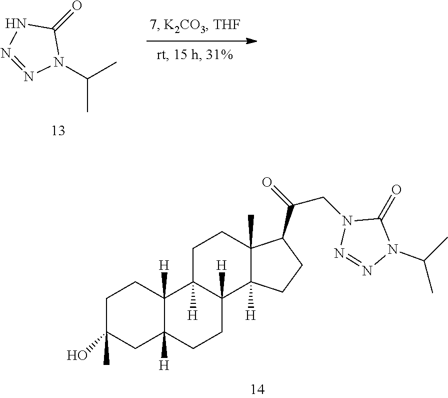

To a suspension of potassium carbonate (697 mg, 5.05 mmol, 10 eq) in tetrahydrofuran (10 mL), was added compound 13 (195 mg, 1.52 mmol, 3.0 eq) and compound 7 (200 mg, 0.505 mmol, 1.0 eq). The mixture was stirred at room temperature for 15 h, monitored by TLC. After completion, the mixture was poured into water (10 mL) and extracted with ethyl acetate (2×20 mL). The combined organic layers were washed with brine (10 mL), dried over anhydrous sodium sulfate, filtered and concentrated under reduced pressure. The residual mixture was purified by prep-HPLC. The desired product 14 was obtained as a white solid, 70 mg, in 31% yield. TLC: dichloromethane/methanol=10/1, 254 nm; Rf (Compound 14)=0.2; LC-MS: 427.30 [M−18]⁻; ¹H NMR (400 MHz, CDCl₃): δ 4.71 (dd, J=42.2, 18.2 Hz, 2H), 4.52-4.44 (m, 1H), 2.78 (dd, J=8.3, 2.7 Hz, 1H), 1.80 (s, 3H), 1.72 (d, J=11.3 Hz, 3H), 1.50 (dd, J=6.7, 3.4 Hz, 8H), 1.36 (d, J=10.4 Hz, 5H), 1.22 (d, J=8.8 Hz, 8H), 1.14-1.10 (m, 3H), 0.93 (s, 3H), 0.84 (d, J=6.7 Hz, 3H).

### Example 5: Synthesis of 1-(tert-butyl)-4-(2-((3R,5R,8R,9R,10S,13S,14S,17S)-3-hydroxy-3,13-dimethylhexadecahydro-1H-cyclopenta[a]phenanthren-17-yl)-2-oxoethyl)-1,4-dihydro-5H-tetrazol-5-one (Compound 17)

### Step 1: Synthesis of 1-(tert-butyl)-1,4-dihydro-5H-tetrazol-5-one (Compound 16)

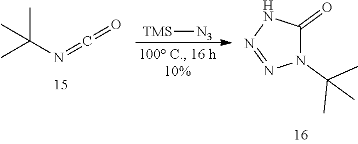

A stirred mixture of 2-isocyanato-2-methylpropane (3.0 g, 30.3 mmol, 1.0 eq) and azidotrimethylsilane (9.0 ml, 67.5 mmol, 2.2 eq) was heated to 100° C. and stirred for 16 h. The progress of the reaction mixture was monitored by TLC. After completion of the reaction, the mixture was cooled to room temperature and concentrated. The residue was diluted with ethyl acetate (10 mL) and extracted with saturated aqueous sodium bicarbonate (3×10 mL). The combined aqueous layers were added 6 M hydrochloric acid to adjust pH<3 with efficient stirring and extracted with ethyl acetate (3×20 mL). The combined organic layers were dried over anhydrous sodium sulfate and concentrated under reduced pressure to afford compound 16 (441 mg, 10%) as a white solid. TLC: dichloromethane/methanol (10:1); Rf: (Compound 16)=0.5; Compound 15: ¹H NMR (400 MHz, CDCl₃) δ 1.34 (s, 9H); Compound 16: ¹H NMR (400 MHz, CDCl₃) δ 1.61 (s, 9H).

### Step 2: Synthesis of 1-(tert-butyl)-4-(2-((3R,5R,8R,9R,10S,13S,14S,17S)-3-hydroxy-3,13-dimethylhexadecahydro-1H-cyclopenta[a]phenanthren-17-yl)-2-oxoethyl)-1,4-dihydro-5H-tetrazol-5-one (Compound 17)

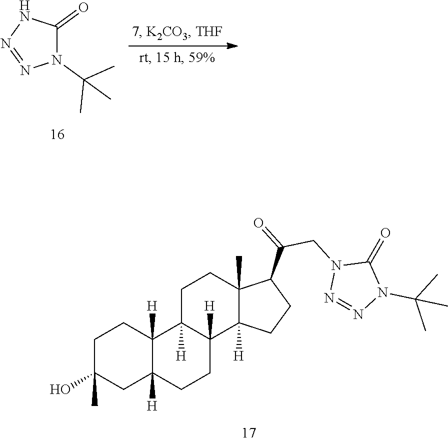

To a suspension of potassium carbonate (1.0 g, 7.25 mmol, 10 eq) in tetrahydrofuran (10 mL), was added compound 16 (322.3 mg, 2.27 mmol, 3.0 eq) and compound 7 (300 mg, 0.76 mmol, 1.0 eq). The mixture was stirred at room temperature for 15 h, monitored by TLC. After completion, the mixture was poured into water (10 mL) and extracted with ethyl acetate (2×20 mL). The combined organic layers were washed with brine (10 mL), dried over anhydrous sodium sulfate, filtered and concentrated under reduced pressure. The residual mixture was purified by prep-HPLC. The desired product 17 was obtained as a white solid, 206 mg, in 59% yield. TLC: petroleum ether/ethyl acetate (3:1); Rf: (Compound 7)=0.4; Rf: (Compound 17)=0.2; LC-MS: 459 (M+1)⁺; ¹H NMR (400 MHz, CDCl₃) δ 4.65 (d, J=4.2 Hz, 2H), 2.58 (t, J=8.9 Hz, 1H), 2.23-2.03 (m, 1H), 1.85-1.77 (m, 8H), 1.62 (s, 9H), 1.48-1.36 (m, 8H), 1.28-1.21 (m, 9H), 0.66 (s, 3H).

### Example 6: Synthesis of 1-cyclopropyl-4-(2-((3R,5R,8R,9R,10S,13S,14S,17S)-3-hydroxy-3,13-dimethylhexadecahydro-1H-cyclopenta[a]phenanthren-17-yl)-2-oxoethyl)-1,4-dihydro-5H-tetrazol-5-one (Compound 20)

### Step 1: Synthesis of 1-cyclopropyl-1,4-dihydro-5H-tetrazol-5-one (Compound 19)

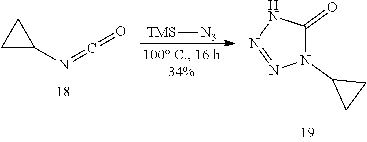 [SMILES: O=C=NC1CC1 ; C[Si](C)(C)N=[N+]=[N-] ; O=c1[nH]nnn1C1CC1]

A stirred mixture of isocyanatocyclopropane (1.0 g, 12.0 mmol, 1.0 eq) and azidotrimethylsilane (3 ml, 22.5 mmol, 2.2 eq) was heated to 100° C. and stirred for 16 h. The progress of the reaction mixture was monitored by TLC. After completion of the reaction, the mixture was cooled to room temperature and concentrated. The residue was diluted with ethyl acetate (10 mL) and extracted with saturated aqueous sodium bicarbonate (3×10 mL). The combined aqueous layers were added 6 M hydrochloric acid to adjust pH<3 with efficient stirring and extracted with ethyl acetate (3×20 mL). The combined organic layers were dried over anhydrous sodium sulfate and concentrated under reduced pressure to afford compound 19 (521 mg, 34%) as a yellow solid. TLC: dichloromethane/methanol (10:1); Rf: (Compound 19)=0.5; Compound 18: ¹H NMR (400 MHz, CDCl₃) δ 2.78-2.70 (m, 1H), 0.69-0.65 (m, 2H), 0.62 (ddd, J=4.1, 3.7, 1.7 Hz, 2H); Compound 19: ¹H NMR (400 MHz, CDCl₃) δ 3.27 (ddd, J=12.9, 7.2, 3.9 Hz, 1H), 1.20-1.10 (m, 4H).

### Step 2: Synthesis of 1-cyclopropyl-4-(2-((3R,5R,8R,9R,10S,13S,14S,17S)-3-hydroxy-3,13-dimethylhexadecahydro-1H-cyclopenta[a]phenanthren-17-yl)-2-oxoethyl)-1,4-dihydro-5H-tetrazol-5-one (Compound 20)

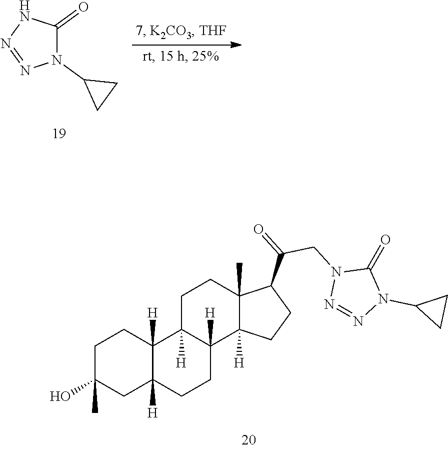

To a suspension of potassium carbonate (690 mg, 5.0 mmol, 10.0 eq) in tetrahydrofuran (10 mL), was added compound 19 (189.2 mg, 1.5 mmol, 3.0 eq) and compound 7 (200 mg, 0.5 mmol, 1.0 eq). The mixture was stirred at room temperature for 15 h, monitored by TLC. After completion, the mixture was poured into water (10 mL) and extracted with ethyl acetate (2×20 mL). The combined organic layers were washed with brine (10 mL), dried over anhydrous sodium sulfate, filtered and concentrated under reduced pressure. The residual mixture was purified by prep-HPLC. The desired product 20 was obtained as a white solid, 56 mg, in 25% yield. TLC: petroleum ether/ethyl acetate (3:1); Rf: (Compound 7)=0.4; Rf: (Compound 20)=0.2; LC-MS: 443 (M+1)⁺; ¹H NMR (400 MHz, CDCl₃) δ 4.67 (d, J=3.4 Hz, 2H), 3.26 (tt, J=7.2, 3.7 Hz, 1H), 2.58 (t, J=9.0 Hz, 1H), 2.25-2.03 (m, 2H), 1.82-1.69 (m, 4H), 1.56 (s, 4H), 1.45-1.37 (m, 8H), 1.25 (m, 8H), 1.19-1.15 (m, 1H), 1.11-1.06 (m, 3H), 0.67 (s, 3H).

### Example 7: Synthesis of 1-(cyclopropylmethyl)-4-(2-((3R,5R,8R,9R,10S,13S,14S,17S)-3-hydroxy-3,13-dimethylhexadecahydro-1H-cyclopenta[a]phenanthren-17-yl)-2-oxoethyl)-1,4-dihydro-5H-tetrazol-5-one (Compound 24)

### Step 1: Synthesis of (isocyanatomethyl)cyclopropane (Compound 22)

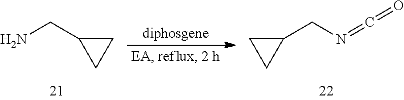 [SMILES: NCC1CC1 ; O=C=NCC1CC1]

To a solution of cyclopropylmethanamine (2.0 g, 28.12 mmol, 1.0 eq) in dry ethyl acetate (6 ml), was added a solution of diphosgene (6.67 g, 33.75 mmol, 1.2 eq) in dry ethyl acetate (6 ml) with a catalytic amount of charcoal. The reaction mixture was stirred at room temperature for 5 min and then heated to reflux until the mixture was clear. The progress of the reaction mixture was monitored by LCMS. After completion of the reaction, the solution was cooled to room temperature, filtered and concentrated under reduced pressure to afford crude compound 22 (3.2 g), which was used for the next step without further purification.

### Step 2: Synthesis of 1-(cyclopropylmethyl)-1,4-dihydro-5H-tetrazol-5-one (Compound 23)

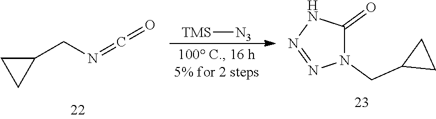 [SMILES: O=C=NCC1CC1 ; C[Si](C)(C)N=[N+]=[N-] ; O=c1[nH]nnn1CC1CC1]

A stirred mixture of compound 22 (3.2 g, crude) and azidotrimethylsilane (10 ml, 75 mmol, 2.2 eq) was heated to 100° C. and stirred for 16 h. The progress of the reaction mixture was monitored by TLC. After completion of the reaction, the mixture was cooled to room temperature and concentrated. The residue was diluted with ethyl acetate (10 mL) and extracted with saturated aqueous sodium bicarbonate (3×10 mL). The combined aqueous layers were added 6 M hydrochloric acid to adjust pH<3 with efficient stirring and extracted with ethyl acetate (3×20 mL). The combined organic layers were dried over anhydrous sodium sulfate and concentrated under reduced pressure to afford compound 23 (210 mg, 5% for two steps) as a colorless oil. ¹H NMR (400 MHz, CDCl₃) δ 3.80 (d, J=7.3 Hz, 2H), 1.28-1.21 (m, 1H), 0.62-0.55 (m, 2H), 0.41 (q, J=4.9 Hz, 2H); TLC: dichloromethane/methanol (10:1); Rf: (Compound 23)=0.5.

### Step 3: Synthesis of 1-(cyclopropylmethyl)-4-(2-((3R,5R,8R,9R,10S,13S,14S,17S)-3-hydroxy-3,13-dimethylhexadecahydro-1H-cyclopenta[a]phenanthren-17-yl)-2-oxoethyl)-1,4-dihydro-5H-tetrazol-5-one (Compound 24)

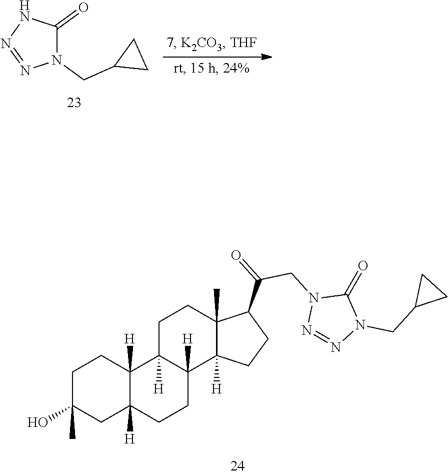

To a suspension of potassium carbonate (1.0 g, 7.6 mmol, 10.0 eq) in tetrahydrofuran (15 mL), was added compound 23 (319.5 mg, 2.28 mmol, 3.0 eq) and compound 7 (300.0 mg, 0.76 mmol, 1.0 eq). The mixture was stirred at room temperature for 15 h, monitored by TLC. After completion, the mixture was poured into water (10 mL) and extracted with ethyl acetate (2×20 mL). The combined organic layers were washed with brine (10 mL), dried over anhydrous sodium sulfate, filtered and concentrated under reduced pressure. The residual mixture was purified by prep-HPLC. The desired product 24 was obtained as a white solid, 82 mg, in 24% yield. TLC: petroleum ether/ethyl acetate (3:1); Rf: (Compound 7)=0.4; Rf: (Compound 24)=0.2; LC-MS: 457 (M+1)⁺; ¹H NMR (400 MHz, CDCl₃) δ 4.70 (d, J=3.2 Hz, 2H), 3.81 (dd, J=7.2, 2.7 Hz, 2H), 2.60 (t, J=9.0 Hz, 1H), 2.24-2.06 (m, 2H), 1.86-1.70 (m, 6H), 1.57 (s, 7H), 1.42 (d, J=13.7 Hz, 5H), 1.27 (t, J=12.6 Hz, 4H), 1.08 (d, J=6.9 Hz, 3H), 0.68 (s, 3H), 0.60 (dd, J=12.4, 5.5 Hz, 2H), 0.46-0.38 (m, 2H).

### Example 8: Synthesis of 1-cyclopentyl-4-(2-((3R,5R,8R,9R,10S,13S,14S,17S)-3-hydroxy-3,13-dimethylhexadecahydro-1H-cyclopenta[a]phenanthren-17-yl)-2-oxoethyl)-1,4-dihydro-5H-tetrazol-5-one (Compound 27)

### Step 1: Synthesis of 1-cyclopentyl-1,4-dihydro-5H-tetrazol-5-one (Compound 26)

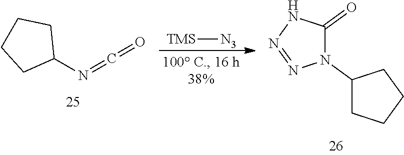 [SMILES: O=C=NC1CCCC1 ; C[Si](C)(C)N=[N+]=[N-] ; O=c1[nH]nnn1C1CCCC1]

A stirred mixture of isocyanatocyclopentane (1.0 g, 9.0 mmol, 1.0 eq) and azidotrimethylsilane (3.0 ml, 22.5 mmol, 2.5 eq) was heated to 100° C. and stirred for 16 h. The progress of the reaction mixture was monitored by TLC. After completion of the reaction, the mixture was cooled to room temperature and concentrated. The residue was diluted with ethyl acetate (10 mL) and extracted with saturated aqueous sodium bicarbonate (3×10 mL). The combined aqueous layers were added 6 M hydrochloric acid to adjust pH<3 with efficient stirring and extracted with ethyl acetate (3×20 mL). The combined organic layers were dried over anhydrous sodium sulfate and concentrated under reduced pressure to afford compound 26 (524 mg, 38%) as a white solid. TLC: dichloromethane/methanol (10:1); Rf: (Compound 26)=0.5; Compound 25: ¹H NMR (400 MHz, CDCl₃) δ 3.95-3.79 (m, 1H), 1.90-1.78 (m, 2H), 1.77-1.71 (m, 2H), 1.67 (dddd, J=9.2, 4.1, 2.5, 1.2 Hz, 2H), 1.64-1.57 (m, 2H); Compound 26: ¹H NMR (400 MHz, CDCl₃) δ 4.68-4.52 (m, 1H), 2.15-2.06 (m, 2H), 2.04-1.97 (m, 2H), 1.94-1.86 (m, 2H), 1.71 (dt, J=8.2, 6.1 Hz, 2H).

### Step 2: Synthesis of 1-cyclopentyl-4-(2-((3R,5R,8R,9R,10S,13S,14S,17S)-3-hydroxy-3,13-dimethylhexadecahydro-1H-cyclopenta[a]phenanthren-17-yl)-2-oxoethyl)-1,4-dihydro-5H-tetrazol-5-one (Compound 27)

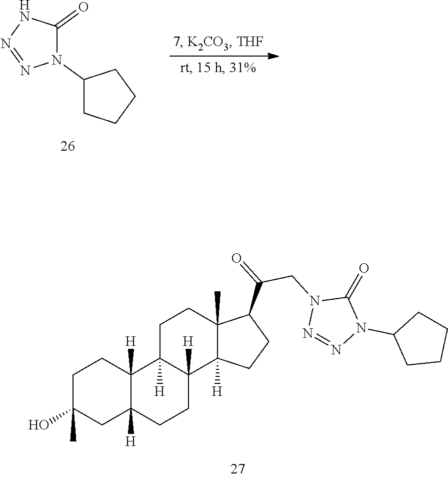

To a suspension of potassium carbonate (690 mg, 5.0 mmol, 10.0 eq) in tetrahydrofuran (10 mL), was added compound 26 (233 mg, 1.5 mmol, 3.0 eq) and compound 7 (200 mg, 0.5 mmol, 1.0 eq). The mixture was stirred at room temperature for 15 h, monitored by TLC. After completion, the mixture was poured into water (10 mL) and extracted with ethyl acetate (2×20 mL). The combined organic layers were washed with brine (10 mL), dried over anhydrous sodium sulfate, filtered and concentrated under reduced pressure. The residual mixture was purified by prep-HPLC. The desired product 27 was obtained as a white solid, 72 mg, in 31% yield. TLC: petroleum ether/ethyl acetate (3:1); Rf: (Compound 7)=0.4; Rf: (Compound 27)=0.2; LC-MS: 471 (M+1)⁺; ¹H NMR (400 MHz, CDCl₃): δ 4.69 (d, J=2.7 Hz, 2H), 2.59 (t, J=8.5 Hz, 1H), 2.09 (ddd, J=29.2, 15.5, 7.9 Hz, 5H), 1.83 (dd, J=32.4, 9.3 Hz, 6H), 1.75-1.65 (m, 8H), 1.48-1.36 (m, 8H), 1.25 (s, 3H), 1.14-1.03 (m, 5H), 0.67 (s, 3H).

### Example 9: Synthesis of 1-cyclohexyl-4-(2-((3R,5R,8R,9R,10S,13S,14S,17S)-3-hydroxy-3,13-dimethylhexadecahydro-1H-cyclopenta[a]phenanthren-17-yl)-2-oxoethyl)-1,4-dihydro-5H-tetrazol-5-one (Compound 30)

### Step 1: Synthesis of 1-cyclohexyl-1,4-dihydro-5H-tetrazol-5-one (Compound 29)

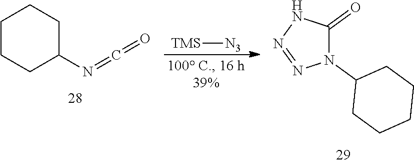 [SMILES: O=C=NC1CCCCC1 ; C[Si](C)(C)N=[N+]=[N-] ; O=c1[nH]nnn1C1CCCCC1]

A stirred mixture of isocyanatocyclohexane (1.0 g, 8.0 mmol, 1.0 eq) and azidotrimethylsilane (3.0 ml, 22.5 mmol, 2.8 eq) was heated to 100° C. and stirred for 16 h. The progress of the reaction mixture was monitored by TLC. After completion of the reaction, the mixture was cooled to room temperature and concentrated. The residue was diluted with ethyl acetate (10 mL) and extracted with saturated aqueous sodium bicarbonate (3×10 mL). The combined aqueous layers were added 6 M hydrochloric acid to adjust pH<3 with efficient stirring and extracted with ethyl acetate (3×20 mL). The combined organic layers were dried over anhydrous sodium sulfate and concentrated under reduced pressure to afford compound 29 (520 mg, 39%) as a white solid. TLC: dichloromethane/methanol (10:1); Rf: (Compound 29)=0.5; Compound 28: ¹H NMR (400 MHz, CDCl₃) δ 3.40 (ddd, J=12.7, 8.8, 3.7 Hz, 1H), 1.95-1.79 (m, 2H), 1.75-1.62 (m, 2H), 1.53-1.37 (m, 3H), 1.36-1.21 (m, 3H); Compound 29: ¹H NMR (400 MHz, CDCl₃) 54.10 (td, J=11.7, 3.9 Hz, 1H), 2.03-1.95 (m, 2H), 1.89 (d, J=15.5 Hz, 2H), 1.79 (ddd, J=34.2, 21.5, 8.1 Hz, 2H), 1.40 (dd, J=25.5, 12.6 Hz, 2H), 1.32-1.19 (m, 2H).

### Step 2: Synthesis of 1-cyclohexyl-4-(2-((3R,5R,8R,9R,10S,13S,14S,17S)-3-hydroxy-3,13-dimethylhexadecahydro-1H-cyclopenta[a]phenanthren-17-yl)-2-oxoethyl)-1,4-dihydro-5H-tetrazol-5-one (Compound 30)

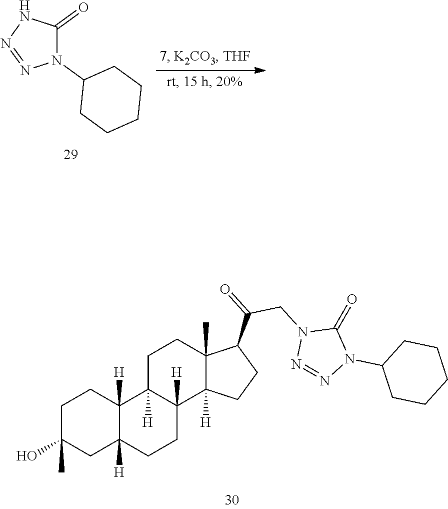

To a suspension of potassium carbonate (690 mg, 5.0 mmol, 10.0 eq) in tetrahydrofuran (10 mL), was added compound 29 (252 mg, 1.5 mmol, 3.0 eq) and compound 7 (200 mg, 0.5 mmol, 1.0 eq). The mixture was stirred at room temperature for 15 h, monitored by TLC. After completion, the mixture was poured into water (10 mL) and extracted with ethyl acetate (2×20 mL). The combined organic layers were washed with brine (10 mL), dried over anhydrous sodium sulfate, filtered and concentrated under reduced pressure. The residual mixture was purified by prep-HPLC. The desired product 30 was obtained as colorless oil, 50 mg, in 20% yield. TLC: petroleum ether/ethyl acetate (3:1); Rf: (Compound 7)=0.4; Rf: (Compound 30)=0.2; LC-MS: 467.35 (M−18)⁻; ¹H NMR (400 MHz, CDCl₃): δ 4.79-4.63 (m, 2H), 4.12-4.04 (m, 1H), 2.77 (dd, J=8.2, 2.7 Hz, 1H), 2.01 (d, J=5.9 Hz, 3H), 1.88 (d, J=9.4 Hz, 3H), 1.78 (dd, J=7.4, 2.8 Hz, 8H), 1.73-1.69 (m, 3H), 1.38 (dd, J=8.7, 4.2 Hz, 6H), 1.31 (s, 2H), 1.24-1.20 (m, 9H), 1.10-1.06 (m, 2H), 0.92 (s, 3H).

### Example 10: Synthesis of 1-(2-((3R,5R,8R,9R,10S,13S,14S,17S)-3-hydroxy-3,13-dimethylhexadecahydro-1H-cyclopenta[a]phenanthren-17-yl)-2-oxoethyl)-4-phenyl-1,4-dihydro-5H-tetrazol-5-one (Compound 33)

### Step 1: Synthesis of 1-phenyl-1,4-dihydro-5H-tetrazol-5-one (Compound 32)

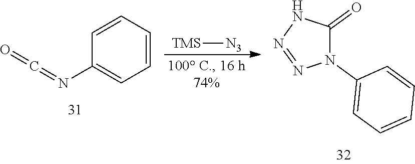 [SMILES: O=C=Nc1ccccc1 ; C[Si](C)(C)N=[N+]=[N-] ; O=c1[nH]nnn1-c1ccccc1]

A stirred mixture of isocyanatobenzene (1.0 g, 8.4 mmol, 1.0 eq) and azidotrimethylsilane (3.0 ml, 22.5 mmol, 2.7 eq) was heated to 100° C. and stirred for 16 h. The progress of the reaction mixture was monitored by TLC. After completion of the reaction, the mixture was cooled to room temperature and concentrated. The residue was diluted with ethyl acetate (10 mL) and extracted with saturated aqueous sodium bicarbonate (3×10 mL). The combined aqueous layers were added 6 M hydrochloric acid to adjust pH<3 with efficient stirring and extracted with ethyl acetate (3×20 mL). The combined organic layers were dried over anhydrous sodium sulfate and concentrated under reduced pressure to afford compound 32 (1.0 g, 74%) as a white solid. TLC: dichloromethane/methanol (10:1); Rf: (Compound 32)=0.7; Compound 31: ¹H NMR (400 MHz, CD₃OD) δ 7.51-7.32 (m, 2H), 7.29-7.15 (m, 2H), 7.03-6.93 (m, 1H); Compound 32: ¹H NMR (400 MHz, CD₃OD) δ 7.89-7.80 (m, 2H), 7.55-7.45 (m, 2H), 7.38 (ddd, J=8.0, 2.2, 1.1 Hz, 1H).

### Step 2: Synthesis of 1-(2-((3R,5R,8R,9R,10S,13S,14S,17S)-3-hydroxy-3,13-dimethylhexadecahydro-1H-cyclopenta[a]phenanthren-17-yl)-2-oxoethyl)-4-phenyl-1,4-dihydro-5H-tetrazol-5-one (Compound 33)

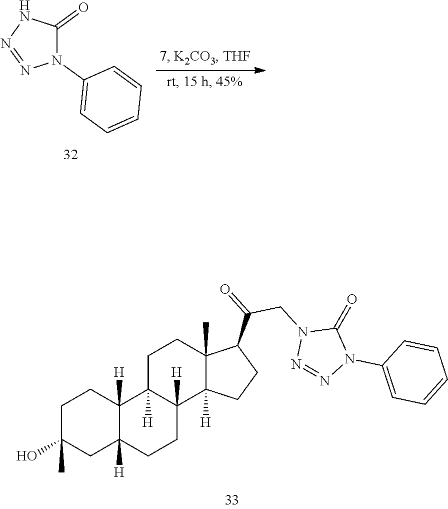

To a suspension of potassium carbonate (1.0 g, 7.25 mmol, 10.0 eq) in tetrahydrofuran (10 mL), was added compound 32 (368.1 mg, 2.27 mmol, 3.0 eq) and compound 7 (300 mg, 0.76 mmol, 1.0 eq). The mixture was stirred at room temperature for 15 h, monitored by TLC. After completion, the mixture was poured into water (10 mL) and extracted with ethyl acetate (2×20 mL). The combined organic layers were washed with brine (10 mL), dried over anhydrous sodium sulfate, filtered and concentrated under reduced pressure. The residual mixture was purified by prep-HPLC. The desired product 33 was obtained as colorless oil, 162 mg, in 45% yield. TLC: petroleum ether/ethyl acetate (3:1); Rf: (Compound 7)=0.4; Rf: (Compound 33)=0.2; LC-MS: 479 (M+1)⁺; ¹H NMR (400 MHz, CDCl₃) δ 7.93 (d, J=8.4 Hz, 2H), 7.48 (t, J=7.6 Hz, 2H), 7.35 (t, J=7.4 Hz, 1H), 4.77 (t, J=10.4 Hz, 2H), 2.62 (d, J=8.9 Hz, 1H), 2.29-2.05 (m, 1H), 1.86-1.68 (m, 3H), 1.47 (dd, J=36.4, 20.0 Hz, 10H), 1.26 (m, 8H), 1.12 (d, J=11.4 Hz, 3H), 0.70 (s, 3H).

### Example 11: Synthesis of 1-(4-fluorophenyl)-4-(2-((3R,5R,8R,9R,10S,13S,14S,17S)-3-hydroxy-3,13-dimethylhexadecahydro-1H-cyclopenta[a]phenanthren-17-yl)-2-oxoethyl)-1,4-dihydro-5H-tetrazol-5-one (Compound 36)

### Step 1: Synthesis of 1-(4-fluorophenyl)-1,4-dihydro-5H-tetrazol-5-one (Compound 35)

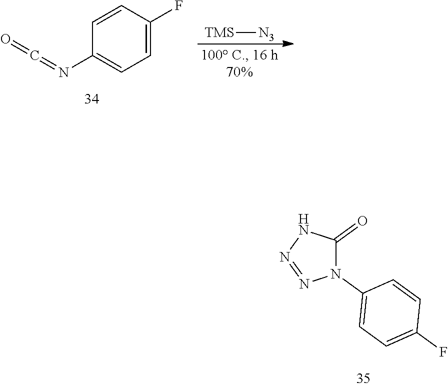 [SMILES: O=C=Nc1ccc(F)cc1 ; C[Si](C)(C)N=[N+]=[N-] ; O=c1[nH]nnn1-c1ccc(F)cc1]

A stirred mixture of compound 34 (1.0 g, 7.29 mmol, 1.0 eq) and azidotrimethylsilane (3 ml, 13.86 mmol, 1.9 eq) was heated to 100° C. and stirred for 16 h. The progress of the reaction mixture was monitored by TLC. After completion, the mixture was cooled to room temperature and concentrated. The residue was diluted with ethyl acetate (10 mL) and extracted with saturated aqueous sodium bicarbonate (3×10 mL). The combined aqueous layers were added 6 M hydrochloric acid to adjusted pH<3 with efficient stirring and extracted with ethyl acetate (3×20 mL). The combined organic layers were dried over anhydrous sodium sulfate and concentrated under reduced pressure to afford compound 35 (925 mg, 70.44%) as a white solid. TLC: dichloromethane/methanol (10:1); R_{f}: (Compound 35)=0.5; ¹H NMR (400 MHz, CDCl₃): δ 7.99-7.79 (m, 2H), 7.22-7.15 (m, 2H).

### Step 2: Synthesis of 1-(4-fluorophenyl)-4-(2-((3R,5R,8R,9R,10S,13S,14S,17S)-3-hydroxy-3,13-dimethylhexadecahydro-1H-cyclopenta[a]phenanthren-17-yl)-2-oxoethyl)-1,4-dihydro-5H-tetrazol-5-one (Compound 36)

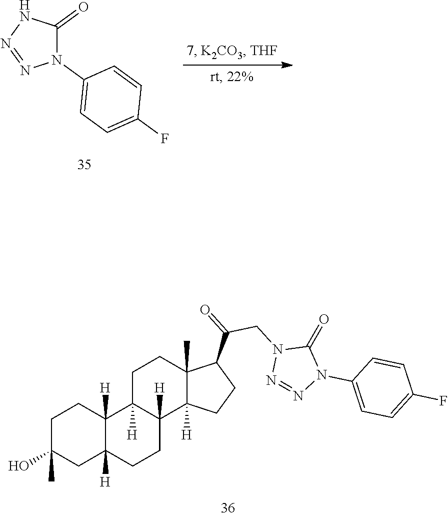

Under similar synthetic procedures for making compound 33, compound 7 (500 mg) reacted with compound 35 to produce compound 36 (136 mg, yield 22%). LC-MS: 479 (M-H₂O+1)⁺; ¹H NMR (400 MHz, CD₃OD): δ 7.94-7.86 (m, 2H), 7.33-7.24 (m, 2H), 5.02 (d, J=18.4 Hz, 2H), 2.79 (d, J=9.0 Hz, 1H), 2.27-2.07 (m, 7H), 1.99 (m, 5H), 1.97-1.66 (m, 13H), 1.57 (s, J=4.1 Hz, 3H), 1.55-0.71 (m, 4H), 0.62 (s, 3H).

### Example 12: Synthesis of 1-(3-fluorophenyl)-4-(2-((3R,5R,8R,9R,10S,13S,14S,17S)-3-hydroxy-3,13-dimethylhexadecahydro-1H-cyclopenta[a]phenanthren-17-yl)-2-oxoethyl)-1,4-dihydro-5H-tetrazol-5-one (Compound 39)

### Step 1: Synthesis of 1-(3-fluorophenyl)-1,4-dihydro-5H-tetrazol-5-one (Compound 38)

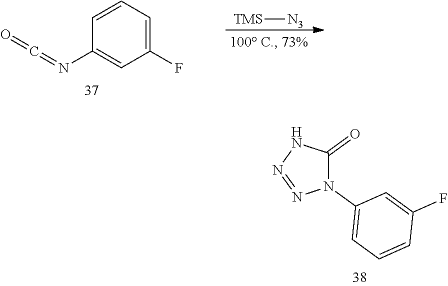 [SMILES: O=C=Nc1cccc(F)c1 ; C[Si](C)(C)N=[N+]=[N-] ; O=c1[nH]nnn1-c1cccc(F)c1]

Under similar synthetic procedures for making compound 35, 1-fluoro-3-isocyanatobenzene (1 g) reacted with azidotrimethylsilane to produce compound 38 (961 mg, yield 73%). ¹H NMR (400 MHz, CD₃OD): δ 7.82-7.67 (m, 2H), 7.61-7.45 (m, 1H), 7.24-6.98 (m, 1H).

### Step 2: Synthesis of 1-(3-fluorophenyl)-4-(2-((3R,5R,8R,9R,10S,13S,14S,17S)-3-hydroxy-3,13-dimethylhexadecahydro-1H-cyclopenta[a]phenanthren-17-yl)-2-oxoethyl)-1,4-dihydro-5H-tetrazol-5-one (Compound 39)

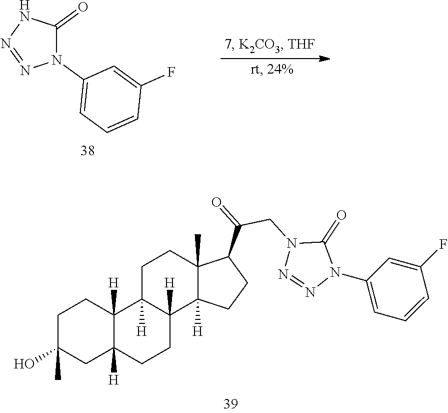

Under similar synthetic procedures for making compound 33, compound 7 (500 mg) reacted with compound 38 to produce compound 39 (148 mg, yield 24%). UC-MS: 479 (M-H₂O+1)⁺; ¹H NMR (400 MHz, CD₃OD): δ 7.81-7.69 (m, 2H), 7.62-7.46 (m, 1H), 7.15 (q, J=8.7 Hz, 1H), 5.06-4.90 (m, 2H), 2.80 (t, J=8.7 Hz, 1H), 2.18 (dd, J=24.8, 15.0 Hz, 2H), 1.95-1.62 (m, 8H), 1.51-1.26 (m, 12H), 1.22 (s, 3H), 1.16-1.07 (m, 4H), 0.69 (s, 3H).

### Example 13: Synthesis of 4-(4-(2-((3R,5R,8R,9R,10S,13S,14S,17S)-3-hydroxy-3,13-dimethylhexadecahydro-1H-cyclopenta[a]phenanthren-17-yl)-2-oxoethyl)-5-oxo-4,5-dihydro-1H-tetrazol-1-yl)benzonitrile (Compound 42)

### Step 1: Synthesis of 4-(5-oxo-4,5-dihydro-1H-tetrazol-1-yl)benzonitrile (Compound 41)

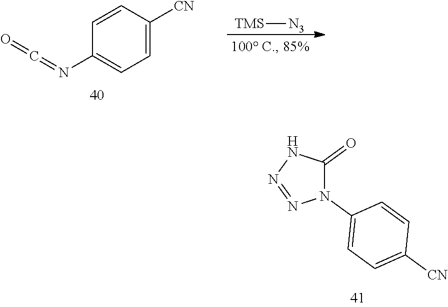 [SMILES: N#Cc1ccc(N=C=O)cc1 ; C[Si](C)(C)N=[N+]=[N-] ; N#Cc1ccc(-n2nn[nH]c2=O)cc1]

Under similar synthetic procedures for making compound 35, 4-isocyanatobenzonitrile (1 g) reacted with azidotrimethylsilane to produce compound 41 (1.1 g, yield 85%). ¹H NMR (400 MHz, CD₃OD): δ 8.27-8.02 (m, 2H), 7.98-7.71 (m, 2H).

### Step 2: Synthesis of 4-(4-(2-((3R,5R,8R,9R,10S,13S,14S,17S)-3-hydroxy-3,13-dimethylhexadecahydro-1H-cyclopenta[a]phenanthren-17-yl)-2-oxoethyl)-5-oxo-4,5-dihydro-1H-tetrazol-1-yl)benzonitrile (Compound 42)

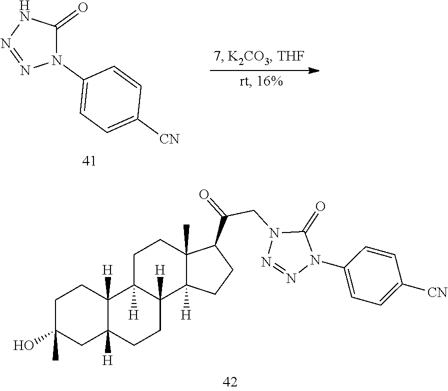

Under similar synthetic procedures for making compound 33, compound 7 (500 mg) reacted with compound 41 to produce compound 42 (102 mg, yield 16%). LC-MS: 504 (M+1)⁺; ¹H NMR (400 MHz, CD₃OD): δ 8.18 (d, J=9.2 Hz, 2H), 7.91 (d, J=9.2 Hz, 2H), 5.01-4.96 (m, 2H), 3.29 (s, 1H), 3.0 (t, J=8.7 Hz, 1H), 2.82-2.77 (m, 3H), 1.73-1.65 (m, 14H), 1.58-1.24 (m, 18H), 1.23-1.15 (m, 13H), 0.68 (s, 3H).

### Example 14: Synthesis of 3-(4-(2-((3R,5R,8R,9R,10S,13S,14S,17S)-3-hydroxy-3,13-dimethylhexadecahydro-1H-cyclopenta[a]phenanthren-17-yl)-2-oxoethyl)-5-oxo-4,5-dihydro-1H-tetrazol-1-yl)benzonitrile (Compound 45)

### Step 1: Synthesis of 3-(5-oxo-4,5-dihydro-1H-tetrazol-1-yl)benzonitrile (Compound 41)

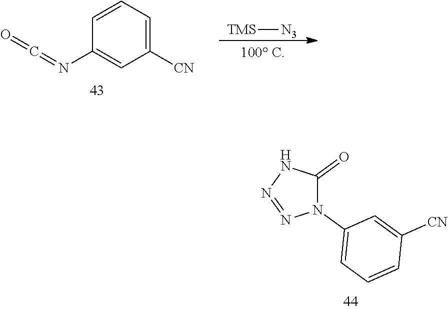 [SMILES: N#Cc1cccc(N=C=O)c1 ; C[Si](C)(C)N=[N+]=[N-] ; N#Cc1cccc(-n2nn[nH]c2=O)c1]

Under similar synthetic procedures for making compound 35, 3-isocyanatobenzonitrile (1 g) reacted with azidotrimethylsilane to produce compound 44, which was used directly to the next step without purification.

### Step 2: Synthesis of 3-(4-(2-((3R,5R,8R,9R,10S,13S,14S,17S)-3-hydroxy-3,13-dimethylhexadecahydro-1H-cyclopenta[a]phenanthren-17-yl)-2-oxoethyl)-5-oxo-4,5-dihydro-1H-tetrazol-1-yl)benzonitrile (Compound 45)

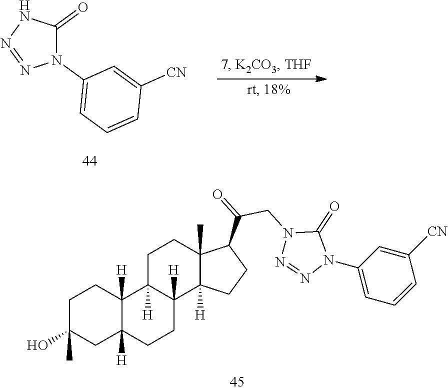

Under similar synthetic procedures for making compound 33, compound 7 (500 mg) reacted with compound 44 to produce compound 45 (115 mg, yield 18%). LC-MS: 504 (M+1)⁺; ¹H NMR (400 MHz, CD₃OD): δ 8.33 (s, 1H), 8.24 (d, J=2.4 Hz, 1H), 7.79-7.71 (m, 2H), 5.07-4.90 (m, 2H), 2.74 (t, J=8.7 Hz, 1H), 2.37-2.17 (m, 3H), 1.85-1.75 (m, 12H), 1.74-1.27 (m, 29H), 1.26-1.15 (m, 15H), 0.69 (s, 3H).

### Example 15: Synthesis of 1-(2-((3R,5R,8R,9R,10S,13S,14S,17S)-3-hydroxy-3,13-dimethylhexadecahydro-1H-cyclopenta[a]phenanthren-17-yl)-2-oxoethyl)-4-(4-methoxyphenyl)-1,4-dihydro-5H-tetrazol-5-one (Compound 48)

### Step 1: Synthesis of 1-(4-methoxyphenyl)-1,4-dihydro-5H-tetrazol-5-one (Compound 47)

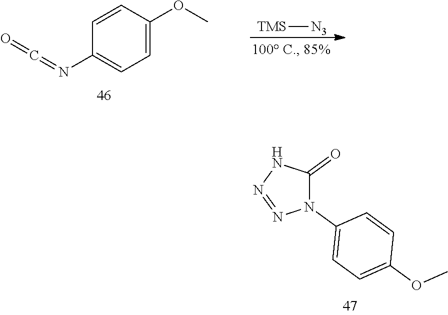

Under similar synthetic procedures for making compound 35, l-isocyanato-4-methoxybenzene (1 g) reacted with azidotrimethylsilane to produce compound 47 (1.1 g, 85%). ¹H NMR (400 MHz, CD₃OD): δ 7.77 (dd, J=9.0, 1.8 Hz, 2H), 7.10-6.90 (m, 2H), 3.84 (d, J=1.7 Hz, 3H).

### Step 2: Synthesis of 1-(2-((3R,5R,8R,9R,10S,13S,14S,17S)-3-hydroxy-3,13-dimethylhexadecahydro-1H-cyclopenta[a]phenanthren-17-yl)-2-oxoethyl)-4-(4-methoxyphenyl)-1,4-dihydro-5H-tetrazol-5-one (Compound 4

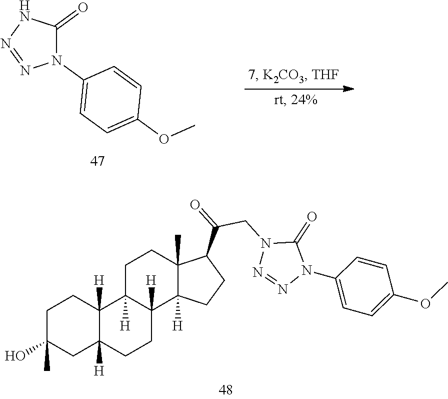

Under similar synthetic procedures for making compound 33, compound 7 (500 mg) reacted with compound 47 to produce compound 48 (148 mg, yield 24%). UC-MS: 491 (M-H₂O+1)⁺; ¹H NMR (400 MHz, CD₃OD): δ 7.83-7.57 (m, 2H), 7.17-6.84 (m, 2H), 5.15-4.88 (m, 2H), 2.79 (t, J=8.9 Hz, 1H), 2.23-2.07 (m, 1H), 1.75 (ddt, J=30.9, 28.5, 11.1 Hz, 10H), 1.54-1.28 (m, 14H), 1.22 (s, J=0.9 Hz, 3H), 1.13 (d, J=13.9 Hz, 4H), 0.68 (s, 3H).

### Example 16: Synthesis of 1-(2-((3R,5R,8R,9R,10S,13S,14S,17S)-3-hydroxy-3,13-dimethylhexadecahydro-1H-cyclopenta[a]phenanthren-17-yl)-2-oxoethyl)-4-(3-methoxyphenyl)-1,4-dihydro-5H-tetrazol-5-one (Compound 51)

### Step 1: Synthesis of 1-(3-methoxyphenyl)-1,4-dihydro-5H-tetrazol-5-one (Compound 50)

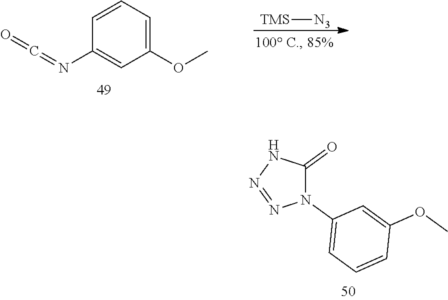

Under similar synthetic procedures for making compound 35, l-isocyanato-3-methoxybenzene (1 g) reacted with azidotrimethylsilane to produce compound 50 (1.1 g, 85%). ¹H NMR (400 MHz, CD₃OD): δ 7.54-7.24 (m, 3H), 6.95 (ddd, J=7.9, 2.5, 1.4 Hz, 1H), 4.83 (s, 3H).

### Step 2: Synthesis of 1-(2-((3R,5R,8R,9R,10S,13S,14S,17S)-3-hydroxy-3,13-dimethylhexadecahydro-1H-cyclopenta[a]phenanthren-17-yl)-2-oxoethyl)-4-(3-methoxyphenyl)-1,4-dihydro-5H-tetrazol-5-one (Compound 51)

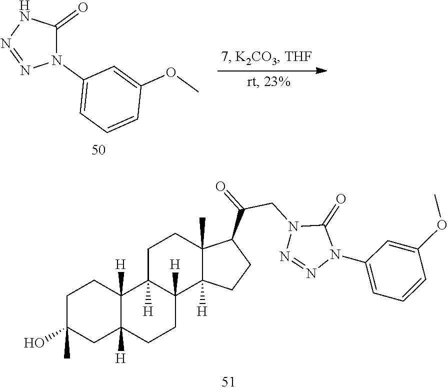

Under similar synthetic procedures for making compound 33, compound 7 (500 mg) reacted with compound 50 to produce compound 51 (148 mg, yield 23%). UC-MS: 510.6 (M+1)⁺; ¹H NMR (400 MHz, CD₃OD): δ 7.48-7.39 (m, 3H), 7.00-6.97 (m, 1H), 5.16 (d, J=18.4 Hz, 1H), 4.84 (d, J=18.8 Hz, 1H), 4.22 (s, 1H), 3.78 (s, 3H), 2.77-2.72 (m, 1H), 2.03-2.00 (m, 2H), 1.69-0.99 (m, 32H), 0.55 (s, 3H).

### Example 17: Synthesis of 1-(2-((3R,5R,8R,9R,10S,13S,14S,17S)-3-hydroxy-3,13-dimethylhexadecahydro-1H-cyclopenta[a]phenanthren-17-yl)-2-oxoethyl)-4-(pyridin-4-yl)-1,4-dihydro-5H-tetrazol-5-one (Compound 55)

### Step 1: Synthesis of 1-(pyridin-4-yl)-1,4-dihydro-5H-tetrazol-5-one (Compound 54)

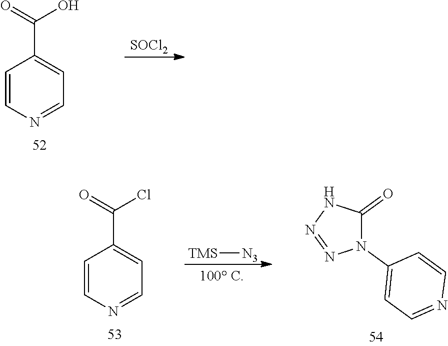

A mixture of compound 52 (2.0 g, 16.25 mmol, 1.0 eq) and SOCl₂ (30 ml) was heated to 85° C. and stirred for 4 h. The progress of the reaction mixture was monitored by LC-MS. After completion, the mixture was cooled to room temperature and concentrated in vacuum to give crude compound 53 (3.2 g), which was used in the next step without further purification.

A mixture of compound 53 (3.2 g, crude) and azidotrimethylsilane (10 ml, 75 mmol, 2.2 eq) was heated to 100° C. and stirred for 16 h. The progress of the reaction mixture was monitored by LC-MS. After completion, the mixture was cooled to room temperature and concentrated in vacuum to give crude compound 54 (1.2 g), which was used in the next step without further purification.

### Step 2: Synthesis of 1-(2-((3R,5R,8R,9R,10S,13S,14S,17S)-3-hydroxy-3,13-dimethylhexadecahydro-1H-cyclopenta[a]phenanthren-17-yl)-2-oxoethyl)-4-(pyridin-4-yl)-1,4-dihydro-5H-tetrazol-5-one (Compound 55)

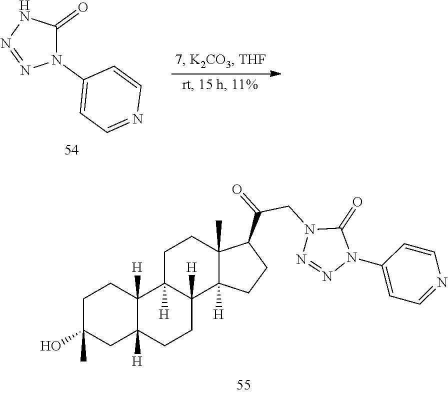

To a suspension of potassium carbonate (2.0 g, 15.2 mmol, 10.0 eq) in tetrahydrofuran (20 mL), was added compound 54 (1.2 g, crude, 7.36 mmol, 5.8 eq) and compound 7 (500.0 mg, 1.26 mmol, 1.0 eq). The mixture was stirred at room temperature for 15 h, monitored by TLC. After completion, the mixture was poured into water (10 mL) and extracted with ethyl acetate (2×20 mL). The combined organic layers were washed with brine (10 mL), dried over anhydrous sodium sulfate, filtered and concentrated under reduced pressure. The residual mixture was purified by prep-HPLC and compound 55 was obtained as a white solid (63.8 mg, 11%). TLC: petroleum ether/ethyl acetate (1:1); Rf: (Compound 7)=0.7; Rf: (Compound 55)=0.1; LC-MS: 480 (M+1)⁺; ¹H NMR (400 MHz, CD₃OD): δ 8.84 (d, J=6.1 Hz, 2H), 8.52-8.27 (m, 2H), 5.25-4.97 (m, 2H), 2.80 (d, J=8.9 Hz, 1H), 2.28-1.99 (m, 3H), 2.00-1.64 (m, 8H), 1.38 (d, J=12.8 Hz, 9H), 1.22 (s, 3H), 1.16-1.10 (m, 4H), 0.69 (s, 3H).

### Example 18: Synthesis of 1-(2-((3R,5R,8R,9R,10S,13S,14S,17S)-3-hydroxy-3,13-dimethylhexadecahydro-1H-cyclopenta[a]phenanthren-17-yl)-2-oxoethyl)-4-(pyridin-3-yl)-1,4-dihydro-5H-tetrazol-5-one (Compound 58)

### Step 1: Synthesis of 1-(pyridin-3-yl)-1,4-dihydro-5H-tetrazol-5-one (Compound 57)

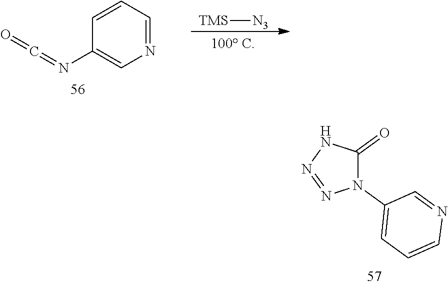 [SMILES: O=C=Nc1cccnc1 ; C[Si](C)(C)N=[N+]=[N-] ; O=c1[nH]nnn1-c1cccnc1]

Under similar synthetic procedures for making compound 35, 3-isocyanatopyridine (1 g) reacted with azidotrimethylsilane to produce compound 57, which was used directly to the next step without purification.

### Step 2: Synthesis of 1-(2-((3R,5R,8R,9R,10S,13S,14S,17S)-3-hydroxy-3,13-dimethylhexadecahydro-1H-cyclopenta[a]phenanthren-17-yl)-2-oxoethyl)-4-(pyridin-3-yl)-1,4-dihydro-5H-tetrazol-5-one (Compound 58)

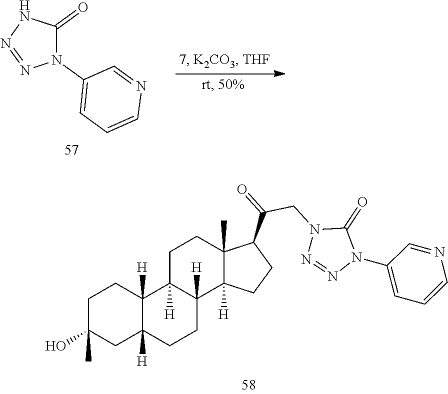

Under similar synthetic procedures for making compound 33, compound 7 (500 mg) reacted with compound 57 to produce compound 58 (304 mg, yield 50%). UC-MS: 480 (M+1)⁺; ¹H NMR (400 MHz, CD₃OD): δ 9.23 (s, 1H), 8.83-8.33 (m, 2H), 7.73 (dd, J=8.4, 4.9 Hz, 1H), 5.24-4.90 (m, 2H), 2.81 (t, J=8.7 Hz, 1H), 2.27-2.09 (m, 3H), 1.95-1.69 (m, 10H), 1.52-1.32 (m, 13H), 1.28 (s, 2H), 1.22 (s, 3H), 1.16-1.11 (m, 2H), 0.69 (s, 3H).

### Example 19: Synthesis of 1-(2-((3R,5R,8R,9R,10S,13S,14S,17S)-3-hydroxy-3,13-dimethylhexadecahydro-1H-cyclopenta[a]phenanthren-17-yl)-2-oxoethyl)-1,4-dihydro-5H-tetrazol-5-one (Compound 60)

### Step 1: Synthesis of 1-(2-((3R,5R,8R,9R,10S,13S,14S,17S)-3-hydroxy-3,13-dimethylhexadecahydro-1H-cyclopenta[a]phenanthren-17-yl)-2-oxoethyl)-4-(4-methoxybenzyl)-1,4-dihydro-5H-tetrazol-5-one (Compound 59)

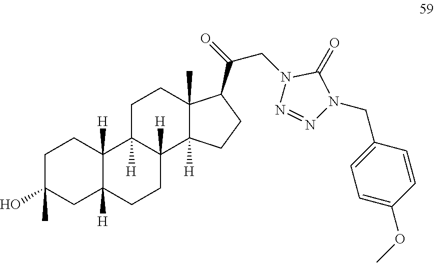

Under similar synthetic procedures for making compound 55, compound 59 was generated from 2-(4-methoxyphenyl)acetic acid.

### Step 2: Synthesis of 1-(2-((3R,5R,8R,9R,10S,13S,14S,17S)-3-hydroxy-3,13-dimethylhexadecahydro-1H-cyclopenta[a]phenanthren-17-yl)-2-oxoethyl)-1,4-dihydro-5H-tetrazol-5-one (Compound 60)

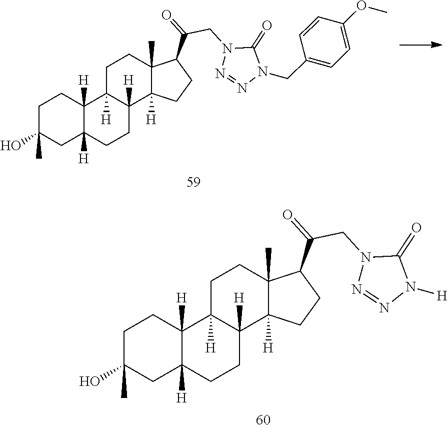

A solution of compound 59 (0.38 mmol, 1.0 eq) and eerie ammonium nitrate (3.8 mmol, 10.0 eq) in acetonitrile (5 mL) and water (2 mL) was stirred at rt for 3 h. The reaction mixture was diluted with EtOAc, washed with water, dried (MgSO₄), and concentrated. The crude product was purified using prep HPLC (C₁₈ column) to afford final compound 60 as a white solid (60 mg). ¹H NMR (400 MHz, CD₃OD) δ 4.90 (s, 2H), 2.80-2.76 (m, 1H), 2.21-2.08 (m, 2H), 1.82-1.74 (m, 3H), 1.72-1.68 (m, 3H), 1.57-1.45 (m, 3H), 1.41-1.36 (m, 7H), 1.28-1.21 (m, 7H), 1.15-1.00 (m, 3H), 0.66 (s, 3H).

### Example 20: Synthesis of 2-(4-(2-((3R,5R,8R,9R,10S,13S,14S,17S)-3-hydroxy-3,13-dimethylhexadecahydro-1H-cyclopenta[a]phenanthren-17-yl)-2-oxoethyl)-5-oxo-4,5-dihydro-1H-tetrazol-1-yl)acetonitrile (Compound 61)

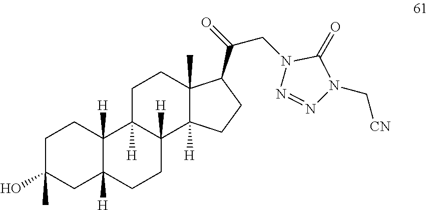

Under similar synthetic procedures for making compound 55, compound 61 (54.1 mg) was generated from 2-cyanoacetic acid. ¹H NMR (400 MHz, CDCl₃) δ 4.87 (s, 2H), 4.73 (s, 2H), 2.62-2.57 (m, 1H), 2.19-2.16 (m, 1H), 2.05-2.00 (m, 1H), 1.82-1.74 (m, 3H), 1.72-1.57 (m, 6H), 1.47-1.35 (m, 7H), 1.25-1.11 (m, 7H), 1.12-1.07 (m, 3H), 0.66 (s, 3H).

### Example 21: Synthesis of 3-(4-(2-((3R,5R,8R,9R,10S,13S,14S,17S)-3-hydroxy-3,13-dimethylhexadecahydro-1H-cyclopenta[a]phenanthren-17-yl)-2-oxoethyl)-5-oxo-4,5-dihydro-1H-tetrazol-1-yl)propanenitrile (Compound 62)

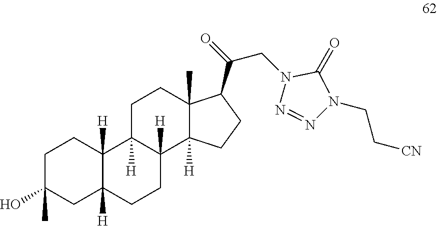

Under similar synthetic procedures for making compound 55, compound 62 (110.9 mg) was generated from 3-cyanopropanoic acid. ¹H NMR (400 MHz, CDCl₃) δ 4.67 (s, 2H), 4.26 (d, J=7.2 Hz, 2H), 2.92 (d, J=7.2 Hz, 2H), 2.59-2.57 (m, 1H), 2.21-2.16 (m, 1H), 2.05-2.00 (m, 1H), 1.82-1.76 (m, 3H), 1.72-1.64 (m, 3H), 1.45 (s, 3H), 1.45-1.33 (m, 7H), 1.14-1.06 (m, 7H), 1.12-1.07 (m, 3H), 0.66 (s, 3H).

### Example 22: Synthesis of 1-(2-((3R,5R,8R,9R,10S,13S,14S,17S)-3-hydroxy-3,13-dimethylhexadecahydro-1H-cyclopenta[a]phenanthren-17-yl)-2-oxoethyl)-4-(trifluoromethyl)-1,4-dihydro-5H-tetrazol-5-one (Compound 63)

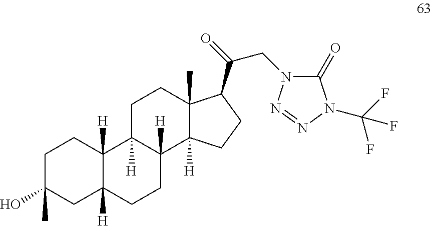

Under similar synthetic procedures for making compound 55, compound 63 (94 mg) was generated from 2,2,2-trifluoroacetic acid. ¹H NMR (400 MHz, CDCl₃) δ 3.86 (s, 2H), 2.51-2.46 (m, 1H), 2.19-2.16 (m, 1H), 1.88-1.77 (m, 4H), 1.73-1.67 (m, 3H), 1.53 (s, 3H), 1.47-1.33 (m, 7H), 1.29-1.20 (s, 7H), 1.10-1.04 (m, 3H), 0.63 (s, 3H).

### Example 23: Synthesis of 1-(2-((3R,5R,8R,9R,10S,13S,14S,17S)-3-hydroxy-3,13-dimethylhexadecahydro-1H-cyclopenta[a]phenanthren-17-yl)-2-oxoethyl)-4-(oxazol-5-yl)-1,4-dihydro-5H-tetrazol-5-one (Compound 64)

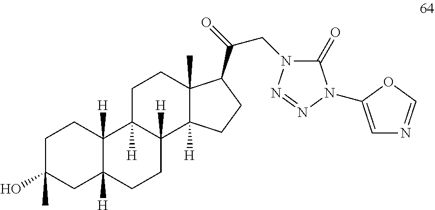

Under similar synthetic procedures for making compound 55, compound 64 (30 mg) was generated from oxazole-5-carboxylic acid. ¹H NMR (400 MHz, CDCl₃) δ 8.05 (s, 1H), 7.86 (s, 1H), 4.96-4.92 (m, 1H), 4.77-4.73 (m, 1H), 2.51-2.46 (m, 1H), 2.19-2.16 (m, 1H), 2.08-2.02 (m, 1H), 1.88-1.77 (m, 3H), 1.73-1.62 (m, 6H), 1.47-1.33 (m, 7H), 1.29-1.20 (s, 7H), 1.10-1.04 (m, 3H), 0.63 (s, 3H).

### Example 24: Synthesis of 1-(2-((3R,5R,8R,9R,10S,13S,14S,17S)-3-hydroxy-3,13-dimethylhexadecahydro-1H-cyclopenta[a]phenanthren-17-yl)-2-oxoethyl)-4-(oxazol-4-yl)-1,4-dihydro-5H-tetrazol-5-one (Compound 65)

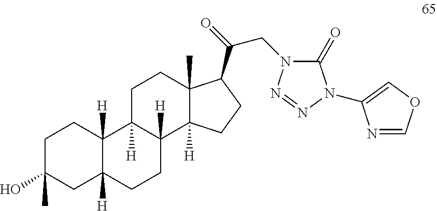

Under similar synthetic procedures for making compound 55, compound 65 (36.6 mg) was generated from oxazole-4-carboxylic acid. ¹H NMR (400 MHz, CDCl₃) δ 8.16 (s, 1H), 7.94 (s, 1H), 4.77 (s, 2H), 2.63-2.58 (m, 1H), 2.24-2.16 (m, 1H), 2.10-2.04 (m, 1H), 1.82-1.61 (m, 8H), 1.64-1.58 (m, 2H), 1.46-1.36 (m, 7H), 1.32-1.22 (m, 7H), 1.14-1.06 (m, 3H), 0.67 (s, 3H).

### Example 25: Synthesis of 1-(2-((3R,5R,8R,9R,10S,13S,14S,17S)-3-hydroxy-3,13-dimethylhexadecahydro-1H-cyclopenta[a]phenanthren-17-yl)-2-oxoethyl)-4-(1H-pyrazol-5-yl)-1,4-dihydro-5H-tetrazol-5-one (Compound 67)

### Step 1: Synthesis of tert-butyl 5-(4-(2-((3R,5R,8R,9R,10S,13S,14S,17S)-3-hydroxy-3,13-dimethylhexadecahydro-1H-cyclopenta[a]phenanthren-17-yl)-2-oxoethyl)-5-oxo-4,5-dihydro-1H-tetrazol-1-yl)-1H-pyrazole-1-carboxylate (Compound 66)

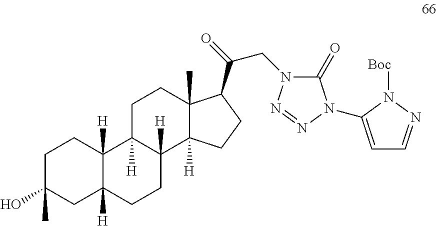

Under similar synthetic procedures for making compound 55, compound 66 was generated from 1-(tert-butoxycarbonyl)-1H-pyrazole-5-carboxylic acid.

### Step 2: Synthesis of 1-(2-((3R,5R,8R,9R,10S,13S,14S,17S)-3-hydroxy-3,13-dimethylhexadecahydro-1H-cyclopenta[a]phenanthren-17-yl)-2-oxoethyl)-4-(1H-pyrazol-5-yl)-1,4-dihydro-5H-tetrazol-5-one (Compound 67)

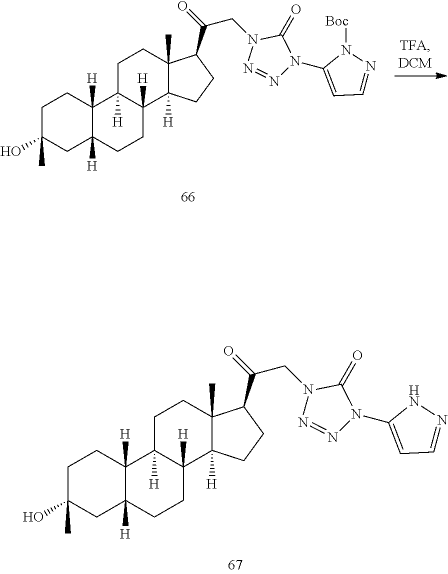

To a solution of compound 66 (0.38 mmol, 1.0 eq) in CH₂Cl₂ (5 mL), was added TFA (0.76 mmol, 2.0 eq), and the reaction mixture was stirred at rt for 1 h. The mixture was washed with NaHCO₃ (aq), dried (MgSO₄), and concentrated. The crude product was purified using prep HPLC (C18 column) to afford final compound 67 as a white solid (45.9 mg). ¹H NMR (400 MHz, CDCl₃) δ 7.71 (s, 1H), 6.67 (s, 1H), 4.75 (s, 2H), 2.51-2.46 (m, 1H), 2.19-2.16 (m, 1H), 2.08-2.02 (m, 1H), 1.88-1.77 (m, 3H), 1.73-1.62 (m, 6H), 1.47-1.33 (m, 7H), 1.29-1.20 (s, 7H), 1.10-1.04 (m, 3H), 0.63 (s, 3H).

### Example 26: Synthesis of 1-(2-((3R,5R,8R,9R,10S,13S,14S,17S)-3-hydroxy-3,13-dimethylhexadecahydro-1H-cyclopenta[a]phenanthren-17-yl)-2-oxoethyl)-4-(1-methyl-1H-pyrazol-5-yl)-1,4-dihydro-5H-tetrazol-5-one (Compound 68)

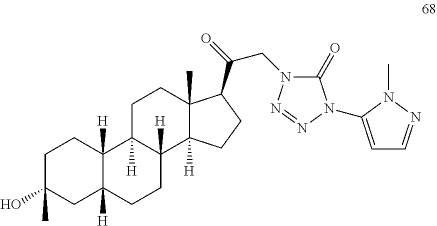

Under similar synthetic procedures for making compound 55, compound 68 (105.2 mg) was generated from 1-methyl-1H-pyrazole-5-carboxylic acid. ¹H NMR (400 MHz, CDCl₃) δ 7.56 (d, J=2.1 Hz, 1H), 6.51 (d, J=2.1 Hz, 1H), 4.79 (s, 2H), 3.86 (s, 3H), 2.61 (d, J=9.0 Hz, 1H), 2.22-2.19 (m, 1H), 2.09-2.06 (m, 1H), 1.95-1.92 (m, 2H), 1.85-1.77 (m, 7H), 1.48-1.38 (m, 7H), 1.30-1.25 (m, 7H), 1.15-1.07 (m, 3H), 0.68 (s, 3H).

### Example 27: Synthesis of 1-(2-((3R,5R,8R,9R,10S,13S,14S,17S)-3-hydroxy-3,13-dimethylhexadecahydro-1H-cyclopenta[a]phenanthren-17-yl)-2-oxoethyl)-4-(1-methyl-1H-pyrazol-3-yl)-1,4-dihydro-5H-tetrazol-5-one (Compound 69)

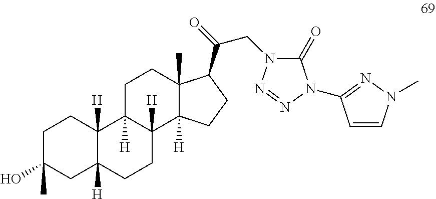

Under similar synthetic procedures for making compound 55, compound 69 (96.5 mg) was generated from 1-methyl-1H-pyrazole-3-carboxylic acid. ¹H NMR (400 MHz, CDCl3) δ 7.40 (d, J=2.3 Hz, 1H), 6.59 (d, J=2.3 Hz, 1H), 4.76 (s, 2H), 3.93 (s, 3H), 2.61 (t, J=8.9 Hz, 1H), 2.21-2.16 (m, 1H), 2.11-2.08 (m, 1H), 1.84-1.77 (m, 3H), 1.74-1.67 (m, 6H), 1.43-1.32 (m, 7H), 1.29-1.20 (m, 7H), 1.13-1.06 (m, 3H), 0.67 (s, 3H).

### Example 28: Synthesis of 1-(2-((3R,5R,8R,9R,10S,13S,14S,17S)-3-hydroxy-3,13-dimethylhexadecahydro-1H-cyclopenta[a]phenanthren-17-yl)-2-oxoethyl)-4-(1H-pyrazol-4-yl)-1,4-dihydro-5H-tetrazol-5-one (Compound 70)

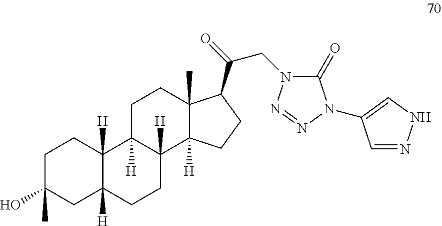

Under similar synthetic procedures for making compound 67, compound 70 (87.1 mg) was generated from 1-(tert-butoxycarbonyl)-1H-pyrazole-4-carboxylic acid. ¹H NMR (400 MHz, CDCl₃) δ 8.16 (s, 2H), 4.79 (s, 2H), 2.65-2.63 (m, 1H), 2.21-2.18 (m, 1H), 2.11-2.08 (m, 1H), 1.84-1.77 (m, 4H), 1.74-1.67 (m, 5H), 1.43-1.32 (m, 7H), 1.29-1.20 (m, 7H), 1.13-1.06 (m, 3H), 0.69 (s, 3H).

### Example 29: Synthesis of 1-(2-((3R,5R,8R,9R,10S,13S,14S,17S)-3-hydroxy-3,13-dimethylhexadecahydro-1H-cyclopenta[a]phenanthren-17-yl)-2-oxoethyl)-4-(1-methyl-1H-pyrazol-4-yl)-1,4-dihydro-5H-tetrazol-5-one (Compound 71)

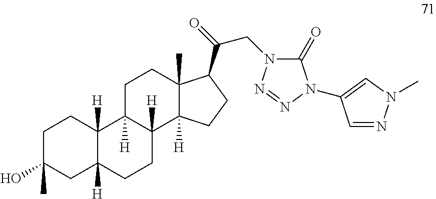

Under similar synthetic procedures for making compound 55, compound 71 (128.7 mg) was generated from 1-methyl-1H-pyrazole-4-carboxylic acid. ¹H NMR (400 MHz, CDCl₃) δ 7.90 (s, 1H), 7.86 (s, 1H), 4.76 (s, 2H), 3.93 (s, 3H), 2.63-2.59 (m, 1H), 2.24-2.16 (m, 1H), 2.10-2.04 (m, 2H), 1.85-1.77 (m, 4H), 1.75-1.70 (m, 3H), 1.65-1.58 (m, 1H), 1.47-1.38 (m, 7H), 1.32-1.22 (m, 7H), 1.14-1.06 (m, 3H), 0.67 (s, 3H).

### Example 30: Patch Clamp Electrophysiology Assay of Recombinant α1β2γ2 GABA_{A} Receptors

Cellular electrophysiology is used to measure the pharmacological properties of GABA_{A} receptor modulators in heterologous cell systems. Each compound is tested for its ability to affect GABA mediated currents at a submaximal agonist dose (GABA EC₂₀=2 μM). HEK293T cells are stably transfected with the α1β2γ2 subunits of the GABA_{A} receptor. Flasks or dishes of HEK293 GABA_{A} cells were maintained in the medium (DMEM, 11% (v/v) heat inactivated FBS, 100 or 200 μg/ml G418, 40 μg/ml Hygromycin B, 80 μg/ml Zeocin) and incubated at 37° C. in a humidified incubator with 5% CO₂. 12 to 24 hours prior to electrophysiological recordings, the cells were plated on glass cover slips placed in culture dishes and maintained under the same incubation and media conditions. The number of cells being plated on the cover slip should reach a confluence rate at which majority of the cells are single.

For the electrophysiological recordings in the study the following external and internal solutions were used (Table 1):

TABLE 1
Composition of external and internal solutions used in
GABA_{A} PAM electrophysiology assay

| | External | Internal |
|---|---|---|
| Reagents | Solution (mM) | Solution (mM) |
| NaCl | 140 | / |
| CsCl | / | 110 |
| KCl | 4.7 | / |
| HEPES | 10 | 10 |
| CaCl₂ | 2 | 1 |
| Glucose | 11 | / |
| MgCl₂ | 1 | 1 |
| EGTA | / | 10 |
| ATP-Na₂ | / | 2 |
| TEA-Cl | / | 25 |
| pH | ~7.4 (adjusted with | ~7.2 (adjusted with |
| | NaOH) | NaOH) |
| Osmolarity | ~300 mOsm | ~305 mOsm |

HEKA EPC 10 USB patch clamp amplifier (from HEKA Elektronik, Germany) was used in the whole cell recording. A cover slip with plenty of single HEK293T-GABA_{A} cells on the surface was removed and placed into a continuously perfused (approximately 1-2 ml/minute) recording chamber mounted on an inverted microscope. GABA_{A} Cl⁻ current was recorded from single cell using standard whole cell recording techniques. 2 μM GABA was used to evoke GABA_{A} Cl⁻ current (2 μM of GABA was the EC₁₀-EC₂₀ value of agonist GABA obtained internally on the stable GABA_{A} cells used in this study). After achieving break-in (whole-cell) configuration, the cell was voltage clamped at a holding potential of −80 mV. Only stable cells meeting the recording parameters (membrane resistance Rm>500 MΩ, access resistance (Ra)<15 MΩ, leak current<100 pA in 90% of the recording time) were used. 2 μM GABA was applied to the cell to induce stable Cl⁻ current first as control for about 2 seconds, test compounds (i.e., compounds of formula (AI), (I), (AII), and (II), or any subgenera thereof) were followed to be applied to the cells for 3 min, then 2 μM GABA was applied to observe the potentiation effect on the GABA_{A} Cl⁻ current. Different doses of test compound from low to high were applied to the same cell when the cell was stable. A cell was considered stable when current size evoked by 2 μM GABA applied to the cell from time to time was similar. To minimize the possible desensitation of GABA_{A} Cl⁻ current, duration between applications of test articles was set to 180 seconds.

Test compounds were dissolved in DMSO to form stock solutions (10 mM). Test compounds were diluted to 0.003, 0.01, 0.03, 0.1, 0.3, 1, 3 and 10 μM in bath solution. AU concentrations of test articles were tested on each cell. Data were analyzed using software provided by HEKA, Microsoft Excel and Graphpad Prism (Table 2).

Test compounds showed very potent activity in the patch clamp electrophysiology assay on recombinant α1β2γ2 GABA_{A} receptors.

TABLE 2
Electrophysiological evaluation of the exemplary
compounds at α1β2γ2 GABA_{A} receptor

| Test Compd. | | |
|---|---|---|
| Example # | EC₅₀ (nM) | Emax (%) |
| Example 2 | 110 | 1105 |
| Example 3 | 94 | 1175 |
| Example 4 | 140 | 1152 |
| Example 5 | 150 | 1642 |
| Example 6 | 660 | 1100 |
| Example 7 | 99 | 1170 |
| Example 8 | 95 | 1824 |
| Example 9 | 1380 | 2532 |
| Example 10 | 27 | 1605 |
| Example 11 | 44 | 795 |
| Example 12 | 9.3 | 1136 |
| Example 13 | 13 | 1276 |
| Example 14 | 7.0 | 974 |
| Example 15 | 5.0 | 1175 |
| Example 16 | 5.4 | 578 |
| Example 17 | 38 | 1541 |
| Example 18 | 39 | 757 |
| Example 19 | 24000 | 1426 |
| Example 20 | 560 | 1232 |
| Example 21 | 290 | 1277 |
| Example 22 | 14 | 1261 |
| Example 23 | 96 | 1492 |
| Example 24 | 64 | 698 |
| Example 25 | 130 | 1399 |
| Example 26 | 64 | 1117 |
| Example 27 | 120 | 1479 |
| Example 28 | 140 | 1551 |
| Example 29 | 280 | 1581 |

### Examples 31: Pharmacokinetic Properties of GABA Modulators

Compounds of the present invention (test compounds) demonstrate good pharmacokinetic properties. Test compounds were dosed to male CD1 mice at 5 mg/kg by oral gavage (p.o.) or 1 mg/kg by intravenous injection (i.v.). Blood samples were taken at 0.083, 0.25, 0.5, 1.0, 3.0, 6.0 and 24 hours post i.v. dosing and at 0.25, 0.5, 1.0, 3.0, 6.0 and 24 hours post p.o. dosing. The plasma concentration of compounds were determined by HPLC. Pharmacokinetic (PK) properties are shown in Table 3.

Test compounds showed high oral bioavailability in the mouse PK study.

TABLE 3
Mouse PK properties of GABA modulators

| | | | | AUCₗₐₛₜ | AUCₗₐₛₜ | Cₘₐₓ | Tₘₐₓ | |
|---|---|---|---|---|---|---|---|---|
| Test | CL (L/ | Vss | T_{1/2} | (hr*ng/ | (hr*ng/ | (ng/mL) | (hr) | F |
| Compd. | hr/kg) | (L/kg) | (hr) i.v. | mL) i.v. | mL) p.o. | p.o. | p.o. | (%) |
| Example 2 | 0.64 | 1.16 | 1.46 | 1476 | 4896 | 1377 | 1.00 | 66.3 |
| Example 3 | 1.46 | 0.93 | 0.57 | 673 | 1332 | 973 | 0.50 | 40.5 |
| Example 4 | 1.49 | 0.88 | 0.54 | 659 | 782 | 808 | 0.25 | 25.8 |
| Example 5 | 1.47 | 8.30 | 7.83 | 634 | 633 | 276 | 0.50 | 20.5 |
| Example 7 | 4.12 | 1.69 | 0.60 | 239 | 326 | 186 | 0.50 | 27.6 |
| Example 8 | 8.41 | 2.38 | 0.73 | 117 | 26 | 22 | 0.66 | 4.48 |
| Example 10 | 0.54 | 2.16 | 4.22 | 1844 | 5507 | 491 | 3.00 | 60.5 |
| Example 11 | 0.81 | 2.42 | 3.90 | 1231 | 3201 | 274 | 1.00 | 52.3 |
| Example 12 | 0.50 | 2.86 | 6.22 | 1885 | 4137 | 388 | 1.00 | 42.4 |
| Example 13 | 0.34 | 2.94 | 8.03 | 2644 | 5760 | 479 | 1.00 | 41.5 |
| Example 14 | 1.23 | 1.92 | 1.44 | 771 | 1116 | 250 | 1.00 | 28.9 |
| Example 15 | 1.80 | 1.76 | 1.05 | 542 | 292 | 106 | 1.00 | 10.8 |
| Example 16 | 1.98 | 1.54 | 0.90 | 500 | 330 | 109 | 0.50 | 15.1 |
| Example 17 | 2.61 | 2.12 | 0.84 | 381 | 1926 | 227 | 3.00 | 101 |
| Example 18 | 3.39 | 1.33 | 0.29 | 270 | 692 | 297 | 1.00 | 50.1 |
| Example 22 | 7.77 | 2.59 | 0.30 | 118 | 203 | 213 | 0.50 | 34.2 |
| Example 24 | 0.93 | 1.87 | 1.57 | 998 | 9179 | 775 | 3.00 | 172 |
| Example 25 | 0.39 | 1.88 | 4.63 | 2477 | 12764 | 1187 | 3.00 | 106 |
| Example 26 | 0.94 | 1.75 | 1.39 | 1013 | 3906 | 827 | 1.00 | 77.1 |
| Example 27 | 0.87 | 1.14 | 1.09 | 1113 | 8772 | 1066 | 1.00 | 153 |
| Example 28 | 0.43 | 2.90 | 6.12 | 2177 | 8415 | 563 | 6.00 | 77.3 |

### Examples 32: Acute PTZ Method

The anticonvulsant effect of test compounds were assessed in the pentylenetetazol-induced seizure assay in mice similar to methods described in Giardina & Gasior (2009) Curr Protoc Pharmacol., Chapter 5. Male CD-1 mice were housed in groups of five under controlled conditions (temperature of 22±2° C. and 12:12 light-dark cycle, lights on at 8:00 am) and water and food were available ad libitum. The mice were housed for 1 week prior to behavioral testing, at which time they weighed 25-35 g. Pentylenetetrazol (PTZ, Sigma) was dissolved in sterile 0.9% saline at a concentration of 12 mg/mL concentration for subcutaneous administration. Test compounds were formulated and administered via oral gavage at 60 minutes prior to PTZ injection. AU solutions were made fresh and were given in a volume of 10 ml/kg body weight.

Mice were acclimated to the test room for at least 30 min before compound administration. Mice were randomized into at least four test groups (vehicle and at least three doses of the test compound) with 10 mice per group. After compound administration, mice were observed for qualitative assessment of sedation for 60 minutes. Following the drug pretreatment time, the mice were injected s.c. with PTZ (120 mg/kg). Immediately following the PTZ injection, mice were individually placed into observation chambers (25×15×15 cm) and a three-channel timer was started. Each mouse was continuously observed for 30 min and the following behaviors were recorded by observers blinded to the treatments: 1) latency to clonic convulsions that persist for 3 sec and followed by an absence of righting reflex 2) latency to tonic convulsions, characterized by the rigid extension of all four limbs that exceeded a 90 degree angle with the body 3) latency to death 4) number of clonic and tonic convulsions. Data are presented as mean±S.E.M and one-way analysis of variance with Dunnetfs or Bonferroni's post-hoc test was used to detect significant differences in latency and number between the vehicle and dose group, p values<0.05 were regarded as statistically significant.

TABLE 4
Effective anticonvulsant for clonic and tonic seizures in PTZ-treated mice.

| Test | Clonic | Tonic | Clonic | Tonic | Onset | |
|---|---|---|---|---|---|---|
| Compd. | Seizure | Seizure | Seizure | Seizure | of death | |
| Example # | Latency (sec) | Latency (sec) | Number | Number | (sec) | Mortality |
| (dose) | (Mean) | (Mean) | (Mean) | (Mean) | (Mean) | (%) |
| Ex. 2 | 1085.4 + 184.6 | 1800 + 0.0 | 0.7 + 0.2 | 0.0 + 0.0 | 1800 + 0.0 | 0.0 |
| (3 mg/kg) | | | | | | |
| Ex. 10 | 1485 + 210.2 | 1800 + 0.0 | 0.3 + 0.2 | 0.0 + 0.0 | 1800 + 0.0 | 0.0 |
| (3 mg/kg) | | | | | | |
| Ex. 11 | 717.4 + 238.5 | 1800 + 0.0 | 0.8 + 0.2 | 0.0 + 0.0 | 1800 + 0.0 | 0.0 |
| (3 mg/kg) | | | | | | |
| Ex. 13 | 1012 + 263.6 | 1800 + 0.0 | 0.7 + 0.3 | 0.0 + 0.0 | 1800 + 0.0 | 0.0 |
| (3 mg/kg) | | | | | | |
| Ex. 17 | 1441 + 184.6 | 1800 + 0.0 | 0.3 + 0.2 | 0.0 + 0.0 | 1800 + 0.0 | 0.0 |
| (3 mg/kg) | | | | | | |
| Ex. 26 | 1547.8 + 139.2 | 1800 + 0.0 | 0.3 + 0.2 | 0.0 + 0.0 | 1800 + 0.0 | 0.0 |
| (3 mg/kg) | | | | | | |
| Ex. 28 | 573.1 + 124.9 | 1586.3 + 109.8 | 1.7 + 0.3 | 0.4 + 0.2 | 1594.7 + 107.0 | 40 |
| (3 mg/kg) | | | | | | |

In Tables 2-4, example numbers correspond to compounds prepared in referenced example numbers. For example, Example 2 corresponds to Compound 8.

Test compounds all showed very potent anticonvulsant effect in the PTZ model.

### Numbered Embodiments

In some embodiments, the present disclosure relates to the following embodiments.

Embodiment 1: A compound of formula (AI) or (AII):

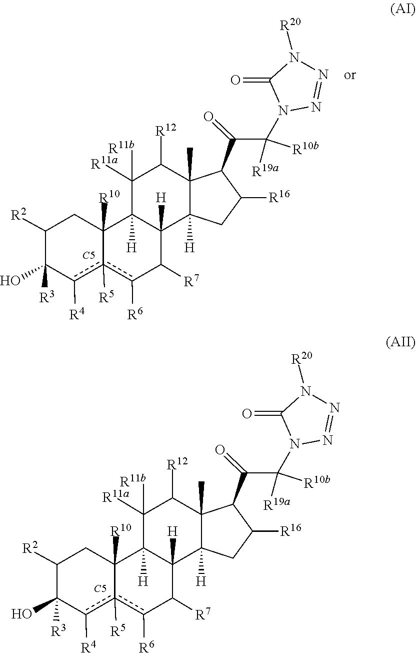

or a pharmaceutically acceptable salt, solvate, ester, or prodrug thereof;
wherein,
[symbol] represents a single or double bond; and
when one of [symbol] is a double bond, the other [symbol] is a single bond and R⁵ is absent;
when both of [symbol] are single bonds, then R⁵ is hydrogen;
R², R⁴, R⁶, R⁷, R^{11a}, R^{11b}, R¹², and R¹⁶ are each independently hydrogen, halogen, cyano, nitro, substituted or unsubstituted alkyl, substituted or unsubstituted alkenyl, substituted or unsubstituted alkynyl, substituted or unsubstituted carbocylyl, substituted or unsubstituted heterocyclyl, substituted or unsubstituted aryl, substituted or unsubstituted heteroaryl, —OR^{A}, —C(═O)R^{A}, —C(═O)OR^{A}, —OC(═O)R^{A}, —OC(═O)OR^{A}, —C(═O)NR^{A}R^{A}, —NR^{A}R^{A}, —NR^{A}C(═O)R^{A}, —OC(═O)NR^{A}R^{A}, —NR^{A}C(═O)OR^{A}, —NR^{A}C(═O)NR^{A}R^{A}, —SR^{A}, —S(═O)R^{A}, —S(═O)₂R^{A}, —S(═O)₂OR^{A}, —OS(═O)₂R^{A}, —S(═O)₂NR^{A}R^{A}, or —NR^{A}S(═O)₂R^{A}, wherein R^{A} is independently hydrogen, substituted or unsubstituted alkyl, substituted or unsubstituted alkenyl, substituted or unsubstituted alkynyl, substituted or unsubstituted carbocylyl, substituted or unsubstituted heterocylyl, substituted or unsubstituted aryl, substituted or unsubstituted heteroaryl, haloalkyl, an oxygen protecting group when attached to oxygen, a sulfur protecting group when attached to sulfur, or a nitrogen protecting group when attached to nitrogen; or two R^{A} groups can be taken together with the atoms to which they are attached to, to form a substituted or unsubstituted heterocylyl or heteroaryl ring;
alternatively, R^{11a} and R^{11b}, taken together with the carbon atom to which they are both attached, form a 3-8 membered saturated, partially saturated, or unsaturated ring optionally containing one or more heteroatoms as a ring member selected from N, O, or S; or R^{11a} and R^{11b} are joined to form an oxo (═O) group;
R³ is hydrogen, substituted or unsubstituted alkyl, substituted or unsubstituted alkenyl, substituted or unsubstituted alkynyl, substituted or unsubstituted carbocylyl, substituted or unsubstituted heterocyclyl, substituted or unsubstituted aryl, or substituted or unsubstituted heteroaryl;
R¹⁰ is hydrogen, halogen, cyano, or substituted or unsubstituted alkyl;
R^{19a} is hydrogen, substituted or unsubstituted alkyl, or —OR^{A19}, wherein R^{A19} is hydrogen, substituted or unsubstituted alkyl, substituted or unsubstituted alkenyl, substituted or unsubstituted alkynyl, or substituted or unsubstituted carbocylyl;
R^{19b} is hydrogen or substituted or unsubstituted alkyl;
alternatively, R^{19a} and R^{19b} are joined to form an oxo (═O) group, or R^{19a} and R^{19b} together with the carbon atom to which they are both attached, form a 3-8 membered saturated, partially saturated, or unsaturated ring optionally containing one or more heteroatoms as a ring member selected from N, O, or S; and
R²⁰ is hydrogen, alkyl, haloalkyl, cycloalkyl, cycloalkylalkyl, heterocyclyl, aryl, C₆-C₁₂ aryl-C₁-C₆ alkyl-, heteroaryl, or heteroaryl-C₁-C₆ alkyl-, wherein alkyl, haloalkyl, cycloalkyl, cycloalkylalkyl, heterocyclyl, aryl, C₆-C₁₂ aryl-C₁-C₆ alkyl-, heteroaryl, and heteroaryl-C₁-C₆ alkyl- can be optionally substituted with substituents selected from substituted or unsubstituted alkyl, substituted or unsubstituted alkenyl, substituted or unsubstituted alkynyl, substituted or unsubstituted carbocylyl, substituted or unsubstituted heterocylyl, haloalkyl, substituted or unsubstituted —C₆-C₁₂ aryl, substituted or unsubstituted 5-12 membered heteroaryl, halogen, nitro, cyano, —OR^{A}, —C(═O)R^{A}, —C(═O)OR^{A}, —OC(═O)R^{A}, —OC(═O)OR^{A}, —C(═O)NR^{A}R^{A}, —NR^{A}R^{A}, —NR^{A}C(═O)R^{A}, —OC(═O)NR^{A}R^{A}, —NR^{A}C(═O)OR^{A}, —NR^{A}C(═O)NR^{A}R^{A}, —SR^{A}, —S(═O)R^{A}, —S(═O)₂R^{A}, —S(═O)₂OR^{A}, —OS(═O)₂R^{A}, —S(═O)₂NR^{A}R^{A}, or —NR^{A}S(═O)₂R^{A}, wherein R^{A} is independently hydrogen or substituted or unsubstituted alkyl, substituted or unsubstituted alkenyl, substituted or unsubstituted alkynyl, substituted or unsubstituted carbocylyl, substituted or unsubstituted heterocylyl, substituted or unsubstituted aryl, substituted or unsubstituted heteroaryl, haloalkyl, an oxygen protecting group when attached to oxygen, a sulfur protecting group when attached to sulfur, or a nitrogen protecting group when attached to nitrogen, or two R^{A} groups can be taken together with the atoms to which they are attached to, to form a substituted or unsubstituted heterocylyl or heteroaryl ring.

Embodiment 2: The compound of Embodiment 1, wherein R³ is C₁₋₆ alkyl optionally substituted with alkoxy or one to three halo groups, and R²⁰ is hydrogen, substituted or unsubstituted —C₁₋₆ alkyl, —C₂₋₆ alkenyl, —C₂₋₆ alkynyl, —C₁₋₆ haloalkyl, —C₃₋₁₂ cycloalkyl, C₃₋₁₂ cycloalkyl(C₁₋₁₆ alkyl)-, heterocyclyl, —C₆-C₁₂ aryl, —C₆-C₁₂ aryl-C₁-C₆ alkyl-, 5-12 membered heteroaryl, or 5-12 membered heteroaryl-C₁-C₆ alkyl-.

Embodiment 3: The compound of Embodiment 1, wherein both of [symbol] are single bonds and R⁵ and R⁴ are in an alpha configuration.

Embodiment 4: The compound of Embodiment 1, wherein both of [symbol] are single bonds and R⁵ and R⁴ are in a beta configuration.

Embodiment 5: The compound of Embodiment 1, wherein both of [symbol] are single bonds and R⁵ and R⁶ are in an alpha configuration.

Embodiment 6: The compound of Embodiment 1, wherein both of [symbol] are single bonds and R⁵ and R⁶ are in a beta configuration.

Embodiment 7: The compound of Embodiment 1, wherein the compound has the structure of Formula (I-A):

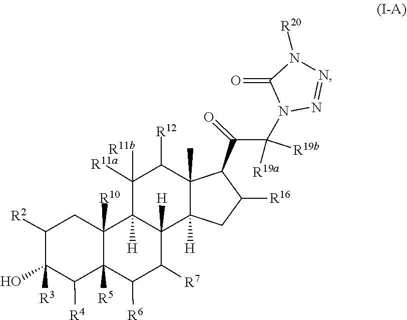

or a pharmaceutically acceptable salt, solvate, ester, or prodrug thereof.

Embodiment 8: The compound of Embodiment 1, wherein the compound has the structure of Formula (I-B):

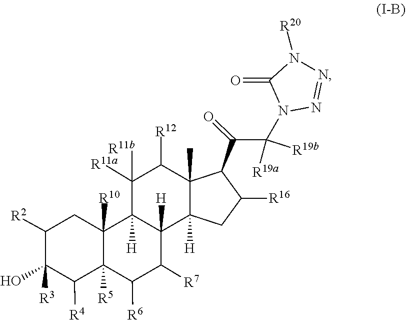

or a pharmaceutically acceptable salt, solvate, ester, or prodrug thereof.

Embodiment 9: The compound of Embodiment 1, wherein the compound has the structure of Formula (II-A):

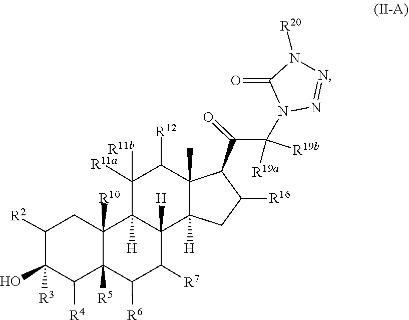

or a pharmaceutically acceptable salt, solvate, ester, or prodrug thereof.

Embodiment 10: The compound of Embodiment 1, wherein the compound has the structure of Formula (II-B):

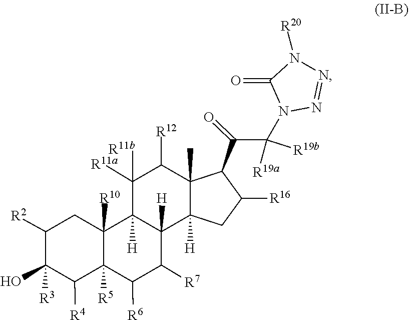

or a pharmaceutically acceptable salt, solvate, ester, or prodrug thereof.

Embodiment 11: The compound of any one of Embodiments 1-10, wherein the nitrogen protecting group, the sulfur protecting group, or the oxygen protecting group is benzyl.

Embodiment 12: The compound of any one of Embodiments 1-11, the substituents are selected from halogen, cyano, nitro, hydroxy, alkyl, haloalkyl, alkenyl, alkynyl, cycloalkyl, cycloalkylalkyl, —(C₁-C₆ alkyl)-OH, carbocylyl, heterocyclyl, aryl, C₆-C₁₂ aryl-(C₁-C₆ alkyl)-, heteroaryl, heteroaryl-(C₁-C₆ alkyl)-, —OR^{A}, —(C₁-C₆ alkyl)-OR^{A}, —C(═O)R^{A}, —C(═O)OR^{A}, —OC(═O)R^{A}, —OC(═O)OR^{A}, —C(═O)NR^{A}R^{A}, —NR^{A}R^{A}, —NR^{A}C(═O)R^{A}, —OC(═O)NR^{A}R^{A}, —NR^{A}C(═O)OR^{A}, —NR^{A}C(═O)NR^{A}R^{A}, —SR^{A}, —S(═O)R^{A}, —S(═O)₂R^{A}, —S(═O)₂OR^{A}, —OS(═O)₂R^{A}, —S(═O)₂NR^{A}R^{A}, or —NR^{A}S(═O)₂R^{A}, wherein R^{A} is independently hydrogen, alkyl, haloalkyl, alkenyl, alkynyl, cycloalkyl, cycloalkylalkyl, carbocylyl, heterocylyl, aryl, C₆-C₁₂ aryl-(C₁-C₆ alkyl)-, heteroaryl, heteroaryl-(C₁-C₆ alkyl)-, an oxygen protecting group when attached to an oxygen, a sulfur protecting group when attached to a sulfur, or a nitrogen protecting group when attached to a nitrogen; or two R^{A} groups can be taken together with the atoms to which they are attached to, to form a heterocylyl or heteroaryl ring.

Embodiment 13: The compound of any one of Embodiments 1-12, wherein the substituents are selected from fluoro, chloro, bromo, cyano, nitro, hydroxy, methoxy, ethoxy, propoxy, isopropoxy, methyl, ethyl, propyl, isopropyl, butyl, i-butyl, s-butyl, t-butyl, —CH₂CN, —CH₂CH₂CN, —CH₂F, —CHF₂, —CF₃, —CH₂OCH₃, —CH₂SCH₃, -methylhydroxy, morpholine, pyrrolidine, piperidine, piperazine, phenyl, benzyl, pyridine, pyrimidine, oxazole, pyrazole, cyclopropyl, cyclobutyl, cyclopentyl, cyclohexyl, —OCH₂F, —OCHF₂, —OCF₃, -ethylmethoxy, -methylcyclopropyl, —OR^{A}, —C(═O)R^{A}, —C(═O)OR^{A}, —OC(═O)R^{A}, —OC(═O)OR^{A}, —C(═O)NR^{A}R^{A}, —NR^{A}R^{A}, —NR^{A}C(═O)R^{A}, —OC(═O)NR^{A}R^{A}, —NR^{A}C(═O)OR^{A}, —NR^{A}C(═O)NR^{A}R^{A}, —SR^{A}, —S(═O)R^{A}, —S(═O)₂R^{A}, —S(═O)₂OR^{A}, —OS(═O)₂R^{A}, —S(═O)₂NR^{A}R^{A}, or —NR^{A}S(═O)₂R^{A}, wherein R^{A} is independently hydrogen, methyl, ethyl, propyl, isopropyl, cyclopropyl, cyclobutyl, cyclopentyl, cyclohexyl, —CH₂F, —CHF₂, —CF₃, phenyl, benzyl, pyridine, pyrimidine, -ethylmethoxy, or -methylcyclopropyl, or two R^{A} groups can be taken together with the atoms to which they are attached to, to form a heterocylyl or heteroaryl ring.

Embodiment 14: The compound of any one of Embodiments 1-13, wherein two R^{A} groups on the same nitrogen atom are taken together to form a substituted or unsubstituted heterocylyl or heteroaryl ring.

Embodiment 15: The compound of any one of Embodiments 1-14, wherein R³ is unsubstituted —C₁₋₆ alkyl, —C₃₋₁₂ cycloalkyl, or C₃₋₁₂ cycloalkyl-C₁₋₆ alkyl-.

Embodiment 16: The compound of any one of Embodiments 1-14, wherein R³ is a C₁₋₆ alkyl substituted with alkoxy.

Embodiment 17: The compound of any one of Embodiments 1-14, wherein R³ is a C₁₋₆ alkyl substituted with one to three halo groups.

Embodiment 18: The compound of any one of Embodiments 1-14, wherein R³ is —CH₃, —CH₂CH₃, —CH₂F, —CHF₂, —CF₃, —CH₂OCH₂CH₃, or —CH₂OCH₃.

Embodiment 19: The compound of Embodiment 18, wherein R³ is —CH₃.

Embodiment 20: The compound of any one of Embodiments 1-14, wherein R² is hydrogen, —OH, —OCH₃, —OCH₂CH₃, —OCH₂CH₂CH₃, —CH₃, —CH₂CH₃, —CH₂CH₂CH₃, substituted or unsubstituted cyclopropyl, fluoro, or chloro.

Embodiment 21: The compound of Embodiment 20, wherein R² is —CH₃ or —OCH₃.

Embodiment 22: The compound of Embodiment 21, wherein R² is —OCH₃.

Embodiment 23: The compound of Embodiment 20, wherein R² is hydrogen.

Embodiment 24: The compound of any one of Embodiments 1-14, wherein R^{11a} and R^{11b} are both hydrogen.

Embodiment 25: The compound of any one of Embodiments 1-14, wherein R^{11a} and R^{11b} together form ═O (oxo).

Embodiment 26: The compound of any one of Embodiments 1-14, wherein R^{11a} is —OH, —OCH₃, —OCH₂CH₃ or —OCH₂CH₃CH₃ and R^{11b} is hydrogen.

Embodiment 27: The compound of any one of Embodiments 1,2, and 11-14, wherein both [symbol] are single bonds and R⁴ and R⁶ are hydrogen.

Embodiment 28: The compound of any one of Embodiments 1,2, and 11-14, wherein one [symbol] is a single bond, and at least one of R⁴ or R⁶ is hydrogen, substituted or unsubstituted —C₁₋₆ alkyl, halogen, —CH₃, or —CF₃.

Embodiment 29: The compound of Embodiment 28, wherein R⁴ or R⁶ is fluoro.

Embodiment 30: The compound of any one of Embodiments 1-14, wherein R²⁰ is hydrogen, substituted or unsubstituted —C₁₋₆ alkyl, —C₂₋₆ alkenyl, —C₂₋₆ alkynyl, —C₁₋₆ haloalkyl, —C₃₋₁₂ cycloalkyl, C₃₋₁₂ cycloalkyl-C₁-C₆ alkyl-, heterocyclyl, —C₆-C₁₂ aryl, C₆-C₁₂ aryl-C₁-C₆ alkyl-, 5-12 membered heteroaryl, or 5-12 membered heteroaryl-C₁-C₆ alkyl-.

Embodiment 31: The compound of Embodiment 30, wherein R²⁰ is —C₁₋₆ alkyl, —C₁₋₆ haloalkyl, —C₃₋₁₂ cycloalkyl, C₃₋₁₂ cycloalkyl-C₁-C₆ alkyl-, —C₆-C₁₂ aryl, or 5-12 membered heteroaryl, which can be optionally substituted with halogen, nitro, cyano, —C₁₋₆ alkyl, —C₃₋₁₂ cycloalkyl, 3-12 membered heterocylyl, —C₆-C₁₂ aryl, 5-12 membered heteroaryl, —OR^{A}, —C(═O)R^{A}, —C(═O)OR^{A}, —OC(═O)R^{A}, —OC(═O)OR^{A}, —C(═O)NR^{A}R^{A}, —NR^{A}R^{A}, —NR^{A}C(═O)R^{A}, —OC(═O)NR^{A}R^{A}, —NR^{A}C(═O)OR^{A}, —NR^{A}C(═O)NR^{A}R^{A}, —SR^{A}, —S(═O)R^{A}, —S(═O)₂R^{A}, —S(═O)₂OR^{A}, —OS(═O)₂R^{A}, —S(═O)₂NR^{A}R^{A}, or —NR^{A}S(═O)₂R^{A}, wherein R^{A} is independently hydrogen or substituted or unsubstituted alkyl, haloalkyl, alkenyl, alkynyl, cycloalkyl, cycloalkylalkyl, carbocylyl, heterocylyl, aryl, C₆-C₁₂ aryl-C₁-C₆ alkyl-, heteroaryl, heteroaryl-C₁-C₆ alkyl-, or two R^{A} groups can be taken together with the atoms to which they are attached to, to form a substituted or unsubstituted heterocylyl or heteroaryl ring.

Embodiment 32: The compound of Embodiment 31, wherein R²⁰ is —C₁₋₆ alkyl, —C₃₋₁₂ cycloalkyl, C₃₋₁₂ cycloalkyl-C₁-C₆ alkyl-, —C₆-C₁₂ aryl, or 5-12 membered heteroaryl, which can be optionally substituted with halogen, cyano, substituted or unsubstituted alkyl, substituted or unsubstituted carbocylyl, 3-12 membered heterocylyl, —C₆-C₁₂ aryl, 5-12 membered heteroaryl, or alkoxy.

Embodiment 33: The compound of Embodiment 32, wherein R²⁰ is substituted or unsubstituted —C₁₋₆ alkyl or C₃₋₁₂ cycloalkyl-C₁-C₆ alkyl-.

Embodiment 34: The compound of Embodiment 33, wherein R²⁰ is —CH₃, —CF₃, —CH₂CH₃, -i-Pr, -n-Pr, -i-Bu, -s-Bu, -t-Bu, —CH₂cyclopropyl. —CH₂CN, or —CH₂CH₂CN.

Embodiment 35: The compound of Embodiment 32, wherein R²⁰ is substituted or unsubstituted —C₃₋₁₂ cycloalkyl.

Embodiment 36: The compound of Embodiment 35, wherein R²⁰ is cyclopropyl, cyclobutyl, cyclopentyl, or cyclohexyl.

Embodiment 37: The compound of Embodiment 32, wherein R²⁰ is substituted or unsubstituted —C₆-C₁₂ aryl.

Embodiment 38: The compound of Embodiment 37, wherein R²⁰ is phenyl.

Embodiment 39: The compound of Embodiment 37, wherein R²⁰ is phenyl substituted with one or more halogen, cyano, or alkoxy.

Embodiment 40: The compound of Embodiment 32, wherein R²⁰ is substituted or unsubstituted 5-12 membered heteroaryl.

Embodiment 41: The compound of Embodiment 40, wherein R²⁰ is pyridine.

Embodiment 42: The compound of Embodiment 40, wherein R²⁰ is pyridine substituted with one or more halogen, cyano, or alkoxy.

Embodiment 43: The compound of Embodiment 30, wherein R²⁰ is oxazole, pyrazole, or N-methylpyrazole.

Embodiment 44: The compound of any one of Embodiments 1-14, wherein R⁷ is hydrogen, —CH₃, —CH₂CH₃, —CH(CH₃)₂, —OH, —OCH₃, or —CH₂OCH₃.

Embodiment 45: The compound of Embodiment 44, wherein R⁷ is hydrogen.

Embodiment 46: The compound of Embodiment 44, wherein R⁷ is —CH₃.

Embodiment 47: The compound of Embodiment 44, wherein R⁷ is —OH or —OCH₃.

Embodiment 48: The compound of Embodiment 44, wherein R⁷ is —CH₂OCH₃.

Embodiment 49: The compound of any one of Embodiments 1-14, wherein R¹² is hydrogen, —CH₃, —CH₂CH₃, —CH(CH₃)₂, —OH, —OCH₃, or —CH₂OCH₃.

Embodiment 50: The compound of Embodiment 49, wherein R¹² is hydrogen.

Embodiment 51: The compound of Embodiment 49, wherein R¹² is —CH₃.

Embodiment 52: The compound of Embodiment 49, wherein R¹² is —OH or —OCH₃.

Embodiment 53: The compound of any one of Embodiments 1-14, wherein R¹⁶ is hydrogen, —CH₃, —CH₂CH₃, —CH(CH₃)₂, —OH, —OCH₃, or —CH₂OCH₃.

Embodiment 54: The compound of Embodiment 53, wherein R¹⁶ is hydrogen.

Embodiment 55: The compound of Embodiment 53, wherein R¹⁶ is —CH₃.

Embodiment 56: The compound of any one of Embodiments 1-14, wherein R¹⁰ is hydrogen, halogen, cyano, —CH₃, —CH₂CH₃, —CH₂F, —CHF₂, —CF₃, —CH₂OCH₃, or —CH₂SCH₃.

Embodiment 57: The compound of Embodiment 56, wherein R¹⁰ is hydrogen.

Embodiment 58: The compound of Embodiment 56, wherein R¹⁰ is —CH₃ or fluoro.

Embodiment 59: The compound of Embodiment 56, wherein R¹⁰ is —CH₂OCH₃.

Embodiment 60: The compound of any one of Embodiments 1-14, wherein R^{19a} and R^{19b} are both hydrogen.

Embodiment 61: The compound of any one of Embodiments 1-14, wherein R^{19a} is —C₁₋₆ alkyl and R^{19b} is hydrogen or —C₁₋₆ alkyl.

Embodiment 62: The compound of Embodiment 61, wherein R^{19a} is methyl and R^{19b} is hydrogen.

Embodiment 63: The compound of any one of Embodiments 1-14, wherein at least one of R², R⁴, R⁵, R⁶, R⁷, R¹⁰, R^{11a}, R^{11b}, R¹², R¹⁶, R^{19a} and R^{19b} is hydrogen.

Embodiment 64: The compound of any one of Embodiments 1-14, wherein R², R⁴, R⁵, R⁶, R⁷, R¹⁰, R^{11a}, R^{11b}, R¹², R¹⁶, R^{19a} and R^{19b} are all hydrogen.

Embodiment 65: The compound of Embodiment 64, wherein R³ is hydrogen, substituted or unsubstituted —C₁₋₆ alkyl, —C₃₋₁₂ cycloalkyl, or C₃₋₁₂ cycloalkyl-C₁₋₆ alkyl-.

Embodiment 66: The compound of Embodiment 65, wherein R³ is methyl.

Embodiment 67: The compound of Embodiment 66, wherein R²⁰ is hydrogen, substituted or unsubstituted —C₁₋₆ alkyl, —C₂₋₆ alkenyl, —C₂₋₆ alkynyl, —C₁₋₆ haloalkyl, —C₃₋₁₂ cycloalkyl, C₃₋₁₂ cycloalkyl-C₁-C₆ alkyl-, heterocyclyl, —C₆-C₁₂ aryl, C₆-C₁₂ aryl-C₁-C₆ alkyl-, 5-12 membered heteroaryl, or 5-12 membered heteroaryl-C₁-C₆ alkyl-.

Embodiment 68: The compound of Embodiment 67, wherein R²⁰ is —C₁₋₆ alkyl, —C₁₋₆ haloalkyl, —C₃₋₁₂ cycloalkyl, C₃₋₁₂ cycloalkyl-C₁-C₆ alkyl-, —C₆-C₁₂ aryl, or 5-12 membered heteroaryl, which can be optionally substituted with halogen, nitro, cyano, substituted or unsubstituted —C₁₋₆ alkyl, —C₃₋₁₂ cycloalkyl, 3-12 membered heterocylyl, —C₆-C₁₂ aryl, 5-12 membered heteroaryl, —OR^{A}, —C(═O)R^{A}, —C(═O)OR^{A}, —OC(═O)R^{A}, —OC(═O)OR^{A}, —C(═O)NR^{A}R^{A}, —NR^{A}R^{A}, —NR^{A}C(═O)R^{A}, —OC(═O)NR^{A}R^{A}, —NR^{A}C(═O)OR^{A}, —NR^{A}C(═O)NR^{A}R^{A}, —SR^{A}, —S(═O)R^{A}, —S(═O)₂R^{A}, —S(═O)₂OR^{A}, —OS(═O)₂R^{A}, —S(═O)₂NR^{A}R^{A}, or —NR^{A}S(═O)₂R^{A}, wherein R^{A} is independently hydrogen or substituted or unsubstituted alkyl, haloalkyl, alkenyl, alkynyl, cycloalkyl, cycloalkylalkyl, carbocylyl, heterocylyl, aryl, C₆-C₁₂ aryl-C₁-C₆ alkyl-, heteroaryl, heteroaryl-C₁-C₆ alkyl-, or two R^{A} groups can be taken together with the atoms to which they are attached to, to form a substituted or unsubstituted heterocylyl or heteroaryl ring.

Embodiment 69: The compound of Embodiment 68, wherein R²⁰ is —C₁₋₆ alkyl, —C₃₋₁₂ cycloalkyl, C₃₋₁₂ cycloalkyl-C₁-C₆ alkyl-, −C₆-C₁₂ aryl, or 5-12 membered heteroaryl, which can be optionally substituted with halogen, cyano, substituted or unsubstituted alkyl, substituted or unsubstituted carbocylyl, 3-12 membered heterocylyl, —C₆-C₁₂ aryl, 5-12 membered heteroaryl, or alkoxy.

Embodiment 70: The compound of Embodiment 69, wherein R²⁰ is substituted or unsubstituted —C₁₋₆ alkyl or C₃₋₁₂ cycloalkyl-C₁-C₆ alkyl-.

Embodiment 71: The compound of Embodiment 70, wherein R²⁰ is —CH₃, —CF₃, —CH₂CH₃, -i-Pr, -n-Pr, -i-Bu, -s-Bu, -t-Bu, —CH₂cyclopropyl, —CH₂CN, or —CH₂CH₂CN.

Embodiment 72: The compound of Embodiment 69, wherein R²⁰ is substituted or unsubstituted —C₃₋₁₂ cycloalkyl.

Embodiment 73: The compound of Embodiment 72, wherein R²⁰ is cyclopropyl, cyclobutyl, cyclopentyl, or cyclohexyl.

Embodiment 74: The compound of Embodiment 69, wherein R²⁰ is substituted or unsubstituted —C₆-C₁₂ aryl.

Embodiment 75: The compound of Embodiment 74, wherein R²⁰ is phenyl.

Embodiment 76: The compound of Embodiment 74, wherein R²⁰ is phenyl substituted with one or more halogen, cyano, or alkoxy.

Embodiment 77: The compound of Embodiment 69, wherein R²⁰ is substituted or unsubstituted 5-12 membered heteroaryl.

Embodiment 78: The compound of Embodiment 77, wherein R²⁰ is pyridine.

Embodiment 79: The compound of Embodiment 77, wherein R²⁰ is pyridine substituted with one or more halogen, cyano, or alkoxy.

Embodiment 80: The compound of Embodiment 67, wherein R²⁰ is oxazole, pyrazole, or N-methylpyrazole.

Embodiment 81: The compound of Embodiment 1, wherein the compound has the structure of Formula (I-E1) or (I-F1):

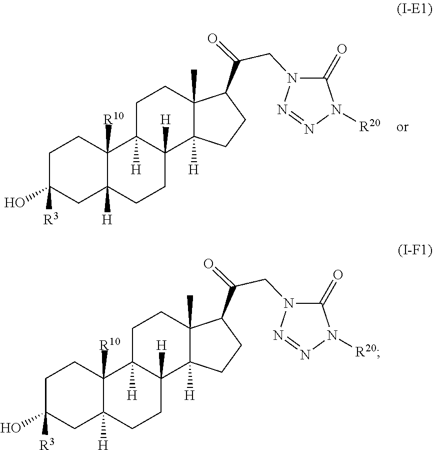

or a pharmaceutically acceptable salt, solvate, ester, or prodrug thereof, wherein:
R³ is hydrogen, —C₁₋₆ alkyl, —C₂₋₆ alkenyl, or —C₂₋₆ alkynyl, each optionally substituted with one to three groups selected from halogen or —C₁₋₆ alkoxy;
R¹⁰ is hydrogen, halogen, cyano, or —C₁₋₆ alkyl wherein —C₁₋₆ alkyl is optionally substituted with halogen or —C₁₋₃ alkoxy; and
R²⁰ is hydrogen, —C₁₋₆ alkyl, —C₂₋₆ alkenyl, —C₂₋₆ alkynyl, —C₁₋₆ haloalkyl, —C₃₋₁₂ cycloalkyl, C₃₋₁₂ cycloalkyl-C₁-C₆ alkyl-, 3-12 membered heterocyclyl, —C₆-C₁₂ aryl, C₆-C₁₂ aryl-C₁-C₆, alkyl-, heteroaryl-C₁-C₆ alkyl-, or 5-12 membered heteroaryl, each of which can be optionally substituted with halogen, nitro, cyano, —C₁₋₆ alkyl, —C₂₋₆ alkenyl, —C₂₋₆ alkynyl, —C₃₋₁₂ cycloalkyl, 3-12 membered heterocylyl, —C₆-C₁₂ aryl, 5-12 membered heteroaryl, —OR^{A}, —C(═O)R^{A}, —C(═O)OR^{A}, —OC(═O)R^{A}, —OC(═O)OR^{A}, —C(═O)NR^{A}R^{A}, —NR^{A}R^{A}, —NR^{A}C(═O)R^{A}, —OC(═O)NR^{A}R^{A}, —NR^{A}C(═O)OR^{A}, —NR^{A}C(═O)NR^{A}R^{A}, —SR^{A}, —S(═O)R^{A}, —S(═O)₂R^{A}, —S(═O)₂OR^{A}, —OS(═O)₂R^{A}, —S(═O)₂NR^{A}R^{A}, or —NR^{A}S(═O)₂R^{A};
wherein R^{A} is independently hydrogen, substituted or unsubstituted alkyl, haloalkyl, alkenyl, alkynyl, cycloalkyl, cycloalkylalkyl, carbocylyl, heterocylyl, aryl, C₆-C₁₂ aryl-C₁-C₆ alkyl-, heteroaryl, heteroaryl-C₁-C₆ alkyl-, an oxygen protecting group when attached to oxygen, a sulfur protecting group when attached to sulfur, or a nitrogen protecting group when attached to nitrogen; or two R^{A} groups can be taken together with the atoms to which they are attached to, to form a substituted or unsubstituted heterocylyl or heteroaryl ring;
wherein the substituents are selected from fluoro, chloro, bromo, cyano, nitro, hydroxy, methoxy, ethoxy, propoxy, isopropoxy, methyl, ethyl, propyl, isopropyl, butyl, i-butyl, s-butyl, t-butyl, —CH₂CN, —CH₂CH₂CN, —CH₂F, —CHF₂, —CF₃, —CH₂OCH₃, —CH₂SCH₃, -methylhydroxy, morpholine, pyrrolidine, piperidine, piperazine, phenyl, benzyl, pyridine, pyrimidine, oxazole, pyrazole, cyclopropyl, cyclobutyl, cyclopentyl, cyclohexyl, —OCH₂F, —OCHF₂, —OCF₃, -ethylmethoxy, -methylcyclopropyl, —OR^{A}, —C(═O)R^{A}, —C(═O)OR^{A}, —OC(═O)R^{A}, —OC(═O)OR^{A}, —C(═O)NR^{A}R^{A}, —NR^{A}R^{A}, —NR^{A}C(═O)R^{A}, —OC(═O)NR^{A}R^{A}, —NR^{A}C(═O)OR^{A}, —NR^{A}C(═O)NR^{A}R^{A}, —SR^{A}, —S(═O)R^{A}, —S(═O)₂R^{A}, —S(═O)₂OR^{A}, —OS(═O)₂R^{A}, —S(═O)₂NR^{A}R^{A}, or —NR^{A}S(═O)₂R^{A}, wherein R^{A} is independently hydrogen, methyl, ethyl, propyl, isopropyl, cyclopropyl, cyclobutyl, cyclopentyl, cyclohexyl, —CH₂F, —CHF₂, —CF₃, phenyl, benzyl, pyridine, pyrimidine, -ethylmethoxy, or -methylcyclopropyl, or two R^{A} groups can be taken together with the atoms to which they are attached to, to form a heterocylyl or heteroaryl ring.

Embodiment 82: The compound of Embodiment 81, wherein R³ is —C₁₋₆ alkyl optionally substituted with one to three groups selected from halogen or —C₁₋₆ alkoxy.

Embodiment 83: The compound of Embodiment 82, wherein R³ is —CH₃, —CH₂CH₃, —CH₂F, —CHF₂, —CF₃, —CH₂OCH₂CH₃, or —CH₂OCH₃.

Embodiment 84: The compound of Embodiment 83, wherein R³ is —CH₃.

Embodiment 85: The compound of any one of Embodiments 81-84, wherein R¹⁰ is hydrogen, halogen, cyano, —CH₃, —CH₂CH₃, —CH₂F, —CHF₂, —CF₃, —CH₂OCH₃, or —CH₂SCH₃.

Embodiment 86: The compound of Embodiment 1, wherein the compound has the structure of Formula (I-G1)

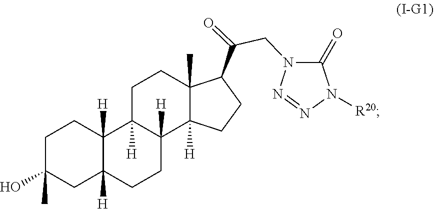

or a pharmaceutically acceptable salt, solvate, ester, or prodrug thereof, wherein:
R²⁰ is hydrogen, —C₁₋₆ alkyl, —C₃₋₁₂ cycloalkyl, C₃₋₁₂ cycloalkyl-C₁₋₆ alkyl-, —C₆-C₁₂ aryl, or 5-12 membered heteroaryl, each of which can be optionally substituted with halogen, nitro, cyano, —C₁₋₆ alkyl, —C₂₋₆ alkenyl, —C₂₋₆ alkynyl, —C₃₋₁₂ cycloalkyl, 3-12 membered heterocylyl, —C₆-C₁₂ aryl, 5-12 membered heteroaryl, —OR^{A}, —C(═O)R^{A}, —C(═O)OR^{A}, —OC(═O)R^{A}, —OC(═O)OR^{A}, —C(═O)NR^{A}R^{A}, —NR^{A}R^{A}, —NR^{A}C(═O)R^{A}, —OC(═O)NR^{A}R^{A}, —NR^{A}C(═O)OR^{A}, —NR^{A}C(═O)NR^{A}R^{A}, —SR^{A}, —S(═O)R^{A}, —S(═O)₂R^{A}, —S(═O)₂OR^{A}, —OS(═O)₂R^{A}, —S(═O)₂NR^{A}R^{A}, or —NR^{A}S(═O)₂R^{A};
wherein R^{A} is independently hydrogen, substituted or unsubstituted alkyl, haloalkyl, alkenyl, alkynyl, cycloalkyl, cycloalkylalkyl, carbocylyl, heterocylyl, aryl, C₆-C₁₂ aryl-C₁-C₆ alkyl-, heteroaryl, heteroaryl-C₁-C₆ alkyl-, an oxygen protecting group when attached to oxygen, a sulfur protecting group when attached to sulfur, or a nitrogen protecting group when attached to nitrogen; or two R^{A} groups can be taken together with the atoms to which they are attached to, to form a substituted or unsubstituted heterocylyl or heteroaryl ring;
wherein the substituents are selected from fluoro, chloro, bromo, cyano, nitro, hydroxy, methoxy, ethoxy, propoxy, isopropoxy, methyl, ethyl, propyl, isopropyl, butyl, i-butyl, s-butyl, t-butyl, —CH₂CN, —CH₂CH₂CN, —CH₂F, —CHF₂, —CF₃, —CH₂OCH₃, —CH₂SCH₃, -methylhydroxy, morpholine, pyrrolidine, piperidine, piperazine, phenyl, benzyl, pyridine, pyrimidine, oxazole, pyrazole, cyclopropyl, cyclobutyl, cyclopentyl, cyclohexyl, —OCH₂F, —OCHF₂, —OCF₃, -ethylmethoxy, -methylcyclopropyl, —OR^{A}, —C(═O)R^{A}, —C(═O)OR^{A}, —OC(═O)R^{A}, —OC(═O)OR^{A}, —C(═O)NR^{A}R^{A}, —NR^{A}R^{A}, —NR^{A}C(═O)R^{A}, —OC(═O)NR^{A}R^{A}, —NR^{A}C(═O)OR^{A}, —NR^{A}C(═O)NR^{A}R^{A}, —SR^{A}, —S(═O)R^{A}, —S(═O)₂R^{A}, —S(═O)₂OR^{A}, —OS(═O)₂R^{A}, —S(═O)₂NR^{A}R^{A}, or —NR^{A}S(═O)₂R^{A}, wherein R^{A} is independently hydrogen, methyl, ethyl, propyl, isopropyl, cyclopropyl, cyclobutyl, cyclopentyl, cyclohexyl, —CH₂F, —CHF₂, —CF₃, phenyl, benzyl, pyridine, pyrimidine, -ethylmethoxy, or -methylcyclopropyl, or two R^{A} groups can be taken together with the atoms to which they are attached to, to form a heterocylyl or heteroaryl ring.

Embodiment 87: The compound of Embodiment 86, wherein R²⁰ is hydrogen, —C₁₋₆ alkyl, —C₂₋₆ alkenyl, —C₂₋₆ alkynyl, —C₁₋₆ haloalkyl, —C₃₋₁₂ cycloalkyl, C₃₋₁₂ cycloalkyl-C₁-C₆ alkyl-, 3-12 membered heterocyclyl, —C₆-C₁₂ aryl, C₆-C₁₂ aryl-C₁-C₆ alkyl-, heteroaryl-C₁-C₆ alkyl-, or 5-12 membered heteroaryl, each of which can be optionally substituted with halogen, nitro, cyano, —C₁₋₆ alkyl, —C₂₋₆ alkenyl, —C₂₋₆ alkynyl, —C₃₋₁₂ cycloalkyl, 3-12 membered heterocylyl, —C₆-C₁₂ aryl, 5-12 membered heteroaryl, —OR^{A}, —C(═O)R^{A}, —C(═O)OR^{A}, —OC(═O)R^{A}, —OC(═O)OR^{A}, —C(═O)NR^{A}R^{A}, —NR^{A}R^{A}, —NR^{A}C(═O)R^{A}, —OC(═O)NR^{A}R^{A}, —NR^{A}C(═O)OR^{A}, —NR^{A}C(═O)NR^{A}R^{A}, —SR^{A}, —S(═O)R^{A}, —S(═O)₂R^{A}, —S(═O)₂OR^{A}, —OS(═O)₂R^{A}, —S(═O)₂NR^{A}R^{A}, or —NR^{A}S(═O)₂R^{A}.

Embodiment 88: The compound of any one of Embodiments 81-87, wherein R²⁰ is hydrogen, —C₁₋₆ alkyl, —C₃₋₁₂ cycloalkyl, C₃₋₁₂ cycloalkyl-C₁-C₆ alkyl-, —C₆-C₁₂ aryl, or 5-12 membered heteroaryl, each of which are optionally substituted.

Embodiment 89: The compound of Embodiment 88, wherein R²⁰ is —CH₃, —CF₃, —CH₂CH₃, -i-Pr, -n-Pr, -i-Bu, -s-Bu, -t-Bu, -CH₂cyclopropyl, —CH₂CN, or —CH₂CH₂CN.

Embodiment 90: The compound of Embodiment 88, wherein R²⁰ is cyclopropyl, cyclobutyl, cyclopentyl, or cyclohexyl.

Embodiment 91: The compound of Embodiment 88, wherein R²⁰ is phenyl.

Embodiment 92: The compound of Embodiment 88, wherein R²⁰ is phenyl substituted with one or more halogen, cyano, or alkoxy.

Embodiment 93: The compound of Embodiment 88, wherein R²⁰ is pyridine.

Embodiment 94: The compound of Embodiment 88, wherein R²⁰ is pyridine substituted with one or more halogen, cyano, or alkoxy.

Embodiment 95: The compound of Embodiment 88, wherein R²⁰ is oxazole, pyrazole, or N-methylpyrazole.

Embodiment 96: The compound of any one of Embodiments 81-88, wherein the nitrogen protecting group, the sulfur protecting group, or the oxygen protecting group is benzyl.

Embodiment 97: The compound of any one of Embodiments 81-88, wherein two R^{A} groups on the same nitrogen atom are taken together to form a substituted or unsubstituted heterocylyl or heteroaryl ring.

Embodiment 98: The compound of any one of Embodiments 1-12, wherein the substituents are selected from fluoro, chloro, bromo, cyano, nitro, hydroxy, methoxy, ethoxy, propoxy, isopropoxy, methyl, ethyl, propyl, isopropyl, —CH₂F, —CHF₂, —CF₃, -methylhydroxy, morpholine, pyrrolidine, piperidine, piperazine, phenyl, benzyl, pyridine, pyrimidine, cyclopropyl, cyclobutyl, cyclopentyl, cyclohexyl, —OCH₂F, —OCHF₂, —OCF₃, -ethylmethoxy, -methylcyclopropyl, —OR^{A}, —C(═O)R^{A}, —C(═O)OR^{A}, —OC(═O)R^{A}, —OC(═O)OR^{A}, —C(═O)NR^{A}R^{A}, —NR^{A}R^{A}, —NR^{A}C(═O)R^{A}, —OC(═O)NR^{A}R^{A}, —NR^{A}C(═O)OR^{A}, —NR^{A}C(═O)NR^{A}R^{A}, —SR^{A}, —S(═O)R^{A}, —S(═O)₂R^{A}, —S(═O)₂OR^{A}, —OS(═O)₂R^{A}, —S(═O)₂NR^{A}R^{A}, or —NR^{A}S(═O)₂R^{A}, wherein R^{A} is independently hydrogen, methyl, ethyl, propyl, isopropyl, cyclopropyl, cyclobutyl, cyclopentyl, cyclohexyl, —CH₂F, —CHF₂, —CF₃, phenyl, benzyl, pyridine, pyrimidine, -ethylmethoxy, or -methylcyclopropyl, or two R^{A} groups can be taken together with the atoms to which they are attached to, to form a heterocylyl or heteroaryl ring.

Embodiment 99: The compound of Embodiments 1,69, or 88, wherein the compound is:

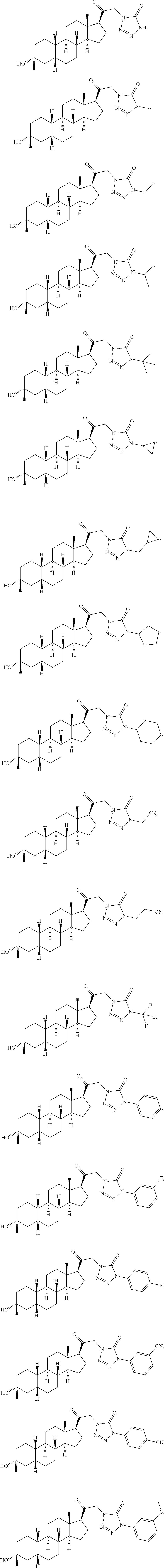

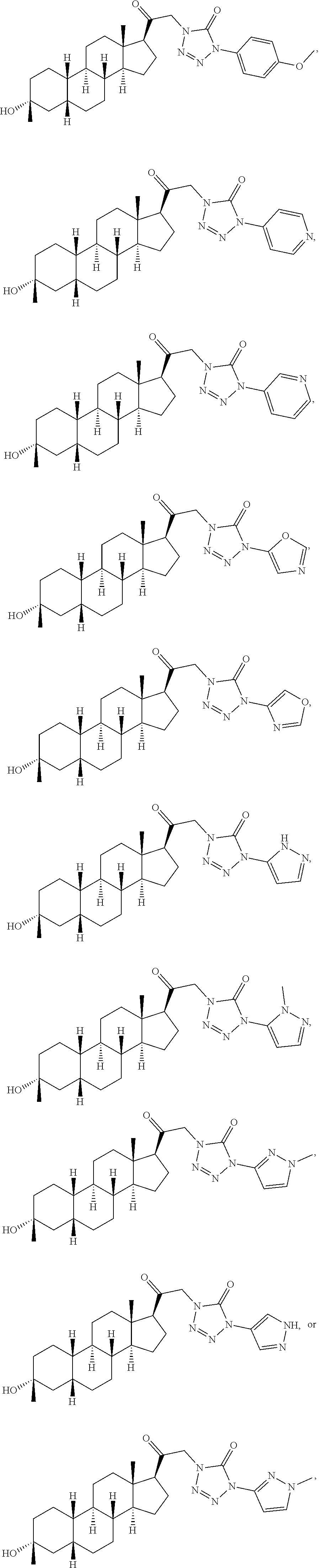

or a pharmaceutically acceptable salt, solvate, ester, or prodrug thereof.

Embodiment 100: A pharmaceutical composition comprising a compound according to any one of Embodiments 1-99, or a pharmaceutically acceptable salt, a solvate, a stereoisomer, or tautomer thereof, and a pharmaceutically acceptable carrier.

Embodiment 101: The pharmaceutical composition of Embodiment 100, further comprising at least one additional therapeutically active agent.

Embodiment 102: A method for treating a CNS-related disorder in a subject in need thereof, comprising administering to the subject an effective amount of a compound of any one of Embodiments 1-99, or a pharmaceutically acceptable salt, a solvate, a stereoisomer, or tautomer thereof.

Embodiment 103: The method of Embodiment 102, wherein the CNS-related disorder is a sleep disorder, a mood disorder, a schizophrenia spectrum disorder, a convulsive disorder, a disorder of memory and/or cognition, a movement disorder, a personality disorder, autism spectrum disorder, pain, traumatic brain injury, a vascular disease, a substance abuse disorder and/or withdrawal syndrome, or tinnitus

Embodiment 104: The method of Embodiment 102, wherein the compound is administered orally, subcutaneously, intravenously, or intramuscularly.

Embodiment 105: The method of Embodiment 102, wherein the compound is administered chronically.

Embodiment 106: A method for inducing sedation and/or anesthesia in a subject in need thereof, comprising administering to the subject an effective amount of a compound of any one of Embodiments 1-99, or a pharmaceutically acceptable salt, a solvate, a stereoisomer, or tautomer thereof.

All publications, patents, and patent applications mentioned in the specification are indicative of the level of those skilled in the art to which this disclosure pertains. All publications, patents, and patent applications are herein incorporated by reference for all purposes to the same extent as if each individual publication or patent application was specifically and individually indicated to be incorporated by reference. Nothing herein is to be construed as an admission that the present disclosure is not entitled to antedate such publication by virtue of prior disclosure. All publications, patents, and patent applications mentioned in the specification are indicative of the level of those skilled in the art to which this disclosure pertains. All publications, patents, and patent applications are herein incorporated by reference for all purposes to the same extent as if each individual publication or patent application was specifically and individually indicated to be incorporated by reference. Nothing herein is to be construed as an admission that the present disclosure is not entitled to antedate such publication by virtue of prior disclosure.

Many modifications and other embodiments of the disclosures set forth herein will come to mind to one skilled in the art to which these disclosures pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. Although specific terms are employed herein, they are used in a generic and descriptive sense only and not for purposes of limitation.

While the disclosure has been described in connection with specific embodiments thereof, it will be understood that it is capable of further modifications and this application is intended to cover any variations, uses, or adaptations of the disclosure following, in general, the principles of the disclosure and including such departures from the present disclosure as come within known or customary practice within the art to which the disclosure pertains and as may be applied to the essential features hereinbefore set forth and as follows in the scope of the appended claims.

What is claimed is:

1. A compound of formula (AI) or (AII): 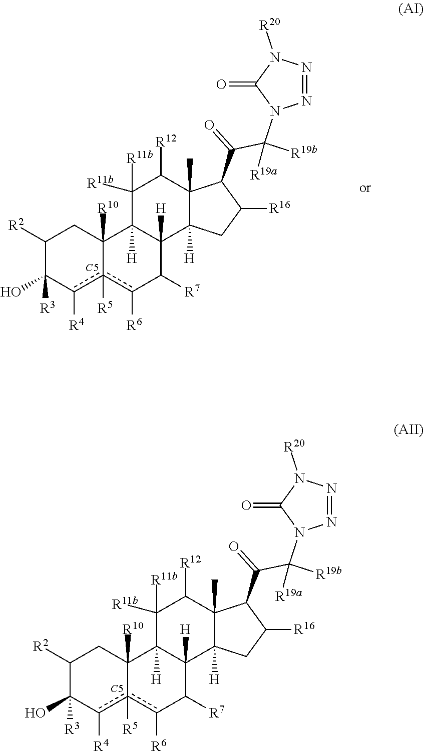
or a pharmaceutically acceptable salt, solvate, ester, or prodrug thereof;
wherein, [symbol]
represents a single or double bond; and
when one of [symbol] is a double bond, the other [symbol] is a single bond and R⁵ is absent;
when both of [symbol] are single bonds, then R⁵ is hydrogen;
R², R⁴, R⁶, R⁷, R^{11a}, R^{11b}, R¹², and R¹⁶ are each independently hydrogen, halogen, cyano, nitro, substituted or unsubstituted alkyl, substituted or unsubstituted alkenyl, substituted or unsubstituted alkynyl, substituted or unsubstituted carbocylyl, substituted or unsubstituted heterocyclyl, substituted or unsubstituted aryl, substituted or unsubstituted heteroaryl, —OR^{A}, —C(═O)R^{A}, —C(═O)OR^{A}, —OC(═O)R^{A}, —OC(═O)OR^{A}, —C(═O)NR^{A}R^{A}, —NR^{A}R^{A}, —NR^{A}C(═O)R^{A}, —OC(═O)NR^{A}R^{A}, —NR^{A}C(═O)OR^{A}, —NR^{A}C(═O)NR^{A}R^{A}, —SR^{A}, —S(═O)R^{A}, —S(═O)₂R^{A}, —S(═O)₂OR^{A}, —OS(═O)₂R^{A}, —S(═O)₂NR^{A}R^{A}, or —NR^{A}S(═O)₂R^{A}, wherein R^{A} is independently hydrogen, substituted or unsubstituted alkyl, substituted or unsubstituted alkenyl, substituted or unsubstituted alkynyl, substituted or unsubstituted carbocylyl, substituted or unsubstituted heterocylyl, substituted or unsubstituted aryl, substituted or unsubstituted heteroaryl, haloalkyl, an oxygen protecting group when attached to oxygen, a sulfur protecting group when attached to sulfur, or a nitrogen protecting group when attached to nitrogen; or two R^{A} groups can be taken together with the atoms to which they are attached to, to form a substituted or unsubstituted heterocylyl or heteroaryl ring;
alternatively, R^{11a} and R^{11b}, taken together with the carbon atom to which they are both attached, form a 3-8 membered saturated, partially saturated, or unsaturated ring optionally containing one or more heteroatoms as a ring member selected from N, O, or S; or R^{11a} and R^{11b} are joined to form an oxo (═O) group;
R³ is hydrogen, substituted or unsubstituted alkyl, substituted or unsubstituted alkenyl, substituted or unsubstituted alkynyl, substituted or unsubstituted carbocylyl, substituted or unsubstituted heterocyclyl, substituted or unsubstituted aryl, or substituted or unsubstituted heteroaryl;
R¹⁰ is hydrogen, halogen, cyano, or substituted or unsubstituted alkyl;
R^{19a} is hydrogen, substituted or unsubstituted alkyl, or —OR^{A19}, wherein R^{A19} is hydrogen, substituted or unsubstituted alkyl, substituted or unsubstituted alkenyl, substituted or unsubstituted alkynyl, or substituted or unsubstituted carbocylyl;
R^{19b} is hydrogen or substituted or unsubstituted alkyl;
alternatively, R^{19a} and R^{19b} are joined to form an oxo (═O) group, or R^{19a} and R^{19b} together with the carbon atom to which they are both attached, form a 3-8 membered saturated, partially saturated, or unsaturated ring optionally containing one or more heteroatoms as a ring member selected from N, O, or S; and
R²⁰ is hydrogen, alkyl, haloalkyl, cycloalkyl, cycloalkylalkyl, heterocyclyl, aryl, C₆-C₁₂ aryl-C₁-C₆ alkyl-, heteroaryl, or heteroaryl-C₁-C₆ alkyl-, wherein alkyl, haloalkyl, cycloalkyl, cycloalkylalkyl, heterocyclyl, aryl, C₆-C₁₂ aryl-C₁-C₆ alkyl-, heteroaryl, and heteroaryl-C₁-C₆ alkyl- can be optionally substituted with substituents selected from substituted or unsubstituted alkyl, substituted or unsubstituted alkenyl, substituted or unsubstituted alkynyl, substituted or unsubstituted carbocylyl, substituted or unsubstituted heterocylyl, haloalkyl, substituted or unsubstituted —C₆-C₁₂ aryl, substituted or unsubstituted 5-12 membered heteroaryl, halogen, nitro, cyano, —OR^{A}, —C(═O)R^{A}, —C(═O)OR^{A}, —OC(═O)R^{A}, —OC(═O)OR^{A}, —C(═O)NR^{A}R^{A}, —NR^{A}R^{A}, —NR^{A}C(═O)R^{A}, —OC(═O)NR^{A}R^{A}, —NR^{A}C(═O)OR^{A}, —NR^{A}C(═O)NR^{A}R^{A}, —SR^{A}, —S(═O)R^{A}, —S(═O)₂R^{A}, —S(═O)₂OR^{A}, —OS(═O)₂R^{A}, —S(═O)₂NR^{A}R^{A}, or —NR^{A}S(═O)₂R^{A}, wherein R^{A} is independently hydrogen or substituted or unsubstituted alkyl, substituted or unsubstituted alkenyl, substituted or unsubstituted alkynyl, substituted or unsubstituted carbocylyl, substituted or unsubstituted heterocylyl, substituted or unsubstituted aryl, substituted or unsubstituted heteroaryl, haloalkyl, an oxygen protecting group when attached to oxygen, a sulfur protecting group when attached to sulfur, or a nitrogen protecting group when attached to nitrogen, or two R^{A} groups can be taken together with the atoms to which they are attached to, to form a substituted or unsubstituted heterocylyl or heteroaryl ring.

2. The compound of claim 1, wherein the compound has the structure of Formula (I-A), Formula (I-B), Formula (II-A), or Formula (II-B): 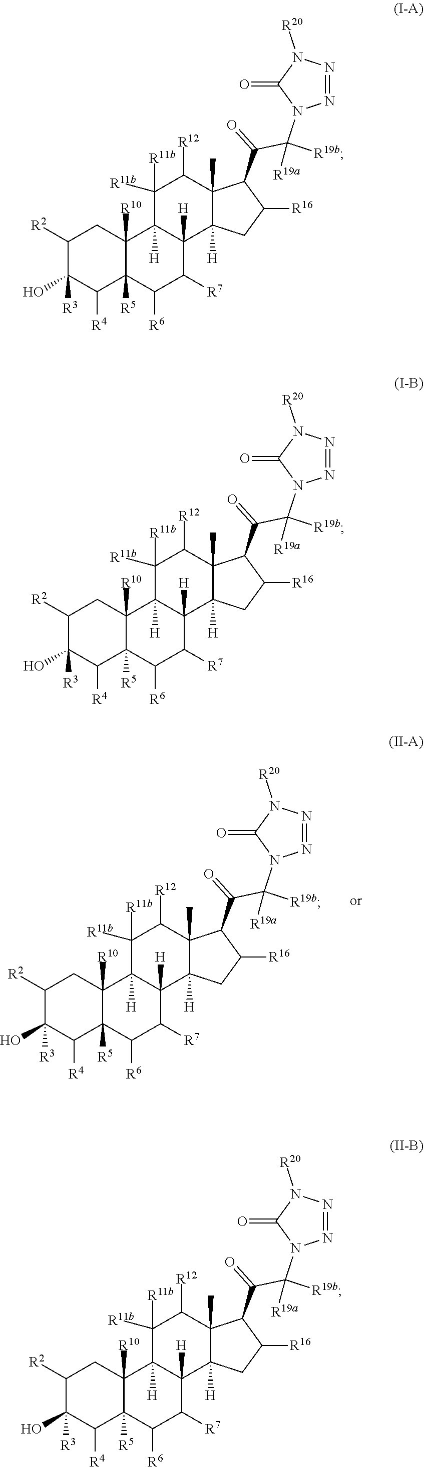
or a pharmaceutically acceptable salt, solvate, ester, or prodrug thereof.

3. The compound of claim 1 or 2, wherein the substituents are selected from fluoro, chloro, bromo, cyano, nitro, hydroxy, methoxy, ethoxy, propoxy, isopropoxy, methyl, ethyl, propyl, isopropyl, butyl, i-butyl, s-butyl, t-butyl, —CH₂CN, —CH₂CH₂CN, —CH₂F, —CHF₂, —CF₃, —CH₂OCH₃, —CH₂SCH₃, -methylhydroxy, morpholine, pyrrolidine, piperidine, piperazine, phenyl, benzyl, pyridine, pyrimidine, oxazole, pyrazole, cyclopropyl, cyclobutyl, cyclopentyl, cyclohexyl, —OCH₂F, —OCHF₂, —OCF₃, -ethylmethoxy, -methylcyclopropyl, —OR^{A}, —C(═O)R^{A}, —C(═O)OR^{A}, —OC(═O)R^{A}, —OC(═O)OR^{A}, —C(═O)NR^{A}R^{A}, —NR^{A}R^{A}, —NR^{A}C(═O)R^{A}, —OC(═O)NR^{A}R^{A}, —NR^{A}C(═O)OR^{A}, —NR^{A}C(═O)NR^{A}R^{A}, —SR^{A}, —S(═O)R^{A}, —S(═O)₂R^{A}, —S(═O)₂OR^{A}, —OS(═O)₂R^{A}, —S(═O)₂NR^{A}R^{A}, or —NR^{A}S(═O)₂R^{A}, wherein R^{A} is independently hydrogen, methyl, ethyl, propyl, isopropyl, cyclopropyl, cyclobutyl, cyclopentyl, cyclohexyl, —CH₂F, —CHF₂, —CF₃, phenyl, benzyl, pyridine, pyrimidine, -ethylmethoxy, or -methylcyclopropyl, or two R^{A} groups can be taken together with the atoms to which they are attached to, to form a heterocylyl or heteroaryl ring.

4. The compound of any one of claims 1-3, wherein R² is hydrogen, —OH, —OCH₃, —OCH₂CH₃, —OCH₂CH₂CH₃, —CH₃, —CH₂CH₃, —CH₂CH₂CH₃, substituted or unsubstituted cyclopropyl, fluoro, or chloro.

5. The compound of any one of claims 1-4, wherein R⁴ and R⁶ are hydrogen.

6. The compound of any one of claims 1-4, wherein at least one of R⁴ or R⁶ is hydrogen, substituted or unsubstituted —C₁₋₆ alkyl, halogen, —CH₃, or —CF₃.

7. The compound of any one of claims 1-6, wherein R⁷ is hydrogen, —CH₃, —CH₂CH₃, —CH(CH₃)₂, —OH, —OCH₃, or —CH₂OCH₃.

8. The compound of any one of claims 1-7, wherein R^{11a} and R^{11b} are both hydrogen.

9. The compound of any one of claims 1-7, wherein R^{11a} and R^{11b} together form ═O (oxo).

10. The compound of any one of claims 1-7, wherein R^{11a} is —OH, —OCH₃, —OCH₂CH₃ or —OCH₂CH₃CH₃ and R^{11b} is hydrogen.

11. The compound of any one of claims 1-10, wherein R¹² is hydrogen, —CH₃, —CH₂CH₃, —CH(CH₃)₂, —OH, —OCH₃, or —CH₂OCH₃.

12. The compound of any one of claims 1-11, wherein R¹⁶ is hydrogen, —CH₃, —CH₂CH₃, —CH(CH₃)₂, —OH, —OCH₃, or —CH₂OCH₃.

13. The compound of any one of claims 1-12, wherein R^{19a} and R^{19b} are both hydrogen; or R^{19a} is —C₁₋₆ alkyl and R^{19b} is hydrogen or —C₁₋₆ alkyl.

14. The compound of claim 1 or 2, wherein R², R⁴, R⁵, R⁶, R⁷, R¹⁰, R^{11a}, R^{11b}, R¹², R¹⁶, R^{19a} and R^{19b} are all hydrogen.

15. The compound of any one of claims 1-14, wherein R³ is hydrogen, substituted or unsubstituted —C₁₋₆ alkyl, —C₃₋₁₂ cycloalkyl, or C₃₋₁₂ cycloalkyl-C₁₋₆ alkyl-.

16. The compound of any one of claims 1-15, wherein R²⁰ is hydrogen, substituted or unsubstituted —C₁₋₆ alkyl, —C₂₋₆ alkenyl, —C₂₋₆ alkynyl, —C₁₋₆ haloalkyl, —C₃₋₁₂ cycloalkyl, C₃₋₁₂ cycloalkyl-C₁-C₆ alkyl-, heterocyclyl, —C₆-C₁₂ aryl, C₆-C₁₂ aryl-C₁-C₆ alkyl-, 5-12 membered heteroaryl, or 5-12 membered heteroaryl-C₁-C₆ alkyl-.

17. The compound of any one of claims 1-16, wherein R¹⁰ is hydrogen, halogen, cyano, —CH₃, —CH₂CH₃, —CH₂F, —CHF₂, —CF₃, —CH₂OCH₃, or —CH₂SCH₃.

18. The compound of claim 1, wherein the compound has the structure of Formula (I-E1) or (I-F1): 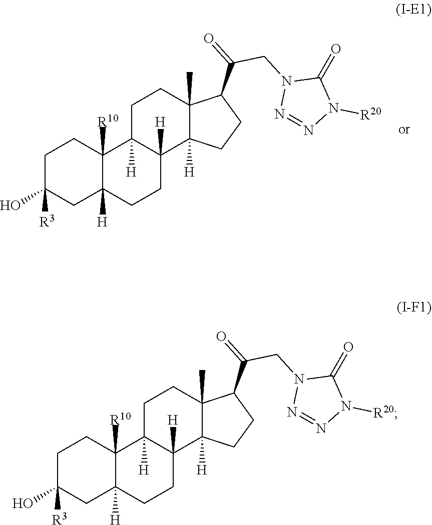
or a pharmaceutically acceptable salt, solvate, ester, or prodrug thereof, wherein:
R³ is hydrogen, —C₁₋₆ alkyl, —C₂₋₆ alkenyl, or —C₂₋₆ alkynyl, each optionally substituted with one to three groups selected from halogen or —C₁₋₆ alkoxy;
R¹⁰ is hydrogen, halogen, cyano, or —C₁₋₆ alkyl wherein —C₁₋₆ alkyl is optionally substituted with halogen or —C₁₋₃ alkoxy; and
R²⁰ is hydrogen, —C₁₋₆ alkyl, —C₂₋₆ alkenyl, —C₂₋₆ alkynyl, —C₁₋₆ haloalkyl, —C₃₋₁₂ cycloalkyl, C₃₋₁₂ cycloalkyl-C₁-C₆ alkyl-, 3-12 membered heterocyclyl, —C₆-C₁₂ aryl, C₆-C₁₂ aryl-C₁-C₆ alkyl-, heteroaryl-C₁-C₆ alkyl-, or 5-12 membered heteroaryl, each of which can be optionally substituted with halogen, nitro, cyano, —C₁₋₆ alkyl, —C₂₋₆ alkenyl, —C₂₋₆ alkynyl, —C₃₋₁₂ cycloalkyl, 3-12 membered heterocylyl, —C₆-C₁₂ aryl, 5-12 membered heteroaryl, —OR^{A}, —C(═O)R^{A}, —C(═O)OR^{A}, —OC(═O)R^{A}, —OC(═O)OR^{A}, —C(═O)NR^{A}R^{A}, —NR^{A}R^{A}, —NR^{A}C(═O)R^{A}, —OC(═O)NR^{A}R^{A}, —NR^{A}C(═O)OR^{A}, —NR^{A}C(═O)NR^{A}R^{A}, —SR^{A}, —S(═O)R^{A}, —S(═O)₂R^{A}, —S(═O)₂OR^{A}, —OS(═O)₂R^{A}, —S(═O)₂NR^{A}R^{A}, or —NR^{A}S(═O)₂R^{A};
wherein R^{A} is independently hydrogen, substituted or unsubstituted alkyl, haloalkyl, alkenyl, alkynyl, cycloalkyl, cycloalkylalkyl, carbocylyl, heterocylyl, aryl, C₆-C₁₂ aryl-C₁-C₆ alkyl-, heteroaryl, heteroaryl-C₁-C₆ alkyl-, an oxygen protecting group when attached to oxygen, a sulfur protecting group when attached to sulfur, or a nitrogen protecting group when attached to nitrogen; or two R^{A} groups can be taken together with the atoms to which they are attached to, to form a substituted or unsubstituted heterocylyl or heteroaryl ring;
wherein the substituents are selected from fluoro, chloro, bromo, cyano, nitro, hydroxy, methoxy, ethoxy, propoxy, isopropoxy, methyl, ethyl, propyl, isopropyl, butyl, i-butyl, s-butyl, t-butyl, —CH₂CN, —CH₂CH₂CN, —CH₂F, —CHF₂, —CF₃, —CH₂OCH₃, —CH₂SCH₃, -methylhydroxy, morpholine, pyrrolidine, piperidine, piperazine, phenyl, benzyl, pyridine, pyrimidine, oxazole, pyrazole, cyclopropyl, cyclobutyl, cyclopentyl, cyclohexyl, —OCH₂F, —OCHF₂, —OCF₃, -ethylmethoxy, -methylcyclopropyl, —OR^{A}, —C(═O)R^{A}, —C(═O)OR^{A}, —OC(═O)R^{A}, —OC(═O)OR^{A}, —C(═O)NR^{A}R^{A}, —NR^{A}R^{A}, —NR^{A}C(═O)R^{A}, —OC(═O)NR^{A}R^{A}, —NR^{A}C(═O)OR^{A}, —NR^{A}C(═O)NR^{A}R^{A}, —SR^{A}, —S(═O)R^{A}, —S(═O)₂R^{A}, —S(═O)₂OR^{A}, —OS(═O)₂R^{A}, —S(═O)₂NR^{A}R^{A}, or —NR^{A}S(═O)₂R^{A}, wherein R^{A} is independently hydrogen, methyl, ethyl, propyl, isopropyl, cyclopropyl, cyclobutyl, cyclopentyl, cyclohexyl, —CH₂F, —CHF₂, —CF₃, phenyl, benzyl, pyridine, pyrimidine, -ethylmethoxy, or -methylcyclopropyl, or two R^{A} groups can be taken together with the atoms to which they are attached to, to form a heterocylyl or heteroaryl ring.

19. The compound of claim 18, wherein R³ is —C₁₋₆ alkyl optionally substituted with one to three groups selected from halogen or —C₁₋₆ alkoxy.

20. The compound of claim 19, wherein R³ is —CH₃, —CH₂CH₃, —CH₂F, —CHF₂, —CF₃, —CH₂OCH₂CH₃, or —CH₂OCH₃.

21. The compound of claim 20, wherein R³ is —CH₃.

22. The compound of any one of claims 18-21, wherein R¹⁰ is hydrogen, halogen, cyano, —CH₃, —CH₂CH₃, —CH₂F, —CHF₂, —CF₃, —CH₂OCH₃, or —CH₂SCH₃.

23. The compound of claim 1, wherein the compound has the structure of Formula (I-G1) 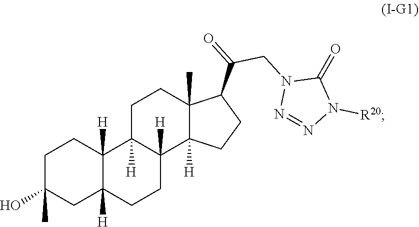
or a pharmaceutically acceptable salt, solvate, ester, or prodrug thereof, wherein:
R²⁰ is hydrogen, —C₁₋₆ alkyl, —C₃₋₁₂ cycloalkyl, C₃₋₁₂ cycloalkyl-C₁₋₆ alkyl-, —C₆-C₁₂ aryl, or 5-12 membered heteroaryl, each of which can be optionally substituted with halogen, nitro, cyano, —C₁₋₆ alkyl, —C₂₋₆ alkenyl, —C₂₋₆ alkynyl, —C₃₋₁₂ cycloalkyl, 3-12 membered heterocylyl, —C₆-C₁₂ aryl, 5-12 membered heteroaryl, —OR^{A}, —C(═O)R^{A}, —C(═O)OR^{A}, —OC(═O)R^{A}, —OC(═O)OR^{A}, —C(═O)NR^{A}R^{A}, —NR^{A}R^{A}, —NR^{A}C(═O)R^{A}, —OC(═O)NR^{A}R^{A}, —NR^{A}C(═O)OR^{A}, —NR^{A}C(═O)NR^{A}R^{A}, —SR^{A}, —S(═O)R^{A}, —S(═O)₂R^{A}, —S(═O)₂OR^{A}, —OS(═O)₂R^{A}, —S(═O)₂NR^{A}R^{A}, or —NR^{A}S(═O)₂R^{A};
wherein R^{A} is independently hydrogen, substituted or unsubstituted alkyl, haloalkyl, alkenyl, alkynyl, cycloalkyl, cycloalkylalkyl, carbocylyl, heterocylyl, aryl, C₆-C₁₂ aryl-C₁-C₆ alkyl-, heteroaryl, heteroaryl-C₁-C₆ alkyl-, an oxygen protecting group when attached to oxygen, a sulfur protecting group when attached to sulfur, or a nitrogen protecting group when attached to nitrogen; or two R^{A} groups can be taken together with the atoms to which they are attached to, to form a substituted or unsubstituted heterocylyl or heteroaryl ring;
wherein the substituents are selected from fluoro, chloro, bromo, cyano, nitro, hydroxy, methoxy, ethoxy, propoxy, isopropoxy, methyl, ethyl, propyl, isopropyl, butyl, i-butyl, s-butyl, t-butyl, —CH₂CN, —CH₂CH₂CN, —CH₂F, —CHF₂, —CF₃, —CH₂OCH₃, —CH₂SCH₃, -methylhydroxy, morpholine, pyrrolidine, piperidine, piperazine, phenyl, benzyl, pyridine, pyrimidine, oxazole, pyrazole, cyclopropyl, cyclobutyl, cyclopentyl, cyclohexyl, —OCH₂F, —OCHF₂, —OCF₃, -ethylmethoxy, -methylcyclopropyl, —OR^{A}, —C(═O)R^{A}, —C(═O)OR^{A}, —OC(═O)R^{A}, —OC(═O)OR^{A}, —C(═O)NR^{A}R^{A}, —NR^{A}R^{A}, —NR^{A}C(═O)R^{A}, —OC(═O)NR^{A}R^{A}, —NR^{A}C(═O)OR^{A}, —NR^{A}C(═O)NR^{A}R^{A}, —SR^{A}, —S(═O)R^{A}, —S(═O)₂R^{A}, —S(═O)₂OR^{A}, —OS(═O)₂R^{A}, —S(═O)₂NR^{A}R^{A}, or —NR^{A}S(═O)₂R^{A}, wherein R^{A} is independently hydrogen, methyl, ethyl, propyl, isopropyl, cyclopropyl, cyclobutyl, cyclopentyl, cyclohexyl, —CH₂F, —CHF₂, —CF₃, phenyl, benzyl, pyridine, pyrimidine, -ethylmethoxy, or -methylcyclopropyl, or two R^{A} groups can be taken together with the atoms to which they are attached to, to form a heterocylyl or heteroaryl ring.

24. The compound of any one of claims 18-23, wherein R²⁰ is hydrogen, —C₁-C₆ alkyl, —C₂₋₆ alkenyl, —C₂₋₆ alkynyl, —C₁₋₆ haloalkyl, —C₃₋₁₂ cycloalkyl, C₃₋₁₂ cycloalkyl-C₁-C₆ alkyl-, 3-12 membered heterocyclyl, —C₆-C₁₂ aryl, C₆-C₁₂ aryl-C₁-C₆, alkyl-, heteroaryl-C₁-C₆ alkyl-, or 5-12 membered heteroaryl, each of which can be optionally substituted with halogen, nitro, cyano, —C₁₋₆ alkyl, —C₂₋₆ alkenyl, —C₂₋₆ alkynyl, —C₃₋₁₂ cycloalkyl, 3-12 membered heterocylyl, —C₆-C₁₂ aryl, 5-12 membered heteroaryl, —OR^{A}, —C(═O)R^{A}, —C(═O)OR^{A}, —OC(═O)R^{A}, —OC(═O)OR^{A}, —C(═O)NR^{A}R^{A}, —NR^{A}R^{A}, —NR^{A}C(═O)R^{A}, —OC(═O)NR^{A}R^{A}, —NR^{A}C(═O)OR^{A}, —NR^{A}C(═O)NR^{A}R^{A}, —SR^{A}, —S(═O)R^{A}, —S(═O)₂R^{A}, —S(═O)₂OR^{A}, —OS(═O)₂R^{A}, —S(═O)₂NR^{A}R^{A}, or —NR^{A}S(═O)₂R^{A}.

25. The compound of any one of claims 18-24, wherein R²⁰ is hydrogen, —C₁₋₆ alkyl, —C₃₋₁₂ cycloalkyl, C₃₋₁₂ cycloalkyl-C₁-C₆ alkyl-, —C₆-C₁₂ aryl, or 5-12 membered heteroaryl, each of which are optionally substituted.

26. The compound of any one of claims 18-25, wherein R²⁰ is —CH₃, —CF₃, —CH₂CH₃, -i-Pr, -n-Pr, -i-Bu, -s-Bu, -t-Bu, —CH₂cyclopropyl, —CH₂CN, or —CH₂CH₂CN.

27. The compound of any one of claims 18-25, wherein R²⁰ is cyclopropyl, cyclobutyl, cyclopentyl, or cyclohexyl.

28. The compound of any one of claims 18-25, wherein R²⁰ is phenyl.

29. The compound of any one of claims 18-25, wherein R²⁰ is phenyl substituted with one or more halogen, cyano, or alkoxy.

30. The compound of any one of claims 18-25, wherein R²⁰ is pyridine.

31. The compound of any one of claims 18-25, wherein R²⁰ is pyridine substituted with one or more halogen, cyano, or alkoxy.

32. The compound of any one of claims 18-25, wherein R²⁰ is oxazole, pyrazole, or N-methylpyrazole.

33. The compound of claim 1, wherein the compound is: 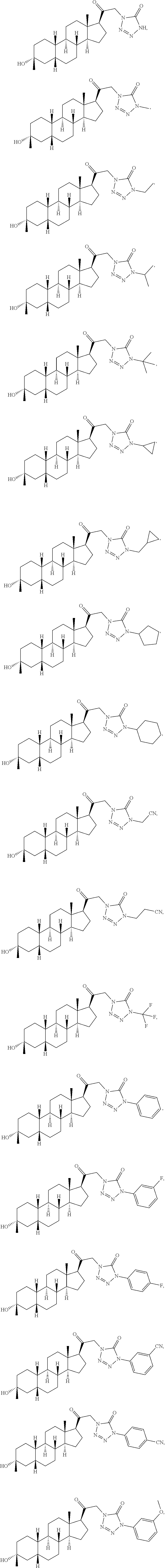 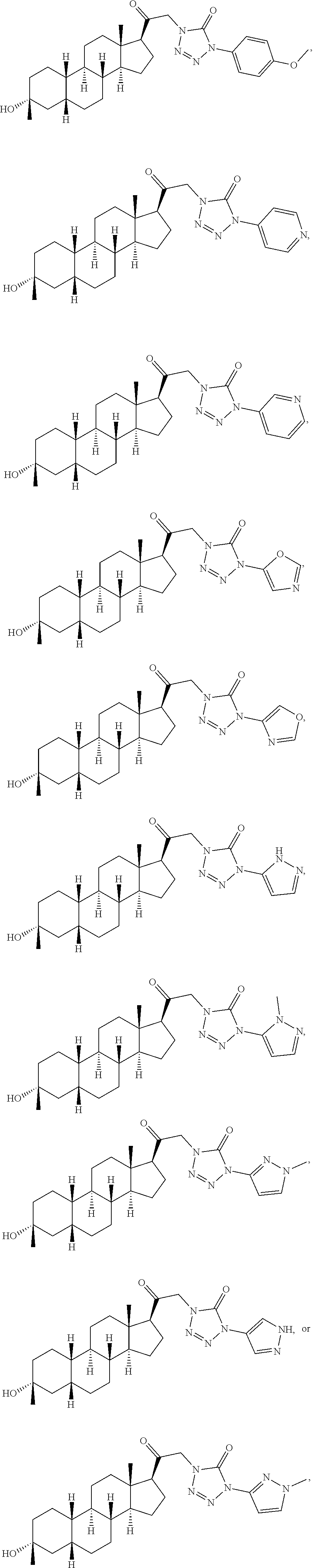
or a pharmaceutically acceptable salt, solvate, ester, or prodrug thereof.

34. A pharmaceutical composition comprising a compound according to any one of claims 1-33, or a pharmaceutically acceptable salt, a solvate, a stereoisomer, or tautomer thereof, and a pharmaceutically acceptable carrier.

35. The pharmaceutical composition of claim 34, further comprising at least one additional therapeutically active agent.

36. A method for treating a CNS-related disorder in a subject in need thereof, comprising administering to the subject an effective amount of a compound of any one of claims 1-33, or a pharmaceutically acceptable salt, a solvate, a stereoisomer, or tautomer thereof.

37. The method of claim 36, wherein the CNS-related disorder is a sleep disorder, a mood disorder, a schizophrenia spectrum disorder, a convulsive disorder, a disorder of memory and/or cognition, a movement disorder, a personality disorder, autism spectrum disorder, pain, traumatic brain injury, a vascular disease, a substance abuse disorder and/or withdrawal syndrome, or tinnitus

38. The method of claim 36, wherein the compound is administered orally, subcutaneously, intravenously, or intramuscularly.

39. The method of claim 36, wherein the compound is administered chronically.

40. A method for inducing sedation and/or anesthesia in a subject in need thereof, comprising administering to the subject an effective amount of a compound of any one of claims 1-33, or a pharmaceutically acceptable salt, a solvate, a stereoisomer, or tautomer thereof.
